(19) United States
(12) Patent Application Publication
(10) Pub. No.: US 2026/0234660 A1
(43) Pub. Date: Aug. 13, 2026

(54) TREATMENT OF MUSCLE RELATED DISORDERS WITH ANTI-HUMAN CACNG1 ANTIBODIES

(71) Applicant: Regeneron Pharmaceuticals, Inc., Tarrytown, NY (US)

(72) Inventors: Michael Stec, Tarrytown, NY (US); Evangelos Pefanis, Somers, NY (US); Christos Kyratsous, Irvington, NY (US); Andrew Baik, Scarsdale, NY (US); Leah Sabin, Goldens Bridge, NY (US); Sven Moller-Tank, White Plains, NY (US)

(21) Appl. No.: 19/155,618

(22) PCT Filed: Feb. 12, 2024
(86) PCT No.: PCT/US2024/015392

§ 371 Date: Aug. 12, 2025

Related U.S. Application Data
(60) Provisional application No. 63/484,675, filed on Feb. 13, 2023.
(60) Provisional application No. 63/494,119, filed on Apr. 4, 2023.
(60) Provisional application No. 63/583,724, filed on Sep. 19, 2023.

(51) Int. Cl. C12N 15/86; A61K 39/00; A61K 39/395; A61K 48/00; A61P 21/00; C07K 14/005; C07K 16/28

(52) U.S. Cl. CPC C12N 15/86; A61K 39/3955; A61K 48/005; A61P 21/00; C07K 14/005; C07K 16/28; A61K 2039/505; C07K 2317/565; C07K 2317/77; C07K 2317/92; C12N 2750/14122; C12N 2750/14143

(57) ABSTRACT

Adeno-associated virus (AAV) particles redirected with antibodies to CACNG1 and carrying a therapeutic nucleotide of interest are provided. Also provided are methods of making and using the AAV particles, e.g., for treating a patient in need thereof or the manufacture of a medicament for same.

### CROSS-REFERENCE TO RELATED APPLICATIONS

This application claims priority to U.S. Provisional Application No. 63/484,675, filed Feb. 13, 2023, U.S. Provisional Application No. 63/494,119, filed Apr. 4, 2023, and U.S. Provisional Application No. 63/583,724, filed Sep. 19, 2023, the disclosures of which are hereby incorporate by reference in their entirety.

### REFERENCE TO A SEQUENCE LISTING SUBMITTED AS AN XML FILE

A Sequence Listing in xml format entitled “11459WO01 Sequence Listing XML,” which was created Feb. 8, 2024, and is 280 Kb, is incorporated herein by reference in its entirety.

### TECHNICAL FIELD

This application is generally directed to human antibodies and antigen-binding fragments of human antibodies that bind human CACNG1 (hCACNG1), and methods of use thereof, e.g., in methods of treating a disorder in a patient in need thereof. The application also relates to antigen-binding molecules comprising at least an antigen-binding fragment of an anti-hCACNG1 antibody, wherein complexation of the antigen-binding molecule to CACNG1 mediates internalization of the antigen-binding molecule/CACNG1 complex. The application further relates to anti-hCACNG1 antibody (or antigen-binding molecules comprising an antigen-binding fragment of an anti-hCACNG1 antibody) conjugated viral vectors comprising a therapeutic nucleotide of interest, which conjugates may be useful in treating muscle related disorders.

### BACKGROUND

Skeletal muscle is the largest organ in the body, comprising ~40% of total body mass. Skeletal muscle is one of the three significant muscle tissues in the human body. Each skeletal muscle consists of thousands of muscle fibers wrapped together by connective tissue sheaths. The individual bundles of muscle fibers in a skeletal muscle are known as fasciculi. The outermost connective tissue sheath surrounding the entire muscle is known as epimysium. The connective tissue sheath covering each fasciculus is known as perimysium, and the innermost sheath surrounding individual muscle fiber is known as endomysium. Each muscle fiber is comprised of a number of myofibrils containing multiple myofilaments.

When bundled together, all the myofibrils get arranged in a unique striated pattern forming sarcomeres which are the fundamental contractile unit of a skeletal muscle. The two most significant myofilaments are actin and myosin filaments arranged distinctively to form various bands on the skeletal muscle.

The primary functions of the skeletal muscle take place via its intrinsic excitation-contraction coupling process. As the muscle is attached to the bone tendons, the contraction of the muscle leads to movement of that bone that allows for the performance of specific movements. The skeletal muscle also provides structural support and helps in maintaining the posture of the body. The skeletal muscle also acts as a storage source for amino acids that can be used by different organs of the body for synthesizing organ-specific proteins. The skeletal muscle also acts as a site of glucose disposal in the form of muscle glycogen. The skeletal muscle also plays a central role in maintaining thermostasis and acts as an energy source during starvation. Thus, skeletal muscle plays key roles in locomotion, thermoregulation, and in controlling whole body metabolism.

In many muscle diseases as well as during normal aging, the size and function of skeletal muscle tissue is reduced, resulting in impaired functional mobility; and in the case of severe muscle diseases, long-term disability and early mortality.

Treatments for muscle wasting and genetic muscle diseases typically consist of broad-acting therapies, such as testosterone therapy for muscle wasting, glucocorticoids for muscular dystrophies, etc. Untargeted delivery of these therapies reduces efficiency of specific muscle uptake, while also causing significant detrimental off-target effects on other organs.

There is a need in the art for new anti-human antibodies, capable of binding a muscle-specific marker and effecting the internalization by muscle cells of a therapeutic payload.

### SUMMARY

Described herein are viral vectors (e.g., adeno-associated viral (AAV) vectors) retargeted with antibodies and antigen-binding fragments thereof that bind to human CACNG1. The retargeted AAV vectors described herein may be useful, inter alia, for specifically directing the internalization of a nucleotide, e.g., encoding a therapeutic protein, to a skeletal muscle cell.

Viral particles as described herein are particularly suited for the targeted introduction of a nucleotide specifically to a muscle cell since the viral capsid or viral capsid protein(s) described herein comprise a targeting ligand that binds a muscle-cell specific surface protein. In some embodiments, a viral capsid or viral capsid protein comprises a first member of a binding pair, associated with its cognate second member of the binding pair, wherein the second member is linked (e.g., fused to) a targeting ligand that binds a muscle-cell specific surface protein. In some embodiments, the targeting ligand is operably linked to the second member, e.g., fused to the second member, optionally via a linker. In some embodiments, a targeting ligand may be a binding moiety, e.g., a natural ligand, antibody, a multispecific binding molecule, etc. In some embodiments, the targeting ligand is an antibody or portion thereof. In some embodiments, the targeting ligand is an antibody comprising a variable domain that binds a muscle-specific surface protein on a muscle cell and a heavy chain constant domain. In some embodiments, the targeting ligand is an antibody comprising a variable domain that binds a muscle-specific surface protein on a target cell and an IgG heavy chain constant domain. In some embodiments, the targeting ligand is an antibody comprising a variable domain that binds a muscle-specific surface protein on a target cell and an IgG heavy chain constant domain, wherein the IgG heavy chain constant domain is operably linked, e.g., via a linker, to a protein (e.g., second member of a protein:protein binding pair) that forms an isopeptide covalent bond with the first member. In some embodiments, a capsid protein described herein comprises a first member comprising SpyTag operably linked to the viral capsid protein, and covalently linked to the SpyTag, an second member comprising SpyCatcher linked to a targeting ligand comprising an antibody variable domain and an IgG heavy chain domain, wherein SpyCatcher and the IgG heavy chain domain are linked via an amino acid linker, e.g., GSGESG (SEQ ID NO:253). In some embodiments, the muscle-specific surfrase protein comprises CACNG1. In some embodiments, the targeting ligand binds CACNG1, e.g., human CACNG1. In some embodiments, the targeting ligand comprises a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 comprising an amino acid sequence of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 as set forth in any one of SEQ ID NOs:1-240.

In some embodiments, a viral particle as described herein, e.g., an AAV particle retargeted with an anti-CACNG1 antibody or fragment thereof as described herein, comprises a nucleotide, e.g., a nucleotide of interest. In some embodiments, the nucleotide of interest encodes a reporter gene. In some embodiments, the nucleotide of interest encodes microdystrophin, e.g., human microdystrophin, e.g., for use in methods of treating Duchenne muscular dystrophy or models thereof and/or for use in the manufacture of a medicament for treating Duchenne muscular dystrophy or models thereof. In some embodiments, the nucleotide of interest encodes Fukutin-related protein (FKRP), e.g., human FKRP, e.g., for use in methods of treating limb girdle muscular dystrophy or models thereof and/or for use in the manufacture of a medicament for treating limb girdle muscular dystrophy or models thereof. In some embodiments, the nucleotide of interest encodes myotubularin (MTM1), e.g., human MTM1, e.g., for use in methods of treating myotubular myopathy or models thereof and/or for use in the manufacture of a medicament for treating myotubular myopathy or models thereof.

An exemplary nucleotide molecule of interest as described herein may comprise a sequence set forth as SEQ ID NO:270, e.g., for use in methods of treating Duchenne muscular dystrophy or models thereof and/or for use in the manufacture of a medicament for treating Duchenne muscular dystrophy or models thereof. An exemplary nucleotide molecule of interest as described herein may comprise a sequence set forth as SEQ ID NO:271, e.g., for use in methods of treating limb girdle muscular dystrophy or models thereof and/or for use in the manufacture of a medicament for treating limb girdle muscular dystrophy or models thereof. An exemplary nucleotide molecule of interest as described herein may comprise a sequence set forth as SEQ ID NO:272, e.g., for use in methods of treating myotubular myopathy or models thereof and/or for use in the manufacture of a medicament for treating myotubular myopathy or models thereof.

### DRAWINGS

The patent or application file contains at least one drawing executed in color. Copies of this patent or patent application publication with color drawing(s) will be provided by the Office upon request and payment of the necessary fee.

FIGS. 1A-1D show in vitro and ex vivo evaluation of CACNG1 antibody properties. Mouse and human myotubes were used as an in vitro model of muscle to evaluate CACNG1 antibody cell binding (FIGS. 1A-1B), and internalization (FIG. 1C). Incubation of live myotubes with anti-CACNG1 antibodies followed by fluorophore-conjugated secondary detection was performed to assess antibody binding (FIGS. 1A-1B). Incubation of myotubes with anti-CACNG1 antibodies, e.g., REGN7854 and other anti-CACNG1 antibodies described herein, followed by duocarmycin-conjugated secondary (2° Ab-cytotoxic drug) was performed to assess antibody internalization via cell killing assay (FIG. 1C). Immunostaining with anti-CACNG1 antibodies in live CACNG1^{Hu/Hu} mouse single myofibers (FIG. 1D, left panel) and unfixed muscle tissue cross sections (FIG. 1D, right panel) show localization of CACNG1 at the myofiber cell surface.

FIG. 2 provides data regarding human myotube acetylcholine-induced calcium flux (relative light units; y-axis) after incubation with different concentrations (0.01 μM, 0.1 μM, 1 μM, and 1 μM; x-axis) of an anti-hCACNG1 antibody (REGN5972, REGN10728, or H2aM31944N), an isotype control antibody (REGN3892, REGN1945, or REGN1097), or with 20 μM nicardipine as a positive control for calcium blocking. The anti-hCACNG1 antibodies tested here do not inhibit acetylcholine-induced calcium flux in human myotubes at these concentrations.

FIG. 3 provides fluorescence immunohistochemistry images taken at 20× magnification of single myofibers ex vivo after isolation from wildtype (“WT”) mice, mice that were homozygous for the deletion of CACNG1 (“KO”), or mice expressing only human CACNG1 (“CACNGHu^{Hu/Hu}”); incubation with an anti-human CACNG1 antibody (H1M31941N or REGN5972), or an isotype control antibody (REGN653 or REGN1945); and labelling with fluorescent-conjugated secondary antibodies. CACNG1 antibodies bound to CACNG1^{Hu/Hu} myofibers, while isotype control antibodies did not.

FIG. 4 provides single plane confocal fluorescence immunohistochemistry images taken at 20× magnification of single myofibers ex vivo after isolation from wildtype (“WT”) mice, mice that were homozygous for the deletion of CACNG1 (“KO”), or mice expressing only human CACNG1 (“CACNG1^{Hu/Hu}”); and incubation with an anti-human CACNG1 antibody (REGN10728) or an isotype control antibody (REGN4439) conjugated with Alexa 647 (A647) fluorophore for 30 minutes, 4 hours or 8 hours. Confocal imaging revealed that fluorophore-conjugated CACNG1 antibody bound to the surface of CACNG1^{Hu/Hu} myofibers after 30 minutes of incubation, and that a portion of CACNG1 antibody was internalized and detected within the myofiber by 4 hours and 8 hours of incubation. Fluorophore-conjugated isotype control antibody was not detected to bind or internalize in CACNG1^{Hu/Hu} myofibers.

FIG. 5 shows the level of androgen receptor (AR) activation in terms of relative light units (RLU; y-axis) after a 24 hour incubation of an LNCaP cell line modified to express luciferase upon androgen receptor activation (AR.Luc) with: dihydrotestosterone (DHT) alone (M608; unconjugated DHT); an anti-hCACNG1 antibody (REGN14570, REGN14571, REGN14572, REGN14573, REGN14574 or REGN14647) conjugated via a VC-PAB linker to DHT (M3004); or an anti-FelD isotype control antibody (REGN3892) conjugated via a VC-PAB linker to DHT (M3004); at varying concentrations (Log[Conc. (M)]; x-axis). Only unconjugated DHT was shown to activate androgen receptor in this assay, while none of the CACNG1 antibodies conjugated to DHT showed any appreciable activation of the androgen receptor in this cell line that does not express hCACNG1.

FIGS. 6A-6I show the level of androgen receptor (AR) activation in terms of relative light units (RLU; y-axis) after a 24 hour (FIGS. 6A-6C), 48 hour (FIGS. 6D-6F), or 72 hour (FIGS. 6G-6I) incubation of a hCACNG1-expressing LNCaP cell line modified to also express luciferase upon androgen receptor activation (hCACNG1.AR.Luc) with: dihydrotestosterone (DHT) alone (M608; unconjugated DHT); an anti-hCACNG1 antibody (REGN14570, REGN14571, REGN14572, REGN14573, REGN14574 or REGN14647) conjugated via a VC-PAB linker to DHT (M3004); or an anti-FelD isotype control antibody (REGN3892) conjugated via a VC-PAB linker to DHT (M3004); at varying concentrations (Log[Conc. (M)]; x-axis). Several CACNG1 antibody-DHT conjugates activated androgen receptor in this hCACNG1 expressing cell line, and while the efficacy and potency of androgen receptor activation was lower than that of unconjugated DHT at 24 hours following treatment, activation of the androgen receptor was sustained at 48 and 72 hours compared to unconjugated DHT.

FIG. 7 provides cryo-fluorescence tomography images of a mouse 6 days after systemic injection of 10 mg/kg of an anti-hCACNG1 antibody conjugated to Alexa 647 (REGN10728 or REGN5972) or an isotype control antibody (REGN4439) conjugated to Alexa 647.

FIGS. 8A-8G provide tiled fluorescence immunohistochemistry images taken at 20× magnification of images of gastrocnemius/plantaris/soleus (FIG. 8A), tibialis anterior (FIG. 8B), diaphragm (FIG. 8C), tongue (FIG. 8D), triceps (FIG. 8E), trapezius (FIG. 8F), or pelvic floor muscle (FIG. 8G) sections of mice expressing only human CACNG1 (“CACNG1^{Hu/Hu}”) after tail vein injection with 10 mg/kg of an anti-human CACNG1 antibody (REGN5972 or REGN10728) or an isotype control antibody (REGN4439) conjugated with Alexa 647 (A647) fluorophore and sacrificed 6 days post injection. Fluorophore-conjugated CACNG1 antibody was detected in all of these skeletal muscles, with REGN10728 displaying a stronger signal in muscles compared to REGN5972. Only low levels of fluorescence were detected in muscles from isotype control and saline injected mice.

FIGS. 9A-9D provide tiled fluorescence immunohistochemistry images taken at 20× magnification of images of liver (FIG. 9A), kidney (FIG. 9B), spleen (FIG. 9C), or brown adipose tissue (FIG. 9D) sections of mice expressing only human CACNG1 (“CACNG1^{Hu/Hu}”) after tail vein injection with 10 mg/kg an anti-human CACNG1 antibody (REGN5972 or REGN10728) or an isotype control antibody (REGN4439) conjugated with Alexa 647 (A647) fluorophore and sacrificed 6 days post injection. Neither fluorophore-conjugated CACNG1 antibody showed appreciable signal in these organs, with Alexa 647 levels similar to isotype and saline injected controls.

FIG. 10 provides a schematic depicting an exemplary experimental timeline (top panel) and photomicrographs showing CACNG1 antibody distribution to the soleus muscle under sedentary and exercise conditions at either a 10 mg/kg or a 50 mg/kg (high) dose (bottom panel). CACNG1 distribution is altered by exercise and dose.

FIG. 11 provides cryo-fluorescence tomography images of a mouse model for Duchenne muscular dystrophy (D2-mdx), limb girdle muscular dystrophy (Fkrp^{P448I}), or myotubular myopathy (MTM1 KO) sacrificed 2 weeks after systemic injection of 5×10¹² viral genome/kg of wildtype AAV9 particles or AAV9 particles containing a W503A mutation retargeted with an anti-hCACNG1 antibody (REGN10717), expressing eGFP under the control of the CAG promoter.

FIG. 12A provides a schematic for the treatment of a D2-mdx mouse with an AAV expressing a nucleotide of interest encoding microdystrophin (μDys), under the control of a CK8 promoter. FIG. 12B provides the levels of μDys mRNA expressed in the quadriceps, gastrocnemius, diaphragm, and liver of D2-mdx mice injected with phosphate buffered saline (PBS), wildtype (WT) AAV9 particles comprising a nucleotide of interest encoding μDys, or AAV9 particles with an N272A mutation retargeted with an anti-hCACNG1 antibody (REGN10717) comprising a nucleotide of interest encoding μDys (y-axis; compared to levels from mice injected with WT AAV9). The left panel of FIG. 12C provides (i) Western Blots detecting μDys or β-actin from quadriceps of D2-mdx mice injected with phosphate buffered saline (PBS), wildtype (WT) AAV9 particles comprising a nucleotide of interest encoding μDys, or AAV9 particles with an N272A mutation retargeted with an anti-hCACNG1 antibody (REGN10717) comprising a nucleotide of interest encoding μDys, and (ii) a graph providing the abundance levels of the proteins is also provided. The right panel of FIG. 12C provides immunohistochemistry images taken of gastrocnemius muscle from untreated wildtype (WT) or D2-mdx mice, or after injection with wildtype (WT) AAV9 particles comprising a nucleotide of interest encoding μDys, or AAV9 particles with an N272A mutation retargeted with an anti-hCACNG1 antibody (REGN10717) comprising a nucleotide of interest encoding μDys and after staining for dystrophin. The left panel of FIG. 12D provides the percent change in serum creatine kinase (CK) 4 weeks after injection of D2-mdx mice with phosphate buffered saline (PBS), wildtype (WT) AAV9 particles comprising a nucleotide of interest encoding μDys, or AAV9 particles with an N272A mutation retargeted with an anti-hCACNG1 antibody (REGN10717) comprising a nucleotide of interest encoding μDys (y-axis; compared to levels at baseline, prior to injection), and the right panel of FIG. 12D provides the maximum grip strength (grams; y-axis) 12 weeks after injection of D2-mdx mice with phosphate buffered saline (PBS), wildtype (WT) AAV9 particles comprising a nucleotide of interest encoding μDys, or AAV9 particles with an N272A mutation retargeted with an anti-hCACNG1 antibody (REGN10717) comprising a nucleotide of interest encoding μDys.

FIG. 13A provides a schematic for the treatment of a Fkrp^{P448L} mouse with an AAV expressing a nucleotide of interest encoding human FKRP (hFKRP) under the control of a CK7 promoter. FIG. 13B provides the levels of hFKRP mRNA expressed in the quadriceps, gastrocnemius, diaphragm, and liver of Fkrp^{P448L} mice injected with phosphate buffered saline (PBS), wildtype (WT) AAV9 particles comprising a nucleotide of interest encoding hFKRP, or AAV9 particles with an N272A mutation retargeted with an anti-hCACNG1 antibody (REGN10717) comprising a nucleotide of interest encoding hFKRP (y-axis; compared to levels from mice injected with WT AAV9). The left panel of FIG. 13C provides immunohistochemistry images taken of a diaphragm from untreated wildtype (WT) or Fkrp^{P448L} mice, or after injection with phosphate buffered saline (PBS), wildtype (WT) AAV9 particles comprising a nucleotide of interest encoding hFKRP, or AAV9 particles with an N272A mutation retargeted with an anti-hCACNG1 antibody (REGN10717) comprising a nucleotide of Interest encoding hFKRP, and after incubation with IIH6 (that stains glycosylated α-dystroglycan), Laminin, and DAPI, and the right panel of FIG. 13C provides the intensity of IIH6 (y-axis) in arbitrary units (top graph) or as a percentage of area within laminin area (bottom graph) of these animals. FIG. 13D provides the maximal treadmill distance (meters; y-axis) run by Fkrp^{P448L} mice seven weeks after injection with phosphate buffered saline (PBS), wildtype (WT) AAV9 particles comprising a nucleotide of interest encoding hFKRP, or AAV9 particles with an N272A mutation retargeted with an anti-hCACNG1 antibody (REGN10717) comprising a nucleotide of interest encoding hFKRP.

FIG. 14A provides a schematic for the treatment of an MTM1 knockout (KO) mouse with an AAV expressing a nucleotide of interest encoding human MTM1 (hMTM1) under the control of a desmin promoter. FIG. 14B provides the levels of hMTM1 mRNA expressed in the quadriceps, gastrocnemius, diaphragm, and liver of MTM1 KO mice injected with phosphate buffered saline (PBS), wildtype (WT) AAV9 particles comprising a nucleotide of interest encoding hMTM1, or AAV9 particles with an N272A mutation retargeted with an anti-hCACNG1 antibody (REGN10717) comprising a nucleotide of interest encoding hMTM1 (y-axis; compared to levels from mice injected with WT AAV9). The left panel of FIG. 14C provides immunohistochemistry images taken of a soleus from untreated wildtype (WT) or MTM1 KO mice, or after injection with wildtype (WT) AAV9 particles comprising a nucleotide of interest encoding hMTM1, or AAV9 particles with an N272A mutation retargeted with an anti-hCACNG1 antibody (REGN10717) comprising a nucleotide of interest encoding hMTM1 and after incubation with Laminin, and DAPI; and the right panel of FIG. 14C provides the percentage of MTM1 KO mice that survive (up to 60 days) when injected at day 32 with PBS, wildtype (WT) AAV9 particles comprising a nucleotide of interest encoding hMTM1, or AAV9 particles with an N272A mutation retargeted with an anti-hCACNG1 antibody (REGN10717) comprising a nucleotide of interest encoding hMTM1.

FIG. 15 provides images of heart GFP expression in Fkrp^{P488L} mice after injection with wildtype AAV9 particles or AAV9 particles with an N272A mutation retargeted with an anti-hCACNG1 antibody (REGN10717) expressing an eGFP nucleotide of interest driven by a CAG promoter (left panel); and the levels of hFKRP mRNA in the heart of Fkrp^{P488L} mice after injection with PBS, wildtype AAV9 particles comprising a nucleotide of interest encoding hFKRP under the control of a CK7 promoter, or AAV9 particles with an N272A mutation retargeted with an anti-hCACNG1 antibody (REGN10717) and comprising a nucleotide of interest encoding hFKRP (right panel).

FIG. 16 provides the rationale and study protocols for determining whether heart transduction by AAV9 particles may be retained with robust skeletal muscle retargeting by conjugating CACNG1 antibodies to non-detargeted AAV9 capsids.

FIGS. 17A-17C provide images of the liver, quadriceps, or heart of C57BL/6 healthy mice (FIG. 17A) or D2-mdx mice (FIGS. 17B-17C) after injection with: wildtype AAV9 particles encapsulating a nucleotide of interest encoding eGFP under the control of a CAG promoter; AAV9 particles retargeted with an anti-hCACNG1 antibody (REGN10717), comprising a detargeting mutation (e.g., W503A), and encapsulating a nucleotide of interest encoding eGFP under the control of a CAG promoter; or WT AAV9 particles (without detargeting mutations) retargeted with an anti-hCACNG1 antibody (REGN10717) and encapsulating a nucleotide of interest encoding eGFP under the control of a CAG promoter, at differing doses of 2×10¹² vg/mouse (high), 4×10¹¹ vg/mouse (mid), or 8×10¹⁰ vg/mouse (low). FIG. 17C depicts the same tissues as FIG. 17B but at a higher magnification.

FIGS. 18A-18B show the level (y-axis) of GFP mRNA expression, relative to a housekeeping gene, Rplp0, in the liver, heart, or quadriceps of C57BL/6 healthy mice (FIG. 18A) or D2-mdx mice (FIG. 18B) after injection with 2×10¹² vg/mouse (high), 4×10¹¹ vg/mouse (mid), or 8×10¹⁰ vg/mouse (low) of: wildtype AAV9 particles encapsulating a nucleotide of interest encoding eGFP under the control of a CAG promoter (AAV WT); WT AAV9 particles (without detargeting mutations) retargeted with anti-hCACNG1 antibodies and encapsulating a nucleotide of interest encoding eGFP under the control of a CAG promoter (AAV WT+anti-CACNG1); AAV9 particles retargeted with an anti-hCACNG1 antibody (REGN10717), comprising a detargeting mutation (e.g., W503A), and encapsulating a nucleotide of interest encoding eGFP under the control of a CAG promoter (AAV W503A+anti-CACNG1); or phosphate buffered saline (PBS) (x-axis).

FIGS. 19A-19B show the level (y-axis) of GFP mRNA expression, relative to a housekeeping gene, Rplp0, in the gastrocnemius muscle, quadriceps, diaphragm, soleus, tibialis anterior, or tongue of C57BL/6 healthy mice (FIG. 19A) or D2-mdx mice (FIG. 19B) after injection with 2×10¹² vg/mouse (high), 4×10¹ vg/mouse (mid), or 8×10¹⁰ vg/mouse (low) of: wildtype AAV9 particles encapsulating a nucleotide of interest encoding eGFP under the control of a CAG promoter (AAV WT), WT AAV9 particles (without detargeting mutations) retargeted with anti-hCACNG1 antibodies and encapsulating a nucleotide of interest encoding eGFP under the control of a CAG promoter (AAV WT+anti-CACNG1); AAV9 particles retargeted with an anti-hCACNG1 antibody (REGN10717), comprising a detargeting mutation (e.g., W503A), and encapsulating a nucleotide of interest encoding eGFP under the control of a CAG promoter (AAV W503A+anti-CACNG1); or phosphate buffered saline (PBS) (x-axis).

FIG. 20 provides an illustrative schematic related to the refinement of detargeting and retargeting of AAV9 viral particles by manipulating the AAV capsid, the retargeting antibody, or both. Such modular design provides flexibility to dial in the degree of detargeting, and the addition of an antibody directs viral particles to novel tissues and cell types, which may be fine-tuned for the treatment of specific diseases.

FIG. 21A provides an illustrative schematic (not to scale) of the single stranded (ss) viral genome comprising from 5′ to 3′: a 141 base pair inverted terminal repeat (ITR), a CAGG promoter, a sequence encoding enhanced green fluorescent protein (GFP), Woodchuck Hepatitis Virus Posttranscriptional Regulatory Element (WPRE), a 32 base pair barcode, human (h) growth hormone (GH) poly A tail, and the 141 base pair ITR. FIG. 21B provides bar graphs that demonstrate enhanced transduction to various muscles in vivo in non-human primates (cynomolgus monkey) after administration of AAV9 viral particles (containing capsid mutations e.g., N272A, W503A, etc.) comprising the viral genome depicted in FIG. 21A, each with a unique barcode, and retargeted with anti-CACNG1 antibodies, compared to wildtype AAV9 viral particles (AAV9) comprising the viral genome depicted in FIG. 21A. Each candidate AAV was packaged with a unique barcoded genome as described in FIG. 21A. Following IV dosing of the 12-candidate barcoded pool, the indicated tissues were collected and relative abundance of each barcode in the total RNA purified from each tissue was assessed using next generation sequencing (NGS). Shown are the percentage of NGS reads (Y-axis) mapped to each barcode and associated capsid in the several tissues (x-axis): liver (left lateral lobe) and a subset of skeletal muscles (diaphragm, biceps brachii, bicep femoris, extensor digitorum longus (EDL), gastrocnemius, intercostals, soleus, tibialis anterior, transverse abdominus, triceps, vastus lateralis, psoas, tongue); normalized to the injected virus pool. The data represented here is the mean of two animals in the study. FIG. 21C shows that systemically delivered detargeted AAV9 conjugated to anti-CACNG1 demonstrates antibody-dependent transduction of skeletal muscles in non-human primates. Here, data from FIG. 21B are plotted as relative expression of mRNA compared to wildtype AAV9 expression (y-axis), depicting enhanced transduction of the diaphragm, psoas, triceps, and intercostals muscles with anti-CACNG1 antibodies #3 and #5.

FIGS. 22A-22C show serum levels of liver enzymes (ALT; FIG. 22A) and complement pathway biomarkers (sC5b-9; FIG. 22B), and markers of thrombotic microangiopathy (platelet counts; FIG. 22C) in non-human primates (cynomolgus monkey) that were either seropositive (sero(+)) or seronegative (sero(−)) to AAV9 at the indicated timepoints (x-axis) following injection of 2×10¹⁴ vg/kg of wildtype AAV9 or AAV9 W503A expressing eGFP under the control of a CAG promoter. Administration of wildtype AAV9 particles resulted in an elevation of ALT (FIG. 22A), an elevation of sC5b-9, a marker for complement terminal membrane attack complex (FIG. 22B), and lowered platelet counts (FIG. 22C), as expected, while administration of AAV9 W503A particles exhibited ALT levels (FIG. 22A), sC5b-9 levels (FIG. 22B), and platelet counts (FIG. 22C) similar to cynomolgus monkeys receiving saline only (negative control). These data suggest that liver-detargeted AAV9 W503A particles provide a safety advantage over liver-tropic wildtype AAV serotypes.

FIGS. 23A-23C show the extent of thrombocytopenia (FIG. 23A; platelet counts), hemolytic anemia (FIG. 23B; red cell distribution width), and impaired kidney filtration (FIG. 23C; serum creatinine) as markers of the thrombotic microangiopathy (TMA) triad in non-human primates (cynomolgus monkey) that were either seropositive (sero(+)) or seronegative (sero(−)) to AAV9 at the indicated timepoints (x-axis) following injection of wildtype AAV9 or AAV9 W503A expressing eGFP under the control of a CAG promoter. Decreases in platelet count are indicative of transient thrombocytopenia, increases in red cell distribution width are a marker of schistocytes that are indicative of mild, transient hemolytic anemia, and elevated serum creatinine levels is a marker of impaired kidney filtration that is indicative of mild, transient acute kidney injury. Wildtype AAV9 dosed monkeys display some symptoms of the TMA triad, but AAV9 W503A dosed monkeys do not.

FIGS. 24A-24B show line (FIG. 24A) and bar (FIG. 24B) graphs depicting serum creatine kinase levels for wildtype mice treated with PBS (50500 Vehicle), and mice comprising a P448L point mutation in the fukutin-related protein (FKRP) as a model of limb-girdle muscular dystrophy type 2I (FKRP^{P448L/P448L}) humanized laminin subunit α2 (LAMA2; Lama2^{HU/HU}) and humanized dystroglycan 1 (DAG1; DAG1^{HU/HU}) treated with PBS and pluronic acid (Vehicle) or varying doses (4E12 vg/kg, 1E13 vg/kg and 5E13 vg/kg) of AAV9 particles comprising an N272A mutation retargeted with an anti-hCACNG1 antibody (REGN10717) encapsulating a nucleotide of interest encoding human FKRP (hFKRP) under the control of a CK7 promoter.

### DESCRIPTION

Provided herein are novel anti-human CACNG1 antibodies, and monovalent antigen binding fragments thereof, which are useful in mediating internalization of CACNG1. The anti-human CACNG1 antibodies, and monovalent antigen binding fragments thereof may be useful, e.g., in the treatment of diseases, as part of multispecific antigen binding protein and/or multidomain therapeutic protein, and/or as an antibody drug conjugate.

The description herein is not limited to particular embodiments, compositions, methods and experimental conditions described, as such embodiments, compositions, methods and conditions may vary. The terminology used herein is for the purpose of describing particular embodiments only, and is not intended to be limiting.

Although any methods and materials similar or equivalent to those described herein can be used in the practice or testing as described herein, some preferred methods and materials are now described. All publications cited herein are incorporated herein by reference to describe in their entirety. Unless defined otherwise, all technical and scientific terms used herein have the same meaning as commonly understood by one of ordinary skill in the art.

The term “about,” when used in reference to a particular recited numerical value, means that the value may vary from the recited value by no more than 1%. For example, the expression “about 100” includes 99 and 101 and all values in between (e.g., 99.1, 99.2, 99.3, 99.4, etc.).

Voltage-dependent calcium channels are generally composed of five subunits. The protein encoded by the CACNG1 gene represents one of these subunits. “CACNG1” includes a protein encoded by the CACNG1 gene, and is one of two known gamma subunit proteins. CACNG1 is part of the skeletal muscle 1,4-dihydropyridine-sensitive calcium channel and is an integral membrane protein that plays a role in excitation-contraction coupling. CACNG1 is part of a functionally diverse eight-member protein subfamily of the PMP-22/EMP/MP20 family and is located in a cluster with two family members that function as transmembrane AMPA receptor regulatory proteins (TARPs). CACNG1 is highly and specifically expressed in skeletal muscle. The gene encoding human CACNG1 (CACNG1) is located on the long arm of chromosome 17. CACNG1 comprises 4 exons and is approximately 12,244 bases long. An exemplary sequence for human CACNG1 gene is assigned NCBI Accession Number NM_0007582.2 (SEQ ID NO:241). An exemplary human CACNG1 protein is assigned UniProt Accession No. 070578 (SEQ ID NO:242).

The phrase “an antibody that binds CACNG1” or an “anti-hCACNG1 antibody” includes an antibody and antigen-binding fragment thereof that specifically recognizes a single CACNG1 molecule. An antibody and antigen-binding fragment thereof as described herein may bind soluble CACNG1 and/or cell surface expressed CACNG1. Soluble CACNG1 includes natural CACNG1 proteins as well as recombinant CACNG1 protein variants that lack a transmembrane domain or are otherwise unassociated with a cell membrane.

The expression “cell surface-expressed CACNG1” refers to one or more CACNG1 protein(s) that is/are expressed on the surface of a cell in vitro or in vivo, such that at least a portion of a CACNG1 protein is exposed to the extracellular side of the cell membrane and is accessible to an antigen-binding portion of an antibody. A “cell surface-expressed CACNG1” can comprise or consist of a CACNG1 protein expressed on the surface of a cell which normally expresses CACNG1 protein. Alternatively, “cell surface-expressed CACNG1” can comprise or consist of a CACNG1 protein expressed on the surface of a cell that normally does not express human CACNG1 on its surface but has been artificially engineered to express CACNG1 on its surface.

The term “antigen-binding molecule” includes an antibody and an antigen-binding fragment of an antibody.

The term “antibody” refers to any antigen-binding molecule or molecular complex comprising at least one complementarity determining region (CDR) that specifically binds to or interacts with a particular antigen (e.g., CACNG1). The term “antibody”, as used herein, includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. The term “high affinity” antibody refers to those antibodies having a binding affinity to their target of at least 10⁻⁹ M, at least 10⁻¹⁰ M; at least 10⁻¹¹ M; or at least 10⁻¹² M, as measured by surface plasmon resonance, e.g., BIACORE™ or solution-affinity ELISA. The term “antibody” may encompass any type of antibody, such as e.g., monoclonal or polyclonal. Moreover, the antibody may be or any origin, such as e.g., mammalian or non-mammalian. In one embodiment, the antibody may be mammalian or avian. In a further embodiment, the antibody may be of human origin and may further be a human monoclonal antibody.

The term “antibody” also includes antigen-binding fragments of full antibody molecules. The terms “antigen-binding portion” of an antibody, “antigen-binding fragment” of an antibody, and the like include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment”.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody as described herein include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; (vii) V_{H}-C_{L}; (viii) VL-C_{H}1; (ix) VL-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody as described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody as described herein using routine techniques available in the art.

In certain embodiments, the anti-hCACNG1 antibodies as described herein are human antibodies. The term “human antibody” refers to antibodies having variable and constant regions derived from human germline immunoglobulin sequences. The human antibodies as described herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” is not intended to include antibodies in which CDR sequences derived from the germline of another mammalian species, such as a mouse, have been grafted onto human framework sequences.

The antibodies as described herein may, in some embodiments, be recombinant human antibodies. The term “recombinant human antibody” is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies may exist in two general forms that are associated with hinge heterogeneity. In one general form, an immunoglobulin molecule comprises a stable four chain construct of approximately 150-160 kDa in which the dimers are held together by an interchain heavy chain disulfide bond. In a second general form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The antibodies as described herein may have one or more mutations in the hinge, C_{H}2 or C_{H}3 region which may be desirable, for example, in production, to improve the yield of the desired antibody form.

The antibodies as described herein may be isolated antibodies. An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, may be considered an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

Also described herein are one-arm antibodies that bind CACNG1. The term “one-arm antibody” refers to an antigen-binding molecule comprising a single antibody heavy chain and a single antibody light chain. The one-arm antibodies as described herein may comprise any of the HCVR/LCVR or CDR amino acid sequences as set forth in Table 1.

The anti-hCACNG1 antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Also described herein are antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies as described herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, an antibody and an antigen-binding fragment that contains one or more germline mutations can be easily tested for one or more desired property such as, improved binding specificity, increased binding affinity, improved or enhanced antagonistic or agonistic biological properties (as the case may be), reduced immunogenicity, etc. In some embodiments, an antibody or an antigen-binding fragment as described herein is obtained in this general manner.

Also described herein are anti-hCACNG1 antibodies comprising variants of any of the HCVR, LCVR, and/or CDR amino acid sequences disclosed herein having one or more conservative substitutions. For example, some embodiments include anti-hCACNG1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein.

A “biologically equivalent portion,” “biologically equivalent variant”, or the like, of a reference nucleic acid sequence or polypeptide sequence disclosed herein includes those sequences that exhibits a similar biological activity as the reference nucleic acid sequence or reference polypeptide sequence. A biologically equivalent portion or variant of a reference nucleic acid sequence includes a shorter nucleic acid than that of the reference nucleic acid which encodes either a polypeptide that is identical to that encoded by the reference nucleic acid sequence or a polypeptide that exhibits the same biological activity as a polypeptide encoded by the reference nucleic acid. The term “portion” refers to at least 5 amino acids or at least 15 nucleotides, but less than the full-length polypeptide or nucleic acid molecule, with at least 75%, 80%, 85%, 90%, 95%, 99%, or 100% identity to a sequence from which the portion is derived. A “portion” encompasses any contiguous segment of amino acids or nucleotides sufficient to determine the reference polypeptide or nucleic acid molecule from which the portion is derived. In some embodiments, a portion comprises at least 5 amino acids or 15 nucleotides with at least 75%, 80%, 85%, 90%, 95%, 99%, or 100% identity to a reference polypeptide or nucleic acid sequence. In some embodiments, a portion comprises at least 10 amino acids or 30 nucleotides with at least 75%, 80%, 85%, 90%, 95%, 99%, or 100% identity to a reference polypeptide or nucleic acid sequence. In some embodiments, a portion comprises at least 15 amino acids or 45 nucleotides with at least 75%, 80%, 85%, 90%, 95%, 99%, or 100% identity to a reference polypeptide or nucleic acid sequence. In some embodiments, a portion comprises at least 20 amino acids or 60 nucleotides with 100% to a reference polypeptide or nucleic acid sequence. In some embodiments, a portion comprises at least 25 amino acids or 75 nucleotides with at least 75%, 80%, 85%, 90%, 95%, 99%, or 100% identity to a reference polypeptide or nucleic acid sequence. In some embodiments, a portion comprises at least 30 amino acids or 90 nucleotides with at least 75%, 80%, 85%, 90%, 95%, 99%, or 100% identity to a reference polypeptide or nucleic acid sequence. In some embodiments, a portion comprises at least 35 amino acids or 105 nucleotides with at least 75%, 80%, 85%, 90%, 95%, 99%, or 100% identity to a reference polypeptide or nucleic acid sequence. In some embodiments, a portion comprises at least 40 amino acids or 120 nucleotides with at least 75%, 80%, 85%, 90%, 95%, 99%, or 100% identity to a reference polypeptide or nucleic acid sequence. In some embodiments, a portion comprises at least 45 amino acids or 135 nucleotides with at least 75%, 80%, 85%, 90%, 95%, 99%, or 100% identity to a reference polypeptide or nucleic acid sequence. In some embodiments, a portion comprises at least 50 amino acids or 150 nucleotides with at least 75%, 80%, 85%, 90%, 95%, 99%, or 100% identity to a reference polypeptide or nucleic acid sequence. In some embodiments, a portion comprises at least 60 amino acids or 180 nucleotides with at least 75%, 80%, 85%, 90%, 95%, 99%, or 100% identity to a reference polypeptide or nucleic acid sequence. In some embodiments, a portion comprises at least 70 amino acids or 210 nucleotides with at least 75%, 80%, 85%, 90%, 95%, 99%, or 100% identity to a reference polypeptide or nucleic acid sequence. In some embodiments, a portion comprises at least 80 amino acids or 240 nucleotides with at least 75%, 80%, 85%, 90%, 95%, 99%, or 100% identity to a reference polypeptide or nucleic acid sequence. In some embodiments, a portion comprises at least 100 amino acids or 300 nucleotides with at least 75%, 80%, 85%, 90%, 95%, 99%, or 100% identity to a reference polypeptide or nucleic acid sequence.

In a non-limiting example, a biologically equivalent variant of a nucleic acid sequence as disclosed herein may be developed via codon optimization of the nucleic acid sequence. “Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge). A skilled artisan will understand that a nucleic acid sequence as disclosed herein encompasses variants thereof, including those variants that differ due to degeneracy of the genetic code and/or codon optimization, and that encode the same or substantially similar amino acid sequence of a biologically equivalent polypeptide.

The phrase “bispecific antibody” includes an antibody capable of selectively binding two or more epitopes. Bispecific antibodies generally comprise two different heavy chains, with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The phrase “heavy chain,” or “immunoglobulin heavy chain” includes an immunoglobulin heavy chain constant region sequence from any organism, and unless otherwise specified includes a heavy chain variable domain. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a CH1 domain, a hinge, a CH2 domain, and a CH3 domain. A functional fragment of a heavy chain includes a fragment that is capable of specifically recognizing an antigen (e.g., recognizing the antigen with a KD in the micromolar, nanomolar, or picomolar range), that is capable of expressing and secreting from a cell, and that comprises at least one CDR.

The phrase “light chain” includes an immunoglobulin light chain constant region sequence from any organism, and unless otherwise specified includes human kappa and lambda light chains. Light chain variable (VL) domains typically include three light chain CDRs and four framework (FR) regions, unless otherwise specified. Generally, a full-length light chain includes, from amino terminus to carboxyl terminus, a VL domain that includes FR1-CDR1-FR2-CDR2-FR3-CDR3-FR4, and a light chain constant domain. Light chains that may be useful include e.g., those, that do not selectively bind either the first or second antigen selectively bound by the antigen-binding protein. Suitable light chains include those that can be identified by screening for the most commonly employed light chains in existing antibody libraries (wet libraries or in silico), where the light chains do not substantially interfere with the affinity and/or selectivity of the antigen-binding domains of the antigen-binding proteins. Suitable light chains include those that can bind one or both epitopes that are bound by the antigen-binding regions of the antigen-binding protein.

The phrase “variable domain” includes an amino acid sequence of an immunoglobulin light or heavy chain (modified as desired) that comprises the following amino acid regions, in sequence from N-terminal to C-terminal (unless otherwise indicated): FR1, CDR1, FR2, CDR2, FR3, CDR3, FR4. A “variable domain” includes an amino acid sequence capable of folding into a canonical domain (VH or VL) having a dual beta sheet structure wherein the beta sheets are connected by a disulfide bond between a residue of a first beta sheet and a second beta sheet.

The phrase “complementarity determining region,” or the term “CDR,” includes an amino acid sequence encoded by a nucleic acid sequence of an organism's immunoglobulin genes that normally (i.e., in a wildtype animal) appears between two framework regions in a variable region of a light or a heavy chain of an immunoglobulin molecule (e.g., an antibody or a T cell receptor). A CDR can be encoded by, for example, a germline sequence or a rearranged or unrearranged sequence, and, for example, by a naive or a mature B cell or a T cell. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The term “antibody fragment”, refers to one or more fragments of an antibody that retain the ability to specifically bind to an antigen. Examples of binding fragments encompassed within the term “antibody fragment” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et al. (1993) PNAS USA 90:6444-6448; Poljak et al. (1994) Structure 2:1121-1123).

The phrase “Fc-containing protein” includes antibodies, bispecific antibodies, immunoadhesins, and other binding proteins that comprise at least a functional portion of an immunoglobulin CH2 and CH3 region. A “functional portion” refers to a CH2 and CH3 region that can bind a Fc receptor (e.g., an FcyR; or an FcRn, i.e., a neonatal Fc receptor), and/or that can participate in the activation of complement. If the CH2 and CH3 region contains deletions, substitutions, and/or insertions or other modifications that render it unable to bind any Fc receptor and also unable to activate complement, the CH2 and CH3 region is not functional.

Fc-containing proteins can comprise modifications in immunoglobulin domains, including where the modifications affect one or more effector function of the binding protein (e.g., modifications that affect FcyR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

For example, and not by way of limitation, the binding protein is an Fc-containing protein and exhibits enhanced serum half-life (as compared with the same Fc-containing protein without the recited modification(s)) and have a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at 428 and/or 433 (e.g., L/R/SI/P/Q or K) and/or 434 (e.g., H/F or Y); or a modification at 250 and/or 428; or a modification at 307 or 308 (e.g., 308F, V308F), and 434. In another example, the modification can comprise a 428L (e.g., M428L) and 434S (e.g., N434S) modification; a 428L, 259I (e.g., V259I), and a 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); a 307 and/or 308 modification (e.g., 308F or 308P).

The term “antigen-binding protein,” as used herein, refers to a polypeptide or protein (one or more polypeptides complexed in a functional unit) that specifically recognizes an epitope on an antigen, such as a cell-specific antigen and/or a target antigen as described herein. An antigen-binding protein may be multi-specific. The term “multi-specific” with reference to an antigen-binding protein means that the protein recognizes different epitopes, either on the same antigen or on different antigens. A multi-specific antigen-binding protein as described herein can be a single multifunctional polypeptide, or it can be a multimeric complex of two or more polypeptides that are covalently or non-covalently associated with one another. The term “antigen-binding protein” includes antibodies or fragments thereof as described herein that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bispecific or a multi-specific antigen-binding molecule with a second binding specificity.

The term “protein” means any amino acid polymer having more than about 20 amino acids covalently linked via amide bonds. Proteins contain one or more amino acid polymer chains, generally known in the art as “polypeptides”. Thus, a polypeptide may be a protein, and a protein may contain multiple polypeptides to form a single functioning biomolecule. Disulfide bridges (i.e., between cysteine residues to form cystine) may be present in some proteins. These covalent links may be within a single polypeptide chain, or between two individual polypeptide chains. For example, disulfide bridges are essential to proper structure and function of insulin, immunoglobulins, protamine, and the like. For a recent review of disulfide bond formation, see Oka and Bulleid, “Forming disulfides in the endoplasmic reticulum,” 1833(11) Biochim Biophys Acta 2425-9 (2013).

As used herein, “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins may be produced using recombinant cell-based production systems, such as the insect baculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation,” 28 Biotechnol Genet Eng Rev. 147-75 (2012).

As used herein, the term “epitope” refers to the portion of the antigen which is recognized by the multi-specific antigen-binding polypeptide. A single antigen (such as an antigenic polypeptide) may have more than one epitope. Epitopes may be defined as structural or functional. Functional epitopes are generally a subset of structural epitopes and are defined as those residues that directly contribute to the affinity of the interaction between the antigen-binding polypeptide and the antigen. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

The term “domain” refers to any part of a protein or polypeptide having a particular function or structure. Preferably, domains as described herein bind to cell-specific or target antigens. Cell-specific antigen- or target antigen-binding domains, and the like, as used herein, include any naturally occurring, enzymatically obtainable, synthetic, or genetically engineered polypeptide or glycoprotein that specifically binds an antigen.

The term “half-body” or “half-antibody”, which are used interchangeably, refers to half of an antibody, which essentially contains one heavy chain and one light chain. Antibody heavy chains can form dimers, thus the heavy chain of one half-body can associate with heavy chain associated with a different molecule (e.g., another half-body) or another Fc-containing polypeptide. Two slightly different Fc-domains may “heterodimerize” as in the formation of bispecific antibodies or other heterodimers, -trimers, -tetramers, and the like. See Vincent and Murini, “Current strategies in antibody engineering: Fc engineering and pH-dependent antigen binding, bispecific antibodies and antibody drug conjugates,” 7 Biotechnol. J. 1444-1450 (20912); and Shimamoto et al., “Peptibodies: A flexible alternative format to antibodies,” 4(5) MAbs 586-91 (2012).

The term “single-chain variable fragment” or “scFv” includes a single chain fusion polypeptide containing an immunoglobulin heavy chain variable region (VH) and an immunoglobulin light chain variable region (VL). In some embodiments, the VH and VL are connected by a linker sequence of 10 to 25 amino acids. ScFv polypeptides may also include other amino acid sequences, such as CL or CH1 regions. ScFv molecules can be manufactured by phage display or made by directly subcloning the heavy and light chains from a hybridoma or B-cell. Ahmad et al., Clinical and Developmental Immunology, volume 2012, article ID 98025 is incorporated herein by reference for methods of making scFv fragments by phage display and antibody domain cloning.

### Adeno-Associated Viruses (AAV)

“AAV” is an abbreviation for adeno-associated virus and may be used to refer to the virus itself or derivatives thereof. AAVs are small, non-enveloped, single-stranded DNA viruses. Generally, a wildtype AAV genome is 4.7 kb and is characterized by two inverted terminal repeats (ITR) and two open reading frames (ORFs), rep and cap. The wildtype rep reading frame encodes four proteins of molecular weight 78 kD (“Rep78”), 68 kD (“Rep68”), 52 kD (“Rep52”) and 40 kD (“Rep 40”). Rep78 and Rep68 are transcribed from the p5 promoter, and Rep52 and Rep40 are transcribed from the p19 promoter. These proteins function mainly in regulating the transcription and replication of the AAV genome. The wildtype cap reading frame encodes three structural (capsid) viral proteins (VPs) having molecular weights of 83-85 kD (VP1), 72-73 kD (VP2) and 61-62 kD (VP3). More than 80% of total proteins in an AAV virion (capsid) comprise VP3; in mature virions VP1, VP2 and VP3 are found at relative abundance of approximately 1:1:10, although ratios of 1:1:8 have been reported. Padron et al. (2005) J. Virology 79:5047-58.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077 (AAV1), AF063497 (AAV1), NC001401 (AAV-2), AF043303 (AAV2), NC_001729 (AAV3), NC_001829 (AAV4), U89790 (AAV4), NC_006152 (AAV5), AF513851 (AAV7), AF513852 (AAV8), and NC_006261 (AAV8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99: 11854; Moris et al. (2004) Virology 33:375-383; US Patent Publication 20170130245; international patent publications WO 00/28061, WO 99/61601, WO 98/11244; and U.S. Pat. No. 6,156,303, each of which is incorporated by reference in its entirety by reference. Table 2 herein provides sequences of various non-primate AAV.

“AAV” encompasses all subtypes and both naturally occurring and modified forms that are well-known in the art. AAV includes primate AAV (e.g., AAV type 1 (AAV1), primate AAV type 2 (AAV2), primate AAV type 3 (AAV3B), primate AAV type 4 (AAV4), primate AAV type 5 (AAV5), primate AAV type 6 (AAV6), primate AAV type 7 (AAV7), primate AAV type 8 (AAV8), primate AAV type 9 (AAV9), AAV10, AAV11, AAV12, AAV13, AAVDJ, Anc80L65, AAV2G9, AAV-LK03, primate AAV type rh10 (AAV rh10), AAV type h10 (AAV h10), AAV type hu11 (AAV hu11), AAV type rh32.33 (AAV rh32.33), AAV retro (AAV retro), AAV PHP.B, AAV PHP.eB, AAV PHP.S, AAV2/8, etc., non-primate animal AAV (e.g., avian AAV (AAAV)) and other non-primate animal AAV such as mammalian AAV (e.g., bat AAV, sea lion AAV, bovine AAV, canine AAV, equine AAV, caprine AAV, and ovine AAV etc.), squamate AAV (e.g., snake AAV, bearded dragon AAV), etc. “Primate AAV” refers to AAV generally isolated from primates. Similarly, “non-primate animal AAV” refers to AAV isolated from non-primate animals.

As used herein, “of a [specified] AAV” in relation to a gene (e.g., rep, cap, etc.), capsid protein (e.g., a VP1 capsid protein, a VP2 capsid protein, a VP3 capsid protein, etc.), region of a capsid protein of a specified AAV (e.g., PLA2 region, VP1-u region, VP1/VP2 common region, VP3 region), nucleotide sequence (e.g., ITR sequence), e.g., a cap gene or capsid protein of AAV etc., encompasses, in addition to the gene or the polypeptide respectively comprising a nucleic acid sequence or amino acid sequence set forth herein for the specified AAV, also variants of the gene or polypeptide, including variants comprising the least number of nucleotides or amino acids required to retain one or more biological functions. As used herein, a variant gene or a variant polypeptide comprises a nucleic acid sequence or amino acid sequence that differs from the nucleic acid sequence or amino acid sequence set forth herein for the gene or polypeptide of a specified AAV, wherein the difference(s) does not generally alter at least one biological function of the gene or polypeptide, and/or the phylogenetic characterization of the gene or polypeptide, e.g., where the difference(s) may be due to degeneracy of the genetic code, isolate variations, length of the sequence, etc. For example, rep gene and the cap gene as used here may encompass rep and cap genes that differ from the wildtype gene in that the genes may encode one or more Rep proteins and Cap proteins, respectively. In some embodiments, a Rep gene encodes at least Rep78 and/or Rep68. In some embodiments, cap gene includes those may differ from the wildtype in that one or more alternative start codons or sequences between one or more alternative start codons are removed such that the cap gene encodes only a single Cap protein, e.g., wherein the VP2 and/or VP3 start codons are removed or substituted such that the cap gene encodes a functional VP1 capsid protein but not a VP2 capsid protein or a VP3 capsid protein. Accordingly, as used herein, a rep gene encompasses any sequence that encodes a functional Rep protein. A cap gene encompasses any sequence that encodes at least one functional cap gene.

It is well-known that the wildtype cap gene expresses all three VP1, VP2, and VP3 capsid proteins from a single open reading frame of the cap gene under control of the p40 promoter found in the rep ORF. The term “capsid protein,” “Cap protein” and the like includes a protein that is part of the capsid of the virus. For adeno-associated viruses, the capsid proteins are generally referred to as VP1, VP2 and/or VP3, and may be encoded by the single cap gene. For AAV, the three AAV capsid proteins are produced in nature an overlapping fashion from the cap ORF alternative translational start codon usage, although all three proteins use a common stop codon. The ORF of a wildtype cap gene encodes from 5′ to 3′ three alternative start codons: “the VP1 start codon,” “the VP2 start codon,” and “the VP3 start codon”; and one “common stop codon”. The largest viral protein, VP1, is generally encoded from the VP1 start codon to the “common stop codon.” VP2 is generally encoded from the VP2 start codon to the common stop codon. VP3 is generally encoded from the VP3 start codon to the common stop codon. Accordingly, VP1 comprises at its N-terminus sequence that it does not share with the VP2 or VP3, referred to as the VP1-unique region (VP1-u). The VP1-u region is generally encoded by the sequence of a wildtype cap gene starting from the VP1 start codon to the “VP2 start codon.” VP1-u comprises a phospholipase A2 domain (PLA2), which may be important for infection, as well as nuclear localization signals which may aid the virus in targeting to the nucleus for uncoating and genome release. The VP1, VP2, and VP3 capsid proteins share the same C-terminal sequence that makes up the entirety of VP3, which may also be referred to herein as the VP3 region. The VP3 region is encoded from the VP3 start codon to the common stop codon. VP2 has an additional ~60 amino acids that it shares with the VP1. This region is called the VP1/VP2 common region.

In some embodiments, one or more of the Cap proteins of the invention may be encoded by one or more cap genes having one or more ORFs. In some embodiments, the VP proteins of the invention may be expressed from more than one ORF comprising nucleotide sequence encoding any combination of VP1, VP2, and/or VP3 by use of separate nucleotide sequences operably linked to at least one expression control sequence for expression in packaging cell, each producing one or more of VP1, VP2, and/or VP3 capsid proteins of the invention. In some embodiments, a VP capsid protein of the invention may be expressed individually from an ORF comprising nucleotide sequence encoding any one of VP1, VP2, or VP3 by use of separate nucleotide sequences operably linked to one expression control sequence for expression in a viral replication cell, each producing only one of VP1, VP2, or VP3 capsid protein. In another embodiment, VP proteins may be expressed from one ORF comprising nucleotide sequences encoding VP1, VP2, and VP3 capsid proteins operably linked to at least one expression control sequence for expression in a viral replication cell, each producing VP1, VP2, and VP3 capsid protein. Accordingly, although amino acid positions provided herein may be provided in relation to the VP1 capsid protein of the referenced AAV, a skilled artisan would be able to respectively and readily determine the position of that same amino acid within the VP2 and/or VP3 capsid protein of the AAV, and the corresponding position of amino acids among different AAV.

The phrase “Inverted terminal repeat” or “ITR” includes symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV particles, e.g., packaging into AAV particles.

AAV ITR comprise recognition sites for replication proteins Rep78 or Rep68. A“D” region of the ITR comprises the DNA nick site where DNA replication initiates and provides directionality to the nucleic acid replication step. An AAV replicating in a mammalian cell typically comprises two ITR sequences.

A single ITR may be engineered with Rep binding sites on both strands of the “A” regions and two symmetrical D regions on each side of the ITR palindrome. Such an engineered construct on a double-stranded circular DNA template allows Rep78 or Rep68 initiated nucleic acid replication that proceeds in both directions. A single ITR is sufficient for AAV replication of a circular particle. In methods of producing an AAV viral particle of the invention, the rep encoding sequence encodes a Rep protein or Rep protein equivalent that is capable of binding an ITR comprised on the transfer plasmid.

The Cap proteins of the invention, when expressed with appropriate Rep proteins by a packaging cell, may encapsidate a transfer plasmid comprising a nucleotide of interest and an even number of two or more ITR sequences. In some embodiments, a transfer plasmid comprises one ITR sequence. In some embodiments, a transfer plasmid comprises two ITR sequences.

Either Rep78 and/or Rep68 bind to unique and known sites on the sequence of the ITR hairpin, and act to break and unwind the hairpin structures on the end of an AAV genome, thereby providing access to replication machinery of the viral replication cell. As is well-known, Rep proteins may be expressed from more than one ORF comprising nucleotide sequence encoding any combination of Rep78, Rep68, Rep 52 and/or Rep40 by use of separate nucleotide sequences operably linked to at least one expression control sequence for expression in a viral replication cell, each producing one or more of Rep78, Rep68, Rep 52 and/or Rep40 Rep proteins. Alternatively, Rep proteins may be expressed individually from an ORF comprising a nucleotide sequence encoding any one of Rep78, Rep68, Rep 52, or Rep40 by use of separate nucleotide sequences operably linked to one expression control sequence for expression in a packaging cell, each producing only one Rep78, Rep68, Rep 52, or Rep40 Rep protein. In another embodiment, Rep proteins may be expressed from one ORF comprising nucleotide sequences encoding Rep78 and Rep52 Rep proteins operably linked to at least one expression control sequence for expression in a viral replication cell each producing Rep78 and Rep52 Rep protein.

In a method of producing an AAV virion, e.g., viral particle, of the invention, a rep encoding sequence and a cap gene of the invention may be provided a single packaging plasmid. However, a skilled artisan will recognize that such proviso is not necessary. Such viral particles may or may not include a genome.

A “chimeric AAV capsid protein” includes an AAV capsid protein that comprises amino acid sequences, e.g., portions, from two or more different AAV and that is capable of forming and/or forms an AAV viral capsid/viral particle. A chimeric AAV capsid protein is encoded by a chimeric AAV capsid gene, e.g., a chimeric nucleotide comprising a plurality, e.g., at least two, nucleic acid sequences, each of which plurality is identical to a portion of a capsid gene encoding a capsid protein of distinct AAV, and which plurality together encodes a functional chimeric AAV capsid protein. Association of a chimeric capsid protein to a specific AAV indicates that the capsid protein comprises one or more portions from a capsid protein of that AAV and one or more portions from a capsid protein of a different AAV. For example, a chimeric AAV2 capsid protein includes a capsid protein comprising one or more portions of a VP1, VP2, and/or VP3 capsid protein of AAV2 and one or more portions of a VP1, VP2, and/or VP3 capsid protein of a different AAV.

The term “portion” refers to at least 5 amino acids or at least 15 nucleotides, but less than the full-length polypeptide or nucleic acid molecule, with 100% identity to a sequence from which the portion is derived, see Penzes (2015) J. General Virol. 2769. A “portion” encompasses any contiguous segment of amino acids or nucleotides sufficient to determine that the polypeptide or nucleic acid molecule form which the portion is derived is “of a [specified] AAV” or has “significant identity” to a particular AAV, e.g., a non-primate animal AAV or remote AAV. In some embodiments, a portion comprises at least 5 amino acids or 15 nucleotides with 100% identity to a sequence associated with the specified AAV. In some embodiments, a portion comprises at least 10 amino acids or 30 nucleotides with 100% identity to a sequence associated with the specified AAV. In some embodiments, a portion comprises at least 15 amino acids or 45 nucleotides with 100% identity to a sequence associated with the specified AAV. In some embodiments, a portion comprises at least 20 amino acids or 60 nucleotides with 100% identity to a sequence associated with the specified AAV. In some embodiments, a portion comprises at least 25 amino acids or 75 nucleotides with 100% identity to a sequence associated with the specified AAV. In some embodiments, a portion comprises at least 30 amino acids or 90 nucleotides with 100% identity to a sequence associated with the specified AAV. In some embodiments, a portion comprises at least 35 amino acids or 105 nucleotides with 100% identity to a sequence associated with the specified AAV. In some embodiments, a portion comprises at least 40 amino acids or 120 nucleotides with 100% identity to a sequence associated with the specified AAV. In some embodiments, a portion comprises at least 45 amino acids or 135 nucleotides with 100% identity to a sequence associated with the specified AAV. In some embodiments, a portion comprises at least 50 amino acids or 150 nucleotides with 100% identity to a sequence associated with the specified AAV. In some embodiments, a portion comprises at least 60 amino acids or 180 nucleotides with 100% identity to a sequence associated with the specified AAV. In some embodiments, a portion comprises at least 70 amino acids or 210 nucleotides with 100% identity to a sequence associated with the specified AAV. In some embodiments, a portion comprises at least 80 amino acids or 240 nucleotides with 100% identity to a sequence associated with the specified AAV. In some embodiments, a portion comprises at least 90 amino acids or 270 nucleotides with 100% identity to a sequence associated with the specified AAV. In some embodiments, a portion comprises at least 100 amino acids or 300 nucleotides with 100% identity to a sequence associated with the specified AAV.

### Modified Virus Capsid Proteins, Viral Particles, Viral Nucleic Acids

In some embodiments, a Cap protein, e.g., a VP1 capsid protein as described herein, a VP2 capsid protein as described herein, and/or a VP3 capsid protein as described herein, is modified to comprise any one or combination of e.g., insertion of a targeting ligand, a chemical modification, a first member of a binding pair, a detectable label, point mutation, etc.

Generally, modification of gene or a polypeptide of a specified AAV, or variants thereof, results in nucleic acid sequence or an amino acid sequence that differs from the nucleic acid sequence or amino acid sequence set forth herein for the specified AAV, wherein the modification alters, confers, or removes one or more biological functions, but does not change the phylogenetic characterization of, the gene or polypeptide as an AAV gene or AAV polypeptide. Modifications may include any one or a combination of: substitution of sequences of a first AAV serotype with sequences of a second AAV serotype to create chimerism; chemical modification; an insertion of: a first member of a binding pair, and/or a point mutation; etc., such that the natural tropism of the capsid protein is reduced to abolished, the tropism of the capsid protein may be more easily redirected, and/or such that the capsid protein comprises a detectable label. Modifications as described herein generally do not alter and preferably decrease the low to no recognition of the modified capsid by pre-existing antibodies found in the general population that were produced during the course of infection with another AAV, e.g., infection with serotypes such as AAV1, AAV2, AAV3, AAV4, AAV5, AAV6, AAV7, AAV8, AAV9, AAV10, AAV11, AAV12, AAV13, AAVDJ, Anc80L65, AAV2G9, AAV-LK03, virions based on such serotypes, virions from currently used AAV gene therapy modalities, or a combination thereof.

### Targeting Ligand

Modifications described herein may pertain to the association (e.g., display, operable linkage, binding) of a targeting ligand to a modified capsid protein and/or capsid comprising a modified capsid protein. Generally, a targeting ligand as described herein binds a surface protein expressed by a mammalian muscle cell, e.g., a protein that is expressed on the surface of a mammalian muscle cell, e.g., a mammalian muscle cell-specific surface protein. In some embodiments, a modified capsid protein and/or modified capsid comprises a targeting ligand that binds mammalian CACNG1, e.g., a human CACNG1.

### Antigen-Binding Molecules

An anti-hCACNG1 antibody and antigen-binding fragment thereof as described herein may be monospecific, bi-specific, or multispecific. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. An anti-hCACNG1 antibody and antigen-binding fragment thereof as described herein can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second or additional binding specificity.

Use of the expression “anti-hCACNG1 antibody” herein is intended to include both monospecific anti-hCACNG1 antibodies as well as bispecific antibodies comprising a CACNG1-binding arm and a “target”-binding arm. Thus, described herein are bispecific antibodies wherein one arm of an immunoglobulin binds human CACNG1, and the other arm of the immunoglobulin is specific for another target molecule. The CACNG1-binding arm can comprise any of the HCVR/LCVR or CDR amino acid sequences as set forth in Table 1 herein.

In certain embodiments, the CACNG1-binding arm binds to human CACNG1 and induces internalization of the CACNG1 and antibody bound thereto. In certain embodiments, the CACNG1-binding arm binds weakly to human CACNG1 and induces internalization of CACNG1 and antibody bound thereto.

In certain exemplary embodiments, the bispecific antigen-binding molecule is a bispecific antibody. Each antigen-binding domain of a bispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A1-HCDR1, A1-HCDR2, and A1-HCDR3; and the CDRs of the second antigen-binding domain may be referred to herein as A2-HCDR1, A2-HCDR2, and A2-HCDR3.

The first antigen-binding domain and the second antigen-binding domain may be directly or indirectly connected to one another to form a bispecific antigen-binding molecule as described herein. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. A “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antigen-binding molecules as described herein will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules as described herein. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antigen-binding molecules as described herein, the multimerizing domains, e.g., Fe domains, may comprise one or more amino acid changes (e.g., insertions, deletions or substitutions) as compared to the wild-type, naturally occurring version of the Fc domain. For example, bispecific antigen-binding molecules may comprise one or more modifications in the Fc domain that results in a modified Fc domain having a modified binding interaction (e.g., enhanced or diminished) between Fe and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fe modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

Also described herein are bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

In certain embodiments, the Fc domain may be chimeric, combining Fe sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 CH2]-[IgG1 CH3]. These and other examples of chimeric Fe domains that can be included in any of the antigen-binding molecules as described herein are described in US Publication 2014/0243504, published Aug. 28, 2014, which is herein incorporated in its entirety. Chimeric Fe domains having these general structural arrangements, and variants thereof, can have altered Fc receptor binding, which in turn affects Fe effector function.

In certain embodiments, an antibody heavy chain as described herein comprises a heavy chain constant region (CH) region that comprises an amino acid sequence at least 95%, at least 96%, at least 97%, at least 98%, at least 99% identical to any one of SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, or SEQ ID NO:255. In some embodiments, the heavy chain constant region (CH) region comprises an amino acid sequence selected from the group consisting of SEQ ID NO:243, SEQ ID NO:244, SEQ ID NO:245, SEQ ID NO:246, SEQ ID NO:247, SEQ ID NO:248, SEQ ID NO:249, SEQ ID NO:250, SEQ ID NO:251, SEQ ID NO:252, SEQ ID NO:253, SEQ ID NO:254, or SEQ ID NO:255.

In some embodiments, an antibody heavy chain as described herein comprises an Fc domain that comprises an amino acid sequence at least 95%, at least 96%, at least 97%, at least 98%, at least 99% identical to any one of SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, or SEQ ID NO:267. In some embodiments, the Fc domain comprises an amino acid sequence selected from the group consisting of SEQ ID NO:256, SEQ ID NO:257, SEQ ID NO:258, SEQ ID NO:259, SEQ ID NO:260, SEQ ID NO:261, SEQ ID NO:262, SEQ ID NO:263, SEQ ID NO:264, SEQ ID NO:265, SEQ ID NO:266, or SEQ ID NO:267.

### Germline Mutations

The anti-hCACNG1 antibodies disclosed herein may comprise one or more amino acid substitutions, insertions and/or deletions in the framework and/or CDR regions of the heavy chain variable domains as compared to the corresponding germline sequences from which the antibodies were derived.

An anti-hCACNG1 antibody and antigen-binding fragment thereof as disclosed herein may be derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), and having weak or no detectable binding to a CACNG1 antigen. Several such exemplary antibodies that recognize CACNG1 are described in Table I herein.

Furthermore, an anti-hCACNG1 antibody and antigen-binding fragment thereof as disclosed herein may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, an antibody or antigen-binding fragment that contains one or more germline mutations can be tested for one or more desired properties such as, improved binding specificity, weak or reduced binding affinity, improved or enhanced pharmacokinetic properties, reduced immunogenicity, etc. In some embodiments, an antibody or antigen-binding fragment as described herein is obtained in this general manner.

Also described herein are anti-hCACNG1 antibodies and antigen-binding fragments thereof comprising variants of any of the HCVR, LCVR, and/or CDR amino acid sequences disclosed herein having one or more conservative substitutions. For example, an anti-hCACNG1 antibody or antigen-binding fragment thereof as described herein may comprise HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein. An antibody and antigen-binding fragment thereof as described herein may comprise one or more amino acid substitutions, insertions and/or deletions in the framework and/or CDR regions of the heavy and light chain variable domains as compared to the corresponding germline sequences from which the individual antigen-binding domains were derived, while maintaining or improving the desired weak-to-no detectable binding to, e.g., CACNG1. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein, i.e., the amino acid substitution maintains or improves the desired weak to no detectable binding affinity in the case of anti-hCACNG1 binding molecules. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

Also described herein are anti-hCACNG1 antibodies and antigen-binding fragments thereof comprising an antigen-binding domain with an HCVR and/or CDR amino acid sequence that is substantially identical to any of the HCVR and/or CDR amino acid sequences disclosed herein, while maintaining or improving the desired weak affinity to CACNG1 antigen. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence as described herein to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

Once obtained, antigen-binding domains that contain one or more germline mutations were tested for decreased binding affinity utilizing one or more in vitro assays. Generally, antibodies that recognize a particular antigen are typically screened for their purpose by testing for high (i.e. strong) binding affinity to the antigen.

Unexpected benefits, for example, improved pharmacokinetic properties and low toxicity to the patient may be realized from further modifying the antibodies as described herein by the methods described herein.

### Binding Properties of the Antibodies

The term “binding” in the context of the binding of an antibody, immunoglobulin, antibody-binding fragment, or Fc-containing protein to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a KD value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody, Ig, antibody-binding fragment, or Fc-containing protein as the analyte (or antiligand). Cell-based binding strategies, such as fluorescent-activated cell sorting (FACS) binding assays, are also routinely used and provide binding characterization data with respect to cell-surface expressed proteins. FACS data correlates well with other methods such as radioligand competition binding and SPR (Benedict, C A, J Immunol Methods. 1997, 201(2):223-31; Geuijen, C A, et al. J Immunol Methods. 2005, 302(1-2):68-77).

Accordingly, an anti-hCACNG1 antibody and antigen-binding fragment thereof as described herein bind to the predetermined antigen or cell surface molecule (receptor) having an affinity corresponding to a K_{D} value that is at least ten-fold lower than its affinity for binding to a non-specific antigen (e.g., BSA, casein). The affinity of an antibody corresponding to a KD value that is equal to or less than ten-fold lower than a non-specific antigen may be considered non-detectable binding, however such an antibody may be paired with a second antigen binding arm for the production of a bispecific antibody as described herein.

The term “K_{D}” or “KD” in molar (M) refers to the dissociation equilibrium constant of a particular antibody-antigen interaction, or the dissociation equilibrium constant of an antibody or antibody-binding fragment binding to an antigen. There is an inverse relationship between K_{D} and binding affinity, therefore the smaller the K_{D} value, the higher, i.e. stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. In some circumstances, a higher binding affinity (or K_{D}) of a particular molecule (e.g. antibody) to its interactive partner molecule (e.g. antigen X) compared to the binding affinity of the molecule (e.g. antibody) to another interactive partner molecule (e.g. antigen Y) may be expressed as a binding ratio determined by dividing the larger K_{D} value (lower, or weaker, affinity) by the smaller K_{D} (higher, or stronger, affinity), for example expressed as 5-fold or 10-fold greater binding affinity, as the case may be.

The term “k_{d}” (see −1 or 1/s) refers to the dissociation rate constant of a particular antibody-antigen interaction, or the dissociation rate constant of an antibody or antibody-binding fragment. Said value is also referred to as the k_{off} value.

The term “kₐ” (M−1×sec−1 or 1/M) refers to the association rate constant of a particular antibody-antigen interaction, or the association rate constant of an antibody or antibody-binding fragment.

The term “K_{A}” (M−1 or 1/M) refers to the association equilibrium constant of a particular antibody-antigen interaction, or the association equilibrium constant of an antibody or antibody-binding fragment. The association equilibrium constant is obtained by dividing the ka by the ka.

The term “EC50” or “EC50” refers to the half maximal effective concentration, which includes the concentration of an antibody which induces a response halfway between the baseline and maximum after a specified exposure time. The EC50 essentially represents the concentration of an antibody where 50% of its maximal effect is observed. In certain embodiments, the EC50 value equals the concentration of an antibody as described herein that gives half-maximal binding to cells expressing CACNG1, as determined by e.g., a FACS binding assay or an androgen receptor activation luciferase assay. Thus, reduced or weaker binding is observed with an increased EC50, or half maximal effective concentration value.

In one embodiment, decreased binding can be defined as an increased EC50 antibody concentration which enables binding to the half-maximal amount of target cells.

### Sequence Variants

An anti-hCACNG1 antibody and antigen-binding fragment as described herein may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germline sequences available from, for example, public antibody sequence databases. The antigen-binding molecules as described herein may comprise antigen-binding domains which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen-binding domains that contain one or more germline mutations can be easily tested for one or more desired property such as, improved binding specificity, increased binding affinity, improved or enhanced antagonistic or agonistic biological properties (as the case may be), reduced immunogenicity, etc. Described herein are antigen-binding molecules comprising one or more antigen-binding domains obtained in this general manner.

Also described herein are antigen-binding molecules wherein one or both antigen-binding domains comprise variants of any of the HCVR, LCVR, and/or CDR amino acid sequences disclosed herein having one or more conservative substitutions. For example, antigen-binding molecules as described herein may comprise an antigen-binding domain having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Antigen-binding molecules as described herein may comprise an antigen-binding domain with an HCVR, LCVR, and/or CDR amino acid sequence that is substantially identical to any of the HCVR, LCVR, and/or CDR amino acid sequences disclosed herein. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

pH-Dependent Binding

Also described herein are anti-hCACNG1 antibodies and antigen-binding fragments thereof with pH-dependent binding characteristics. For example, an anti-hCACNG1 as described herein may exhibit reduced binding to CACNG1 at acidic pH as compared to neutral pH. Alternatively, anti-hCACNG1 antibodies as described herein may exhibit enhanced binding to CACNG1 at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. The expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding . . . at acidic pH as compared to neutral pH” is expressed in terms of a ratio of the K_{D} value of the antibody binding to its antigen at acidic pH to the K_{D} value of the antibody binding to its antigen at neutral pH (or vice versa). For example, an antibody or antigen-binding fragment thereof may be regarded as exhibiting “reduced binding to CACNG1 at acidic pH as compared to neutral pH” for purposes of the description herein if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment as described herein can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0 or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained. Antibodies Comprising Fc Variants

In some embodiments, an anti-hCACNG1 antibody and antigen-binding fragment thereof (including a multispecific antigen-binding molecule and a multidomain therapeutic protein comprising an anti-hCACNG1 antibody or an antigen-binding fragment thereof) is provided comprising an Fc domain comprising one or more mutations which enhance or diminish antibody binding to the FcRn receptor, e.g., at acidic pH as compared to neutral pH. For example, antibodies as described herein may comprise a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, an anti-hCACNG1 antibody and antigen-binding fragment as described herein may comprise an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the description herein.

### Biological Characteristics of the Antibodies and Bispecific Antigen-Binding Molecules

Also described herein is an antibody and antigen-binding fragment thereof that binds human CACNG1 with high, medium or low affinity, depending on the therapeutic context and particular targeting properties that are desired. For example, in the context of a bispecific antigen-binding molecule, wherein one arm binds CACNG1 and another arm binds a target antigen (e.g., a tumor associated antigen), it may be desirable for the target antigen-binding arm to bind the target antigen with high affinity while the anti-hCACNG1 arm binds CACNG1 with only moderate or low affinity. In this manner, preferential targeting of the antigen-binding molecule to cells expressing the target antigen may be achieved while avoiding general/untargeted CACNG1 binding and the consequent adverse side effects associated therewith.

Also described herein are antibodies, antigen-binding fragments, and bispecific antibodies thereof that bind human CACNG1 with weak (i.e. low) or even no detectable affinity. In some embodiments, an antibody and antigen-binding fragment thereof as described herein binds human CACNG1 (e.g., at 37° C.) with a K_{D} of greater than about 100 nM as measured by surface plasmon resonance. In some embodiments, an antibody or antigen-binding fragment as described herein binds CACNG1 with a K_{D} of greater than about greater than about 110 nM, at least 120 nM, greater than about 130 nM, greater than about 140 nM, greater than about 150 nM, at least 160 nM, greater than about 170 nM, greater than about 180 nM, greater than about 190 nM, greater than about 200 nM, greater than about 250 nM, greater than about 300 nM, greater than about 400 nM, greater than about 500 nM, greater than about 600 nM, greater than about 700 nM, greater than about 800 nM, greater than about 900 nM, or greater than about 1 μM, or with no detectable affinity, as measured by surface plasmon resonance (e.g., mAb-capture or antigen-capture format), or a substantially similar assay.

### Epitope Mapping and Related Technologies

The epitope on CACNG1 to which an anti-hCACNG1 antibody and antigen-binding fragment thereof as described herein may consist of a single contiguous sequence of 3 or more (e.g., 3, 4, 5, 6, 7, 8, 9, 10, 11, 12, 13, 14, 15, 16, 17, 18, 19, 20 or more) amino acids of a CACNG1 protein. Alternatively, the epitope may consist of a plurality of non-contiguous amino acids (or amino acid sequences) of CACNG1. The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstances, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antigen-binding domain of an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. X-ray crystallography of the antigen/antibody complex may also be used for epitope mapping purposes.

Also described herein are anti-hCACNG1 antibodies that bind to the same epitope as any of the specific exemplary antibodies described herein (e.g., antibodies comprising any of the amino acid sequences as set forth in Table 1 herein). Likewise, also described herein are anti-hCACNG1 antibodies that compete for binding to CACNG1 with any of the specific exemplary antibodies described herein (e.g., antibodies comprising any of the amino acid sequences as set forth in Table 1 herein).

One can easily determine whether a particular antigen-binding molecule (e.g., antibody) or antigen-binding domain thereof binds to the same epitope as, or competes for binding with, a reference antigen-binding molecule as described herein by using routine methods known in the art. For example, to determine if a test antibody binds to the same epitope on CACNG1 as a reference bispecific antigen-binding molecule as described herein, the reference bispecific molecule is first allowed to bind to a CACNG1 protein. Next, the ability of a test antibody to bind to the CACNG1 molecule is assessed. If the test antibody is able to bind to CACNG1 following saturation binding with the reference bispecific antigen-binding molecule, it can be concluded that the test antibody binds to a different epitope of CACNG1 than the reference bispecific antigen-binding molecule. On the other hand, if the test antibody is not able to bind to the CACNG1 molecule following saturation binding with the reference bispecific antigen-binding molecule, then the test antibody may bind to the same epitope of CACNG1 as the epitope bound by the reference bispecific antigen-binding molecule as described herein. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with some embodiments described herein, two antigen-binding proteins bind to the same (or overlapping) epitope if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins are deemed to bind to the same epitope if essentially all amino acid mutations in the antigen that reduce or eliminate binding of one antigen-binding protein reduce or eliminate binding of the other. Two antigen-binding proteins are deemed to have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a CACNG1 protein under saturating conditions followed by assessment of binding of the test antibody to the CACNG1 molecule. In a second orientation, the test antibody is allowed to bind to a CACNG1 molecule under saturating conditions followed by assessment of binding of the reference antigen-binding molecule to the CACNG1 molecule. If, in both orientations, only the first (saturating) antigen-binding molecule is capable of binding to the CACNG1 molecule, then it is concluded that the test antibody and the reference antigen-binding molecule compete for binding to CACNG1. As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### Preparation of Antigen-Binding Domains and Construction of Binding Molecules

Antigen-binding domains specific for particular antigens can be prepared by any antibody generating technology known in the art. Once obtained, two different antigen-binding domains, specific for two different antigens (e.g., CACNG1 and a target antigen), can be appropriately arranged relative to one another to produce a bispecific antigen-binding molecule as described herein using routine methods. (A discussion of exemplary bispecific antibody formats that can be used to construct the bispecific antigen-binding molecules as described herein is provided elsewhere herein). In certain embodiments, one or more of the individual components (e.g., heavy and light chains) of the antigen-binding molecules as described herein are derived from chimeric, humanized or fully human antibodies. Methods for making such antibodies are well known in the art. For example, one or more of the heavy and/or light chains of the antigen-binding molecules as described herein can be prepared using VELOCIMMUNE™ technology. Using VELOCIMMUNE™ technology (or any other human antibody generating technology), high affinity chimeric antibodies to a particular antigen (e.g., CACNG1) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the antigen-binding molecules as described herein.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to isolate heavy chain and light chain variable regions to produce fully human bispecific antigen-binding molecules. As such, the fully human bispecific antigen-binding molecules comprise two different heavy chains that associate with the same light chain. (See, e.g., US 2011/0195454). Fully human refers to an antibody, or antigen-binding fragment or immunoglobulin domain thereof, comprising an amino acid sequence encoded by a DNA derived from a human sequence over the entire length of each polypeptide of the antibody or antigen-binding fragment or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g., compared to any wild-type human immunoglobulin regions or domains.

Bispecific antigen-binding molecules may be constructed with one heavy chain having a modified Fc domain that abrogates its binding to Protein A, thus enabling a purification method that yields a heterodimeric protein. See, for example, U.S. Pat. No. 8,586,713. As such, the bispecific antigen-binding molecules comprise a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation/modification that reduces or abolishes Protein A binding such as an H95R modification (by IMGT exon numbering; H435R by EU numbering). The second C_{H}3 may further comprise a Y96F modification (by IMGT; Y436F by EU).

### Bioequivalents

Antigen-binding molecules having amino acid sequences that vary from those of the exemplary molecules disclosed herein but that retain the ability to bind CACNG1 are also described herein. Such variant molecules may comprise one or more additions, deletions, or substitutions of amino acids when compared to parent sequence, but exhibit biological activity that is essentially equivalent to that of the described bispecific antigen-binding molecules.

Antigen-binding molecules that are bioequivalent to any of the exemplary antigen-binding molecules set forth herein are also described. Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antigen-binding proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding proteins may include variants of the exemplary bispecific antigen-binding molecules set forth herein comprising amino acid changes which modify the glycosylation characteristics of the molecules, e.g., mutations which eliminate or remove glycosylation.

### Species Selectivity and Species Cross-Reactivity

In some embodiments, antigen-binding molecules as described herein bind to human CACNG1 but not to CACNG1 from other species. Also described herein are antigen-binding molecules that bind to human CACNG1 and to CACNG1 from one or more non-human species.

In some embodiments, antigen-binding molecules as described herein that bind to human CACNG1 may bind, or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CACNG1.

Non-limiting examples of targeting ligands that bind CACNG1 include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “targeting ligand,” as used herein. In non-limiting embodiments, an anti-CACNG1 targeting ligand that binds CACNG1 useful for retargeting viral capsids as described herein comprise comprises an scFv. As a non-limiting example, an scFv sequences in V_{L}-(Gly₄Ser)₃-V_{H} format useful for retargeting viral capsids as described herein may comprise a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 that is 90%, 95%, 97%, 98%, 99% or 100% identical, respectively, to any one of the amino acid sequences of a heavy chain variable domain, light chain variable domain, heavy chain variable domain/light chain variable domain pair, HCDR1, HCDR2, HCDR3, LCDR1, LCDR2, LCDR3, and/or set of HCDR1-HCDR2-HCDR3-LCDR1-LCDR2-LCDR3 as set forth in any one of SEQ ID NOs:1-240.

A targeting ligand that binds a mammalian muscle cell-specific surface protein may be associated with (e.g., displayed by, operably linked to, bound to) a modified AAV capsid protein and resulting AAV capsids according to well-known methods, e.g., a direct approach in which the targeting ligand is directly inserted into (e.g., using recombinatorial methods) according to well-known methods. See, e.g., Stachler et al. (2006), supra; White et al. (2004), supra; Girod et al. (1999), supra; Grifman et al. (2001), supra; Shi et al. (2001), supra; Shi and Bartlett (2003), supra. A targeting ligand that binds a mammalian muscle cell-specific surface protein may be coupled to a modified AAV capsid protein and resulting AAV capsids using well-known chemical linkers, e.g., wherein the AAV capsid protein may be chemically modified to comprise a dibenzocycootyne group or an azide group, and optionally wherein a targeting ligand as described herein is attached to the dibenzocycootyne group or the azide group, see, e.g., U.S. 2022/028234, incorporated herein by reference in its entirety; wherein targeting ligand is covalently linked to a primary amino acid group of an AAV capsid protein, e.g., via a —CSNH— bond, etc. In some embodiments, a modified capsid as described herein comprises a targeting ligand, e.g., an anti-CACNG1 antibody or binding portion thereof, directly inserted into or coupled to it according to well-known direct recombinatorial methods.

### Binding Pairs

In some embodiments, a targeting ligand that binds a mammalian muscle cell-specific surface protein may be associated with (e.g., displayed by, operably linked to, bound to) a modified AAV capsid protein and resulting AAV capsids according to indirect recombinatorial approaches, wherein the AAV capsid protein is modified to comprise a first member of a binding pair (e.g., a heterologous scaffold), and optionally wherein the first member of the binding pair is linked to (e.g., covalently or non-covalently bound to) a second cognate member of the binding pair (e.g., an adaptor), further optionally wherein the second cognate member of the binding pair is fused to the targeting ligand. Non-limiting and exemplary binding pairs are listed in Buning and Srivastava (2019) Mol. Ther. Methods Cin Dev 12:248-265.

Accordingly, in some embodiments, modifications of a capsid protein as described herein include those that generally result from modifications at the genetic level, e.g., via modification of a cap gene, such as modifications that insert first member of a binding pair (e.g., a protein:protein binding pair, a protein:nucleic acid binding pair), a detectable label, etc., for display by the Cap protein.

In some embodiments, the first member forms a binding pair with an immunoglobulin constant domain. In some embodiments, the first member forms a binding pair with a metal ion, e.g., Ni²⁺, Co²⁺, Cu²⁺, Zn²⁺, Fe³⁺, etc. In some embodiments, the first member is selected from the group consisting of Streptavidin, Strep II, HA, L14, 4C-RGD, LH, and Protein A.

In some embodiments, the binding pair comprises an enzyme:nucleic acid binding pair. In some embodiments, the first member comprises a HUH-endonuclease or HUH-tag and the second member comprises a nucleic acid binding domain. In some embodiments, the first member comprises a HUH tag. See, e.g., U.S. 2021/0180082, incorporated herein in its entirety by reference.

In some embodiments, a capsid protein of the invention comprises at least a first member of a peptide:peptide binding pair.

In some embodiments, each of a first member and a second member of a peptide:peptide binding pair comprises an intein. See, e.g., Wagner et al., (2021) Adv. Sci. 8: 2004018 (1 of 22); Muik et al. (2017) Biomaterials 144: 84, each of which is incorporated herein in its entirety by reference.

In some embodiments, a first member is a B cell epitope, e.g., is between about 1 amino acid and about 35 amino acids in length, and forms a binding pair with an antibody paratope, e.g., an immunoglobulin variable domain. In some embodiments, a capsid protein of the invention may be modified to comprise a detectable label as a first member of a binding pair. Many detectable labels are known in the art. (See, e.g.: Nilsson et al. (1997) “Affinity fusion strategies for detection, purification, and immobilization of modified proteins” Protein Expression and Purification 11: 1-16, Terpe et al. (2003) “Overview of tag protein fusions: From molecular and biochemical fundamentals to commercial systems” Applied Microbiology and Biotechnology 60:523-533, and references therein). Detectable labels include, but are not limited to, a polyhistidine detectable labels (e.g., a His-6, His-8, or His-10) that binds immobilized divalent cations (e.g., Ni²⁺), a biotin moiety (e.g., on an in vivo biotinylated polypeptide sequence) that binds immobilized avidin, a GST (glutathione S-transferase) sequence that binds immobilized glutathione, an S tag that binds immobilized S protein, an antigen that binds an immobilized antibody or domain or fragment thereof (including, e.g., T7, myc, FLAG, and B tags that bind corresponding antibodies), a FLASH Tag (a high detectable label that couples to specific arsenic based moieties), a receptor or receptor domain that binds an immobilized ligand (or vice versa), protein A or a derivative thereof (e.g., Z) that binds immobilized IgG, maltose-binding protein (MBP) that binds immobilized amylose, an albumin-binding protein that binds immobilized albumin, a chitin binding domain that binds immobilized chitin, a calmodulin binding peptide that binds immobilized calmodulin, and a cellulose binding domain that binds immobilized cellulose. Another exemplary detectable label is a SNAP-tag. In some embodiments, a detectable label disclosed herein comprises a detectable label recognized by an antibody paratope, wherein the detectable label and the antibody paratope form a protein:protein binding pair.

In some embodiments, a capsid protein of the invention comprises a first member of a protein:protein binding pair comprising a detectable label, which may also be used for the detection and/or isolation of the Cap protein and/or as a first member of a protein:protein binding pair. In some embodiments, a detectable label acts as a first member of a protein:protein binding pair for the binding of a targeting ligand comprising a multispecific binding protein that may bind both the detectable label and a target expressed by a cell of interest. In some embodiments, a Cap protein of the invention comprises a first member of a protein:protein binding pair comprising c-myc (Use of a detectable label as a first member of a protein:protein binding pair is described in, e.g., WO2019006043, incorporated herein in its entirety by reference.

In some embodiments, a capsid protein comprises a first member of a protein:protein binding pair, wherein the protein:protein binding pair forms a covalent isopeptide bond. In some embodiments, the first member of a peptide:peptide binding pair is covalently bound via an isopeptide bond to a cognate second member of the peptide:peptide binding pair, and optionally wherein the cognate second member of the peptide:peptide binding pair is fused with a targeting ligand, which targeting ligand binds a target expressed by a cell of interest. In some embodiments, the protein:protein binding pair may be selected from the group consisting of SpyTag:SpyCatcher, SpyTag002:SpyCatcher002, SpyTag003:SpyCatcher003, SpyTag:KTag, Isopeptag:pilin-C, and SnoopTag:SnoopCatcher. In some embodiments, wherein the first member is SpyTag (or a biologically equivalent portion or variant thereof) and the protein (second cognate member) is SpyCatcher (or a biologically equivalent portion or variant thereof). In some embodiments, wherein the first member is SpyTag (or a biologically equivalent portion or variant thereof) and the protein (second cognate member) is KTag (or a biologically equivalent or variant thereof). In some embodiments, wherein the first member is KTag (or a biologically equivalent portion or variant thereof) and the protein (second cognate member) is SpyTag (or a biologically equivalent portion or variant thereof). In some embodiments, wherein the first member is SnoopTag (or a biologically equivalent portion or variant thereof) and the protein (second cognate member) is SnoopCatcher (or a biologically equivalent portion or variant thereof). In some embodiments, wherein the first member is Isopeptag (or a biologically equivalent portion or variant thereof) and the protein (second cognate member) is Pilin-C (or a biologically equivalent portion or variant thereof). In some embodiments, wherein the first member is SpyTag002 (or a biologically equivalent portion or variant thereof) and the protein (second cognate member) is SpyCatcher002 (or a biologically equivalent portion or variant thereof). In some embodiments, wherein the first member is SpyTag003 (or a biologically equivalent portion or variant thereof) and the protein (second cognate member) is SpyCatcher003 (or a biologically equivalent portion or variant thereof). In some embodiments, a Cap protein of the invention comprises a SpyTag, or a biologically equivalent portion or variant thereof. Use of a first member of a protein:protein binding pair is described in WO2019006046, incorporated herein in its entirety.

The phrase “operably linked”, as used herein, includes a physical juxtaposition (e.g., in three-dimensional space) of components or elements that interact, directly or indirectly with one another, or otherwise coordinate with each other to participate in a biological event, which juxtaposition achieves or permits such interaction and/or coordination. To give but one example, a regulatory element (e.g., an expression control sequence) in a nucleic acid is said to be “operably linked” to a coding sequence when it is located relative to the coding sequence such that its presence or absence impacts expression and/or activity of the coding sequence. In many embodiments, “operable linkage” involves covalent linkage of relevant components or elements with one another. Those skilled in the art will readily appreciate that, in some embodiments, covalent linkage is not required to achieve effective operable linkage. For example, proteins operably linked together may be associated with each other, e.g., via a covalent bond or a non-covalent bond. As a non-limiting example, a capsid protein as described herein may be operably linked to a targeting ligand, where the capsid protein is non-covalently bound to the targeting ligand, or covalently bound to the targeting ligand, optionally with or without a scaffold and/or adaptor between the capsid protein and the targeting ligand. As another example, in some embodiments, nucleic acid regulatory elements that are operably linked with coding sequences that they control are contiguous with the nucleotide of interest. Alternatively or additionally, in some embodiments, one or more such regulatory elements acts in trans or at a distance to control a coding sequence of interest. In some embodiments, the term “regulatory element” as used herein refers to polynucleotide sequences which are necessary and/or sufficient to affect the expression and processing of coding sequences to which they are ligated. In some embodiments, a regulatory element may be or comprise appropriate transcription initiation, termination, promoter and/or enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and/or, in some embodiments, sequences that enhance protein secretion. In some embodiments, one or more regulatory elements are preferentially or exclusively active in a particular host cell or organism, or type thereof. To give but one example, in prokaryotes, regulatory elements may typically include promoter, ribosomal binding site, and transcription termination sequence; in eukaryotes, in many embodiments, regulatory elements may typically include promoters, enhancers, and/or transcription termination sequences. Those of ordinary skill in the art will appreciate from context that, in many embodiments, the term “regulatory elements” refers to components whose presence is essential for expression and processing, and in some embodiments includes components whose presence is advantageous for expression (including, for example, leader sequences, targeting sequences, and/or fusion partner sequences).

“Retargeting” or “redirecting” may include a scenario in which the wildtype particle targets several cells within a tissue and/or several organs within an organism, and general targeting of the tissue or organs is reduced or abolished by insertion of the heterologous amino acid, and retargeting to more a specific cell in the tissue or a specific organ in the organism is achieved with the targeting ligand (e.g., via a targeting ligand) that binds a marker expressed by the specific cell. Such retargeting or redirecting may also include a scenario in which the wildtype particle targets a tissue, and targeting of the tissue is reduced to or abolished by insertion of the heterologous amino acid, and retargeting to a completely different tissue is achieved with the targeting ligand.

“Specific binding pair,” “binding pair,” “protein:protein binding pair” and the like includes two members (e.g., a first member (e.g., a first polypeptide) and a second cognate member (e.g., a second polypeptide)) that interact to form a bond (e.g., a non-covalent bond between a first member epitope and a second member antigen-binding portion of an antibody that recognizes the epitope; a covalent bond between e.g., proteins capable of forming isopeptide bonds; split inteins that recognize each other and, through the process of protein trans-splicing, mediate ligation of the flanking proteins and their own removal). In some embodiments, the term “cognate” refers to components that function together. Epitopes and cognate antibodies thereto, particularly epitopes that may also act as a detectable label (e.g., c-myc) are well-known in the art. Specific protein:protein binding pairs capable of interacting to form a covalent isopeptide bond are reviewed in Veggiani et al. (2014) Trends Biotechnol. 32:506, and include peptide:peptide binding pairs such as SpyTag:SpyCatcher, SpyTag002:SpyCatcher002; SpyTag:KTag, isopeptag:pilin C, SnoopTag:SnoopCatcher, etc., and variants thereof, e.g., SpyTag003:SpyCatcher003. Generally, a first member of a protein:protein binding pair refers to member of a protein:protein binding pair, which is generally less than 30 amino acids in length, and which forms a spontaneous covalent isopeptide bond with the second cognate protein, wherein the second cognate protein is generally larger, but may also be less than 30 amino acids in length such as in the SpyTag:KTag system.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly i.e., between two peptide/protein molecules, e.g., between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. In preferred embodiments of the invention, an isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The SpyTag:SpyCatcher system is described in U.S. Pat. No. 9,547,003 and Zaveri et al. (2012) PNAS 109:E690-E697, each of which is incorporated herein in its entirety by reference, and is derived from the CnaB2 domain of the Streptococcus pyogenes fibronecting-binding protein FbaB. By splitting the domain, Zakeri et al. obtained a peptide “SpyTag” having the sequence AHIVMVDAYKPTK (SEQ ID NO:243) which forms an amide bond to its cognate protein “SpyCatcher,” a 112 amino acid polypeptide having the amino acid sequence set forth in SEQ ID NO:244. (Zakeri (2012), supra). An additional specific binding pair derived from CnaB2 domain is SpyTag:KTag, which forms an isopeptide bond in the presence of SpyLigase. (Fierer (2014) PNAS 111:E1176-1181) SpyLigase was engineered by excising the β strand from SpyCatcher that contains a reactive lysine, resulting in KTag, 10-residue first member of a protein:protein binding pair having the amino acid sequence ATHIKFSKRD (SEQ ID NO:245). The SpyTag002:SpyCatcher002 system is described in Keeble et al (2017) Angew Chem Int Ed Engl 56:16521-25, incorporated herein in its entirety by reference. SpyTag002 has the amino acid sequence VPTIVMVDAYKRYK, set forth as SEQ ID NO:255, and binds SpyCatcher002. SpyTag003 has the amino acid sequence RGVPHIVMVDAYKRYK, set forth as SEQ ID NO:259, and binds SpyCatcher003.

The SnoopTag:SnoopCatcher system is described in Veggiani (2016) PNAS 113:1202-07. The D4 Ig-like domain of RrgA, an adhesion from Streptococcus pneumoniae, was split to form SnoopTag (residues 734-745) and SnoopCatcher (residues 749-860). Incubation of SnoopTag and SnoopCatcher results in a spontaneous isopeptide bond that is specific between the complementary proteins. Veggiani (2016)), supra.

The isopeptag:pilin-C specific binding pair was derived from the major pilin protein Spy0128 from Streptococcus pyogenes. (Zakeir and Howarth (2010) J. Am. Chem. Soc. 132:4526-27). Isopeptag has the amino acid sequence TDKDMTITFTNKKDAE, set forth as SEQ ID NO:254, and binds pilin-C (residues 18-299 of Spy0128). Incubation of SnoopTag and SnoopCatcher results in a spontaneous isopeptide bond that is specific between the complementary proteins. Zakeir and Howarth (2010), supra.

The term “detectable label” includes a polypeptide sequence that is a member of a specific binding pair, e.g., that specifically binds via a non-covalent bond with another polypeptide sequence, e.g., an antibody paratope, with high affinity. Exemplary and non-limiting detectable labels include hexahistidine tag, FLAG tag, Strep II tag, streptavidin-binding peptide (SBP) tag, calmodulin-binding peptide (CBP), glutathione S-transferase (GST), maltose-binding protein (MBP), S-tag, HA tag, and the myc tag from c-myc (SEQ ID NO:246). (Reviewed in Zhao et al. (2013) J. Analytical Meth. Chem. 1-8; incorporated herein by reference). A common detectable label for primate AAV is the B1 epitope (SEQ ID NO:247). Some AAV capsid proteins described herein, which do not naturally comprise the B1 epitope, may be modified herein to comprise a B1 epitope. Generally, AAV capsid proteins described herein may comprise a sequence with substantial homology to the B1 epitope within the last 10 amino acids of the capsid protein. Accordingly, in some embodiments, a non-primate AAV capsid protein of the invention may be modified with one but less than five point mutations within the last 10 amino acids of the capsid protein such that the AAV capsid protein comprises a B1 epitope.

The term “target cells” includes any cells in which expression of a nucleotide of interest is desired. Preferably, target cells exhibit a receptor on their surface that allows the cell to be targeted with a targeting ligand, as described below.

The term “transduction” or “infection” or the like refers to the introduction of a nucleic acid into a target cell nucleus by a viral particle. The term efficiency in relation to transduction or the like, e.g., “transduction efficiency” refers to the fraction (e.g., percentage) of cells expressing a nucleotide of interest after incubation with a set number of viral particles comprising the nucleotide of interest. Well-known methods of determining transduction efficiency include flow cytometry of cells transduced with a fluorescent reporter gene, RT-PCR for expression of the nucleotide of interest, etc.

Generally “reference” viral capsid protein/capsid/particle are identical to test viral capsid protein/capsid/particle but for the change for which the effect is to be tested. For example, to determine the effect, e.g., on transduction efficiency, of inserting a first member of a specific binding pair into a test viral particle, the transduction efficiencies of the test viral particle (in the absence or presence of an appropriate targeting ligand) can be compared to the transduction efficiencies of a reference viral particle (in the absence or presence of an appropriate targeting ligand if necessary) which is identical to the test viral particle in every instance (e.g., additional point mutations, nucleotide of interest, numbers of viral particles and target cells, etc.) except for the presence of a first member of a specific binding pair. In some embodiments, a reference viral capsid protein is one that is able to form a capsid with a second viral capsid protein modified to comprise at least a first member of a protein:protein binding pair, where the reference viral capsid protein does not comprise the first member of a protein:protein binding pair, preferably wherein the capsid formed by the reference viral capsid protein and the modified viral capsid protein is a mosaic capsid.

In some embodiments, a first member of a protein:protein binding pair and/or detectable label is operably linked to (translated in frame with, chemically attached to, and/or displayed by) a Cap protein of the invention via a first or second linker, e.g., an amino acid spacer that is at least one amino acid in length. In some embodiments, the first member of a protein:protein binding pair is flanked by a first and/or second linker, e.g., a first and/or second amino acid spacer, each of which spacer is at least one amino acid in length.

In some embodiments, the first and/or second linkers are not identical. In some embodiments, the first and/or second linker is each independently one or two amino acids in length. In some embodiments, the first and/or second linker is each independently one, two or three amino acids in length. In some embodiments, the first and/or second linker is each independently one, two, three, or four amino acids in length. In some embodiments, the first and/or second linker is each independently one, two, three, four, or five amino acids in length. In some embodiments, the first and/or second linker are each independently one, two, three, four, or five amino acids in length. In some embodiments, the first and/or second linker is each independently one, two, three, four, five, or six amino acids in length. In some embodiments, the first and/or second linker is each independently one, two, three, four, five, six, or seven amino acids in length. In some embodiments, the first and/or second linker is each independently one, two, three, four, five, six, seven, or eight amino acids in length. In some embodiments, the first and/or second linker is each independently one, two, three, four, five, six, seven, eight or nine amino acids in length. In some embodiments, the first and or second linker is each independently one, two, three, four, five, six, seven, eight, nine, or ten amino acids in length. In some embodiments, the first and or second linker is each independently one, two, three, four, five, six, seven, eight, nine, ten, or more amino acids in length.

In some embodiments, the first and second linkers are identical in sequence and/or in length and are each one amino acid in length. In some embodiments, the first and second linkers are identical in length, and are each one amino acid in length. In some embodiments, the first and second linkers are identical in length, and are each two amino acids in length. In some embodiments, the first and second linkers are identical in length, and are each three amino acids in length. In some embodiments, the first and second linkers are identical in length, and are each four amino acids in length, e.g., the linker is GLSG (SEQ ID NO:248). In some embodiments, the first and second linkers are identical in length, and are each five amino acids in length. In some embodiments, the first and second linkers are identical in length, and are each six amino acids in length, e.g., the first and second linkers each comprise a sequence of GLSGSG (SEQ ID NO:249). In some embodiments, the first and second linkers are identical in length, and are each seven amino acids in length. In some embodiments, the first and second linkers are identical in length, and are each eight amino acids in length, e.g., the first and second linkers each comprise a sequence of GLSGLSGS (SEQ ID NO:250). In some embodiments, the first and second linkers are identical in length, and are each nine amino acids in length. In some embodiments, the first and second linkers are identical in length, and are each ten amino acids in length, e.g., the first and second linkers each comprise a sequence of GLSGLSGLSG (SEQ ID NO:251) or GLSGGSGLSG (SEQ ID NO:252). In some embodiments, the first and second linkers are identical in length, and are each more than ten amino acids in length.

Generally, a first member of a protein:protein binding pair amino acid sequence as described herein, e.g., comprising a first member of a specific binding pair by itself or in combination with one or more linkers, is between about 5 amino acids to about 50 amino acids in length. In some embodiments, the first member of a protein:protein binding pair amino acid sequence is at least 5 amino acids in length. In some embodiments, the first member of a protein:protein binding pair amino acid sequence is 6 amino acids in length. In some embodiments, the first member of a protein:protein binding pair amino acid sequence is 7 amino acids in length. In some embodiments, the first member of a protein:protein binding pair amino acid sequence is 8 amino acids in length. In some embodiments, the first member of a protein:protein binding pair amino acid sequence is 9 amino acids in length. In some embodiments, the first member of a protein:protein binding pair amino acid sequence is 10 amino acids in length. In some embodiments, the first member of a protein:protein binding pair amino acid sequence is 11 amino acids in length. In some embodiments, the first member of a protein:protein binding pair amino acid sequence is 12 amino acids in length. In some embodiments, the first member of a protein:protein binding pair amino acid sequence is 13 amino acids in length. In some embodiments, the first member of a protein:protein binding pair amino acid sequence is 14 amino acids in length. In some embodiments, the first member of a protein:protein binding pair amino acid sequence is 15 amino acids in length. In some embodiments, the first member of a protein:protein binding pair amino acid sequence is 16 amino acids in length. In some embodiments, the first member of a protein:protein binding pair amino acid sequence is 17 amino acids in length. In some embodiments, the first member of a protein:protein binding pair amino acid sequence is 18 amino acids in length. In some embodiments, the first member of a protein:protein binding pair amino acid sequence is 19 amino acids in length. In some embodiments, the first member of a protein:protein binding pair amino acid sequence is 20 amino acids in length. In some embodiments, the first member of a protein:protein binding pair amino acid sequence is 21 amino acids in length. In some embodiments, the first member of a protein:protein binding pair amino acid sequence is 22 amino acids in length. In some embodiments, the first member of a protein:protein binding pair amino acid sequence is 23 amino acids in length. In some embodiments, the first member of a protein:protein binding pair amino acid sequence is 24 amino acids in length. In some embodiments, the first member of a protein:protein binding pair amino acid sequence is 25 amino acids in length. In some embodiments, the first member of a protein:protein binding pair amino acid sequence is 26 amino acids in length. In some embodiments, the first member of a protein:protein binding pair amino acid sequence is 27 amino acids in length. In some embodiments, the first member of a protein:protein binding pair amino acid sequence is 28 amino acids in length. In some embodiments, the first member of a protein:protein binding pair amino acid sequence is 29 amino acids in length. In some embodiments, the first member of a protein:protein binding pair amino acid sequence is 30 amino acids in length. In some embodiments, the first member of a protein:protein binding pair amino acid sequence is 31 amino acids in length. In some embodiments, the first member of a protein:protein binding pair amino acid sequence is 32 amino acids in length. In some embodiments, the first member of a protein:protein binding pair amino acid sequence is 33 amino acids in length. In some embodiments, the first member of a protein:protein binding pair amino acid sequence is 34 amino acids in length. In some embodiments, the first member of a protein:protein binding pair amino acid sequence is 35 amino acids in length. In some embodiments, the first member of a protein:protein binding pair amino acid sequence is 36 amino acids in length. In some embodiments, the first member of a protein:protein binding pair amino acid sequence is 37 amino acids in length. In some embodiments, the first member of a protein:protein binding pair amino acid sequence is 38 amino acids in length. In some embodiments, the first member of a protein:protein binding pair amino acid sequence is 39 amino acids in length. In some embodiments, the first member of a protein:protein binding pair amino acid sequence is 40 amino acids in length. In some embodiments, the first member of a protein:protein binding pair amino acid sequence is 41 amino acids in length. In some embodiments, the first member of a protein:protein binding pair amino acid sequence is 42 amino acids in length. In some embodiments, the first member of a protein:protein binding pair amino acid sequence is 43 amino acids in length. In some embodiments, the first member of a protein:protein binding pair amino acid sequence is 44 amino acids in length. In some embodiments, the first member of a protein:protein binding pair amino acid sequence is 45 amino acids in length. In some embodiments, the first member of a protein:protein binding pair amino acid sequence is 46 amino acids in length. In some embodiments, the first member of a protein:protein binding pair amino acid sequence is 47 amino acids in length. In some embodiments, the first member of a protein:protein binding pair amino acid sequence is 48 amino acids in length. In some embodiments, the first member of a protein:protein binding pair amino acid sequence is 49 amino acids in length. In some embodiments, the first member of a protein:protein binding pair amino acid sequence is 50 amino acids in length.

### Modified Capsids Comprising Modified Capsid Proteins

In some embodiments a viral capsid comprising a modified viral capsid protein as described herein is a mosaic capsid, e.g., comprises at least two sets of VP1, VP2, and/or VP3 proteins, each set of which is encoded by a different cap gene. A mosaic capsid herein generally refers to a mosaic of a first viral capsid protein modified to comprise a first member of a binding pair and a second corresponding viral capsid protein lacking the first member of a binding pair. In relation to a mosaic capsid, the second viral capsid protein lacking the first member of a binding pair may be referred to as a reference capsid protein encoded by a reference cap gene. In some mosaic capsid embodiments, preferably when the VP1, VP2, and/or VP3 capsid proteins modified with a first member of protein:protein pair is not a chimeric capsid protein, a VP1, VP2, and/or VP3 reference capsid protein may comprise an amino acid sequence identical to that of the viral VP1, VP2, and/or VP3 capsid protein modified with a first member of a binding pair, except that the reference capsid protein lacks the first member of a binding pair. In some mosaic capsid embodiments, a VP1, VP2, and/or VP3 reference capsid protein corresponds to the viral VP1, VP2, and/or VP3 capsid protein modified with a first member of a binding pair, except that the reference capsid protein lacks the first member of a binding pair. In some embodiments, a VP1 reference capsid protein corresponds to the viral VP1 capsid protein modified with a first member of a binding pair, except that the reference capsid protein lacks the first member of a binding pair. In some embodiments, a VP2 reference capsid protein corresponds to the viral VP2 capsid protein modified with a first member of a binding pair, except that the reference capsid protein lacks the first member of a binding pair. In some embodiments, a VP3 reference capsid protein corresponds to the viral VP3 capsid protein modified with a first member of a binding pair, except that the reference capsid protein lacks the first member of a binding pair. In some mosaic capsid embodiments comprising a chimeric VP1, VP2, and/or VP3 capsid protein further modified to comprise a first member of a binding pair, a reference protein may be a corresponding capsid protein from which portions thereof form part of the chimeric capsid protein. As a non-limiting example in some embodiments, mosaic capsid comprising a chimeric AAV2/AAAV VP1 capsid protein modified to comprise a first member of a binding pair may further comprise as a reference capsid protein: an AAV2 VP1 capsid protein lacking the first member, an AAAV VP1 capsid protein lacking the first member, a chimeric AAV2/AAAV VP1 capsid protein lacking the first member. Similarly, in some embodiments, a mosaic capsid comprising a chimeric AAV2/AAAV VP2 capsid protein modified to comprise a first member of a binding pair may further comprise as a reference capsid protein: an AAV2 VP2 capsid protein lacking the first member, an AAAV VP1 capsid protein lacking the first member, a chimeric AAV2/AAAV VP2 capsid protein lacking the first member. In some embodiments, a mosaic capsid comprising a chimeric AAV2/AAAV VP3 capsid protein modified to comprise a first member of a binding pair may further comprise as a reference capsid protein: an AAV2 VP2 capsid protein lacking the first member, an AAAV VP1 capsid protein lacking the first member, a chimeric AAV2/AAAV VP3 capsid protein lacking the first member. In some mosaic capsid embodiments, a reference capsid protein may be any capsid protein so long as it that lacks the first member of the binding pair and is able to form a capsid with the first capsid protein modified with the first member of a binding pair.

Generally, mosaic particles may be generated by transfecting mixtures of the modified and reference Cap genes into production cells at the indicated ratios. The protein subunit ratios, e.g., modified VP protein:unmodified VP protein ratios, in the particle may, but do not necessarily, stoichiometrically reflect the ratios of the at least two species of the cap gene encoding the first capsid protein modified with a first member of a binding pair and the one or more reference cap genes, e.g., modified cap gene:reference cap gene(s) transfected into packaging cells. In some embodiments, the protein subunit ratios in the particle do not stoichiometrically reflect the modified cap gene:reference cap gene(s) ratio transfected into packaging cells.

In some mosaic viral particle embodiments, the protein subunit ratio ranges from about 1:59 to about 59:1. In some mosaic viral particle embodiments, the protein subunit is at least about 1:1 (e.g., the mosaic viral particle comprises about 30 modified capsid proteins and about 30 reference capsid protein). In some mosaic viral particle embodiments, the protein subunit ratio is at least about 1:2 (e.g., the mosaic viral particle comprises about 20 modified capsid proteins and about 40 reference capsid proteins). In some mosaic viral particle embodiments, the protein subunit ratio is at least about 3:5. In some mosaic viral particle embodiments, the protein subunit ratio is at least about 1:3 (e.g., the mosaic viral particle comprises about 15 modified capsid proteins and about 45 reference capsid proteins). In some mosaic viral particle embodiments, the protein subunit ratio is at least about 1:4 (e.g., the mosaic viral particle comprises about 12 modified capsid proteins and 48 reference capsid proteins). In some mosaic viral particle embodiments, the protein subunit ratio is at least about 1:5 (e.g., the mosaic viral particle comprises 10 modified capsid proteins and 50 reference capsid proteins). In some mosaic viral particle embodiments, the protein subunit ratio is at least about 1:6. In some mosaic viral particle embodiments, the protein subunit ratio is at least about 1:7. In some mosaic viral particle embodiments, the protein subunit ratio is at least about 1:8. In some mosaic viral particle embodiments, the protein subunit ratio is at least about 1:9 (e.g., the mosaic viral particle comprises about 6 modified capsid proteins and about 54 reference capsid proteins). In some mosaic viral particle embodiments, the protein subunit ratio is at least about 1:10. In some mosaic viral particle embodiments, the protein subunit ratio is at least about 1:11 (e.g., the mosaic viral particle comprises about 5 modified capsid proteins and about 55 reference capsid proteins). In some mosaic viral particle embodiments, the protein subunit ratio is at least about 1:12. In some mosaic viral particle embodiments, the protein subunit ratio is at least about 1:13. In some mosaic viral particle embodiments, the protein subunit ratio is at least about 1:14 (e.g., the mosaic viral particle comprises about 4 modified capsid proteins and about 56 reference capsid proteins). In some mosaic viral particle embodiments, the protein subunit ratio is at least about 1:15. In some mosaic viral particle embodiments, the protein subunit ratio is at least about 1:19 (e.g., the mosaic viral particle comprises about 3 modified capsid proteins and about 57 reference capsid proteins). In some mosaic viral particle embodiments, the protein subunit ratio is at least about 1:29 (e.g., the mosaic viral particle comprises about 2 modified capsid proteins and about 58 reference capsid proteins). In some mosaic viral particle embodiments, the protein subunit ratio is at least about 1:59. In some mosaic viral particle embodiments, the protein subunit ratio is at least about 2:1 (e.g., the mosaic viral particle comprises about 40 modified capsid proteins and about 20 reference capsid proteins). In some mosaic viral particle embodiments, the protein subunit ratio is at least about 5:3. In some mosaic viral particle embodiments, the protein subunit ratio is at least about 3:1 (e.g., the mosaic viral particle comprises about 45 modified capsid proteins and about 15 reference capsid proteins). In some mosaic viral particle embodiments, the protein subunit ratio is at least about 4:1 (e.g., the mosaic viral particle comprises about 48 modified capsid proteins and 12 reference capsid proteins). In some mosaic viral particle embodiments, the protein subunit ratio is at least about 5:1 (e.g., the mosaic viral particle comprises 50 modified capsid proteins and 10 reference capsid proteins). In some mosaic viral particle embodiments, the protein subunit ratio is at least about 6:1. In some mosaic viral particle embodiments, the protein subunit ratio is at least about 7:1. In some mosaic viral particle embodiments, the protein subunit ratio is at least about 8:1. In some mosaic viral particle embodiments, the protein subunit ratio is at least about 9:1 (e.g., the mosaic viral particle comprises about 54 modified capsid proteins and about 6 reference capsid proteins). In some mosaic viral particle embodiments, the protein subunit ratio is at least about 10:1. In some mosaic viral particle embodiments, the protein subunit ratio is at least about 11:1 (e.g., the mosaic viral particle comprises about 55 modified capsid proteins and about 5 reference capsid proteins). In some mosaic viral particle embodiments, the protein subunit ratio is at least about 12:1. In some mosaic viral particle embodiments, the protein subunit ratio is at least about 13:1. In some mosaic viral particle embodiments, the protein subunit ratio is at least about 14:1 (e.g., the mosaic viral particle comprises about 56 modified capsid proteins and about 4 reference capsid proteins). In some mosaic viral particle embodiments, the protein subunit ratio is at least about 15:1. In some mosaic viral particle embodiments, the protein subunit ratio is at least about 19:1 (e.g., the mosaic viral particle comprises about 57 modified capsid proteins and about 3 reference capsid proteins). In some mosaic viral particle embodiments, the protein subunit ratio is at least about 29:1 (e.g., the mosaic viral particle comprises about 58 modified capsid proteins and about 2 reference capsid proteins). In some mosaic viral particle embodiments, the protein subunit ratio is at least about 59:1.

In some non-mosaic viral particle embodiments, the protein subunit ratio may be 1:0 wherein each capsid protein of the non-mosaic viral particle is modified with a first member of a binding pair. In some non-mosaic viral particle embodiments, the protein subunit ratio may be 0:1 wherein each capsid protein of the non-mosaic viral particle is not modified with a first member of a binding pair.

### Insertion Sites

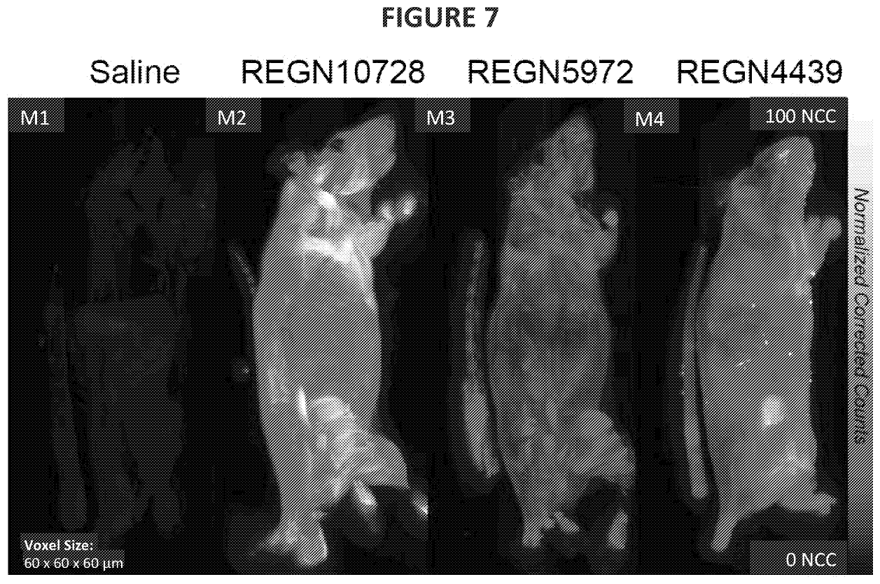

Due to the high conservation of at least large stretches and the large member of closely related family members, the corresponding insertion sites for AAV other than the enumerated AAV can be identified by performing an amino acid alignment or by comparison of the capsid structures. See, e.g., Rutledge et al. (1998) J. Virol. 72:309-19; Mietzsch et al. (2019) Viruses 11, 362, 1-34, and U.S. Pat. No. 9,624,274 for exemplary alignments of different AAV capsid proteins, each of which is incorporated herein by reference in its entirety. For example, Mietzcsh et al. (2019) provide an overlay of ribbons from different dependoparvovirus at FIG. 7, depicting the variable regions VR I to VR IX. Using such structural analysis as described therein, and sequence analysis, a skilled artisan may determine which amino acids within the variable region correspond to amino acid sequence of AAV that can accommodate the insertion of, e.g., a targeting ligand as described herein, a first member of a binding pair and/or detectable label.

Generally, the targeting ligand, first member of a binding pair, and/or detectable label may be inserted into a variable region or variable loop of an AAV capsid protein, a GH loop of an AAV capsid protein, etc.

In some embodiments, the first member of a binding pair and/or detectable label is inserted in a VP1 capsid protein of a non-primate animal AAV after an amino acid position corresponding with an amino acid position selected from the group consisting of G453 of AAV2 capsid protein VP1, N587 of AAV2 capsid protein VP1, G453 of AAV9 capsid protein VP1, and A589 of AAV9 capsid protein VP1. In some embodiments, the first member of a binding pair and/or detectable label is inserted in a VP1 capsid protein of a non-primate animal AAV between amino acids that correspond with N587 and R588 of an AAV2 VP1 capsid. Additional suitable insertion sites of a non-primate animal VP1 capsid protein include those corresponding to I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713 and I-716 of the VP1 capsid protein of AAV2 (Wu et al. (2000) J. Virol. 74:8635-8647). A modified virus capsid protein as described herein may be a non-primate animal capsid protein comprising a first member of a binding pair and/or detectable label inserted into a position corresponding with a position of an AAV2 capsid protein selected from the group consisting of I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713, I-716, and a combination thereof. Additional suitable insertion sites of a non-primate animal AAV that include those corresponding to I-587 or I-590 of AAV1, I-589 of AAV1, I-585 of AAV3, I-584 or I-585 of AAV4, and I-575 or I-585 of AAV5. In some embodiments, a modified virus capsid protein as described herein may be a non-primate animal capsid protein comprising a targeting ligand, first member of a binding pair and/or detectable label inserted into a position corresponding with a position selected from the group consisting of I-587 (AAV1), I-589 (AAV1), I-585 (AAV3), I-585 (AAV4), I-585 (AAV5), and a combination thereof.

In some embodiments, the first member of a binding pair and/or detectable label is inserted in a VP1 capsid protein of a non-primate animal AAV after an amino acid position corresponding with an amino acid position selected from the group consisting of I444 of an avian AAV capsid protein VP1, I580 of an avian AAV capsid protein VP1, I573 of a bearded dragon AAV capsid protein VP1, I436 of a bearded dragon AAV capsid protein VP1, I429 of a sea lion AAV capsid protein VP1, I430 of a sea lion AAV capsid protein VP1, I431 of a sea lion AAV capsid protein VP1, I432 of a sea lion AAV capsid protein VP1, I433 of a sea lion AAV capsid protein VP1, I434 of a sea lion AAV capsid protein VP1, I436 of a sea lion AAV capsid protein VP1, I437 of a sea lion AAV capsid protein VP1, and I565 of a sea lion AAV capsid protein VP1.

The nomenclature I-###, I# or the like herein refers to the insertion site (I) with ### naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s) N- or C-terminal of the given amino acid. Additionally, the positions referred to herein are relative to the VP1 protein encoded by an AAV capsid gene, and corresponding positions (and point mutations thereof) may be easily identified for the VP2 and VP3 capsid proteins encoding by the capsid gene by performing a sequence alignment of the VP1, VP2 and VP3 proteins encoded by the appropriate AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site 1-587. Therefore, the present invention encompasses structural genes of AAV with corresponding insertions in the VP1, VP2 and/or VP3 proteins.

Also provided herein are nucleic acids that encode a VP3 capsid protein of the invention. AAV capsid proteins may be, but are not necessarily, encoded by overlapping reading frames of the same gene with staggered start codons. In some embodiments, a nucleic acid that encodes a VP3 capsid protein of the invention does not also encode a VP2 capsid protein or VP1 capsid protein of the invention. In some embodiments, a nucleic acid that encodes a VP3 capsid protein of the invention may also encode a VP2 capsid protein of the invention but does not also encode a VP1 capsid of the invention. In some embodiments, a nucleic acid that encodes a VP3 capsid protein of the invention may also encode a VP2 capsid protein of the invention and a VP1 capsid of the invention.

In some embodiments, a viral capsid comprising the modified viral capsid protein comprising the first and second members of a binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a binding pair, e.g., comprises a control capsid protein. In some embodiments, a viral capsid comprising a modified viral capsid protein as described herein bound to the first and second members of a binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a binding pair linked to a targeting ligand exhibits a transduction efficiency that is 99% greater than the transduction efficiency of a control viral capsid.

In some embodiments, a viral capsid comprising the modified viral capsid protein comprising the first and second members of a binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a binding pair, e.g., comprises a control capsid protein. In some embodiments, a viral capsid comprising a modified viral capsid protein as described herein bound to the first and second members of a binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a binding pair linked to a targeting ligand exhibits a transduction efficiency that is 99% greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 1.5-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 2-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 3-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 4-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 5-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 6-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 7-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 8-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 9-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 10-fold greater than the transduction efficiency of a control viral capsid. In some embodiments, a viral capsid comprising a modified viral capsid protein as described herein bound to an appropriate the first and second members of a binding pair linked to a targeting ligand exhibits a transduction efficiency that is at least 20-fold greater than the transduction efficiency of a control capsid. In some embodiments, a viral capsid comprising a modified viral capsid protein as described herein bound to an appropriate the first and second members of a binding pair linked to a targeting ligand exhibits a transduction efficiency that is at least 30-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 40-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 50-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 60-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 70-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 80-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 90-fold greater than the transduction efficiency of a control viral capsid. In some embodiments, a viral capsid comprising a modified viral capsid protein as described herein bound to the first and second members of a binding pair linked to a targeting ligand exhibits a transduction efficiency that is at least 100-fold greater than the transduction efficiency of a control viral capsid In some embodiments, a viral particle of the invention comprising a viral capsid protein comprising an amino acid sequence of a capsid protein of a non-primate animal AAV, a remote AAV, or a combination thereof, and optionally comprising a first and second members of a binding pair (e.g., wherein the second member is operably linked to a targeting ligand, comprises a multispecific binding protein, etc.) is better able to evade neutralization by pre-existing antibodies in serum isolated from a human patient compared to an appropriate control viral particle (e.g., comprising a viral capsid of an AAV serotype from which a portion is included in the viral capsid of the invention, e.g., as part of the viral capsid protein comprising an amino acid sequence of a capsid protein of a non-primate animal AAV, a remote AAV, or a combination thereof), which also optionally comprises a first and second members of a binding pair (e.g., wherein the second member is operably linked to a targeting ligand, comprises a multispecific binding protein, etc.). In some embodiments, a viral particle of the invention comprising a viral capsid protein comprising an amino acid sequence of a capsid protein of a non-primate animal AAV, a remote AAV, or a combination thereof requires at least 2-fold more total IVIG or IgG for neutralization (e.g., 50% or more infection inhibition) compared to an appropriate control viral particle, e.g., (e.g., a viral particle of the invention has an IC50 value that is at least 2-fold that of a control virus particle).

In some embodiments of the invention comprising a detectable label, a targeting ligand comprises a multispecific binding molecule comprising (i) an antibody paratope that specifically binds the detectable label and (ii) a second binding domain that specifically binds a receptor, which may be conjugated to the surface of a bead (e.g., for purification) or expressed by a target cell. Accordingly, a multispecific binding molecule comprising (i) an antibody paratope that specifically binds the detectable label and (ii) a second binding domain that specifically binds a receptor targets the viral particle. Such “targeting” or “directing” may include a scenario in which the wildtype viral particle targets several cells within a tissue and/or several organs within an organism, which broad targeting of the tissue or organs is reduced to abolished by insertion of the detectable label, and which retargeting to more specific cells in the tissue or more specific organ in the organism is achieved with the multispecific binding molecule. Such retargeting or redirecting may also include a scenario in which the wildtype viral particle targets a tissue, which targeting of the tissue is reduced to abolished by insertion of the detectable label, and which retargeting to a completely different tissue is achieved with the multispecific binding molecule. An antibody paratope as described herein generally comprises at a minimum a complementarity determining region (CDR) that specifically recognizes the detectable label, e.g., a CDR3 region of a heavy and/or light chain variable domain. In some embodiments, a multispecific binding molecule comprises an antibody (or portion thereof) that comprises the antibody paratope that specifically binds the detectable label. For example, a multispecific binding molecule may comprise a single domain heavy chain variable region or a single domain light chain variable region, wherein the single domain heavy chain variable region or single domain light chain variable region comprises an antibody paratope that specifically binds the detectable label. In some embodiments, a multispecific binding molecule may comprise an Fv region, e.g., a multispecific binding molecule may comprise an scFv, that comprises an antibody paratope that specifically binds the detectable label. In some embodiments, a multispecific binding molecule as described herein comprises an antibody paratope that specifically binds c-myc (SEQ ID NO:246).

One embodiment of the present invention is a multimeric structure comprising a modified viral capsid protein of the present invention. A multimeric structure comprises at least 5, preferably at least 10, more preferably at least 30, most preferably at least 60 modified viral capsid proteins comprising a first member of a specific binding pair as described herein. They can form regular viral capsids (empty viral particles) or viral particles (capsids encapsidating a nucleotide of interest). The formation of viral particles comprising a viral genome is a highly preferred feature for use of the modified viral capsids described herein.

A further embodiment of the present invention is the use of at least one modified viral capsid protein and/or a nucleic acid encoding same, preferably at least one multimeric structure (e.g., viral particle) for the manufacture of and use in transfer of a nucleotide of interest to a target cell.

### Methods of Use and Making

A further embodiment of the modified viral capsids described herein is their use for delivering a nucleotide of interest, e.g., a reporter gene or a therapeutic gene, to a target cell. Generally, packaging of a nucleotide of interest comprises replacing an AAV genome between AAV ITR sequences with a gene of interest to create a transfer plasmid, which is then encapsulated in an AAV capsid according to well-known methods Thus, a modified viral capsid as described herein may encapsulate a transfer plasmid and/or a nucleotide of interest, which may generally comprise 5′ and 3′ inverted terminal repeat (ITR) sequences flanking a gene of interest, e.g., reporter gene(s) or therapeutic gene(s), or a portion of the gene of interest (which may be under the control of a viral or non-viral promoter). According to well-known methods of packaging AAV viral particles, the modified viral capsids, the 5′ ITR, and the 3′ ITR need not be of the same AAV serotype. In one embodiment, a transfer plasmid and/or nucleotide of interest comprises from 5′ to 3′: a 5′ ITR, a promoter, a gene (e.g., a reporter and/or therapeutic gene) and a 3′ ITR.

Genes of interest disclosed herein include, but are not limited to, genes encoding microdystrophin, FKRP, and MTM1, e.g., genes encoding human microdystrophin, human FKRP, and human MTM1. A non-limiting sequence that encodes microdystrophin is set forth as SEQ ID NO:270. A non-limiting sequence that encodes FKRP is set forth as SEQ ID NO:271. A non-limiting sequence that encodes MTM1 is set forth as SEQ ID NO:272. Genes of interest as described herein also include, but are not limited to, biologically equivalent portions or variants of the genes disclosed herein. For example, a gene of interest may comprise a biologically equivalent portion or variant of the sequence set forth as SEQ ID NO:270. A gene of interest may comprise a biologically equivalent portion or variant of the sequence set forth as SEQ ID NO:271. A gene of interest may comprise a biologically equivalent portion or variant of the sequence set forth as SEQ ID 272.

A consideration for AAV transfer plasmid design is that a wildtype AAV genome is ~4.7 kb. Thus, included herein are the well-known strategies that provide for packaging nucleotides of interest that exceed the packaging capacity of an individual AAV. Such strategies include, but are not limited to dual-vector strategies that exploit ITR-mediated recombination to express genes of interest that are larger than a wildtype AAV genome by way of transcript splicing across intermolecularly recombined ITRs from two complementary vector genomes, vector recombination by homology, RNA trans-splicing, and/or protein “trans-splicing” via split intein designs. See, e.g., Nakai, H. et al. (2000) Nat. Biotechnol. 18:527-532; Sun, L. (2000) Nat. Med. 6: 599-602 (2000); Ghosh, A., et al. (2008)Mol. Ther. 16:124-130 (2008); Lai, Y (2005) Nat. Biotechnol. 23:1435-1439; Chew, W. L. et al. (2016) Nat. Methods 13:868-874; Li, J. (2008) Hum. Gene Ther. 19:958-964, each of which reference is incorporated herein in its entirety by reference.

Dual AAV vector strategies to transfer of a large gene into target cells have been described, which rely on different mechanisms including, but not limited to, trans-splicing, including overlapping regions in the dual vectors, and a hybrid of the two. Tornabene and Trapani (2020) Human Gene Ther. 31:47-56; see also U.S. Pat. No. 8,236,557, each of which is incorporated herein by reference in its entirety.

A trans-splicing approach takes advantage of the ability of AAV ITR sequences to concatemerized to reconstitute full-length genomes, wherein each of two or more viral capsids respectively encapsulate one of two or more transfer plasmids, each of which transfer plasmid comprises a portion of the gene of interest. For example, in a dual vector approach, the two transfer plasmids may be designed as follows: the 5′-transfer plasmid comprises the promoter, the 5′ portion of the coding sequence of the gene of interest, and a splicing donor (SD) signal; the 3′-transfer plasmid comprises a splicing acceptor (SA) signal, the 3′ portion of the gene of interest, and the polyA signal. Upon tail-to-head ITR-mediated concatemerization of the two AAV genomes, the SD and SA signals will allow splicing of the recombined genome.

A large gene of interest is also split when taking an overlapping region approach. In the overlapping region approach, the 5′ and 3′ portions (and thus the 5′ transfer plasmid and 3′ transfer plasmid) share a recombinogenic sequence, e.g., region of homology, e.g., each portion comprises an overlapping sequence. The gene of interest is made whole in a targeted cell via homologous recombination mediated by the recombinogenic sequence, e.g., homology/overlapping region.

In a hybrid approach, the 5′-transfer plasmid and 3′-transfer plasmid each comprise a highly recombinogenic sequence, wherein the recombinogenic sequence is placed downstream of an SD signal of a 5′ portion of the coding sequence of the gene of interest and upstream of an SA signal of a 3′ portion of the coding sequence of the gene of interest. In this hybrid system, the gene of interest may be made whole either via ITR-mediated concatemerization and splicing and/or by homologous recombination.

Trans-splicing at the RNA or protein levels may also be utilized. In an RNA trans-splicing approach, two transfer plasmids may respectively encode for 5′ and 3′ fragments of the pre-mRNA of a large gene and share an intronic hybridization domain that can favor trans-splicing, leading to joining of the two half-transcripts into an intact full-length mRNA.

Protein trans-splicing occurs post-translationally and is catalyzed by intervening proteins called split-inteins. Split-inteins are expressed as two independent polypeptides (N-intein and C-intein) at the extremities of two host proteins. The N-intein and C-intein polypeptides remain catalytically inactive until they encounter each other. Upon encountering each other, each intein precisely excises itself from the host protein while mediating ligation of the N- and C-host polypeptides via a peptide bond. Split-intein use has been used in AAV-based delivery of therapeutic genes of interest in muscle, liver, and retinal diseases. For example, on co-delivery of two halves of the mini-dystrophin cDNA fused to N- and C-intein coding sequences, efficient production of the two polypeptides was shown. Li et al. (2008) Hum Gene Ther 19:958-64. Similarly, AAV-split-inteins have been widely used for the expression and ligation of the clustered regularly interspaced short palindromic repeats (CRISPR)-Cas9 nuclease.

The above dual vector approaches are well-known in the art. See, e.g., Tornabene and Trapani (2020), supra; U.S. Pat. No. 8,236,557. Thus, in some embodiments, a modified viral capsid described herein encapsulates a nucleotide of interest, wherein the nucleotide of interest comprises a portion of a gene of interest. In some embodiments, a nucleotide of interest comprising a portion of a gene of interest further comprises a splicing donor signal or a splicing acceptor signal and/or a recombinogenic sequence. In some embodiments, a nucleotide of interest comprising a portion of a gene of interest comprises an intronic hybridization domain encoding sequence. In some embodiments, a nucleotide of interest comprising a portion of a gene of interest comprises a N-intein or C-intein encoding sequence.

Design of the transfer plasmid/nucleotide of interest includes including one or more regulatory elements, e.g., promoter and/or enhancer elements, that will control expression of the gene of interest. Non-limiting examples of useful promoters include, e.g., cytomegalovirus (CMV)-promoter, the spleen focus forming virus (SFFV)-promoter, the elongation factor I alpha (EF1a)-promoter (the 1.2 kb EFla-promoter or the 0.2 kb EFla-promoter), the chimeric EF 1 a/IF4-promoter, the phospho-glycerate kinase (PGK)-promoter, and biologically equivalent portions or variants thereof. An internal enhancer may also be present in the viral construct to increase expression of the gene of interest. For example, the CMV enhancer (Karasuyama et al. 1989. J. Exp. Med. 169:13, which is incorporated herein by reference in its entirety) may be used. In some embodiments, tissue specific regulatory elements, e.g., a muscle specific promoter and/or regulatory element may be used to drive the expression of the gene of interest, e.g., muscle-specific promoters based on skeletal muscle α-actin, muscle creatine kinase, and desmin genes, as well as other genes expressed in muscles. A non-limiting example of an actin gene that prevails in adult muscle is the human skeletal muscle α-actin gene (HSA). The HSA promoter, and biologically equivalent portions or variants thereof, and other the regulatory regions of the homologous chicken, rat, and bovine genes have been used in vitro and in vivo in transgenic animal models and AAV mediated gene transfer. Skopenkova et al. Acta Naturae 13:47-58. In some embodiments, an enhancer, e.g., CMV enhancer, can be used in combination with a promoter of an actin gene, e.g., the chicken β-actin promoter, and biologically equivalent portions or variants thereof. The use of muscle-specific regulatory elements based on the muscle creatine kinase gene (MCK) has also been employed for muscle gene therapy treatments, such as Duchenne muscular dystrophy (DMD) and limb-girdle muscular dystrophy (LGMD). See, e.g., Salva, M. Z. et al. (2007) Mol. Ther. 15: 320-329, incorporated herein in its entirety by reference. In some embodiments, a transfer plasmid and/or nucleotide of interest herein comprises an enhancer and/or promoter of MCK, or a biologically equivalent portion or variant thereof, wherein the enhancer and/or promoter of MCK drives expression of the gene of interest. In some embodiments, the MCK enhancer and/or promoter, or biologically equivalent portion or variant thereof, is selected from the group consisting of CK6, MHCK7, dMCK, tMCK, CK8, and CK8e. In some embodiments, a transfer plasmid and/or nucleotide of interest herein comprises an enhancer and/or promoter element that recruits RNA Polymerase II, wherein the enhancer and/or promoter of MCK (or a biologically equivalent portion or variant thereof) drives expression of the gene of interest. In some embodiments, a transfer plasmid and/or nucleotide of interest herein comprises an enhancer and/or promoter element that recruits RNA Polymerase III, wherein the enhancer and/or promoter of MCK (or a biologically equivalent portion or variant thereof drives expression of the gene of interest. In some embodiments, a transfer plasmid and/or nucleotide of interest herein comprises a desmin promoter, or a biologically equivalent portion or variant thereof. In some embodiments, a transfer plasmid and/or nucleotide of interest herein comprises a human-myosin heavy chain gene (αMHC) promoter, or a biologically equivalent portion or variant thereof. In some embodiments, a transfer plasmid and/or nucleotide of interest herein comprises an MLC promoter, or a biologically equivalent portion or variant thereof, e.g., a CMV-IE enhancer ligated to a rat MLC promoter. In some embodiments, a transfer plasmid and/or nucleotide of interest herein comprises a ΔUSEx3 promoter, or a biologically equivalent portion or variant thereof, which is based on a human troponin I (TNN1) gene. In some embodiments, a transfer plasmid and/or nucleotide of interest herein comprises a unc45b promoter, or a biologically equivalent portion or variant thereof.

In some embodiments, bidirectional promoters and/or vectors have also been employed for delivery of dual therapeutic gene cassettes. An example of this is the bidirectional chicken β-actin ubiquitous promoter that drives the simultaneous expression of the hexosaminidase α- and β-subunits of the HexA enzyme, the two respective genes involved in Tay-Sachs and Sandhoff diseases. Lahey, et al. (2020) Mol. Ther. 28: 2150-2160, incorporated herein in its entirety by reference. In some embodiments, a transfer plasmid and/or nucleotide of interest herein comprises a bidirectional promoter, wherein the bidirectional promoter drives the expression of two different genes of interest.

A variety of reporter genes (or detectable moieties) can be encapsidated in a multimeric structure comprising the modified viral capsid proteins described herein. Exemplary reporter genes include, for example, Q-galactosidase (encoded lacZ gene), Green Fluorescent Protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof. The methods described herein demonstrate the construction of targeting particles that employ the use of a reporter gene that encodes green fluorescent protein, however, persons of skill upon reading this disclosure will understand that the viral capsids described herein can be generated in the absence of a reporter gene or with any reporter gene known in the art.

A variety of therapeutic genes can also be encapsidated in a multimeric structure comprising the modified viral capsid proteins described herein, e.g., as part of a transfer particle. Non-limiting examples of a therapeutic gene include those that encode a toxin (e.g., a suicide gene), a therapeutic antibody or fragment thereof, a CRISPR/Cas system or portion(s) thereof, antisense RNA, siRNA, shRNA, etc.

A further embodiment of the present invention is a process for the preparation of a modified capsid protein, the method comprising the steps of:
a. expressing a nucleic acid encoding the modified capsid protein under suitable conditions, and
b. isolating the expressed capsid protein of step a).

In some embodiments, a viral particle as described herein comprises a mosaic capsid, e.g., a capsid comprising capsid proteins genetically modified as described herein (in the absence or presence of a covalent bond with a targeting ligand) in a certain ratio with reference capsid proteins. A method for making such a mosaic viral particle comprises:
a. expressing a nucleic acid encoding the modified capsid protein and a nucleotide encoding a reference capsid protein at a ratio (wt/wt) of at least about 60:1 to about 1:60, e.g., 2:1, 1:1, 3:5, 1:2, 1:3, etc. under suitable conditions, and
b. isolating the expressed capsid protein of step a).

In some embodiments, a composition described herein comprises, or a method described herein combines, a modified cap gene: reference cap gene (or combination of reference cap genes) at a ratio that ranges from at least about 1:60 to about 60:1, e.g., 2:1, 1:1, 3:5, 1:2, 1:3, etc. In some embodiments, the ratio is at least about 1:2. In some embodiments, the ratio is at least about 1:3. In some embodiments, the ratio is at least about 1:4. In some embodiments, the ratio is at least about 1:5. In some embodiments, the ratio is at least about 1:6. In some embodiments, the ratio is at least about 1:7. In some embodiments, the ratio is at least about 1:8. In some embodiments, the ratio is at least about 1:9. In some embodiments, the ratio is at least about 1:10. In some embodiments, the ratio is at least about 1:11. In some embodiments, the ratio is at least about 1:12. In some embodiments, the ratio is at least about 1:13. In some embodiments, the ratio is at least about 1:14. In some embodiments, the ratio is at least about 1:15. In some embodiments, the ratio is at least about 1:16. In some embodiments, the ratio is at least about 1:17. In some embodiments, the ratio is at least about 1:18. In some embodiments, the ratio is at least about 1:19. In some embodiments, the ratio is at least about 1:20. In some embodiments, the ratio is at least about 1:25. In some embodiments, the ratio is at least about 1:30. In some embodiments, the ratio is at least about 1:35. In some embodiments, the ratio is at least about 1:40. In some embodiments, the ratio is at least about 1:45. In some embodiments, the ratio is at least about 1:50. In some embodiments, the ratio is at least about 1:55. In some embodiments, the ratio is at least about 1:60. In some embodiments, the ratio is at least about 2:1. In some embodiments, the ratio is at least about 3:1. In some embodiments, the ratio is at least about 4:1. In some embodiments, the ratio is at least about 5:1. In some embodiments, the ratio is at least about 6:1. In some embodiments, the ratio is at least about 7:1. In some embodiments, the ratio is at least about 8:1. In some embodiments, the ratio is at least about 9:1. In some embodiments, the ratio is at least about 10:1. In some embodiments, the ratio is at least about 11:1. In some embodiments, the ratio is at least about 12:1. In some embodiments, the ratio is at least about 13:1. In some embodiments, the ratio is at least about 14:1. In some embodiments, the ratio is at least about 15:1. In some embodiments, the ratio is at least about 16:1. In some embodiments, the ratio is at least about 17:1. In some embodiments, the ratio is at least about 18:1. In some embodiments, the ratio is at least about 19:1. In some embodiments, the ratio is at least about 20:1. In some embodiments, the ratio is at least about 25:1. In some embodiments, the ratio is at least about 30:1. In some embodiments, the ratio is at least about 35:1. In some embodiments, the ratio is at least about 40:1. In some embodiments, the ratio is at least about 45:1. In some embodiments, the ratio is at least about 50:1. In some embodiments, the ratio is at least about 55:1. In some embodiments, the ratio is at least about 60:1.

In some embodiments, VP protein subunit ratios in the mosaic viral particle may, but do not necessarily, stoichiometrically reflect the ratios of modified cap gene:reference cap gene. As a non-limiting exemplary embodiment, a mosaic capsid formed according to the method may be considered to, but does not necessarily, have a modified capsid protein:reference capsid protein ratio similar to the ratio (wt:wt) of nucleic acids encoding same used to produce the mosaic capsid. In some embodiments, a mosaic capsid comprises a protein subunit ratio of about 1:59 to about 59:1.

Further embodiments of the present invention is a method for altering the tropism of a virus, the method comprising the steps of: (a) inserting a nucleic acid encoding an amino acid sequence into a nucleic acid sequence encoding an viral capsid protein to form a nucleotide sequence encoding a genetically modified capsid protein comprising the amino acid sequence and/or (b) culturing a packaging cell in conditions sufficient for the production of viral particles, wherein the packaging cell comprises the nucleic acid. A further embodiment of the present invention is a method for displaying a targeting ligand on the surface of a capsid protein, the method comprising the steps of: (a) expressing a nucleic acid encoding a modified viral capsid protein as described herein (and optionally with a nucleotide encoding a reference capsid protein) under suitable conditions, wherein the nucleic acid encodes a capsid protein comprising a first member of a specific binding pair, (b) isolating the expressed capsid protein comprising a first member of a specific binding pair of step (a) or capsid comprising same, and (c) incubating the capsid protein or capsid with a second cognate member of the specific binding pair under conditions suitable for allowing the formation of an isopeptide bond between the first and second member, wherein the second cognate member of the specific binding pair is fused with a targeting ligand.

In some embodiments, the packaging cell further comprises a helper plasmid and/or a transfer plasmid comprising a nucleotide of interest. In some embodiments, the methods further comprise isolating self-complementary adeno-associated viral particles from culture supernatant. In some embodiments, the methods further comprise lysing the packaging cell and isolating single-stranded adeno-associated viral particles from the cell lysate. In some embodiments, the methods further comprise (a) clearing cell debris, (b) treating the supernatant containing viral particles with nucleases, e.g., DNase I and MgCl₂, (c) concentrating viral particles, (d) purifying the viral particles, and (e) any combination of (a)-(d).

Packaging cells useful for production of the viral particles described herein include, e.g., animal cells permissive for the virus, or cells modified to be permissive for the virus; or the packaging cell construct, for example, with the use of a transformation agent such as calcium phosphate. Non-limiting examples of packaging cell lines useful for producing viral particles described herein include, e.g., human embryonic kidney 293 (HEK-293) cells (e.g., American Type Culture Collection [ATCC] No. CRL-1573), HEK-293 cells that contain the SV40 Large T-antigen (HEK-293T or 293T), HEK293T/17 cells, human sarcoma cell line HT-1080 (CCL-121), lymphoblast-like cell line Raji (CCL-86), glioblastoma-astrocytoma epithelial-like cell line U87-MG (HTB-14), T-lymphoma cell line HuT78 (TIB-161), NIH/3T3 cells, Chinese Hamster Ovary cells (CHO) (e.g., ATCC Nos. CRL9618, CCL61, CRL9096), HeLa cells (e.g., ATCC No. CCL-2), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, CAP cells, CAP-T cells, and the like.

L929 cells, the FLY viral packaging cell system outlined in Cosset et al (1995) J Virol 69, 7430-7436, NS0 (murine myeloma) cells, human amniocytic cells (e.g., CAP, CAP-T), yeast cells (including, but not limited to, S. cerevisiae, Pichia pastoris), plant cells (including, but not limited to, Tobacco NT1, BY-2), insect cells (including but not limited to SF9, S2, SF21, Tni (e.g. High 5)) or bacterial cells (including, but not limited to, E. coli).

For additional packaging cells and systems, packaging techniques and particles for packaging the nucleic acid genome into the pseudotyped viral particle see, for example, Polo, et al, Proc Natl Acad Sci USA, (1999) 96:4598-4603. Methods of packaging include using packaging cells that permanently express the viral components, or by transiently transfecting cells with plasmids.

Further embodiments include methods comprising contacting a modified Cap protein as described herein with the targeting vector in conditions sufficient to operably link the modified Cap protein with the targeting vector, e.g., in conditions sufficient to promote association of the targeting vector to the modified Cap protein, e.g., via chemical linkage and/or association of first and second members of a specific binding pair, wherein the first member is inserted into the modified Cap protein the first member and the targeting vector is fused to the second member of the specific binding pair.

Further embodiments include methods of redirecting a virus and/or delivering a reporter or therapeutic gene to a target cell, the method comprising a method for transducing cells in vitro (e.g., ex vivo) or in vivo, the method comprising the steps of: contacting the target cell with a viral particle comprising a capsid described herein, wherein the capsid comprises a targeting ligand that specifically binds a receptor expressed by the target cell. In some embodiments, the target cell is in vitro (e.g., ex vivo). In other embodiments, the target cell is in vivo in a subject, e.g., a human.

### Target Cells

A wide variety of cells may be targeted in order to deliver a nucleotide of interest using a modified viral particle as disclosed herein. The target cells will generally be chosen based upon the nucleotide of interest and the desired effect.

In some embodiments, a nucleotide of interest may be delivered to enable a target cell to produce a protein that makes up for a deficiency in an organism, such as an enzymatic deficiency, or immune deficiency, such as X-linked severe combined immunodeficiency. Thus, in some embodiments, cells that would normally produce the protein in the animal are targeted. In other embodiments, cells in the area in which a protein would be most beneficial are targeted.

In other embodiments, a nucleotide of interest, such as a gene encoding an siRNA, may inhibit expression of a particular gene in a target cell. The nucleotide of interest may, for example, inhibit expression of a gene involved in a pathogen life cycle. Thus, cells susceptible to infection from the pathogen or infected with the pathogen may be targeted. In other embodiments, a nucleotide of interest may inhibit expression of a gene that is responsible for production of a toxin in a target cell.

In other embodiments a nucleotide of interest may encode a toxic protein that kills cells in which it is expressed. In this case, tumor cells or other unwanted cells may be targeted.

In still other embodiments a nucleotide of interest that encodes a therapeutic protein.

Once a particular population of target cells is identified in which expression of a nucleotide of interest is desired, a target receptor is selected that is specifically expressed on that population of target cells. The target receptor may be expressed exclusively on that population of cells or to a greater extent on that population of cells than on other populations of cells. The more specific the expression, the more specifically delivery can be directed to the target cells. Depending on the context, the desired amount of specificity of the marker (and thus of the gene delivery) may vary. For example, for introduction of a toxic gene, a high specificity is most preferred to avoid killing non-targeted cells. For expression of a protein for harvest, or expression of a secreted product where a global impact is desired, less marker specificity may be needed.

As discussed above, the target receptor may be any receptor for which a targeting ligand can be identified or created. Preferably the target receptor is a peptide or polypeptide, such as a receptor. However, in other embodiments the target receptor may be a carbohydrate or other molecule that can be recognized by a binding partner. If a binding partner, e.g., ligand, for the target receptor is already known, it may be used as the affinity molecule. However, if a binding molecule is not known, antibodies to the target receptor may be generated using standard procedures. The antibodies can then be used as a targeting ligand.

Thus, target cells may be chosen based on a variety of factors, including, for example, (1) the application (e.g., therapy, expression of a protein to be collected, and conferring disease resistance) and (2) expression of a marker with the desired amount of specificity.

Target cells are not limited in any way and include both germline cells and cell lines and somatic cells and cell lines. When the target cells are germline cells, the target cells are preferably selected from the group consisting of single-cell embryos and embryonic stem cells (ES).

### Therapeutic Formulation and Administration

Also described herein are pharmaceutical compositions comprising the antigen-binding molecules as described herein. In some embodiments, pharmaceutical compositions may be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antigen-binding molecule administered to a patient may vary depending upon the age and the size of the patient, target disease, conditions, route of administration, and the like. The preferred dose is typically calculated according to body weight or body surface area. When an antigen-binding molecule as described herein is used for therapeutic purposes in an adult patient, it may be advantageous to intravenously administer the antigen-binding molecule as described herein normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a bispecific antigen-binding molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition as described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition as described herein can be delivered subcutaneously or intravenously with a standard needle and syringe. In addition, with respect to subcutaneous delivery, a pen delivery device readily has applications in delivering a pharmaceutical composition as described herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition as described herein. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition as described herein include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic and Diagnostic Uses Thereof

Disclosed herein are also methods comprising administering to a subject in need thereof a therapeutic composition comprising an anti-hCACNG1 antibody, antigen-binding fragment thereof or an antibody-drug conjugate comprising an anti-hCACNG1 antibody (e.g., an anti-hCACNG1 antibody, or ADC comprising any of the HCVR/LCVR or CDR sequences as set forth in Table 1 herein). The therapeutic composition can comprise any of the anti-hCACNG1 antibodies, antigen-binding fragments thereof, or ADCs disclosed herein, and a pharmaceutically acceptable carrier or diluent.

The antibodies, antigen-binding fragment thereof, or an antibody-drug conjugate comprising an anti-hCACNG1 antibody as described herein may be useful, inter alia, for the treatment, prevention and/or amelioration of any disease or disorder associated with skeletal muscle tissue. For example, the antibodies and ADCs as described herein may be useful for the treatment of muscle wasting disorders (e.g., cachexia, glucocorticoid-induced muscle loss, heart failure induced muscle loss, HIV wasting, disuse, aging, etc.) and/or muscular dystrophies/myopathies.

The anti-hCACNG1 antibodies as described herein have various utilities. For example, in some embodiments, anti-hCACNG1 antibodies as described herein may be used in diagnostic assays for CACNG1, e.g., detecting its expression in specific cells, tissues, etc., e.g., as a reagent to identify/label skeletal muscle fibers. Various diagnostic and prognostic assay techniques known in the art may be used, such as competitive binding assays, direct or indirect sandwich assays and immunoprecipitation assays conducted in either heterogeneous or homogeneous phases (Zola (1987) Monoclonal Antibodies: A Manual of Techniques, CRC Press, Inc. pp. 147-1581). The antibodies used in the assays can be labeled with a detectable moiety. The detectable moiety should be capable of producing, either directly or indirectly, a detectable signal. Any method known in the art for conjugating the antibody to the detectable moiety may be employed.

In another embodiment, provided is a method of treatment of a disease, such as a muscle wasting disorder. The method may include the step of providing an antibody or CACNG1 antigen-binding fragment thereof, as described above, to a subject requiring said treatment.

### Example 1: Exemplary CACNG1 Antibodies

### Generation of Anti-Human CACNG1 Antibodies

Anti-human CACNG1 antibodies were obtained by immunizing a mouse (e.g., an engineered mouse comprising DNA encoding human immunoglobulin heavy and human kappa light chain variable regions), with human CACNG1.

Following immunization, splenocytes were harvested from each mouse and either (1) fused with mouse myeloma cells to preserve their viability and form hybridoma cells and screened for human CACNG1 specificity, or (2) B-cell sorted (as described in US 2007/0280945A1) using a either a human CACNG1 fragment as the sorting reagent that binds and identifies reactive antibodies (antigen-positive B cells).

Chimeric antibodies to human CACNG1 were initially isolated having a human variable region and a mouse constant region using, e.g., VELOCIMMUNE technology as described in U.S. Pat. Nos. 7,105,348; 8,642,835; and 9,622,459, each of which is incorporated herein by reference.

In some antibodies, for testing purposes, mouse constant regions were replaced with a desired human constant region, for example wild-type human CH or modified human CH (e.g. IgG1, IgG2 or IgG4 isotypes), and light chain constant region (CL), to generate a fully human anti-hCACNG1 antibody, or antigen binding portion thereof. While the constant region selected may vary according to specific use, high affinity antigen-binding and target specificity characteristics reside in the variable region.

Certain biological properties of the exemplary anti-human CACNG1 antibodies generated in accordance with the methods of this Example are described in detail in the Examples set forth below.

### Heavy and Light Chain Variable Region Amino Acid and Nucleic Acid Sequences of Anti-hCACNG1 Antibodies

Table 1 sets forth sequence identifiers of a nucleic acid (NA) sequence encoding, and in parentheses an amino acid (AA) sequence of, a heavy or light chain variable region (HCVR or LCVR, respectively), or a heavy or light chain CDR (HCDR and LCDR, respectively) of selected anti-hCACNG1 antibodies used to generate the therapeutic anti-hCACNG1 proteins disclosed herein.

TABLE 1
anti-hCACNG1 Sequence Identifiers

| | SEQ ID NOs: | | | | | | | |
|---|---|---|---|---|---|---|---|---|
| | HCVR | HCDR1 | HCDR2 | HCDR3 | LCVR | LCDR1 | LCDR2 | LCDR3 |
| Antibody | NA | NA | NA | NA | NA | NA | NA | NA |
| Designation | (AA) | (AA) | (AA) | (AA) | (AA) | (AA) | (AA) | (AA) |
| 1 | 1 | 3 | 5 | 7 | 9 | 11 | 13 | 15 |
| 31929/14647/10728 | (2) | (4) | (6) | (8) | (10) | (12) | (14) | (16) |
| 2 | 17 | 19 | 21 | 23 | 25 | 27 | 29 | 31 |
| 14570/10715 | (18) | (20) | (22) | (24) | (26) | (28) | (30) | (32) |
| 3 | 33 | 35 | 37 | 39 | 41 | 43 | 45 | 47 |
| 14572/10717 | (34) | (36) | (38) | (40) | (42) | (44) | (46) | (48) |
| 4 | 49 | 51 | 53 | 55 | 57 | 59 | 61 | 63 |
| 14571/10716 | (50) | (52) | (54) | (56) | (58) | (60) | (62) | (64) |
| 5 | 65 | 67 | 69 | 71 | 73 | 75 | 77 | 79 |
| 14574/10783 | (66) | (68) | (70) | (72) | (74) | (76) | (78) | (80) |
| 6 | 81 | 83 | 85 | 87 | 89 | 91 | 93 | 95 |
| 31944 | (82) | (84) | (86) | (88) | (90) | (92) | (94) | (96) |
| 7 | 97 | 99 | 101 | 103 | 105 | 107 | 109 | 111 |
| 31265/5972 | (98) | (100) | (102) | (104) | (106) | (108) | (110) | (112) |
| 8 | 113 | 115 | 117 | 119 | 121 | 123 | 125 | 127 |
| 31941 | (114) | (116) | (118) | (120) | (122) | (124) | (126) | (128) |
| 9 | 129 | 131 | 133 | 135 | 137 | 139 | 141 | 143 |
| 7660 | (130) | (132) | (134) | (136) | (138) | (140) | (142) | (144) |
| 10 | 145 | 147 | 149 | 151 | 153 | 155 | 157 | 159 |
| 9909 | (146) | (148) | (150) | (152) | (154) | (156) | (158) | (160) |
| 11 | 161 | 163 | 165 | 167 | 169 | 171 | 173 | 175 |
| 14573/10713 | (162) | (164) | (166) | (168) | (170) | (172) | (174) | (176) |
| 12 | 177 | 179 | 181 | 183 | 185 | 187 | 189 | 191 |
| 7854 | (178) | (180) | (182) | (184) | (186) | (188) | (190) | (192) |

31929/10728 (wildtype hIgG1)/14647 (hIgG1 N180Q)
HCVR Nucleic Acid Sequence (SEQ ID NO: 1)
CAGGTGCAGCTGGTGGAGTCTGGGGGAGGCGTGGTCCAGCCTGGGAGGTCCCTGAGA
CTCTCCTGTACAGCGTCTGGAATCACCTTCAGAAATTATGGCATGCACTGGGTCCGCCA
GGCTCCAGGCAAGGGGCTGGAGTGGGTGGCAGTTATGTGGTATGATGGAAGTAATAA
GTACTATGCAGACTCCGTGAAGGGCCGTTTCACCATCTCCGGAGACAATTCCAAGGTG
TATCTGCAAATGAACAGCCTGAGAGCCGAGGACACGGCTGTATATTACTGTGCGAGAA
GGGGCACTATAAGAACAGCTGCCCCTTTTGACTACTGGGGTCAGGGAACCCTGGTCAC
CGTCTCCTCA
HCVR Amino Acid Sequence (SEQ ID NO: 2)
QVQLVESGGGVVQPGRSLRLSCTASGITFRNYGMHWVRQAPGKGLEWVAVMWYDGSNK
YYADSVKGRFTISGDNSKVYLQMNSLRAEDTAVYYCARRGTIRTAAPFDYWGQGTLVTV
SS
HCDR1 Nucleic Acid Sequence (SEQ ID NO: 3)
GGA ATC ACC TTC AGA AAT TAT GGC
HCDR1 Amino Acid Sequence (SEQ ID NO: 4)
G I T F R N Y G
HCDR2 Nucleic Acid Sequence (SEQ ID NO: 5)
ATG TGG TAT GAT GGA AGT AAT AAG
HCDR2 Amino Acid Sequence (SEQ ID NO: 6)
M W Y D G S N K
HCDR3 Nucleic Acid Sequence (SEQ ID NO: 7)
GCG AGA AGG GGC ACT ATA AGA ACA GCT GCC CCT TTT GAC TAC
HCDR3 Amino Acid Sequence (SEQ ID NO: 8)
A R R G T I R T A A P F D Y
LCVR Nucleic Acid Sequence (SEQ ID NO: 9)
GACATCCAGATGACCCAGTCTCCATCCTCCCTGTCTGCATCTGTAGGAGACAGAGTCA
CCATCACTTGCCGGGCAAGTCAGAGCATTAGCAGCTATTTAAATTGGTATCAGCAGAA
ACCAGGGAAAGCCCCTAAGCTCCTGATCTATGCTGCATCCAGTTTGCAAAGTGGGGTC
CCGTCAAGGTTCAGTGGCAGTGGATCTGGGACAGATTTCACTCTCACCATCAGCAGTC
TGCAACCTGAAGATTTTGCAACTTACTACTGTCAACAGAGTTACAGTACCCCTCCGATC
ACCTTCGGCCAAGGGACACGACTGGAGATTAAA
LCVR Amino Acid Sequence (SEQ ID NO: 10)
DIQMTQSPSSLSASVGDRVTITCRASQSISSYLNWYQQKPGKAPKLLIYAASSLQSGVPSRF
SGSGSGTDFTLTISSLQPEDFATYYCQQSYSTPPITFGQGTRLEIK
LCDR1 Nucleic Acid Sequence (SEQ ID NO: 11)
CAG AGC ATT AGC AGC TAT
LCDR1 Amino Acid Sequence (SEQ ID NO: 12)
Q S I S S Y
LCDR2 Nucleic Acid Sequence (SEQ ID NO: 13)
GCT GCA TCC
LCDR2 Amino Acid Sequence (SEQ ID NO: 14)
A A S
LCDR3 Nucleic Acid Sequence (SEQ ID NO: 15)
CAA CAG AGT TAC AGT ACC CCT CCG ATC ACC
LCDR3 Amino Acid Sequence (SEQ ID NO: 16)
Q Q S Y S T P P I T
HC Nucleic Acid Sequence (SEQ ID NO: 193)
CAGGTGCAGCTGGTGGAGTCTGGGGGAGGCGTGGTCCAGCCTGGGAGGTCCCTGAGA
CTCTCCTGTACAGCGTCTGGAATCACCTTCAGAAATTATGGCATGCACTGGGTCCGCCA
GGCTCCAGGCAAGGGGCTGGAGTGGGTGGCAGTTATGTGGTATGATGGAAGTAATAA
GTACTATGCAGACTCCGTGAAGGGCCGTTTCACCATCTCCGGAGACAATTCCAAGGTG
TATCTGCAAATGAACAGCCTGAGAGCCGAGGACACGGCTGTATATTACTGTGCGAGAA
GGGGCACTATAAGAACAGCTGCCCCTTTTGACTACTGGGGTCAGGGAACCCTGGTCAC
CGTCTCCTCAGCCTCCACCAAGGGCCCATCGGTCTTCCCCCTGGCGCCCTGCTCCAGGA
GCACCTCCGAGAGCACAGCCGCCCTGGGCTGCCTGGTCAAGGACTACTTCCCCGAACC
GGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGGT
GGACAAGAGAGTTGAGTCCAAATATGGTCCCCCATGCCCACCGTGCCCAGCACCAGGC
GGTGGCGGACCATCAGTCTTCCTGTTCCCCCCAAAACCCAAGGACACTCTCATGATCTC
CCGGACCCCTGAGGTCACGTGCGTGGTGGTGGACGTGAGCCAGGAAGACCCCGAGGT
CCAGTTCAACTGGTACGTGGATGGCGTGGAGGTGCATAATGCCAAGACAAAGCCGCG
GGAGGAGCAGTTCAACAGCACGTACCGTGTGGTCAGCGTCCTCACCGTCCTGCACCAG
GACTGGCTGAACGGCAAGGAGTACAAGTGCAAGGTCTCCAACAAAGGCCTCCCGTCCT
CCATCGAGAAAACCATCTCCAAAGCCAAAGGGCAGCCCCGAGAGCCACAGGTGTA
CACCCTGCCCCCATCCCAGGAGGAGATGACCAAGAACCAGGTCAGCCTGACCTGCCTG
GTCAAAGGCTTCTACCCCAGCGACATCGCCGTGGAGTGGGAGAGCAATGGGCAGCCG
GAGAACAACTACAAGACCACGCCTCCCGTGCTGGACTCCGACGGC
TCCTTCTTCCTCTACAGCAGGCTCACCGTGGACAAGAGCAGGTGGCAGGAGGGGAATG
TCTTCTCATGCTCCGTGATGCATGAGGCTCTGCACAACCACTACACACAGAAGTCCCTC
TCCCTGTCTCTGGGTAAATGA
HC Amino Acid Sequence (SEQ ID NO: 194)
QVQLVESGGGVVQPGRSLRLSCTASGITFRNYGMHWVRQAPGKGLEWVAVMWYDGSNK
YYADSVKGRFTISGDNSKVYLQMNSLRAEDTAVYYCARRGTIRTAAPFDYWGQGTLVTV
SSASTKGPSVFPLAPCSRSTSESTAALGCLVKDYFPEPVTVSWNSGALTSGVHTFPAVLQSS
GLYSLSSVVTVPSSSL
GTKTYTCNVDHKPSNTKVDKRVESKYGPPCPPCPAPGGGGPSVFLFPPKPKDTLMISRTPE
VTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYT
QKSLSLSLGK
*underlined and bolded asparagine (N) may be mutated to a glutamine (Q) for
conjugation by transglutaminase, see, e.g., SEQ ID NO: 269
LC Nucleic Acid Sequence (SEQ ID NO: 195)
GACATCCAGATGACCCAGTCTCCATCCTCCCTGTCTGCATCTGTAGGAGACAGAGTCA
CCATCACTTGCCGGGCAAGTCAGAGCATTAGCAGCTATTTAAATTGGTATCAGCAGAA
ACCAGGGAAAGCCCCTAAGCTCCTGATCTATGCTGCATCCAGTTTGCAAAGTGGGGTC
CCGTCAAGGTTCAGTGGCAGTGGATCTGGGACAGATTTCACTCTCACCATCAGCAGTC
TGCAACCTGAAGATTTTGCAACTTACTACTGTCAACAGAGTTACAGTACCCCTCCGATC
ACCTTCGGCCAAGGGACACGACTGGAGATTAAACGAACTGTGGCTGCACCATCTGTCT
TCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACG
CCTGCGAAGTCACCCATCAGGGCCTGAGCTCGCCCGTCACAAAGAGCTTCAACAGGGG
AGAGTGTTAG
LC Amino Acid Sequence (SEQ ID NO: 196)
DIQMTQSPSSLSASVGDRVTITCRASQSISSYLNWYQQKPGKAPKLLIYAASSLQSGVPSRF
SGSGSGTDFTLTISSLQPEDFATYYCQQSYSTPPITFGQGTRLEIKRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNRGEC
10715 (wildtype hIgG1)/14570 (IgG1 N180Q):
HCVR Nucleic Acid Sequence (SEQ ID NO: 17)
CAGGTGCAGCTACAGCAGTGGGGCGCAGGACTGTTGAAGCCTTCGGCGACCCTGTCCC
GCACCTGCGCTGTCTATGGTGGGTCCTTCAGTGGTTACTACTGGAACTGGATCCGCCAG
TCCCCAGGGAAGGGGCTGGAATGGATTGGGGAAATCCTTCATAGTGGAAGAACCAAC
TACAACCCGTCCCTCAAGAGTCGAGTCACCATATCAGTAGACACGTCCAAGAACCAGT
TCTCCCTGAAGCTGACCTCTGTGACCGCCGCGGACACGGCTGTATATTACTGTGCGGG
AAGGATAGCAGCTCGTCACGGCTGGTTCGACCCCTGGGGCCAGGGAACCCTGGTCACC
GTCTCCTCA
HCVR Amino Acid Sequence (SEQ ID NO: 18)
QVQLQQWGAGLLKPSATLSRTCAVYGGSFSGYYWNWIRQSPGKGLEWIGEILHSGRTNY
NPSLKSRVTISVDTSKNQFSLKLTSVTAADTAVYYCAGRIAARHGWFDPWGQGTLVTVSS
HCDR1 Nucleic Acid Sequence (SEQ ID NO: 19)
GGT GGG TCC TTC AGT GGT TAC TAC
HCDR1 Amino Acid Sequence (SEQ ID NO: 20)
G G S F S G Y Y
HCDR2 Nucleic Acid Sequence (SEQ ID NO: 21)
ATC CTT CAT AGT GGA AGA ACC
HCDR2 Amino Acid Sequence (SEQ ID) NO: 22)
I L H S G R T
HCDR3 Nucleic Acid Sequence (SEQ ID NO: 23)
GCG GGA AGG ATA GCA GCT CGT CAC GGC TGG TTC GAC CCC
HCDR3 Amino Acid Sequence (SEQ ID NO: 24)
A G R I A A R H G W F D P
LCVR Nucleic Acid Sequence (SEQ ID NO: 25)
GACATCCAGATGACCCAGTCTCCATCTTCCGTGTCTACATCTGTAGGAGACAGAGTCA
CCATCTCTTGTCGGGCGAGTCAGGATATTCGCAAGTGGTTAGCCTGGTATCAACAGAA
ACCAGGAAAAGCCCCTAAACTCCTGATCTATGCTACATCCAGTTTGCAAAGTGGGGTC
CCTTCAAGGTTCAGCGGCAGTGGATCTGGGACAGATTTCACTCTCACCATCAGCAGCC
TGCAGCCTGAGGATTTTGCAACTTACTTTTGTCAACAGGCTAACAGTTTCCCGTTCACT
TTTGGCCAGGGGACCAAGCTGGAGATCAAA
LCVR Amino Acid Sequence (SEQ ID NO: 26)
DIQMTQSPSSVSTSVGDRVTISCRASQDIRKWLAWYQQKPGKAPKLLIYATSSLQSGVPSRF
SGSGSGTDFTLTISSLQPEDFATYFCQQANSFPFTFGQGTKLEIK
LCDR1 Nucleic Acid Sequence (SEQ ID NO: 27)
CAG GAT ATT CGC AAG TGG
LCDR1 Amino Acid Sequence (SEQ ID NO: 28)
Q D I R K W
LCDR2 Nucleic Acid Sequence (SEQ ID NO: 29)
GCT ACA TCC
LCDR2 Amino Acid Sequence (SEQ ID NO: 30)
A T S
LCDR3 Nucleic Acid Sequence (SEQ ID NO: 31)
CAA CAG GCT AAC AGT TTC CCG TTC ACT
LCDR3 Amino Acid Sequence (SEQ ID NO: 32)
Q Q A N S F P F T
HC Nucleic Acid Sequence (SEQ ID NO: 197)
CAGGTGCAGCTACAGCAGTGGGGCGCAGGACTGTTGAAGCCTTCGGCGACCCTGTCCC
GCACCTGCGCTGTCTATGGTGGGTCCTTCAGTGGTTACTACTGGAACTGGATCCGCCAG
TCCCCAGGGAAGGGGCTGGAATGGATTGGGGAAATCCTTCATAGTGGAAGAACCAAC
TACAACCCGTCCCTCAAGAGTCGAGTCACCATATCAGTAGACACGTCCAAGAACCAGT
TCTCCCTGAAGCTGACCTCTGTGACCGCCGCGGACACGGCTGTATATTACTGTGCGGG
AAGGATAGCAGCTCGTCACGGCTGGTTCGACCCCTGGGGCCAGGGAACCCTGGTCACC
GTCTCCTCAGCCTCCACCAAGGGCCCATCGGTCTTCCCCCTGGCGCCCTGCTCCAGGAG
CACCTCCGAGAGCACAGCCGCCCTGGGCTGCCTGGTCAAGGACTACTTCCCCGAACCG
GTGACGGTGTCGTGGAACTCAGGCGCCCTGACCAGCGGCGTGCACACCTTCCCGGCTG
TCCTACAGTCCTCAGGACTCTACTCCCTCAGCAGCGTGGTGACCGTGCCCTCCAGCAGC
TTGGGCACGAAGACCTACACCTGCAACGTAGATCACAAGCCCAGCAACACCAAGGTG
GACAAGAGAGTTGAGTCCAAATATGGTCCCCCATGCCCACCCTGCCCAGCACCTGAGT
TCCTGGGGGGACCATCAGTCTTCCTGTTCCCCCCAAAACCCAAGGACACTCTCATGATC
TCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCA
GGACTGGCTGAACGGCAAGGAGTACAAGTGCAAGGTCTCCAACAAAGGCCTCCCGTC
CTCCATCGAGAAAACCATCTCCAAAGCCAAAGGGCAGCCCCGAGAGCCACAGGT
GTACACCCTGCCCCCATCCCAGGAGGAGATGACCAAGAACCAGGTCAGCCTGACCTGC
CTGGTCAAAGGCTTCTACCCCAGCGACATCGCCGTGGAGTGGGAGAGCAATGGGCAGC
CGGAGAACAACTACAAGACCACGCCTCCCGTGCTGGACTCCGACGGCTCCTTCTTCCT
CTACAGCAGGCTCACCGTGGACAAGAGCAGGTGGCAGGAGGGGAATGTCTTCTCATGC
TCCGTGATGCATGAGGCTCTGCACAACCACTACACACAGAAGTCCCTCTCCCTGTCTCT
GGGTAAATGA
HC Amino Acid Sequence (SEQ ID NO: 198)
QVQLQQWGAGLLKPSATLSRTCAVYGGSFSGYYWNWIRQSPGKGLEWIGEILHSGRTNY
NPSLKSRVTISVDTSKNQFSLKLTSVTAADTAVYYCAGRIAARHGWFDPWGQGTLVTVSS
ASTKGPSVFPLAPCSRSTSESTAALGCLVKDYFPEPVTVSWNSGALTSGVHTFPAVLQSSGL
YSLSSVVTVPSSSL
GTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYT
QKSLSLSLGK
*underlined and bolded asparagine (N) may be mutated to a glutamine (Q) for
conjugation by transglutaminase, see, e.g., SEQ ID NO: 269
LC Nucleic Acid Sequence (SEQ ID NO: 199)
GACATCCAGATGACCCAGTCTCCATCTTCCGTGTCTACATCTGTAGGAGACAGAGTCA
CCATCTCTTGTCGGGCGAGTCAGGATATTCGCAAGTGGTTAGCCTGGTATCAACAGAA
ACCAGGAAAAGCCCCTAAACTCCTGATCTATGCTACATCCAGTTTGCAAAGTGGGGTC
CCTTCAAGGTTCAGCGGCAGTGGATCTGGGACAGATTTCACTCTCACCATCAGCAGCC
TGCAGCCTGAGGATTTTGCAACTTACTTTTGTCAACAGGCTAACAGTTTCCCGTTCACT
TTTGGCCAGGGGACCAAGCTGGAGATCAAACGAACTGTGGCTGCACCATCTGTCTTCA
TCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAAT
CGGGTAACTCCCAGGAGAGTGTCACAGAGCAGGACAGCAAGGACAGCACCTACAGCC
TCAGCAGCACCCTGACGCTGAGCAAAGCAGACTACGAGAAACACAAAGTCTACGCCT
GCGAAGTCACCCATCAGGGCCTGAGCTCGCCCGTCACAAAGAGCTTCAACAGGGGAG
AGTGTTAG
LC Amino Acid Sequence (SEQ ID NO: 200)
DIQMTQSPSSVSTSVGDRVTISCRASQDIRKWLAWYQQKPGKAPKLLIYATSSLQSGVPSRF
SGSGSGTDFTLTISSLQPEDFATYFCQQANSFPFTFGQGTKLEIKRTVAAPSVFIFPPSDEQLK
SGTASVVCLLNNFYPREAKVQWKVDNALQSGNSQESVTEQDSKDSTYSLSSTLTLSKADY
EKHKVYACEVTHQGLSSPVTKSFNRGEC
10717 (wildtype hIgG1)/14572 (hIgG1 N180Q)
HCVR Nucleic Acid Sequence (SEQ ID NO: 33)
CAGGTGCAGCTGGTGGAGTCTGGGGGAGGCGTGGTCCAGCCTGGGAGGTCCCTGAGA
CTCTCCTGTGCAGCGTCTGGATTCACCTTCAGTACATATGGCATGCACTGGGTCCGCCA
GGCTCCAGGCAAGGGGCTGGAGTGGGTGGCAGTTATTTGGCATGATGGAAGTGATAA
ATATTATGTAGACTCCGTGAAGGGCCGATTCTCCATCGCCAGAGACAATTCCAAGAAC
ACGCTTTATCTGCAAATGAATAGTCTGAGAGTCGAGGACACGGGTATATATTACTGTG
CGAGAAGGGGTATACGTGGAACCGTTTTTGACCACTGGGGCCTGGGAACCCTGGTCAC
CGTCTCCTCA
HCVR Amino Acid Sequence (SEQ ID NO: 34)
QVQLVESGGGVVQPGRSLRLSCAASGFTFSTYGMHWVRQAPGKGLEWVAVIWHDGSDK
YYVDSVKGRFSIARDNSKNTLYLQMNSLRVEDTGIYYCARRGIRGTVFDHWGLGTLVTVS
S
HCDR1 Nucleic Acid Sequence (SEQ ID NO: 35)
GGA TTC ACC TTC AGT ACA TAT GGC
HCDR1 Amino Acid Sequence (SEQ ID NO: 36)
G F T F S T Y G
HCDR2 Nucleic Acid Sequence (SEQ ID NO: 37)
ATT TGG CAT GAT GGA AGT GAT AAA
HCDR2 Amino Acid Sequence (SEQ ID NO: 38)
I W H D G S D K
HCDR3 Nucleic Acid Sequence (SEQ ID NO: 39)
GCG AGA AGG GGT ATA CGT GGA ACC GTT TTT GAC CAC
HCDR3 Amino Acid Sequence (SEQ ID NO: 40)
A R R G I R G T V F D H
LCVR Nucleic Acid Sequence (SEQ ID NO: 41)
GACATCCAGATGACCCAGTCTCCTTCCACCCTGTCTGCATCTGTAGGAGACAGAGTCA
CCCTCACTTGTCGGGCCAGTCAGAGTATTAGTAACAAGTTGGCCTGGTATCAGCAGAA
ACCAGGGAAAGCCCCTAACCTCCTGATCTATAAGGCGTCTAATTTAGAAAGTGGGGTC
CCATCAAGGTTCAGCGGCAGTGGATCTGGGACAGAATTCACTCTCACCATCAGCAGCC
TGCAGCCTGATGATTTTGCAACTTATTACTGCCAACAGTATAATAGTTATTCGTGGACG
TTCGGCCAAGGGACCAAGGTGGAAATCAAA
LCVR Amino Acid Sequence (SEQ ID NO: 42)
DIQMTQSPSTLSASVGDRVTLTCRASQSISNKLAWYQQKPGKAPNLLIYKASNLESGVPSR
FSGSGSGTEFTLTISSLQPDDFATYYCQQYNSYSWTFGQGTKVEIK
LCDR1 Nucleic Acid Sequence (SEQ ID NO: 43)
CAG AGT ATT AGT AAC AAG
LCDR1 Amino Acid Sequence (SEQ ID NO: 44)
Q S I S N K
LCDR2 Nucleic Acid Sequence (SEQ ID NO: 45)
AAG GCG TCT
LCDR2 Amino Acid Sequence (SEQ ID NO: 46)
K A S
LCDR3 Nucleic Acid Sequence (SEQ ID NO: 47)
CAA CAG TAT AAT AGT TAT TCG TGG ACG
LCDR3 Amino Acid Sequence (SEQ ID NO: 48)
Q Q Y N S Y S W T
HC Nucleic Acid Sequence (SEQ ID NO: 201)
CAGGTGCAGCTGGTGGAGTCTGGGGGAGGCGTGGTCCAGCCTGGGAGGTCCCTGAGA
CTCTCCTGTGCAGCGTCTGGATTCACCTTCAGTACATATGGCATGCACTGGGTCCGCCA
GGCTCCAGGCAAGGGGCTGGAGTGGGTGGCAGTTATTTGGCATGATGGAAGTGATAA
ATATTATGTAGACTCCGTGAAGGGCCGATTCTCCATCGCCAGAGACAATTCCAAGAAC
ACGCTTTATCTGCAAATGAATAGTCTGAGAGTCGAGGACACGGGTATATATTACTGTG
CGAGAAGGGGTATACGTGGAACCGTTTTTGACCACTGGGGCCTGGGAACCCTGGTCAC
CGTCTCCTCAGCCTCCACCAAGGGCCCATCGGTCTTCCCCCTGGCGCCCTGCTCCAGGA
GCACCTCCGAGAGCACAGCCGCCCTGGGCTGCCTGGTCAAGGACTACTTCCCCGAACC
GGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAG
CTTGGGCACGAAGACCTACACCTGCAACGTAGATCACAAGCCCAGCAACACCAAGGT
GGACAAGAGAGTTGAGTCCAAATATGGTCCCCCATGCCCACCCTGCCCAGCACCTGAG
TTCCTGGGGGGACCATCAGTCTTCCTGTTCCCCCCAAAACCCAAGGACACTCTCATGAT
CTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGC
GGGAGGAGCAGTTCAACAGCACGTACCGTGTGGTCAGCGTCCTCACCGTCCTGCACCA
GGACTGGCTGAACGGCAAGGAGTACAAGTGCAAGGTCTCCAACAAAGGCCTCCCGTC
CTCCATCGAGAAAACCATCTCCAAAGCCAAAGGGCAGCCCCGAGAGCCACAGGTGTA
CACCCTGCCCCCATCCCAGGAGGAGATGACCAAGAACCAGGTCAGCCTGACCTGCCTG
GTCAAAGGCTTCTACCCCAGCGACATCGCCGTGGAGTGGGAGAGCAATGGGCAGCCG
GAGAACAACTACAAGACCACGCCTCCCGTGCTGGACTCCGACGGCTCCTTCTTCCTCTA
CAGCAGGCTCACCGTGGACAAGAGCAGGTGGCAGGAGGGGAATGTCTTCTCATGCTCC
GTGATGCATGAGGCTCTGCACAACCACTACACACAGAAGTCCCTCTCCCTGTCTCTGG
GTAAATGA
HC Amino Acid Sequence (SEQ ID NO: 202)
QVQLVESGGGVVQPGRSLRLSCAASGFTFSTYGMHWVRQAPGKGLEWVAVIWHDGSDK
YYVDSVKGRFSIARDNSKNTLYLQMNSLRVEDTGIYYCARRGIRGTVFDHWGLGTLVTVS
SASTKGPSVFPLAPCSRSTSESTAALGCLVKDYFPEPVTVSWNSGALTSGVHTFPAVLQSSG
LYSLSSVVTVPSSSL
GTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYT
QKSLSLSLGK
*underlined and bolded asparagine (N) may be mutated to a glutamine (Q) for
conjugation by transglutaminase, see, e.g., SEQ ID NO: 269
LC Nucleic Acid Sequence (SEQ ID NO: 203)
GACATCCAGATGACCCAGTCTCCTTCCACCCTGTCTGCATCTGTAGGAGACAGAGTCA
CCCTCACTTGTCGGGCCAGTCAGAGTATTAGTAACAAGTTGGCCTGGTATCAGCAGAA
ACCAGGGAAAGCCCCTAACCTCCTGATCTATAAGGCGTCTAATTTAGAAAGTGGGGTC
CCATCAAGGTTCAGCGGCAGTGGATCTGGGACAGAATTCACTCTCACCATCAGCAGCC
TGCAGCCTGATGATTTTGCAACTTATTACTGCCAACAGTATAATAGTTATTCGTGGACG
TTCGGCCAAGGGACCAAGGTGGAAATCAAACGAACTGTGGCTGCACCATCTGTCTTCA
TCTTCCCGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCCTCCAAT
CGGGTAACTCCCAGGAGAGTGTCACAGAGCAGGACAGCAAGGACAGCACCTACAGCC
TCAGCAGCACCCTGACGCTGAGCAAAGCAGACTACGAGAAACACAAAGTCTACGCCT
GCGAAGTCACCCATCAGGGCCTGAGCTCGCCCGTCACAAAGAGCTTCAACAGGGGAG
AGTGTTAG
LC Amino Acid Sequence (SEQ ID NO: 204)
DIQMTQSPSTLSASVGDRVTLTCRASQSISNKLAWYQQKPGKAPNLLIYKASNLESGVPSR
FSGSGSGTEFTLTISSLQPDDFATYYCQQYNSYSWTFGQGTKVEIKRTVAAPSVFIFPPSDEQ
LKSGTASVVCLLNNFYPREAKVQWKVDNALQSGNSQESVTEQDSKDSTYSLSSTLTLSKA
DYEKHKVYACEVTHQGLSSPVTKSFNRGEC
10716 (wildtype hIgG1)/14571 (hlgG1 N180Q)
HCVR Nucleic Acid Sequence (SEQ ID NO: 49)
CAGGTGCAGCTGCAGGAGTCGGGCCCAGGACTGGTGAAGCCTTCGGAGACCCTGTCCC
TCACCTGCACTGTCTCTGGTGACTCCATCAATAATTACTACTGGACCTGGCTCCGGCAG
CCCCCAGGGAAGGGACTGGAGTGGATTGGTTATATCTATTACAGTGGGAGCGCCAACT
ACAACCCCTCCCTCAAGAGTCGAGTCACCATATCAGTAGACACGTCCAAGAACCAGTT
CTCCCTGAAGCTAAATTCTGTGACCGCTGCGGACACGGCCGTGTATTACTGTGCGAGA
GGGGCGGTCAAGTACTTCCGGCATTGGGGCCAGGGCACCCTGGTCACCGTCTCCTCA
HCVR Amino Acid Sequence (SEQ ID NO: 50)
QVQLQESGPGLVKPSETLSLTCTVSGDSINNYYWTWLRQPPGKGLEWIGYIYYSGSANYNP
SLKSRVTISVDTSKNQFSLKLNSVTAADTAVYYCARGAVKYFRHWGQGTLVTVSS
HCDR1 Nucleic Acid Sequence (SEQ ID NO: 51)
GGT GAC TCC ATC AAT AAT TAC TAC
HCDR1 Amino Acid Sequence (SEQ ID NO: 52)
G D S I N N Y Y
HCDR2 Nucleic Acid Sequence (SEQ ID NO: 53)
ATC TAT TAC AGT GGG AGC GCC
HCDR2 Amino Acid Sequence (SEQ ID NO: 54)
I Y Y S G S A
HCDR3 Nucleic Acid Sequence (SEQ ID NO: 55)
GCG AGA GGG GCG GTC AAG TAC TTC CGG CAT
HCDR3 Amino Acid Sequence (SEQ ID NO: 56)
A R G A V K Y F R H
LCVR Nucleic Acid Sequence (SEQ ID NO: 57)
GAAATTGTGTTGACGCAGTCTCCGGGCACCCTCTCTTTGTCTCCAGGGGAAAGAGCCA
CCCTCTCCTGCAGGGCCAGTCAGACTATTAACCACAACAACTTAGCCTGGTACCAGCA
GAGACCTGGCCAGGCTCCCAGGCTCCTCATCTATGGTGCATCCAACAGGGCCACTGCC
ATCCCAGACAGGTTCAGTGGCAGTGGGTCTGGGACAGACTTCACTCTCACCATCAGCA
GACTGGAGCCTGAAGATTTTGAAGTGTATTCTTGTCAGCAGTATGGTAGCTTGCCGCTC
ACTTTCGGCGGAGGGACCAAGGTGGAGATCAAA
LCVR Amino Acid Sequence (SEQ ID NO: 58)
EIVLTQSPGTLSLSPGERATLSCRASQTINHNNLAWYQQRPGQAPRLLIYGASNRATAIPDR
FSGSGSGTDFTLTISRLEPEDFEVYSCQQYGSLPLTFGGGTKVEIK
LCDR1 Nucleic Acid Sequence (SEQ ID NO: 59)
CAG ACT ATT AAC CAC AAC AAC
LCDR1 Amino Acid Sequence (SEQ ID NO: 60)
Q T I N H N N
LCDR2 Nucleic Acid Sequence (SEQ ID NO: 61)
GGT GCA TCC
LCDR2 Amino Acid Sequence (SEQ ID NO: 62)
G A S
LCDR3 Nucleic Acid Sequence (SEQ ID NO: 63)
CAG CAG TAT GGT AGC TTG CCG CTC ACT
LCDR3 Amino Acid Sequence (SEQ ID NO: 64)
Q Q Y G S L P L T
HC Nucleic Acid Sequence (SEQ ID NO: 205)
CAGGTGCAGCTGCAGGAGTCGGGCCCAGGACTGGTGAAGCCTTCGGAGACCCTGTCCC
TCACCTGCACTGTCTCTGGTGACTCCATCAATAATTACTACTGGACCTGGCTCCGGCAG
CCCCCAGGGAAGGGACTGGAGTGGATTGGTTATATCTATTACAGTGGGAGCGCCAACT
ACAACCCCTCCCTCAAGAGTCGAGTCACCATATCAGTAGACACGTCCAAGAACCAGTT
CTCCCTGAAGCTAAATTCTGTGACCGCTGCGGACACGGCCGTGTATTACTGTGCGAGA
GGGGCGGTCAAGTACTTCCGGCATTGGGGCCAGGGCACCCTGGTCACCGTCTCCTCAG
CCTCCACCAAGGGCCCATCGGTCTTCCCCCTGGCGCCCTGCTCCAGGAGCACCTCCGA
GAGCACAGCCGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGCAGCTTGGGCAC
GAAGACCTACACCTGCAACGTAGATCACAAGCCCAGCAACACCAAGGTGGACAAGAG
AGTTGAGTCCAAATATGGTCCCCCATGCCCACCCTGCCCAGCACCTGAGTTCCTGGGG
GGACCATCAGTCTTCCTGTTCCCCCCAAAACCCAAGGACACTCTCATGATCTCCCGGAC
CCCTGAGGTCACGTGCGTGGTGGTGGACGTGAGCCAGGAAGACCCCGAGGTCCAGTTC
AACTGGTACGTGGATGGCGTGGAGGTGCATAATGCCAAGACAAAGCCGCGGGAGGAG
CAGTTCAACAGCACGTACCGTGTGGTCAGCGTCCTCACCGTCCTGCACCAGGACTGGC
TGAACGGCAAGGAGTACAAGTGCAAGGTCTCCAACAAAGGCCTCCCGTCCTCCATCGA
GAAAACCATCTCCAAAGCCAAAGGGCAGCCCCGAGAGCCACAGGTGTACACCCTGCC
CCCATCCCAGGAGGAGATGACCAAGAACCAGGTCAGCCTGACCTGCCTGGTCAAAGG
CTTCTACCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAGGC
TCACCGTGGACAAGAGCAGGTGGCAGGAGGGGAATGTCTTCTCATGCTCCGTGATGCA
TGAGGCTCTGCACAACCACTACACACAGAAGTCCCTCTCCCTGTCTCTGGGTAAATGA
HC Amino Acid Sequence (SEQ ID NO: 206)
QVQLQESGPGLVKPSETLSLTCTVSGDSINNYYWTWLRQPPGKGLEWIGYIYYSGSANYNP
SLKSRVTISVDTSKNQFSLKLNSVTAADTAVYYCARGAVKYFRHWGQGTLVTVSSASTKG
PSVFPLAPCSRSTSESTAALGCLVKDYFPEPVTVSWNSGALTSGVHTFPAVLQSSGLYSLSS
VVTVPSSSL
GTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYT
QKSLSLSLGK
*underlined and bolded asparagine (N) may be mutated to a glutamine (Q) for
conjugation by transglutaminase, see, e.g., SEQ ID NO: 269
LC Nucleic Acid Sequence (SEQ ID NO: 207)
GAAATTGTGTTGACGCAGTCTCCGGGCACCCTCTCTTTGTCTCCAGGGGAAAGAGCCA
CCCTCTCCTGCAGGGCCAGTCAGACTATTAACCACAACAACTTAGCCTGGTACCAGCA
GAGACCTGGCCAGGCTCCCAGGCTCCTCATCTATGGTGCATCCAACAGGGCCACTGCC
ATCCCAGACAGGTTCAGTGGCAGTGGGTCTGGGACAGACTTCACTCTCACCATCAGCA
GACTGGAGCCTGAAGATTTTGAAGTGTATTCTTGTCAGCAGTATGGTAGCTTGCCGCTC
ACTTTCGGCGGAGGGACCAAGGTGGAGATCAAACGAACTGTGGCTGCACCATCTGTCT
TCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACG
CCTGCGAAGTCACCCATCAGGGCCTGAGCTCGCCCGTCACAAAGAGCTTCAACAGGGG
AGAGTGTTAG
LC Amino Acid Sequence (SEQ ID NO: 208)
EIVLTQSPGTLSLSPGERATLSCRASQTINHNNLAWYQQRPGQAPRLLIYGASNRATAIPDR
FSGSGSGTDFTLTISRLEPEDFEVYSCQQYGSLPLTFGGGTKVEIKRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNRGEC
10783 (wildtype hIgG1)/14574 (hIgG1 N180Q)
HCVR Nucleic Acid Sequence (SEQ ID NO: 65)
CAGGTGCAGCTGGTGGAGTCTGGGGGAGGCGTGGTCCAGCCTGGGACGTCCCTGAGAC
TCTCCTGTGCAGCGTCAGGATTCACCTTCAGTAGCTATGGCATGCACTGGGTCCGCCAG
GCTCCAGGCAAGGGGCTGGAGTGGGTGGCAGTTATATGGATTGATGGAAGTAATAAAT
ATTATGCAGACTCCGTGAAGGGCCGATTCACCATCTCCAGAGACAATTCCAAGAACAC
GCTGTATCTGCAAATGAACAGCCTGAGAGCCGAGGACACGGCTGTGTATTACTGTGCG
AGAAGGGGGGGTATAGTAGTAGCTGCCCCCTTTGACTACTGGGGCCAGGGAACCCTGG
TCACCGTCTCCTCA
HCVR Amino Acid Sequence (SEQ ID NO: 66)
QVQLVESGGGVVQPGTSLRLSCAASGFTFSSYGMHWVRQAPGKGLEWVAVIWIDGSNKY
YADSVKGRFTISRDNSKNTLYLQMNSLRAEDTAVYYCARRGGIVVAAPFDYWGQGTLVT
VSS
HCDR1 Nucleic Acid Sequence (SEQ ID NO: 67)
GGA TTC ACC TTC AGT AGC TAT GGC
HCDR1 Amino Acid Sequence (SEQ ID NO: 68)
G F T F S S Y G
HCDR2 Nucleic Acid Sequence (SEQ ID NO: 69)
ATA TGG ATT GAT GGA AGT AAT AAA
HCDR2 Amino Acid Sequence (SEQ ID NO: 70)
I W I D G S N K
HCDR3 Nucleic Acid Sequence (SEQ ID NO: 71)
GCG AGA AGG GGG GGT ATA GTA GTA GCT GCC CCC TTT GAC TAC
HCDR3 Amino Acid Sequence (SEQ ID NO: 72)
A R R G G I V V A A P F D Y
LCVR Nucleic Acid Sequence (SEQ ID NO: 73)
GACATCCAGATGACCCAGTCTCCATCCTCCCTGTCTGCATCTGTAGGAGACAGAGTCA
CCATCACTTGCCGGGCAAGTCAGAGCATTAGCAGCTATTTAAATTGGTATCAGCAGAA
ACCAGGGAAAGCCCCTAAGCTCCTGATCTATGCTGCATCCAGTTTGCAAAGTGGGGTC
CCGTCAAGGTTCAGTGGCAGTGGATCTGGGACAGATTTCACTCTCACCATCAGCAGTC
TGCAACCTGAAGATTTTGCAACTTACTACTGTCAACAGAGTTACAGTACCCCTCCGATC
ACCTTCGGCCAAGGGACACGACTGGAGATTAAA
LCVR Amino Acid Sequence (SEQ ID NO: 74)
DIQMTQSPSSLSASVGDRVTITCRASQSISSYLNWYQQKPGKAPKLLIYAASSLQSGVPSRF
SGSGSGTDFTLTISSLQPEDFATYYCQQSYSTPPITFGQGTRLEIK
LCDR1 Nucleic Acid Sequence (SEQ ID NO: 75)
CAG AGC ATT AGC AGC TAT
LCDR1 Amino Acid Sequence (SEQ ID NO: 76)
Q S I S S Y
LCDR2 Nucleic Acid Sequence (SEQ ID NO: 77)
GCT GCA TCC
LCDR2 Amino Acid Sequence (SEQ ID NO: 78)
A A S
LCDR3 Nucleic Acid Sequence (SEQ ID NO: 79)
CAA CAG AGT TAC AGT ACC CCT CCG ATC ACC
LCDR3 Amino Acid Sequence (SEQ ID NO: 80)
Q Q S Y S T P P I T
HC Nucleic Acid Sequence (SEQ ID NO: 209)
CAGGTGCAGCTGGTGGAGTCTGGGGGAGGCGTGGTCCAGCCTGGGACGTCCCTGAGAC
TCTCCTGTGCAGCGTCAGGATTCACCTTCAGTAGCTATGGCATGCACTGGGTCCGCCAG
GCTCCAGGCAAGGGGCTGGAGTGGGTGGCAGTTATATGGATTGATGGAAGTAATAAAT
ATTATGCAGACTCCGTGAAGGGCCGATTCACCATCTCCAGAGACAATTCCAAGAACAC
GCTGTATCTGCAAATGAACAGCCTGAGAGCCGAGGACACGGCTGTGTATTACTGTGCG
AGAAGGGGGGGTATAGTAGTAGCTGCCCCCTTTGACTACTGGGGCCAGGGAACCCTGG
TCACCGTCTCCTCAGCCTCCACCAAGGGCCCATCGGTCTTCCCCCTGGCGCCCTGCTCC
AGGAGCACCTCCGAGAGCACAGCCGCCCTGGGCTGCCTGGTCAAGGACTACTTCCCCG
AACCGGTGACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACCA
AGGTGGACAAGAGAGTTGAGTCCAAATATGGTCCCCCATGCCCACCCTGCCCAGCACC
TGAGTTCCTGGGGGGACCATCAGTCTTCCTGTTCCCCCCAAAACCCAAGGACACTCTCA
TGATCTCCCGGACCCCTGAGGTCACGTGCGTGGTGGTGGACGTGAGCCAGGAAGACCC
CGAGGTCCAGTTCAACTGGTACGTGGATGGCGTGGAGGTGCATAATGCCAAGACAAA
GCCGCGGGAGGAGCAGTTCAACAGCACGTACCGTGTGGTCAGCGTCCTCACCGTCCTG
CACCAGGACTGGCTGAACGGCAAGGAGTACAAGTGCAAGGTCTCCAACAAAGGCCTC
CCGTCCTCCATCGAGAAAACCATCTCCAAAGCCAAAGGGCAGCCCCGAGAGCCACAG
GTGTACACCCTGCCCCCATCCCAGGAGGAGATGACCAAGAACCAGGTCAGCCTGACCT
GCCTGGTCAAAGGCTTCTACCCCAGCGACATCGCCGTGGAGTGGGAGAGCAATGGGCA
GCCGGAGAACAACTACAAGACCACGCCTCCCGTGCTGGACTCCGACGGCTCCTTCTTC
CTCTACAGCAGGCTCACCGTGGACAAGAGCAGGTGGCAGGAGGGGAATGTCTTCTCAT
GCTCCGTGATGCATGAGGCTCTGCACAACCACTACACACAGAAGTCCCTCTCCCTGTCT
CTGGGTAAATGA
HC Amino Acid Sequence (SEQ ID NO: 210)
QVQLVESGGGVVQPGTSLRLSCAASGFTFSSYGMHWVRQAPGKGLEWVAVIWIDGSNKY
YADSVKGRFTISRDNSKNTLYLQMNSLRAEDTAVYYCARRGGIVVAAPFDYWGQGTLVT
VSSASTKGPSVFPLAPCSRSTSESTAALGCLVKDYFPEPVTVSWNSGALTSGVHTFPAVLQS
SGLYSLSSVVTVPSSSL
GTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYT
QKSLSLSLGK
*underlined and bolded asparagine (N) may be mutated to a glutamine (Q) for
conjugation by transglutaminase, see, e.g., SEQ ID NO: 269
LC Nucleic Acid Sequence (SEQ ID NO: 211)
GACATCCAGATGACCCAGTCTCCATCCTCCCTGTCTGCATCTGTAGGAGACAGAGTCA
CCATCACTTGCCGGGCAAGTCAGAGCATTAGCAGCTATTTAAATTGGTATCAGCAGAA
ACCAGGGAAAGCCCCTAAGCTCCTGATCTATGCTGCATCCAGTTTGCAAAGTGGGGTC
CCGTCAAGGTTCAGTGGCAGTGGATCTGGGACAGATTTCACTCTCACCATCAGCAGTC
TGCAACCTGAAGATTTTGCAACTTACTACTGTCAACAGAGTTACAGTACCCCTCCGATC
ACCTTCGGCCAAGGGACACGACTGGAGATTAAACGAACTGTGGCTGCACCATCTGTCT
TCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACG
CCTGCGAAGTCACCCATCAGGGCCTGAGCTCGCCCGTCACAAAGAGCTTCAACAGGGG
AGAGTGTTAG
LC Amino Acid Sequence (SEQ ID NO: 212)
DIQMTQSPSSLSASVGDRVTITCRASQSISSYLNWYQQKPGKAPKLLIYAASSLQSGVPSRF
SGSGSGTDFTLTISSLQPEDFATYYCQQSYSTPPITFGQGTRLEIKRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNRGEC
31944
HCVR Nucleic Acid Sequence (SEQ ID NO: 81)
CAG GTG CAG TTG GTG GAG TCT GGG GGA GGC GTG GTC CAG CCT GGG AGG TCC
CTG AGA CTC TCC TGT GAA GCG TCT GGA ATC ACC TTC AGA AAC TAT GGC ATG
CAC TGG GTC CGC CAG GCT CCA GGC AAG GGG CTG GAG TGG GTG GCA GTT ATG
TGG TAT GAT GGA AGT AAT AAA TAC TAC GCA GAC TCC GTG AAG GGC CGA TTC
ACC ATC TCC AGA GAC AAT TCC AAG AAC ACG GTG TAT CTG CAA ATG AAC AGC
CTG AGA GCC GAA GAC ACG GCT GTG TAT TAC TGT GCG AGA CGG GGT CAT ATA
GCA ACA GCT GCT CCC TTT GAC TAC TGG GGC CAG GGA ACC CTG GTC ACC GTC
TCC TCA
HCVR Amino Acid Sequence (SEQ ID NO: 82)
QVQLVESGGGVVQPGRSLRLSCEASGITFRNYGMHWVRQAPGKGLEWVAVMWYDGSNK
YYADSVKGRFTISRDNSKNTVYLQMNSLRAEDTAVYYCARRGHIATAAPFDYWGQGTLV
TVSS
HCDR1 Nucleic Acid Sequence (SEQ ID NO: 83)
GGA ATC ACC TTC AGA AAC TAT GGC
HCDR1 Amino Acid Sequence (SEQ ID NO: 84)
G I T F R N Y G
HCDR2 Nucleic Acid Sequence (SEQ ID NO: 85)
atg tgg tat gat gga agt aat aaa
HCDR2 Amino Acid Sequence (SEQ ID NO: 86)
M W Y D G S N
HCDR3 Nucleic Acid Sequence (SEQ ID NO: 87)
GCG AGA CGG GGT CAT ATA GCA ACA GCT GCT CCC TTT GAC TAC
HCDR3 Amino Acid Sequence (SEQ ID NO: 88)
A R R G H I A T A A P F D
LCVR Nucleic Acid Sequence (SEQ ID NO: 89)
GACATCCAGATGACCCAGTCTCCATCCTCCCTGTCTGCATCCGTAGGAGACAGAGTCA
CCATCAGTTGCCGGGCAAGTCAGAGCATTAGTAGTTATTTAAATTGGTATCAGCAGAA
ACCAGGGAAAGCCCCTAAGGTCCTGATGTATGCTGCATCCAGTTTGCAAAGTGGGGTC
CCATCAAGGTTCAGTGGCAGTGGATCTGGGACAGATTTCACTCTCACCATCAGCAGTC
TGCAACCTGAGGATTTTGCAACTTACTACTGTCAACAGAGTTACAGTACCCCTCCGATC
ACCTTCGGCCAAGGGACACGACTGGAGATTAAA
LCVR Amino Acid Sequence (SEQ ID NO: 90)
DIQMTQSPSSLSASVGDRVTISCRASQSISSYLNWYQQKPGKAPKVLMYAASSLQSGVPSR
FSGSGSGTDFTLTISSLQPEDFATYYCQQSYSTPPITFGQGTRLEIK
LCDR1 Nucleic Acid Sequence (SEQ ID NO: 91)
CAG AGC ATT AGT AGT TAT
LCDR1 Amino Acid Sequence (SEQ ID NO: 92)
Q S I S S Y
LCDR2 Nucleic Acid Sequence (SEQ ID NO: 93)
GCT GCA TCC
LCDR2 Amino Acid Sequence (SEQ ID NO: 94)
A A S
LCDR3 Nucleic Acid Sequence (SEQ ID NO: 95)
CAA CAG AGT TAC AGT ACC CCT CCG ATC ACC
LCDR3 Amino Acid Sequence (SEQ ID NO: 96)
Q Q S Y S T P P I T
HC Nucleic Acid Sequence (SEQ ID NO: 213)
CAG GTG CAG TTG GTG GAG TCT GGG GGA GGC GTG GTC CAG CCT GGG AGG TCC
CTG AGA CTC TCC TGT GAA GCG TCT GGA ATC ACC TTC AGA AAC TAT GGC ATG
CAC TGG GTC CGC CAG GCT CCA GGC AAG GGG CTG GAG TGG GTG GCA GTT ATG
TGG TAT GAT GGA AGT AAT AAA TAC TAC GCA GAC TCC GTG AAG GGC CGA TTC
ACC ATC TCC AGA GAC AAT TCC AAG AAC ACG GTG TAT CTG CAA ATG AAC AGC
CTG AGA GCC GAA GAC ACG GCT GTG TAT TAC TGT GCG AGA CGG GGT CAT ATA
GCA ACA GCT GCT CCC TTT GAC TAC TGG GGC CAG GGA ACC CTG GTC ACC GTC
TCC TCA
GCCAAAACAACAGCCCCATCGGTCTATCCACTGGCCCCTGTGTGTGGAGATACAACTG
GCTCCTCGGTGACTCTAGGATGCCTGGTCAAGGGTTATTTCCCTGAGCCAGTGACCTTG
ACCTGGAACTCTGGATCCCTGTCCAGTGGTGTGCACACCTTCCCAGCTGTCCTGCAGTC
TGACCTCTACACCCTCAGCAGCTCAGTGACTGTAACCTCGAGCACCTGGCCCAGCCAG
TCCATCACCTGCAATGTGGCCCACCCGGCAAGCAGCACCAAGGTGGACAAGAAAATTG
AGCCCAGAGGGCCCACAATCAAGCCCTGTCCTCCATGCAAATGCCCAGCACCTAACCT
CTTGGGTGGACCATCCGTCTTCATCTTCCCTCCAAAGATCAAGGATGTACTCATGATCT
CCCTGAGCCCCATAGTCACATGTGTGGTGGTGGATGTGAGCGAGGATGACCCAGATGT
CCAGATCAGCTGGTTTGTGAACAACGTGGAAGTACACACAGCTCAGACACAAACCCAT
AGAGAGGATTACAACAGTACTCTCCGGGTGGTCAGTGCCCTCCCCATCCAGCACCAGG
ACTGGATGAGTGGCAAGGAGTTCAAATGCAAGGTCAACAACAAAGACCTCCCAGCGC
CCATCGAGAGAACCATCTCAAAACCCAAAGGGTCAGTAAGAGCTCCACAGGTATATGT
CTTGCCTCCACCAGAAGAAGAGATGACTAAGAAACAGGTCACTCTGACCTGCATGGTC
ACAGACTTCATGCCTGAAGACATTTACGTGGAGTGGACCAACAACGGGAAAACAGAG
CTAAACTACAAGAACACTGAACCAGTCCTGGACTCTGATGGTTCTTACTTCATGTACAG
CAAGCTGAGAGTGGAAAAGAAGAACTGGGTGGAAAGAAATAGCTACTCCTGTTCAGT
GGTCCACGAGGGTCTGCACAATCACCACACGACTAAGAGCTTCTCCCGGACTCCGGGT
AAATGA
HC Amino Acid Sequence (SEQ ID NO: 214)
QVQLVESGGG VVQPGRSLRL SCEASGITFR NYGMHWVRQA PGKGLEWVAV
MWYDGSNKYY ADSVKGRFTI SRDNSKNTVY LQMNSLRAED TAVYYCARRG
HIATAAPFDY WGQGTLVTVS S
AKTTAPSVYPLAPVCGDTTGSSVTLGCLVKGYFPEPVTLTWNSGSLSSGVHTFPAVLQSDL
YTLSSSVTVTSSTWPSQSITCNVAHPASSTKVDKKIEPRGPTIKPCPPCKCPAPNLLGGPSVFI
FPPKIKDVLMISLSPIVTCVVVDVSEDDPDVQISWFVNNVEVHTAQTQTHREDYNSTLRVV
SALPIQHQDWMSGKEFKCKVNNKDLPAPIERTISKPKGSVRAPQVYVLPPPEEEMTKKQVT
LTCMVTDFMPEDIYVEWTNNGKTELNYKNTEPVLDSDGSYFMYSKLRVEKKNWVERNSY
SCSVVHEGLHNHHTTKSFSRTPGK
LC Nucleic Acid Sequence (SEQ ID NO: 215)
GACATCCAGATGACCCAGTCTCCATCCTCCCTGTCTGCATCCGTAGGAGACAGAGTCA
CCATCAGTTGCCGGGCAAGTCAGAGCATTAGTAGTTATTTAAATTGGTATCAGCAGAA
ACCAGGGAAAGCCCCTAAGGTCCTGATGTATGCTGCATCCAGTTTGCAAAGTGGGGTC
CCATCAAGGTTCAGTGGCAGTGGATCTGGGACAGATTTCACTCTCACCATCAGCAGTC
TGCAACCTGAGGATTTTGCAACTTACTACTGTCAACAGAGTTACAGTACCCCTCCGATC
ACCTTCGGCCAAGGGACACGACTGGAGATTAAACGAGCTGATGCTGCACCAACTGTAT
CCATCTTCCCACCATCCAGTGAGCAGTTAACATCTGGAGGTGCCTCAGTCGTGTGCTTC
TTGAACAACTTCTACCCCAAAGACATCAATGTCAAGTGGAAGATTGATGGCAGTGAAC
GACAAAATGGCGTCCTGAACAGTTGGACTGATCAGGACAGCAAAGACAGCACCTACA
GCATGAGCAGCACCCTCACGTTGACCAAGGACGAGTATGAACGACATAACAGCTATAC
CTGTGAGGCCACTCACAAGACATCAACTTCACCCATTGTCAAGAGCTTCAACAGGGGA
GAGTGTTGA
LC Amino Acid Sequence (SEQ ID NO: 216)
DIQMTQSPSS LSASVGDRVT ISCRASQSIS SYLNWYQQKP GKAPKVLMYA
ASSLQSGVPS RFSGSGSGTD FTLTISSLQP EDFATYYCQQ SYSTPPITFG QGTRLEIK
RADAAPTVSIFPPSSEQLTSGGASVVCFLNNFYPKDINVKWKIDGSERQNGVLNSWTDQDS
KDSTYSMSSTLTLTKDEYERHNSYTCEATHKTSTSPIVKSFNRGEC
31265 (wildtype hIgG1)/5972 (hIgG1 N180Q)
HCVR Nucleic Acid Sequence (SEQ ID NO: 97)
CAGGTGCAGCTGGTGGAGTCTGGGGGAGGCGTGGTCCAGCCTGGGAGGTCCCTGAGA
CTCTCCTGTACAGCGTCTGGATTCACCTTCCGTTCCTATGGCATGCACTGGGTCCGCCA
GGCTCCAGGCAAGGGGCTGGAGTGGGTGTCAGTTATTTGGATTGATGGAAATAATATA
TACTATGCAGACTCCGTGAAGGGCCGATTCACCATCTCCAGAGACAATTCCAAGAACA
CGCTGTATCTGCAAATGGACAGCCTGAGAGCCGAGGACACGGCTGTTTATTACTGTGC
GAGAAGACTGGCTATAACATCAGCTGCCCCCTTTGACTACTGGGGCCAGGGAACCCTG
GTCACCGTCTCCTCA
HCVR Amino Acid Sequence (SEQ ID NO: 98)
QVQLVESGGGVVQPGRSLRLSCTASGFTFRSYGMHWVRQAPGKGLEWVSVIWIDGNNIY
YADSVKGRFTISRDNSKNTLYLQMDSLRAEDTAVYYCARRLAITSAAPFDYWGQGTLVTV
SS
HCDR1 Nucleic Acid Sequence (SEQ ID NO: 99)
GGA TTC ACC TTC CGT TCC TAT GGC
HCDR1 Amino Acid Sequence (SEQ ID NO: 100)
G F T F R S Y G
HCDR2 Nucleic Acid Sequence (SEQ ID NO: 101)
ATT TGG ATT GAT GGA AAT AAT ATA
HCDR2 Amino Acid Sequence (SEQ ID NO: 102)
I W I D G N N I
HCDR3 Nucleic Acid Sequence (SEQ ID NO: 103)
GCG AGA AGA CTG GCT ATA ACA TCA GCT GCC CCC TTT GAC TAC
HCDR3 Amino Acid Sequence (SEQ ID NO: 104)
A R R L A I T S A A P F D Y
LCVR Nucleic Acid Sequence (SEQ ID NO: 105)
GACATCCAGATGACCCAGTCTCCATCCTCCCTGTCTGCATCTGTAGGAGACAGAGTCA
CCATCACTTGCCGGGCAAGTCAGAGCATTAGCAGCTATTTAAATTGGTATCAGCAGAA
ACCAGGGAAAGCCCCTAAGCTCCTGATCTATGCTGCATCCAGTTTGCAAAGTGGGGTC
CCGTCAAGGTTCAGTGGCAGTGGATCTGGGACAGATTTCACTCTCACCATCAGCAGTC
TGCAACCTGAAGATTTTGCAACTTACTACTGTCAACAGAGTTACAGTACCCCTCCGATC
ACCTTCGGCCAAGGGACACGACTGGAGATTAAA
LCVR Amino Acid Sequence (SEQ ID NO: 106)
DIQMTQSPSSLSASVGDRVTITCRASQSISSYLNWYQQKPGKAPKLLIYAASSLQSGVPSRF
SGSGSGTDFTLTISSLQPEDFATYYCQQSYSTPPITFGQGTRLEIK
LCDR1 Nucleic Acid Sequence (SEQ ID NO: 107)
CAG AGC ATT AGC AGC TAT
LCDR1 Amino Acid Sequence (SEQ ID NO: 108)
Q S I S S Y
LCDR2 Nucleic Acid Sequence (SEQ ID NO: 109)
GCT GCA TCC
LCDR2 Amino Acid Sequence (SEQ ID NO: 110)
A A S
LCDR3 Nucleic Acid Sequence (SEQ ID NO: 111)
CAA CAG AGT TAC AGT ACC CCT CCG ATC ACC
LCDR3 Amino Acid Sequence (SEQ ID NO: 112)
Q Q S Y S T P P I T
HC Nucleic Acid Sequence (SEQ ID NO: 217)
CAGGTGCAGCTGGTGGAGTCTGGGGGAGGCGTGGTCCAGCCTGGGAGGTCCCTGAGA
CTCTCCTGTACAGCGTCTGGATTCACCTTCCGTTCCTATGGCATGCACTGGGTCCGCCA
GGCTCCAGGCAAGGGGCTGGAGTGGGTGTCAGTTATTTGGATTGATGGAAATAATATA
TACTATGCAGACTCCGTGAAGGGCCGATTCACCATCTCCAGAGACAATTCCAAGAACA
CGCTGTATCTGCAAATGGACAGCCTGAGAGCCGAGGACACGGCTGTTTATTACTGTGC
GAGAAGACTGGCTATAACATCAGCTGCCCCCTTTGACTACTGGGGCCAGGGAACCCTG
GTCACCGTCTCCTCAGCCTCCACCAAGGGCCCATCGGTCTTCCCCCTGGCGCCCTGCTC
CAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCC
AGCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGCAC
CTGAGTTCCTGGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAAA
GCCGCGGGAGGAGCAGTTCAACAGCACGTACCGTGTGGTCAGCGTCCTCACCGTCCTG
CACCAGGACTGGCTGAACGGCAAGGAGTACAAGTGCAAGGTCTCCAACAAAGGCCTC
CCGTCCTCCATCGAGAAAACCATCTCCAAAGCCAAAGGGCAGCCCCGAGAGCCACAG
GTGTACACCCTGCCCCCATCCCAGGAGGAGATGACCAAGAACCAGGTCAGCCTGACCT
GCCTGGTCAAAGGCTTCTACCCCAGCGACATCGCCGTGGAGTGGGAGAGCAATGGGCA
GCCGGAGAACAACTACAAGACCACGCCTCCCGTGCTGGACTCCGACGGCTCCTTCTTC
CTCTACAGCAGGCTCACCGTGGACAAGAGCAGGTGGCAGGAGGGGAATGTCTTCTCAT
GCTCCGTGATGCATGAGGCTCTGCACAACCACTACACACAGAAGTCCCTCTCCCTGTCT
CTGGGTAAATGA
HC Amino Acid Sequence (SEQ ID NO: 218)
QVQLVESGGGVVQPGRSLRLSCTASGFTFRSYGMHWVRQAPGKGLEWVSVIWIDGNNIY
YADSVKGRFTISRDNSKNTLYLQMDSLRAEDTAVYYCARRLAITSAAPFDYWGQGTLVTV
SSASTKGPSVFPLAPCSRSTSESTAALGCLVKDYFPEPVTVSWNSGALTSGVHTFPAVLQSS
GLYSLSSVVTVPSSSL
GTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYT
QKSLSLSLGK
*underlined and bolded asparagine (N) may be mutated to a glutamine (Q) for
conjugation by transglutaminase, see, e.g., SEQ ID NO: 269
LC Nucleic Acid Sequence (SEQ ID NO: 219)
GACATCCAGATGACCCAGTCTCCATCCTCCCTGTCTGCATCTGTAGGAGACAGAGTCA
CCATCACTTGCCGGGCAAGTCAGAGCATTAGCAGCTATTTAAATTGGTATCAGCAGAA
ACCAGGGAAAGCCCCTAAGCTCCTGATCTATGCTGCATCCAGTTTGCAAAGTGGGGTC
CCGTCAAGGTTCAGTGGCAGTGGATCTGGGACAGATTTCACTCTCACCATCAGCAGTC
TGCAACCTGAAGATTTTGCAACTTACTACTGTCAACAGAGTTACAGTACCCCTCCGATC
ACCTTCGGCCAAGGGACACGACTGGAGATTAAACGAACTGTGGCTGCACCATCTGTCT
TCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACG
CCTGCGAAGTCACCCATCAGGGCCTGAGCTCGCCCGTCACAAAGAGCTTCAACAGGGG
AGAGTGTTAG
LC Amino Acid Sequence (SEQ ID NO: 220)
DIQMTQSPSSLSASVGDRVTITCRASQSISSYLNWYQQKPGKAPKLLIYAASSLQSGVPSRF
SGSGSGTDFTLTISSLQPEDFATYYCQQSYSTPPITFGQGTRLEIKRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNRGEC
31941
HCVR Nucleic Acid Sequence (SEQ ID NO: 113)
CAGGTTCAGCTGGTGCAGTCTGGAGCTGAGGTGAAGAAGCCTGGGGCCTCAGTGAAG
GTCTCCTGCAAGGCTTCTGGTTACGCCTTCACCACCTATGGTATCACCTGGGTGCGACA
GGCCCCTGGACAAGGACTTGAGTGGATGGGATGGATCAGCGCTTACAATGGAAATAC
AAACTATGCAGAGAAGGTCCAGGGCAGATTCACCATGACCACAGACACATCCACGAA
TACAGCCTACATGGAGCTGAGGAGCCTGAGATCCGACGACACGGCCGTGTATTTCTGT
GCGAGAAAGGGTCACTATGGTTCGGGGACTTATTATAACCCCTTTGGTTTTGATTTTTG
GGGCCAAGGGACAATGGTCACCGTCTCTTCA
HCVR Amino Acid Sequence (SEQ ID NO: 114)
QVQLVQSGAEVKKPGASVKVSCKASGYAFTTYGITWVRQAPGQGLEWMGWISAYNGNT
NYAEKVQGRFTMTTDTSTNTAYMELRSLRSDDTAVYFCARKGHYGSGTYYNPFGFDFWG
QGTMVTVSS
HCDR1 Nucleic Acid Sequence (SEQ ID NO: 115)
ggt tac gcc ttc acc acc tat ggt
HCDR1 Amino Acid Sequence (SEQ ID NO: 116)
G Y A F T T Y G
HCDR2 Nucleic Acid Sequence (SEQ ID NO: 117)
atc agc gct tac aat gga aat aca
HCDR2 Amino Acid Sequence (SEQ ID NO: 118)
I S A Y N G N
HCDR3 Nucleic Acid Sequence (SEQ ID NO: 119)
GCG AGA AAG GGT CAC TAT GGT TCG GGG ACT TAT TAT AAC CCC TTT GGT TTT GAT
TTT
HCDR3 Amino Acid Sequence (SEQ ID NO: 120)
C A R K G H Y G S G T Y Y N P F G F D
LCVR Nucleic Acid Sequence (SEQ ID NO: 121)
GAAATTATGTTGATGCAGTCTCCAGGCACCCTGTCTTTGTCTCCAGGGGAAAGAGCCA
CCCTCTCCTGCAGGGCCAGTCAGAGTGTTAGCAGCAGCTACTTAGCCTGGTACCAACA
GAAACCTGGCCAGGCTCCCAGGCTCCTCATCTATGGTGCATCCAGCAGGGCCACTGAC
ATCCCAGACAGGTTCAGTGGCAGTGGGTCTGGGACAGACTTCACTCTCACCATCAGCA
GACTGGAGCCTGAAGATTTTGCAGTTTATTTCTGTCAGCAGTATTATGGCTCACCTTGG
ACGTTCGGCCAAGGGACCAAGGTGGAAATCAAG
LCVR Amino Acid Sequence (SEQ ID NO: 122)
EIMLMQSPGTLSLSPGERATLSCRASQSVSSSYLAWYQQKPGQAPRLLIYGASSRATDIPDR
FSGSGSGTDFTLTISRLEPEDFAVYFCQQYYGSPWTFGQGTKVEIK
LCDR1 Nucleic Acid Sequence (SEQ ID NO: 123)
cag agt gtt agc agc agc tac
LCDR1 Amino Acid Sequence (SEQ ID NO: 124)
Q S V S S S Y
LCDR2 Nucleic Acid Sequence (SEQ ID NO: 125)
ggt gca tcc
LCDR2 Amino Acid Sequence (SEQ ID NO: 126)
G A
LCDR3 Nucleic Acid Sequence (SEQ ID NO: 127)
cag cag tat tat ggc tca cct tgg acg
LCDR3 Amino Acid Sequence (SEQ ID NO: 128)
C Q Q Y Y G S P W
HC Nucleic Acid Sequence (SEQ ID NO: 221)
CAGGTTCAGCTGGTGCAGTCTGGAGCTGAGGTGAAGAAGCCTGGGGCCTCAGTGAAG
GTCTCCTGCAAGGCTTCTGGTTACGCCTTCACCACCTATGGTATCACCTGGGTGCGACA
GGCCCCTGGACAAGGACTTGAGTGGATGGGATGGATCAGCGCTTACAATGGAAATAC
AAACTATGCAGAGAAGGTCCAGGGCAGATTCACCATGACCACAGACACATCCACGAA
TACAGCCTACATGGAGCTGAGGAGCCTGAGATCCGACGACACGGCCGTGTATTTCTGT
GCGAGAAAGGGTCACTATGGTTCGGGGACTTATTATAACCCCTTTGGTTTTGATTTTTG
GGGCCAAGGGACAATGGTCACCGTCTCTTCAGCCAAAACGACACCCCCATCTGTCTAT
CCACTGGCCCCTGGATCTGCTGCCCAAACTAACTCCATGGTGACCCTGGGATGCCTGGT
CAAGGGCTATTTCCCTGAGCCAGTGACAGTGACCTGGAACTCTGGATCCCTGTCCAGC
GGTGTGCACACCTTCCCAGCTGTCCTGCAGTCTGACCTCTACACTCTGAGCAGCTCAGT
GACTGTCCCCTCCAGCACCTGGCCCAGCGAGACCGTCACCTGCAACGTTGCCCACCCG
GCCAGCAGCACCAAGGTGGACAAGAAAATTGTGCCCAGGGATTGTGGTTGTAAGCCTT
GCATATGTACAGTCCCAGAAGTATCATCTGTCTTCATCTTCCCCCCAAAGCCCAAGGAT
GTGCTCACCATTACTCTGACTCCTAAGGTCACGTGTGTTGTGGTAGACATCAGCAAGG
ATGATCCCGAGGTCCAGTTCAGCTGGTTTGTAGATGATGTGGAGGTGCACACAGCTCA
GACGCAACCCCGGGAGGAGCAGTTCAACAGCACTTTCCGCTCAGTCAGTGAACTTCCC
ATCATGCACCAGGACTGGCTCAATGGCAAGGAGTTCAAATGCAGGGTCAACAGTGCA
GCTTTCCCTGCCCCCATCGAGAAAACCATCTCCAAAACCAAAGGCAGACCGAAGGCTC
CACAGGTGTACACCATTCCACCTCCCAAGGAGCAGATGGCCAAGGATAAAGTCAGTCT
GACCTGCATGATAACAGACTTCTTCCCTGAAGACATTACTGTGGAGTGGCAGTGGAAT
GGGCAGCCAGCGGAGAACTACAAGAACACTCAGCCCATCATGGACACAGATGGCTCT
TACTTCGTCTACAGCAAGCTCAATGTGCAGAAGTCCAACTGGGAGGCAGGAAATACTT
TCACCTGCTCTGTGTTACATGAGGGCCTGCACAACCACCATACTGAGAAGTCCCTCTCC
CACTCTCCTGGTAAATGA
HC Amino Acid Sequence (SEQ ID NO: 222)
QVQLVQSGAEVKKPGASVKVSCKASGYAFTTYGITWVRQAPGQGLEWMGWISAYNGNT
NYAEKVQGRFTMTTDTSTNTAYMELRSLRSDDTAVYFCARKGHYGSGTYYNPFGFDFWG
QGTMVTVSSAKTTPPSVYPLAPGSAAQTNSMVTLGCLVKGYFPEPVTVTWNSGSLSSGVH
TFPAVLQSDLYTLSSSVTVPSSTWPSETVTCNVAHPASSTKVDKKIVPRDCGCKPCICTVPE
VSSVFIFPPKPKDVLTITLTPKVTCVVVDISKDDPEVQFSWFVDDVEVHTAQTQPREEQFNS
TFRSVSELPIMHQDWLNGKEFKCRVNSAAFPAPIEKTISKTKGRPKAPQVYTIPPPKEQMAK
DKVSLTCMITDFFPEDITVEWQWNGQPAENYKNTQPIMDTDGSYFVYSKLNVQKSNWEA
GNTFTCSVLHEGLHNHHTEKSLSHSPGK
LC Nucleic Acid Sequence (SEQ ID NO: 223)
GAAATTATGTTGATGCAGTCTCCAGGCACCCTGTCTTTGTCTCCAGGGGAAAGAGCCA
CCCTCTCCTGCAGGGCCAGTCAGAGTGTTAGCAGCAGCTACTTAGCCTGGTACCAACA
GAAACCTGGCCAGGCTCCCAGGCTCCTCATCTATGGTGCATCCAGCAGGGCCACTGAC
ATCCCAGACAGGTTCAGTGGCAGTGGGTCTGGGACAGACTTCACTCTCACCATCAGCA
GACTGGAGCCTGAAGATTTTGCAGTTTATTTCTGTCAGCAGTATTATGGCTCACCTTGG
ACGTTCGGCCAAGGGACCAAGGTGGAAATCAAGCGAGCTGATGCTGCACCAACTGTAT
CCATCTTCCCACCATCCAGTGAGCAGTTAACATCTGGAGGTGCCTCAGTCGTGTGCTTC
TTGAACAACTTCTACCCCAAAGACATCAATGTCAAGTGGAAGATTGATGGCAGTGAAC
GACAAAATGGCGTCCTGAACAGTTGGACTGATCAGGACAGCAAAGACAGCACCTACA
GCATGAGCAGCACCCTCACGTTGACCAAGGACGAGTATGAACGACATAACAGCTATAC
CTGTGAGGCCACTCACAAGACATCAACTTCACCCATTGTCAAGAGCTTCAACAGGGGA
GAGTGTTGA
LC Amino Acid Sequence (SEQ ID NO: 224)
EIMLMQSPGT LSLSPGERAT LSCRASQSVS SSYLAWYQQK PGQAPRLLIY GASSRATDIP
DRFSGSGSGT DFTLTISRLE PEDFAVYFCQ QYYGSPWTFG QGTKVEIK
RADAAPTVSIFPPSSEQLTSGGASVVCFLNNFYPKDINVKWKIDGSERQNGVLNSWTDQDS
KDSTYSMSSTLTLTKDEYERHNSYTCEATHKTSTSPIVKSFNRGEC
7660
HCVR Nucleic Acid Sequence (SEQ ID NO: 129)
GAGGTGCAGCTGGTGGAGTCTGGGGGAGGCTTGGTCCAGCCGGGGGGGTCCCTGAAA
CTCTCCTGTACAGCCTCTGGGTTGACCCTCAGTGACTCTGCTATGCACTGGGTCCGCCA
GGCTTCCGGGAAAGGGCTGGAGTGGGTTGGCCGTATAAGAAATAAGGCTAATAGGTA
CGCGACAGAATATGCTGCGTCGGTGAAAGGCAGGTTCACCATTTCAAGAGATGATTCA
AAGAACACGGCGTATCTACAAATGAACAGCCTGAAAACCGAGGACACGGCCGTGTAT
TATTGTACTAGAAACTGGAAGATTTTCCTCTTTGACTACTGGGGCCAGGGAACCCTGGT
CACCGTCTCCTCA
HCVR Amino Acid Sequence (SEQ ID NO: 130)
EVQLVESGGGLVQPGGSLKLSCTASGLTLSDSAMHWVRQASGKGLEWVGRIRNKANRYA
TEYAASVKGRFTISRDDSKNTAYLQMNSLKTEDTAVYYCTRNWKIFLFDYWGQGTLVTV
SS
HCDR1 Nucleic Acid Sequence (SEQ ID NO: 131)
GGG TTG ACC CTC AGT GAC TCT GCT
HCDR1 Amino Acid Sequence (SEQ ID NO: 132)
G L T L S D S A
HCDR2 Nucleic Acid Sequence (SEQ ID NO: 133)
ATA AGA AAT AAG GCT AAT AGG TAC GCG ACA
HCDR2 Amino Acid Sequence (SEQ ID NO: 134)
I R N K A N R Y A T
HCDR3 Nucleic Acid Sequence (SEQ ID NO: 135)
ACT AGA AAC TGG AAG ATT TTC CTC TTT GAC TAC
HCDR3 Amino Acid Sequence (SEQ ID NO: 136)
T R N W K I F L F D Y
LCVR Nucleic Acid Sequence (SEQ ID NO: 137)
GAAATTGTGTTGACGCAGTCTCCAGGCACCCTGACTTTGTCTCCAGGGGAAAGAGCCA
CCCTCTCCTGCAGGGCCAGTCAGAGTGTTGGCAGCAAATACTTAGCCTGGTTCCAGCA
GAAACGTGGCCAGGCTCCCAGGCTCCTCATCTATGGTGCATCCAGCAGGACCAGTGGC
ATCCCCGACAGGATCAGTGGCAGTGGGTCAGGGACAGACTTCACTCTCACCATCAGCA
GACTGGAGCCTGAAGATTTTGCAGTGTATTACTGTCAGCAGTATGGAAGTTCACCCTG
GACGTTCGGCCAAGGGACCAAGGTGGAAATCAAA
LCVR Amino Acid Sequence (SEQ ID NO: 138)
EIVLTQSPGTLTLSPGERATLSCRASQSVGSKYLAWFQQKRGQAPRLLIYGASSRTSGIPDRI
SGSGSGTDFTLTISRLEPEDFAVYYCQQYGSSPWTFGQGTKVEIK
LCDR1 Nucleic Acid Sequence (SEQ ID NO: 139)
CAG AGT GTT GGC AGC AAA TAC
LCDR1 Amino Acid Sequence (SEQ ID NO: 140)
Q S V G S K Y
LCDR2 Nucleic Acid Sequence (SEQ ID NO: 141)
GGT GCA TCC
LCDR2 Amino Acid Sequence (SEQ ID NO: 142)
G A S
LCDR3 Nucleic Acid Sequence (SEQ ID NO: 143)
CAG CAG TAT GGA AGT TCA CCC TGG ACG
LCDR3 Amino Acid Sequence (SEQ ID NO: 144)
Q Q Y G S S P W T
HC Nucleic Acid Sequence (SEQ ID NO: 225)
GAGGTGCAGCTGGTGGAGTCTGGGGGAGGCTTGGTCCAGCCGGGGGGGTCCCTGAAA
CTCTCCTGTACAGCCTCTGGGTTGACCCTCAGTGACTCTGCTATGCACTGGGTCCGCCA
GGCTTCCGGGAAAGGGCTGGAGTGGGTTGGCCGTATAAGAAATAAGGCTAATAGGTA
CGCGACAGAATATGCTGCGTCGGTGAAAGGCAGGTTCACCATTTCAAGAGATGATTCA
AAGAACACGGCGTATCTACAAATGAACAGCCTGAAAACCGAGGACACGGCCGTGTAT
TATTGTACTAGAAACTGGAAGATTTTCCTCTTTGACTACTGGGGCCAGGGAACCCTGGT
CACCGTCTCCTCAGCCTCCACCAAGGGCCCATCGGTCTTCCCCCTGGCGCCCTGCTCCA
GGAGCACCTCCGAGAGCACAGCCGCCCTGGGCTGCCTGGTCAAGGACTACTTCCCCGA
ACCGGTGACGGTGTCGTGGAACTCAGGCGCCCTGACCAGCGGCGTGCACACCTTCCCG
GCTGTCCTACAGTCCTCAGGACTCTACTCCCTCAGCAGCGTGGTGACCGTGCCCTCCAG
CAGCTTGGGCACGAAGACCTACACCTGCAACGTAGATCACAAGCCCAGCAACACCAA
GGTGGACAAGAGAGTTGAGTCCAAATATGGTCCCCCATGCCCACCCTGCCCAGCACCT
GAGTTCCTGGGGGGACCATCAGTCTTCCTGTTCCCCCCAAAACCCAAGGACACTCTCAT
GATCTCCCGGACCCCTGAGGTCACGTGCGTGGTGGTGGACGTGAGCCAGGAAGACCCC
GAGGTCCAGTTCAACTGGTACGTGGATGGCGTGGAGGTGCATAATGCCAAGACAAAG
CCGCGGGAGGAGCAGTTCAACAGCACGTACCGTGTGGTCAGCGTCCTCACCGTCCTGC
ACCAGGACTGGCTGAACGGCAAGGAGTACAAGTGCAAGGTCTCCAACAAAGGCCTCC
CGTCCTCCATCGAGAAAACCATCTCCAAAGCCAAAGGGCAGCCCCGAGAGCCACAGG
TGTACACCCTGCCCCCATCCCAGGAGGAGATGACCAAGAACCAGGTCAGCCTGACCTG
CCTGGTCAAAGGCTTCTACCCCAGCGACATCGCCGTGGAGTGGGAGAGCAATGGGCAG
CCGGAGAACAACTACAAGACCACGCCTCCCGTGCTGGACTCCGACGGCTCCTTCTTCC
TCTACAGCAGGCTCACCGTGGACAAGAGCAGGTGGCAGGAGGGGAATGTCTTCTCATG
CTCCGTGATGCATGAGGCTCTGCACAACCACTACACACAGAAGTCCCTCTCCCTGTCTC
TGGGTAAATGA
HC Amino Acid Sequence (SEQ ID NO: 226)
EVQLVESGGGLVQPGGSLKLSCTASGLTLSDSAMHWVRQASGKGLEWVGRIRNKANRYA
TEYAASVKGRFTISRDDSKNTAYLQMNSLKTEDTAVYYCTRNWKIFLFDYWGQGTLVTV
SSASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYSRLTVDKSRWQEG
NVFSCSVMHEALHNHYTQKSLSLSLGK
LC Nucleic Acid Sequence (SEQ ID NO: 227)
GAAATTGTGTTGACGCAGTCTCCAGGCACCCTGACTTTGTCTCCAGGGGAAAGAGCCA
CCCTCTCCTGCAGGGCCAGTCAGAGTGTTGGCAGCAAATACTTAGCCTGGTTCCAGCA
GAAACGTGGCCAGGCTCCCAGGCTCCTCATCTATGGTGCATCCAGCAGGACCAGTGGC
ATCCCCGACAGGATCAGTGGCAGTGGGTCAGGGACAGACTTCACTCTCACCATCAGCA
GACTGGAGCCTGAAGATTTTGCAGTGTATTACTGTCAGCAGTATGGAAGTTCACCCTG
GACGTTCGGCCAAGGGACCAAGGTGGAAATCAAACGAACTGTGGCTGCACCATCTGTC
TTCATCTTCCCGCCATCTGATGAGCAGTTGAAATCTGGAACTGCCTCTGTTGTGTGCCT
GCTGAATAACTTCTATCCCAGAGAGGCCAAAGTACAGTGGAAGGTGGATAACGCCCTC
CAATCGGGTAACTCCCAGGAGAGTGTCACAGAGCAGGACAGCAAGGACAGCACCTAC
AGCCTCAGCAGCACCCTGACGCTGAGCAAAGCAGACTACGAGAAACACAAAGTCTAC
GCCTGCGAAGTCACCCATCAGGGCCTGAGCTCGCCCGTCACAAAGAGCTTCAACAGGG
GAGAGTGTTAG
LC Amino Acid Sequence (SEQ ID NO: 228)
EIVLTQSPGTLTLSPGERATLSCRASQSVGSKYLAWFQQKRGQAPRLLIYGASSRTSGIPDRI
SGSGSGTDFTLTISRLEPEDFAVYYCQQYGSSPWTFGQGTKVEIKRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNRGEC
9909
HCVR Nucleic Acid Sequence (SEQ ID NO: 145)
GAGGTGCAGCTGTTGGAGTCTGGGGGAGGCTTGGTACAGCCGGGGGGGTCCCTGAGA
CTCTCCTGTGCAGCCTCTGGATTCACCTTTAACAACTATGGCATGAGCTGGGTCCGCCA
GGGTCCAGGGAAGGGGCTGGAGTGGGTCTCATCTATTAGTGGTAGTGGTGGTACCACA
TTCTACGCAGACTCCGTGAAGGGCCGGTTCACCATCTCCAGAGACAATTCCAAGAACA
CGCTGTATCTGCAAATGAACAGCCTGAGAGCCGAGGACACGGCCGTATATTACTGTGG
CAAAGGAGGATATTGTAGTAGTAGCGGCTGCCGTCACTACGGTATGGACGTCTGGGGC
CAAGGGACCACGGTCACCGTCTCCTCA
HCVR Amino Acid Sequence (SEQ ID NO: 146)
EVQLLESGGGLVQPGGSLRLSCAASGFTFNNYGMSWVRQGPGKGLEWVSSISGSGGTTFY
ADSVKGRFTISRDNSKNTLYLQMNSLRAEDTAVYYCGKGGYCSSSGCRHYGMDVWGQG
TTVTVSS
HCDR1 Nucleic Acid Sequence (SEQ ID NO: 147)
GGA TTC ACC TTT AAC AAC TAT GGC
HCDR1 Amino Acid Sequence (SEQ ID NO: 148)
G F T F N N Y G
HCDR2 Nucleic Acid Sequence (SEQ ID NO: 149)
ATT AGT GGT AGT GGT GGT ACC ACA
HCDR2 Amino Acid Sequence (SEQ ID NO: 150)
S G S G G T
HCDR3 Nucleic Acid Sequence (SEQ ID NO: 151)
GGC AAA GGA GGA TAT TGT AGT AGT AGC GGC TGC CGT CAC TAC GGT ATG GAC
GTC
HCDR3 Amino Acid Sequence (SEQ ID NO: 152)
C G K G G Y C S S S G C R H
LCVR Nucleic Acid Sequence (SEQ ID NO: 153)
CAGTCTGTGCTGACTCAGCCACCCTCAGCGTCTGGGACCCCCGGGCAGAGGGTCACCA
TCTCTTGTTCTGGAAGCAGCTCCAACATCGGAAATAATTATATATACTGGTACCAGCGG
CTCCCAGGAACGACCCCCAAACTCCTCATCTATAGGAATAATCAGCGGCCCTCAGGGG
TCCCTGACCGATTCTCTGGCTCCAAGTCTGGCACCTCAGCCTCCCTGGCCATCAGTGGG
CTCCGGTCCGAGGATGAGGCTGATTATTACTGTGCAGCATGGGATGACACCCTGAGTG
GGTATGTCTTCGGAACTGGGACCAAGGTCACCGTCCTA
LCVR Amino Acid Sequence (SEQ ID NO: 154)
QSVLTQPPSASGTPGQRVTISCSGSSSNIGNNYIYWYQRLPGTTPKLLIYRNNQRPSGVPDRF
SGSKSGTSASLAISGLRSEDEADYYCAAWDDTLSGYVFGTGTKVTVL
LCDR1 Nucleic Acid Sequence (SEQ ID NO: 155)
AGC TCC AAC ATC GGA AAT AAT TAT
LCDR1 Amino Acid Sequence (SEQ ID NO: 156)
S S N I G N N Y
LCDR2 Nucleic Acid Sequence (SEQ ID NO: 157)
agg aat aat
LCDR2 Amino Acid Sequence (SEQ ID NO: 158)
R N
LCDR3 Nucleic Acid Sequence (SEQ ID NO: 159)
GCA GCA TGG GAT GAC ACC CTG AGT GGG TAT GTC
LCDR3 Amino Acid Sequence (SEQ ID NO: 160)
C A A W D D T L S G Y
HC Nucleic Acid Sequence (SEQ ID NO: 229)
GAGGTGCAGCTGTTGGAGTCTGGGGGAGGCTTGGTACAGCCGGGGGGGTCCCTGAGA
CTCTCCTGTGCAGCCTCTGGATTCACCTTTAACAACTATGGCATGAGCTGGGTCCGCCA
GGGTCCAGGGAAGGGGCTGGAGTGGGTCTCATCTATTAGTGGTAGTGGTGGTACCACA
TTCTACGCAGACTCCGTGAAGGGCCGGTTCACCATCTCCAGAGACAATTCCAAGAACA
CGCTGTATCTGCAAATGAACAGCCTGAGAGCCGAGGACACGGCCGTATATTACTGTGG
CAAAGGAGGATATTGTAGTAGTAGCGGCTGCCGTCACTACGGTATGGACGTCTGGGGC
CAAGGGACCACGGTCACCGTCTCCTCAGCCAAAACAACAGCCCCATCGGTCTATCCAC
TGGCCCCTGTGTGTGGAGATACAACTGGCTCCTCGGTGACTCTAGGATGCCTGGTCAA
GGGTTATTTCCCTGAGCCAGTGACCTTGACCTGGAACTCTGGATCCCTGTCCAGTGGTG
TGCACACCTTCCCAGCTGTCCTGCAGTCTGACCTCTACACCCTCAGCAGCTCAGTGACT
GTAACCTCGAGCACCTGGCCCAGCCAGTCCATCACCTGCAATGTGGCCCACCCGGCAA
GCAGCACCAAGGTGGACAAGAAAATTGAGCCCAGAGGGCCCACAATCAAGCCCTGTC
CTCCATGCAAATGCCCAGCACCTAACCTCTTGGGTGGACCATCCGTCTTCATCTTCCCT
CCAAAGATCAAGGATGTACTCATGATCTCCCTGAGCCCCATAGTCACATGTGTGGTGG
TGGATGTGAGCGAGGATGACCCAGATGTCCAGATCAGCTGGTTTGTGAACAACGTGGA
AGTACACACAGCTCAGACACAAACCCATAGAGAGGATTACAACAGTACTCTCCGGGTG
GTCAGTGCCCTCCCCATCCAGCACCAGGACTGGATGAGTGGCAAGGAGTTCAAATGCA
AGGTCAACAACAAAGACCTCCCAGCGCCCATCGAGAGAACCATCTCAAAACCCAAAG
GGTCAGTAAGAGCTCCACAGGTATATGTCTTGCCTCCACCAGAAGAAGAGATGACTAA
GAAACAGGTCACTCTGACCTGCATGGTCACAGACTTCATGCCTGAAGACATTTACGTG
GAGTGGACCAACAACGGGAAAACAGAGCTAAACTACAAGAACACTGAACCAGTCCTG
GACTCTGATGGTTCTTACTTCATGTACAGCAAGCTGAGAGTGGAAAAGAAGAACTGGG
TGGAAAGAAATAGCTACTCCTGTTCAGTGGTCCACGAGGGTCTGCACAATCACCACAC
GACTAAGAGCTTCTCCCGGACTCCGGGTAAATGA
HC Amino Acid Sequence (SEQ ID NO: 230)
EVQLLESGGGLVQPGGSLRLSCAASGFTFNNYGMSWVRQGPGKGLEWVSSISGSGGTTFY
ADSVKGRFTISRDNSKNTLYLQMNSLRAEDTAVYYCGKGGYCSSSGCRHYGMDVWGQG
TTVTVSSAKTTAPSVYPLAPVCGDTTGSSVTLGCLVKGYFPEPVTLTWNSGSLSSGVHTFP
AVLQSDLYTLSSSVTVTSSTWPSQSITCNVAHPASSTKVDKKIEPRGPTIKPCPPCKCPAPNL
LGGPSVFIFPPKIKDVLMISLSPIVTCVVVDVSEDDPDVQISWFVNNVEVHTAQTQTHREDY
NSTLRVVSALPIQHQDWMSGKEFKCKVNNKDLPAPIERTISKPKGSVRAPQVYVLPPPEEE
MTKKQVTLTCMVTDFMPEDIYVEWTNNGKTELNYKNTEPVLDSDGSYFMYSKLRVEKK
NWVERNSYSCSVVHEGLHNHHTTKSFSRTPGK
LC Nucleic Acid Sequence (SEQ ID NO: 231)
CAGTCTGTGCTGACTCAGCCACCCTCAGCGTCTGGGACCCCCGGGCAGAGGGTCACCA
TCTCTTGTTCTGGAAGCAGCTCCAACATCGGAAATAATTATATATACTGGTACCAGCGG
CTCCCAGGAACGACCCCCAAACTCCTCATCTATAGGAATAATCAGCGGCCCTCAGGGG
TCCCTGACCGATTCTCTGGCTCCAAGTCTGGCACCTCAGCCTCCCTGGCCATCAGTGGG
CTCCGGTCCGAGGATGAGGCTGATTATTACTGTGCAGCATGGGATGACACCCTGAGTG
GGTATGTCTTCGGAACTGGGACCAAGGTCACCGTCCTACGAGCTGATGCTGCACCAAC
TGTATCCATCTTCCCACCATCCAGTGAGCAGTTAACATCTGGAGGTGCCTCAGTCGTGT
GCTTCTTGAACAACTTCTACCCCAAAGACATCAATGTCAAGTGGAAGATTGATGGCAG
TGAACGACAAAATGGCGTCCTGAACAGTTGGACTGATCAGGACAGCAAAGACAGCAC
CTACAGCATGAGCAGCACCCTCACGTTGACCAAGGACGAGTATGAACGACATAACAG
CTATACCTGTGAGGCCACTCACAAGACATCAACTTCACCCATTGTCAAGAGCTTCAAC
AGGGGAGAGTGTTGA
LC Amino Acid Sequence (SEQ ID NO: 232)
QSVLTQPPSASGTPGQRVTISCSGSSSNIGNNYIYWYQRLPGTTPKLLIYRNNQRPSGVPDRF
SGSKSGTSASLAISGLRSEDEADYYCAAWDDTLSGYVFGTGTKVTVLRADAAPTVSIFPPS
SEQLTSGGASVVCFLNNFYPKDINVKWKIDGSERQNGVLNSWTDQDSKDSTYSMSSTLTL
TKDEYERHNSYTCEATHKTSTSPIVKSFNRGEC
10713 (wildtype hIgG1)/14573 (hIgG1 N180Q)
HCVR Nucleic Acid Sequence (SEQ ID NO: 161)
GAGGTGCAGCTGGTGGAGTCTGGGGGAAACTTGGTACAGCCTGGGGGGTCCCTGAGA
CTCTCCTGTGCAGCCTCTGGATTCACCTTTACCAGCCATGCCATGAACTGGGTCCGCCA
GGCTCCAGGGAAGGGGCTGGAGTGGGTCTCAGTTATTACTGGTAGAGGTTTTGACACA
CACTACGCTGACTCCGTGAAGGGCCGGTTCACCATCTCCAGAGACATTTCCAAAAACA
CGCTGTATCTGCAAATGAACAGCCTGAGAGCCGAGGACACGGCCGTTTATTACTGTGC
GAAAGGTCTCTATGATTCGGGGAATTATTATATCGATTACTGGGGCCAGGGAACCCTG
GTCACCGTCTCCTCA
HCVR Amino Acid Sequence (SEQ ID NO: 162)
EVQLVESGGNLVQPGGSLRLSCAASGFTFTSHAMNWVRQAPGKGLEWVSVITGRGFDTH
YADSVKGRFTISRDISKNTLYLQMNSLRAEDTAVYYCAKGLYDSGNYYIDYWGQGTLVT
VSS
HCDR1 Nucleic Acid Sequence (SEQ ID NO: 163)
GGA TTC ACC TTT ACC AGC CAT GCC
HCDR1 Amino Acid Sequence (SEQ ID NO: 164)
G F T F T S H A
HCDR2 Nucleic Acid Sequence (SEQ ID NO: 165)
ATT ACT GGT AGA GGT TTT GAC ACA
HCDR2 Amino Acid Sequence (SEQ ID NO: 166)
I T G R G F D T
HCDR3 Nucleic Acid Sequence (SEQ ID NO: 167)
GCG AAA GGT CTC TAT GAT TCG GGG AAT TAT TAT ATC GAT TAC
HCDR3 Amino Acid Sequence (SEQ ID NO: 168)
A K G L Y D S G N Y Y I D Y
LCVR Nucleic Acid Sequence (SEQ ID NO: 169)
CAGTCTGTGTTGACGCAGCCGCCCTCAGTGTCTGCGGCCCCAGGACAGAAGGTCACCA
TCTCCTGCTCTGGAAGCAGCTCCAACATTGGGAATAATTATGTTTCCTGGTACCAGCAG
CTCCCAGGAACAGCCCCCAAACTCCTCATTTATGACAATAATAAGCGACCCTCAGGGA
TTCCTGACCGATTCTCTGGCTCCAAGTCTGGCACGTCAGCCACCCTGGGCATCACCGGA
CTCCAGACTGGGGACGAGGCCGATTATTACTGCGGAACATGGGATCTCAGCCTGAGTT
TCAATTGGGTGTTCGGCGGAGGGACCAAGCTGACCGTCCTA
LCVR Amino Acid Sequence (SEQ ID NO: 170)
QSVLTQPPSVSAAPGQKVTISCSGSSSNIGNNYVSWYQQLPGTAPKLLIYDNNKRPSGIPDR
FSGSKSGTSATLGITGLQTGDEADYYCGTWDLSLSFNWVFGGGTKLTVL
LCDR1 Nucleic Acid Sequence (SEQ ID NO: 171)
AGC TCC AAC ATT GGG AAT AAT TAT
LCDR1 Amino Acid Sequence (SEQ ID NO: 172)
S S N I G N N Y
LCDR2 Nucleic Acid Sequence (SEQ ID NO: 173)
GAC AAT AAT
LCDR2 Amino Acid Sequence (SEQ ID NO: 174)
D N N
LCDR3 Nucleic Acid Sequence (SEQ ID NO: 175)
GGA ACA TGG GAT CTC AGC CTG AGT TTC AAT TGG GTG
LCDR3 Amino Acid Sequence (SEQ ID NO: 176)
G T W D L S L S F N W V
HC Nucleic Acid Sequence (SEQ ID NO: 233)
GAGGTGCAGCTGGTGGAGTCTGGGGGAAACTTGGTACAGCCTGGGGGGTCCCTGAGA
CTCTCCTGTGCAGCCTCTGGATTCACCTTTACCAGCCATGCCATGAACTGGGTCCGCCA
GGCTCCAGGGAAGGGGCTGGAGTGGGTCTCAGTTATTACTGGTAGAGGTTTTGACACA
CACTACGCTGACTCCGTGAAGGGCCGGTTCACCATCTCCAGAGACATTTCCAAAAACA
CGCTGTATCTGCAAATGAACAGCCTGAGAGCCGAGGACACGGCCGTTTATTACTGTGC
GAAAGGTCTCTATGATTCGGGGAATTATTATATCGATTACTGGGGCCAGGGAACCCTG
GTCACCGTCTCCTCAGCCTCCACCAAGGGCCCATCGGTCTTCCCCCTGGCGCCCTGCTC
CAGGAGCACCTCCGAGAGCACAGCCGCCCTGGGCTGCCTGGTCAAGGACTACTTCCCC
GAACCGGTGACGGTGTCGTGGAACTCAGGCGCCCTGACCAGCGGCGTGCACACCTTCC
CGGCTGTCCTACAGTCCTCAGGACTCTACTCCCTCAGCAGCGTGGTGACCGTGCCCTCC
AGCAGCTTGGGCACGAAGACCTACACCTGCAACGTAGATCACAAGCCCAGCAACACC
AAGGTGGACAAGAGAGTTGAGTCCAAATATGGTCCCCCATGCCCACCCTGCCCAGCAC
CTGAGTTCCTGGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACC
CCGAGGTCCAGTTCAACTGGTACGTGGATGGCGTGGAGGTGCATAATGCCAAGACAAA
GCCGCGGGAGGAGCAGTTCAACAGCACGTACCGTGTGGTCAGCGTCCTCACCGTCCTG
CACCAGGACTGGCTGAACGGCAAGGAGTACAAGTGCAAGGTCTCCAACAAAGGCCTC
CCGTCCTCCATCGAGAAAACCATCTCCAAAGCCAAAGGGCAGCCCCGAGAGCCACAG
GTGTACACCCTGCCCCCATCCCAGGAGGAGATGACCAAGAACCAGGTCAGCCTGACCT
GCCTGGTCAAAGGCTTCTACCCCAGCGACATCGCCGTGGAGTGGGAGAGCAATGGGCA
GCCGGAGAACAACTACAAGACCACGCCTCCCGTGCTGGACTCCGACGGCTCCTTCTTC
CTCTACAGCAGGCTCACCGTGGACAAGAGCAGGTGGCAGGAGGGGAATGTCTTCTCAT
GCTCCGTGATGCATGAGGCTCTGCACAACCACTACACACAGAAGTCCCTCTCCCTGTCT
CTGGGTAAATGA
HC Amino Acid Sequence (SEQ ID NO: 234)
EVQLVESGGNLVQPGGSLRLSCAASGFTFTSHAMNWVRQAPGKGLEWVSVITGRGFDTH
YADSVKGRFTISRDISKNTLYLQMNSLRAEDTAVYYCAKGLYDSGNYYIDYWGQGTLVT
VSSASTKGPSVFPLAPCSRSTSESTAALGCLVKDYFPEPVTVSWNSGALTSGVHTFPAVLQS
SGLYSLSSVVTVPSSSL
GTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYT
QKSLSLSLGK
*underlined and bolded asparagine (N) may be mutated to a glutamine (Q) for
conjugation by transglutaminase, see, e.g., SEQ ID NO: 269
LC Nucleic Acid Sequence (SEQ ID NO: 235)
CAGTCTGTGTTGACGCAGCCGCCCTCAGTGTCTGCGGCCCCAGGACAGAAGGTCACCA
TCTCCTGCTCTGGAAGCAGCTCCAACATTGGGAATAATTATGTTTCCTGGTACCAGCAG
CTCCCAGGAACAGCCCCCAAACTCCTCATTTATGACAATAATAAGCGACCCTCAGGGA
TTCCTGACCGATTCTCTGGCTCCAAGTCTGGCACGTCAGCCACCCTGGGCATCACCGGA
CTCCAGACTGGGGACGAGGCCGATTATTACTGCGGAACATGGGATCTCAGCCTGAGTT
TCAATTGGGTGTTCGGCGGAGGGACCAAGCTGACCGTCCTAGGCCAGCCCAAGGCCGC
CCCCTCCGTGACCCTGTTCCCCCCCTCCTCCGAGGAGCTGCAGGCCAACAAGGCCACC
CTGGTGTGCCTGATCTCCGACTTCTACCCCGGCGCCGTGACCGTGGCCTGGAAGGCCG
ACTCCTCCCCCGTGAAGGCCGGCGTGGAGACCACCACCCCCTCCAAGCAGTCCAACAA
CAAGTACGCCGCCTCCTCCTACCTGTCCCTGACCCCCGAGCAGTGGAAGTCCCACCGG
TCCTACTCCTGCCAGGTGACCCACGAGGGCTCCACCGTGGAGAAGACCGTGGCCCCCA
CCGAGTGCTCCTGA
LC Amino Acid Sequence (SEQ ID NO: 236)
QSVLTQPPSVSAAPGQKVTISCSGSSSNIGNNYVSWYQQLPGTAPKLLIYDNNKRPSGIPDR
FSGSKSGTSATLGITGLQTGDEADYYCGTWDLSLSFNWVFGGGTKLTVLGQPKAAPSVTL
FPPSSEELQANKATLVCLISDFYPGAVTVAWKADSSPVKAGVETTTPSKQSNNKYAASSYL
SLTPEQWKSHRSYSCQVTHEGSTVEKTVAPTECS
7854
HCVR Nucleic Acid Sequence (SEQ ID NO: 177)
CAGGTTCAGCTGGTGCAGTCTGGAGCTGAGGTGAAGAAGCCTGGGGCCTCAGTGAAG
GTCTCCTGCAAGGCTTCTGGTTACGCCTTCACCACCTATGGTATCACCTGGGTGCGACA
GGCCCCTGGACAAGGACTTGAGTGGATGGGATGGATCAGCGCTTACAATGGAAATAC
AAACTATGCAGAGAAGGTCCAGGGCAGATTCACCATGACCACAGACACATCCACGAA
TACAGCCTACATGGAGCTGAGGAGCCTGAGATCCGACGACACGGCCGTGTATTTCTGT
GCGAGAAAGGGTCACTATGGTTCGGGGACTTATTATAACCCCTTTGGTTTTGATTTTTG
GGGCCAAGGGACAATGGTCACCGTCTCTTCA
HCVR Amino Acid Sequence (SEQ ID NO: 178)
QVQLVQSGAEVKKPGASVKVSCKASGYAFTTYGITWVRQAPGQGLEWMGWISAYNGNT
NYAEKVQGRFTMTTDTSTNTAYMELRSLRSDDTAVYFCARKGHYGSGTYYNPFGFDFWG
QGTMVTVSS
HCDR1 Nucleic Acid Sequence (SEQ ID NO: 179)
GGT TAC GCC TTC ACC ACC TAT GGT
HCDR1 Amino Acid Sequence (SEQ ID NO: 180)
G Y A F T T Y G
HCDR2 Nucleic Acid Sequence (SEQ ID NO: 181)
ATC AGC GCT TAC AAT GGA AAT ACA
HCDR2 Amino Acid Sequence (SEQ ID NO: 182)
I S A Y N G N T
HCDR3 Nucleic Acid Sequence (SEQ ID NO: 183)
GCG AGA AAG GGT CAC TAT GGT TCG GGG ACT TAT TAT AAC CCC TTT GGT TTT GAT
TTT
HCDR3 Amino Acid Sequence (SEQ ID NO: 184)
A R K G H Y G S G T Y Y N P F G F D F
LCVR Nucleic Acid Sequence (SEQ ID NO: 185)
GAAATTGTGTTGACGCAGTCTCCAGGCACCCTGTCTTTGTCTCCAGGGGAAAGAGCCA
CCCTCTCCTGCAGGGCCAGTCAGAGTGTTAGCAGCAGCTACTTAGCCTGGTACCAACA
GAAACCTGGCCAGGCTCCCAGGCTCCTCATCTATGGTGCATCCAGCAGGGCCACTGGC
ATCCCAGACAGGTTCAGTGGCAGTGGGTCTGGGACAGACTTCACTCTCACCATCAGCA
GACTGGAGCCTGAAGATTTTGCTTTGTATTTCTGTCAGCAGTATTATGGCTCACCTTGG
ACGTTCGGCCAAGGGACCAAGGTGGAAATCAAA
LCVR Amino Acid Sequence (SEQ ID NO: 186)
EIVLTQSPGTLSLSPGERATLSCRASQSVSSSYLAWYQQKPGQAPRLLIYGASSRATGIPDRF
SGSGSGTDFTLTISRLEPEDFALYFCQQYYGSPWTFGQGTKVEIK
LCDR1 Nucleic Acid Sequence (SEQ ID NO: 187)
CAG AGT GTT AGC AGC AGC TAC
LCDR1 Amino Acid Sequence (SEQ ID NO: 188)
Q S V S S S Y
LCDR2 Nucleic Acid Sequence (SEQ ID NO: 189)
GGT GCA TCC
LCDR2 Amino Acid Sequence (SEQ ID NO: 190)
G A S
LCDR3 Nucleic Acid Sequence (SEQ ID NO: 191)
CAG CAG TAT TAT GGC TCA CCT TGG ACG
LCDR3 Amino Acid Sequence (SEQ ID NO: 192)
Q Q Y Y G S P W T
HC Nucleic Acid Sequence (SEQ ID NO: 237)
CAGGTTCAGCTGGTGCAGTCTGGAGCTGAGGTGAAGAAGCCTGGGGCCTCAGTGAAG
GTCTCCTGCAAGGCTTCTGGTTACGCCTTCACCACCTATGGTATCACCTGGGTGCGACA
GGCCCCTGGACAAGGACTTGAGTGGATGGGATGGATCAGCGCTTACAATGGAAATAC
AAACTATGCAGAGAAGGTCCAGGGCAGATTCACCATGACCACAGACACATCCACGAA
TACAGCCTACATGGAGCTGAGGAGCCTGAGATCCGACGACACGGCCGTGTATTTCTGT
GCGAGAAAGGGTCACTATGGTTCGGGGACTTATTATAACCCCTTTGGTTTTGATTTTTG
GGGCCAAGGGACAATGGTCACCGTCTCTTCAGCCTCCACCAAGGGCCCATCGGTCTTC
CCCCTGGCGCCCTGCTCCAGGAGCACCTCCGAGAGCACAGCCGCCCTGGGCTGCCTGG
TCAAGGACTACTTCCCCGAACCGGTGACGGTGTCGTGGAACTCAGGCGCCCTGACCAG
CGGCGTGCACACCTTCCCGGCTGTCCTACAGTCCTCAGGACTCTACTCCCTCAGCAGCG
TGGTGACCGTGCCCTCCAGCAGCTTGGGCACGAAGACCTACACCTGCAACGTAGATCA
CAAGCCCAGCAACACCAAGGTGGACAAGAGAGTTGAGTCCAAATATGGTCCCCCATG
CCCACCCTGCCCAGCACCTGAGTTCCTGGGGGGACCATCAGTCTTCCTGTTCCCCCCAA
AACCCAAGGACACTCTCATGATCTCCCGGACCCCTGAGGTCACGTGCGTGGTGGTGGA
CGTGAGCCAGGAAGACCCCGAGGTCCAGTTCAACTGGTACGTGGATGGCGTGGAGGT
GCATAATGCCAAGACAAAGCCGCGGGAGGAGCAGTTCAACAGCACGTACCGTGTGGT
CAGCGTCCTCACCGTCCTGCACCAGGACTGGCTGAACGGCAAGGAGTACAAGTGCAAG
GTCTCCAACAAAGGCCTCCCGTCCTCCATCGAGAAAACCATCTCCAAAGCCAAAGGGC
AGCCCCGAGAGCCACAGGTGTACACCCTGCCCCCATCCCAGGAGGAGATGACCAAGA
ACCAGGTCAGCCTGACCTGCCTGGTCAAAGGCTTCTACCCCAGCGACATCGCCGTGGA
GTGGGAGAGCAATGGGCAGCCGGAGAACAACTACAAGACCACGCCTCCCGTGCTGGA
CTCCGACGGCTCCTTCTTCCTCTACAGCAGGCTCACCGTGGACAAGAGCAGGTGGCAG
GAGGGGAATGTCTTCTCATGCTCCGTGATGCATGAGGCTCTGCACAACCACTACACAC
AGAAGTCCCTCTCCCTGTCTCTGGGTAAATGA
HC Amino Acid Sequence (SEQ ID NO: 238)
QVQLVQSGAEVKKPGASVKVSCKASGYAFTTYGITWVRQAPGQGLEWMGWISAYNGNT
NYAEKVQGRFTMTTDTSTNTAYMELRSLRSDDTAVYFCARKGHYGSGTYYNPFGFDFWG
QGTMVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFLYSRLTVDKSR
WQEGNVFSCSVMHEALHNHYTQKSLSLSLGK
LC Nucleic Acid Sequence (SEQ ID NO: 239)
GAAATTGTGTTGACGCAGTCTCCAGGCACCCTGTCTTTGTCTCCAGGGGAAAGAGCCA
CCCTCTCCTGCAGGGCCAGTCAGAGTGTTAGCAGCAGCTACTTAGCCTGGTACCAACA
GAAACCTGGCCAGGCTCCCAGGCTCCTCATCTATGGTGCATCCAGCAGGGCCACTGGC
ATCCCAGACAGGTTCAGTGGCAGTGGGTCTGGGACAGACTTCACTCTCACCATCAGCA
GACTGGAGCCTGAAGATTTTGCTTTGTATTTCTGTCAGCAGTATTATGGCTCACCTTGG
ACGTTCGGCCAAGGGACCAAGGTGGAAATCAAACGAACTGTGGCTGCACCATCTGTCT
TCATCTTCCCGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAACGCCCTCC
AATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACG
CCTGCGAAGTCACCCATCAGGGCCTGAGCTCGCCCGTCACAAAGAGCTTCAACAGGGG
AGAGTGTTAG
LC Amino Acid Sequence (SEQ ID NO: 240)
EIVLTQSPGTLSLSPGERATLSCRASQSVSSSYLAWYQQKPGQAPRLLIYGASSRATGIPDRF
SGSGSGTDFTLTISRLEPEDFALYFCQQYYGSPWTFGQGTKVEIKRTVAAPSVFIFPPSDEQL
KSGTASVVCLLNNFYPREAKVQWKVDNALQSGNSQESVTEQDSKDSTYSLSSTLTLSKAD
YEKHKVYACEVTHQGLSSPVTKSFNRGEC
Human (h) IgG1 N180Q Amino Acid Sequence (SEQ ID NO:269)
GTKTYTCNVDHKPSNTKVDKRVESKYGPPCPPCPAPEFLGGPSVFLFPPKPKDTLMISRTPE
VTCVVVDVSQEDPEVQFNWYVDGVEVHNAKTKPREEQFQSTYRVVSVLTVLHQDWLNG
KEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYT
QKSLSLSLGK

### Example 2: Biacore Binding Kinetics of Anti-CACNG1 Monoclonal Antibodies to Human CACNG1 Nanodisc in an Antigen Capture Format at 25° C.

Equilibrium dissociation constants (K_{D} values) for nanodisc embedded human CACNG1 expressed with a C-terminal PADRE-Flag-His tag (human CACNG1 nanodisc) binding to purified anti-hCACNG1 antibodies were determined using a real-time surface plasmon resonance biosensor technology with a Biacore T200 instrument. The CM5 Biacore sensor surface was derivatized by amine coupling with a monoclonal mouse anti-His antibody (Cytiva; Marlborough, MA). All Biacore binding studies were performed in a buffer composed of 0.01M HEPES, 0.15M NaCl, 1 mM CaCl₂), 0.5 mM MgCl₂, pH 7.4 (HBS-N++ running buffer). Different concentrations of anti-hCACNG1 antibodies (ranging from 300 nM to 12 nM in 5-fold serial dilutions) prepared in HBS-N++ running buffer were injected over the captured human CACNG1 nanodisc at a flow rate of 30 μL/minute. Antibody association was monitored for 2 minutes while dissociation was monitored in HBS-N++ running buffer for 5 minutes. At the end of each cycle, the human CACNG1 nanodisc capture surface was regenerated using three 10 second injections of 10 mM Gly pH1.5. All binding kinetics experiments were performed at 25° C.

The specific SPR-Biacore sensorgrams were obtained by a double referencing procedure. The double referencing was performed by first subtracting the signal of each injection over a reference surface (anti-His) from the signal over the experimental surface (anti-His captured human CACNG1 nanodisc) thereby removing contributions from refractive index changes. In addition, running buffer injections were performed to allow subtraction of the signal changes resulting from the dissociation of captured antibodies from the coupled anti-His surface. Kinetic association (ka) and dissociation (k_{d}) rate constants were determined by fitting the real-time sensorgrams to a 1:1 binding model using Scrubber v2.0c curve fitting software. Binding dissociation equilibrium constants (K_{D}) and dissociative half-lives (t_{1/2}) were calculated from the kinetic rate constants as:

$${K}_{D} ( M ) = \frac{kd}{ka} , and t 1 / 2 ( min ) = \frac{ln ( 2 )}{60 * kd}$$

Anti-hCACNG1 antibodies kinetic results are presented in Table 2. As shown in Table 2, all of the antibodies bound to surface-captured human CACNG1 nanodisc with several antibodies binding with single digit nM or triple digit pM affinities.

TABLE 2
Kinetic and Equilibrium Binding Parameters of anti-hCACNG1 antibodies
to Surface-captured human CACNG1 nanodisc at 25° C.

| | CACNG1 | | | | | |
|---|---|---|---|---|---|---|
| | nanodisc | mAb Bound | | | | |
| | Captured | at 300 nM | | | | t1/2 |
| | (RU) | (RU) | ka (1/Ms) | kd (1/s) | KD (M) | (min) |
| H2aM31929N/REGN10728 | 122 ± 0.7 | 25 | 7.50E+05 | 5.59E−04 | 7.45E−10 | 20.7 |
| H2aM31944N | 112 ± 0.6 | 16 | 2.31E+05 | 3.95E−03 | 1.71E−08 | 2.9 |
| H4H31265P2/REGN5972 | 140 ± 1.3 | 14 | 1.04E+05 | 8.40E−03 | 8.10E−08 | 1.4 |
| H2aM31941N | 126 ± 0.9 | 19 | 3.42E+05 | 4.06E−04 | 1.19E−09 | 28.4 |
| REGN7660 | 209 ± 1.9 | 23 | 1.93E+06 | 2.43E−02 | 1.26E−08 | 0.5 |
| REGN9909 | 191 ± 3.3 | 22 | 3.31E+05 | 1.37E−03 | 4.15E−09 | 8.4 |
| REGN10713 | 167 ± 1 | 53 | 7.42E+04 | 4.72E−05 | 6.35E−10 | 245.0 |
| REGN10715 | 159 ± 1.5 | 31 | 1.52E+05 | 3.01E−04 | 1.98E−09 | 38.4 |
| REGN10716 | 152 ± 1.9 | 31 | 7.30E+05 | 8.36E−05 | 1.14E−10 | 138.2 |
| REGN10717 | 148 ± 1.9 | 7 | 2.94E+05 | 3.04E−02 | 1.04E−07 | 0.4 |
| REGN10783 | 139 ± 0.6 | 9 | 1.36E+05 | 1.27E−02 | 9.35E−08 | 0.9 |

### Example 3: In Vitro and Ex Vivo Screening of Purified CACNG1 Antibodies Using Human and Mouse Myotubes

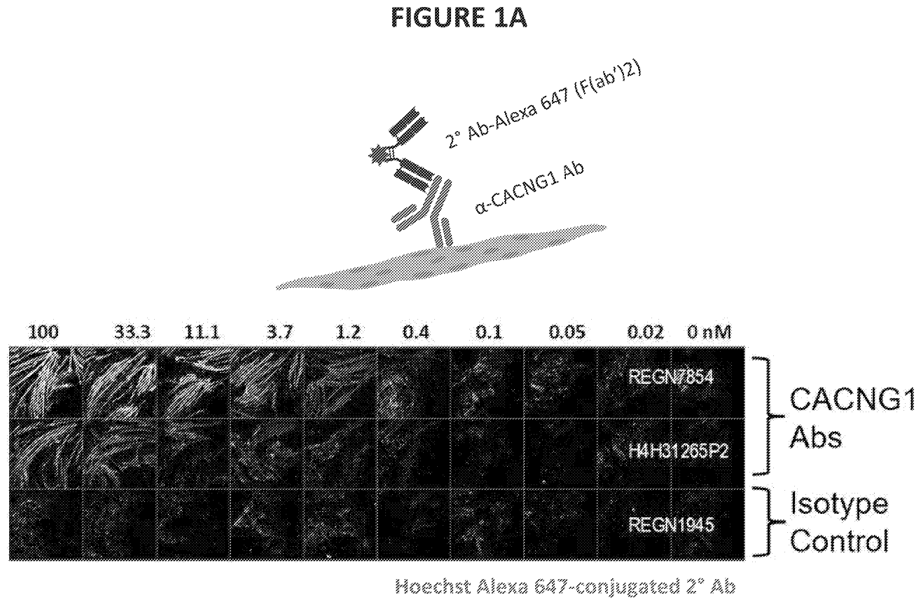

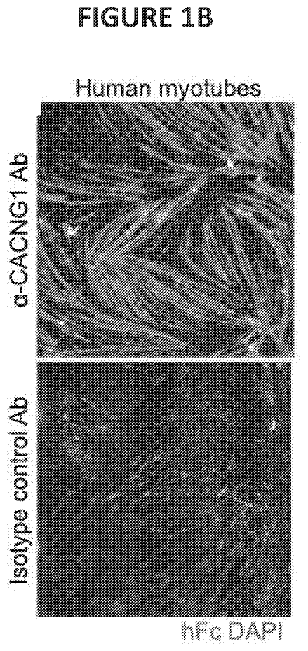

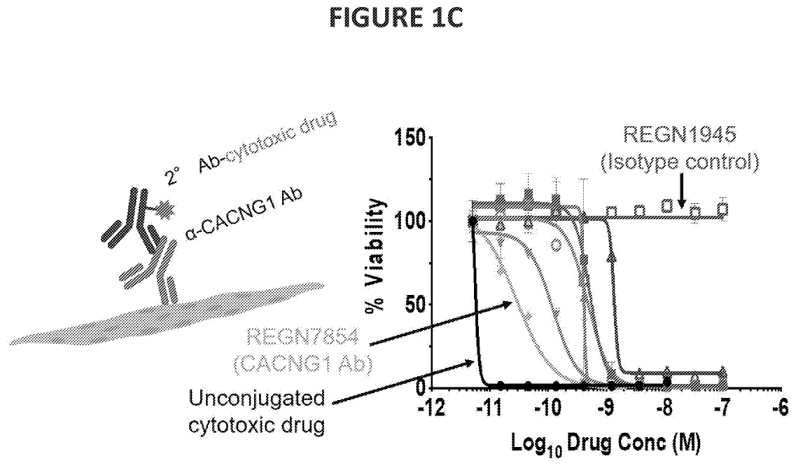

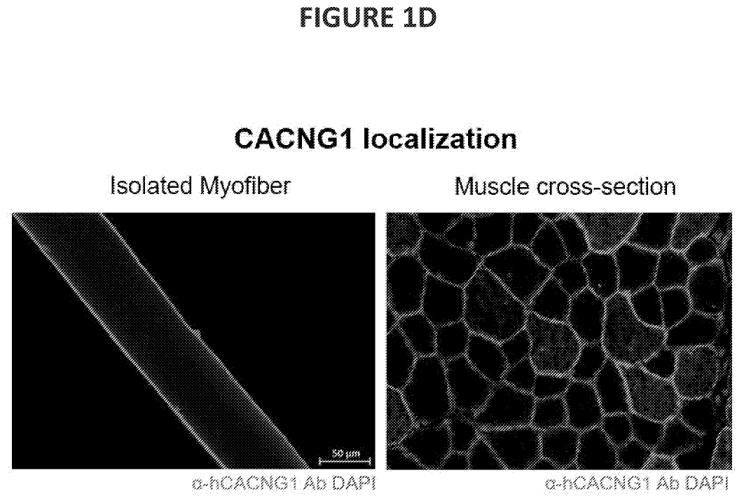

A total of 43 purified CACNG1 antibodies from two immunization campaigns were screened in vitro using human and mouse myotubes. Incubation of live myotubes with CACNG1 antibodies followed by fluorophore-conjugated secondary detection was performed to assess antibody binding (FIG. 1A). Live staining of human myotubes with 25 nM of anti-CACNG1 antibody followed by fluorophore-conjugated secondary antibody detection displayed robust binding compared to isotype control (FIG. 1B). Incubation of myotubes with CACNG1 antibodies followed by duocarmycin-conjugated secondary was performed to assess antibody internalization via cell killing assay (FIG. 1C).

Immunostaining for CACNG1 in CACNG1^{Hu/Hu} mouse single myofibers and muscle tissue cross sections (FIG. 1D) confirmed that CACNG1 is expressed at the cell surface of the myofiber.

### Example 4: Binding of Anti-hCACNG1 Monoclonal Antibodies to Mouse or Human Myotubes, and Effect of Binding on Calcium Flux by Human Myoblasts

CACNG1 is the γ1 subunit of the skeletal muscle specific L-type calcium channel, (dihydropyridine receptor), though genetic deletion of CACNG1 appears to have no major impact on skeletal muscle function. To determine whether antibodies that bind CACNG1 impact muscle function, a calcium flux assay was performed on human myotubes that were incubated with CACNG1 antibodies to determine whether these antibodies affect acetylcholine-induced calcium release.

Human skeletal myoblasts (Cook Myosite, Inc.) were plated at 10,000 cells/well of a 96-well plate and were differentiated for 7 days into myotubes. On the final day of differentiation, media was replaced with 50 μL of FLIPR calcium 5 dye with probenecid (Invitrogen) and 50 uL of assay buffer (0.1% BSA-DMEM) per well. CACNG1 and isotype control antibodies were serial diluted in assay buffer and added to the cells and incubated at 37° C. in a 5% CO₂ incubator for 1 hour prior to the calcium flux assay. Nicardipine hydrochloride (Sigma) was added to untreated wells to serve as a control for calcium channel blockade. After 1 hour, acetylcholine (Sigma) was added at a 20 uM final concentration to induce myotube calcium release, and plates were assayed on a FLIPR Tetra (Molecular Devices, LLC).

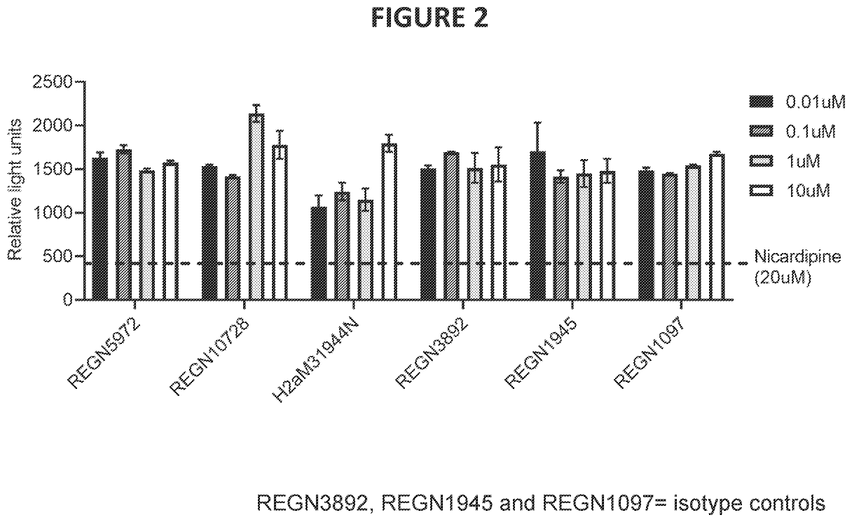

As expected, nicardipine substantially reduced human myotube calcium release, while none of the CACNG1 antibodies or isotype control antibodies had any major impact on calcium flux (FIG. 2). Overall, this demonstrates that anti-hCACNG1 antibodies bind, and are internalized, but do not block calcium release in human myotubes cultured in vitro.

### Example 5: CACNG1 Antibody Binding and Internalization in Myofibers Ex Vivo

Following confirmation of CACNG1 antibody binding to human myotubes, a subset of antibodies was tested for binding to fully mature myofibers ex vivo. Single myofibers were isolated from either wildtype mice, mice that were homozygous for the deletion of CACNG1 (referred to as CACNG1 knockout mice), or mice that were homozygous for the expression of human CACNG1 in place of mouse CACNG1 (referred to as CACNG1^{Hu/Hu}). The gastrocnemius muscle was removed, collagenase digested, and single myofibers were isolated, washed, and incubated overnight at 37° C. at 5% C02, in DMEM+10% horse serum. Following overnight incubation, single myofibers were incubated with 100 nM of each CACNG1 antibody or an isotype control antibody for 30 minutes. Myofibers were then washed twice in DMEM+10% horse serum, and subsequently incubated with 10 ug/mL of fluorescent-conjugated secondary antibodies for 30 minutes, washed twice in DMEM+10% horse serum, and then fixed with 4% paraformaldehyde (PFA) for 15 minutes at room temperature. Single fibers were then washed twice with PBS, stained with Hoechst for 5 minutes, washed once more with PBS, then transferred to microscope slides, coverslipped, and imaged using a Zeiss LSM 710 confocal microscope.

Separately, to determine whether an anti-human CACNG1 antibody as described herein can internalize in myofibers ex vivo, Alexa 647 (A647) fluorophore was directly conjugated to the CACNG1 antibody and an isotype control antibody. Single myofibers were isolated, washed, and incubated overnight. The following day, myofibers were incubated with 100 nM of the A647-conjugated antibody for 30 minutes, 4 hours, or 8 hours, and were then washed twice with PBS. Myofibers were then fixed with 4% PFA for 15 minutes, stained with Hoechst for 5 minutes, washed once more with PBS, then transferred to microscope slides, coverslipped, and imaged using a Zeiss LSM 710 confocal microscope.

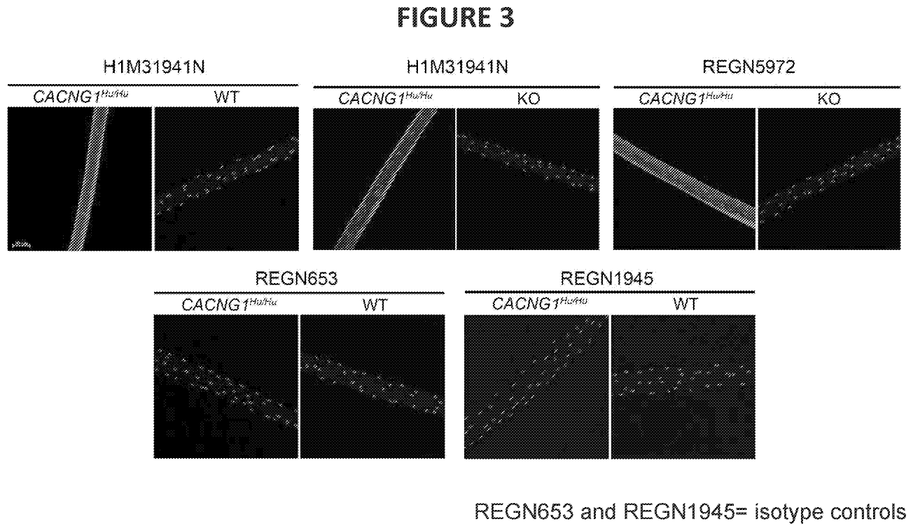

Two CACNG1 antibodies, H1M31941N and REGN5972, demonstrated binding to CACNG1^{Hu/Hu} myofibers ex vivo and did not bind to wildtype or CACNG1 knockout myofibers. Isotype control antibodies did not bind to either CACNG1^{Hu/Hu} myofibers or wildtype myofibers. (FIG. 3).

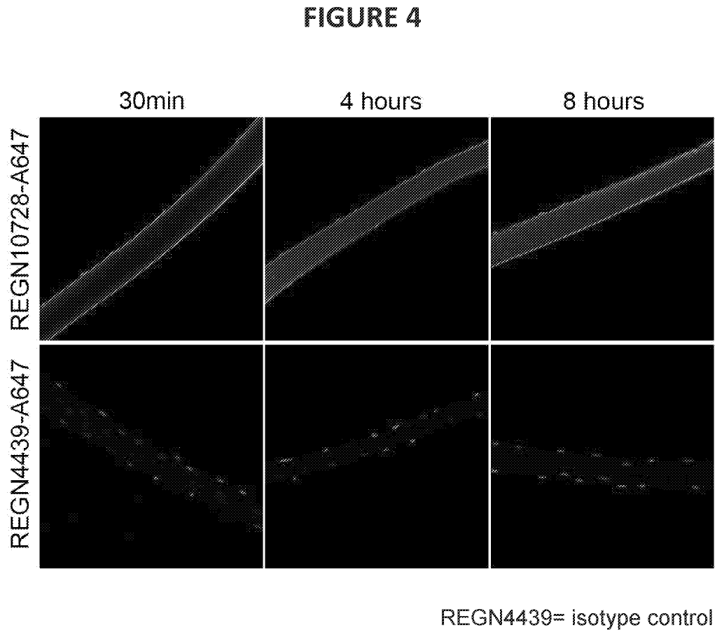

Single plane confocal imaging revealed that the fluorophore-conjugated CACNG1 antibody, REGN10728, bound to the surface of the myofiber following 30 minutes of incubation, and that it was internalized within the myofiber by as early as 4 hours (FIG. 4).

In conclusion, CACNG1 antibodies can bind to the surface of single myofibers ex vivo. Additionally, tracking of a fluorophore conjugated CACNG1 antibody demonstrates initial binding to the surface of the myofiber, followed by internalization into the myofiber after several hours.

### Example 6: CACNG1 Antibody-DHT Conjugate Androgen Reporter Assay

CACNG1 is the γ1 subunit of the dihydropyridine receptor that is expressed specifically in skeletal muscle. Therefore, antibodies generated against CACNG1 could be used to deliver conjugated therapeutic payloads specifically to skeletal muscle to enhance therapeutic efficacy in muscle and reduce off-target toxicity. For example, conjugation of the potent metabolite of testosterone, dihydrotestosterone (DHT), to CACNG1 antibodies, may allow for androgen receptor signaling in muscle, leading to increased muscle mass and function. Here, CACNG1 antibodies conjugated to a linker with DHT payload were tested in an androgen receptor (AR) reporter cell line to determine whether these antibody conjugates can specifically activate the AR in CACNG1-expressing cells in vitro.

To assess signaling via the AR, LNCaP cells were transfected with lentivirus (Qiagen; ARE.Luc Cignal Lenti) to generate a stable cell line that expressed AR-luciferase reporter (AR.Luc). A subset of these selected cells was transduced to express human CACNG1 and further selected, with this cell line being referred to as hCACNG1.AR.Luc.

For the bioassay, AR.Luc or hCACNG1.AR.Luc cells were plated at 5,000 cells/well in OptiMEM and 0.5% charcoal-stripped FBS in PDL-coated 96-well plates. Cells were then incubated for 24, 48, or 27 hours with CACNG1 antibodies or isotype control antibody conjugated to DHT (via M3004 linker-payload), or unconjugated DHT alone (M608). All antibodies were conjugated with DHT at a drug-antibody-ratio (DAR) of ~4. After the respective timepoints, cells were lysed and incubated with One-GLO buffer, and luminescence was read on an Envision plate reader. Relative luminescence units (RLU) were plotted against log concentration in mol/L, adjusted for DAR.

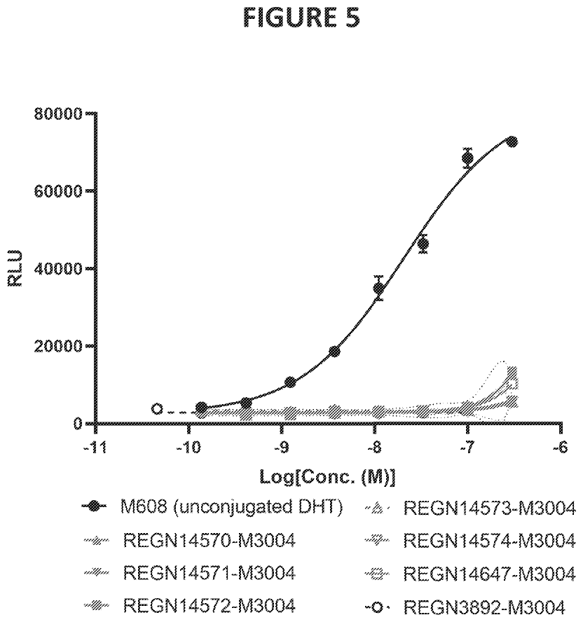

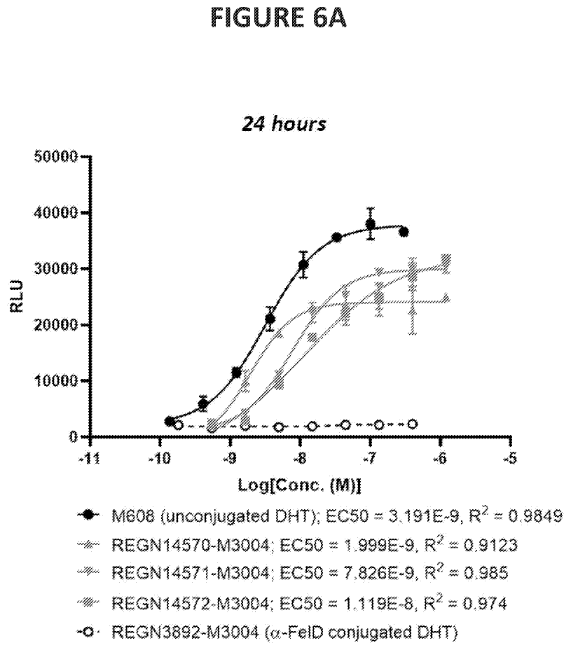

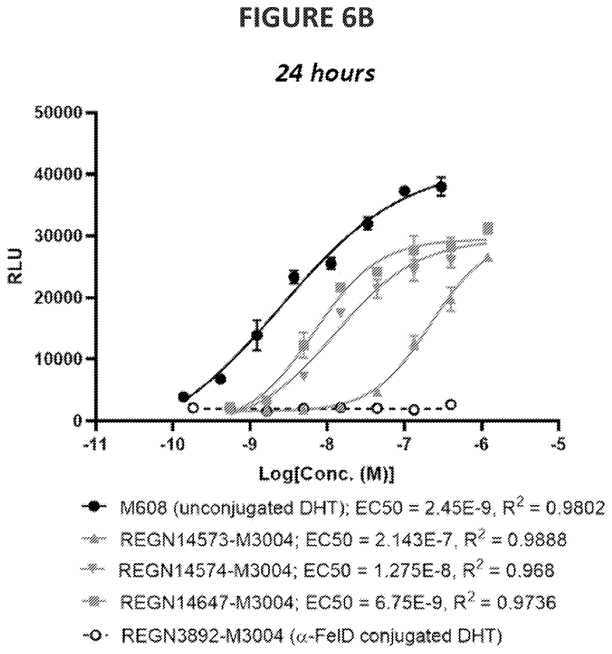

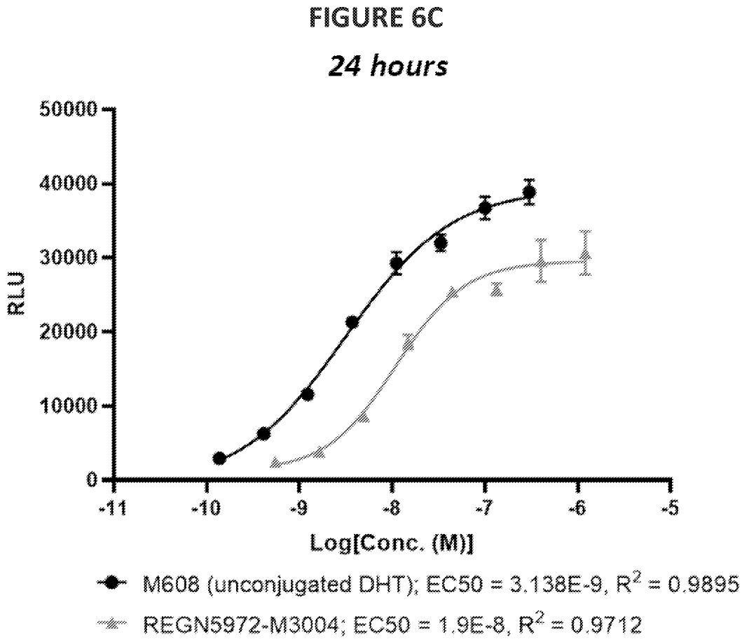

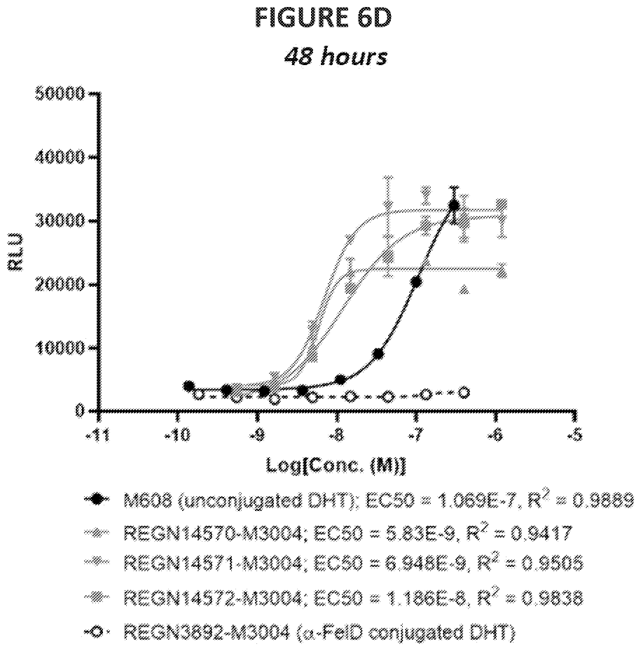

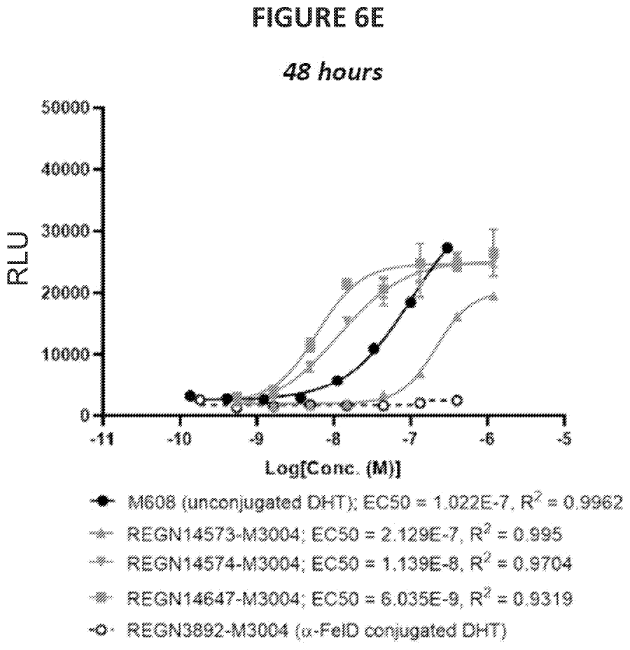

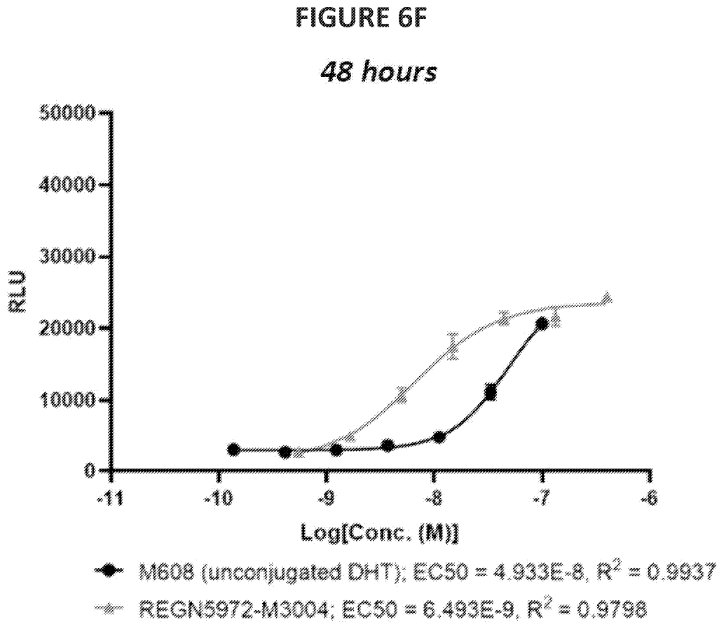

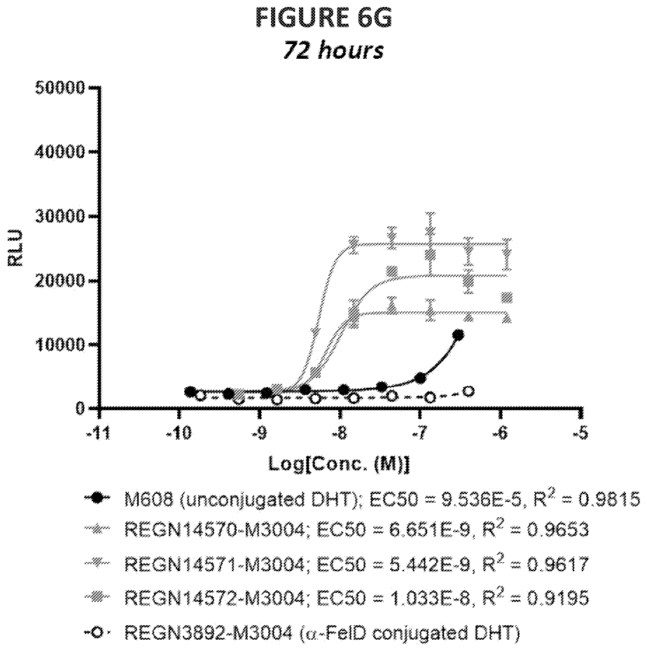

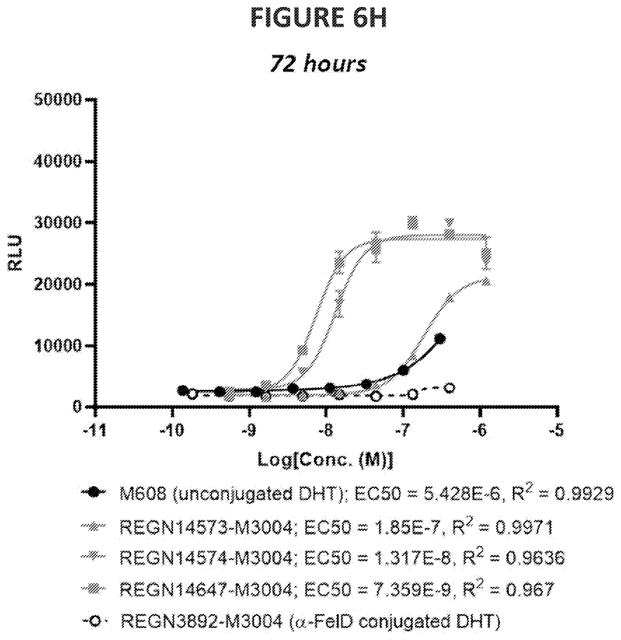

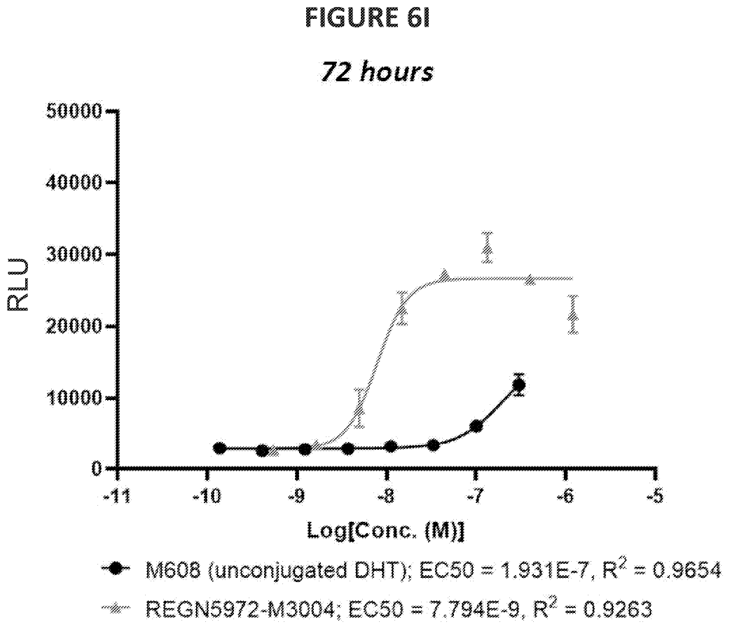

Unconjugated DHT (M608) activated AR in both AR.Luc (FIG. 5) and hCACNG1.AR.Luc cell lines (FIG. 6), whereas DHT conjugated to an isotype control antibody (REGN3892-M3004) did not activate AR in either of these cell lines. Several CACNG1 antibody-DHT conjugates activated AR only in the hCACNG1.AR.Luc cell line (FIG. 6), but not in the AR.Luc cell line (FIG. 5). While the efficacy and potency of AR activation of CACNG1 antibody-DHT conjugates was lower than that of unconjugated DHT at 24 hours following treatment (FIGS. 6A-6C), activation of the AR was sustained at 48 and 72 hours with these conjugates, whereas unconjugated AR signal decreased substantially at these timepoints (FIGS. 6D-6I). Overall, these data demonstrate that conjugation of DHT to CACNG1 antibodies allows for specific activation of the AR in cells expressing hCACNG1, and that DHT conjugated to CACNG1 antibodies maintains sustained AR signaling over several days in hCACNG1-expressing cells in vitro.

### Example 7: In Vivo Biodistribution

To determine whether CACNG1 antibodies can specifically target skeletal muscle in vivo, CACNG1 antibodies (REGN5972 and REGN10728) and an isotype control antibody (REGN4439) were conjugated with Alexa Fluor 647 fluorescent dye, and were tail vein injected into mice (n=1/group) that were homozygous for the expression of human CACNG1 in place of mouse CACNG1 (referred to as CACNG1^{Hu/Hu}) at a dose of 10 mg/kg, or with saline control. Six days following injection, mice were cryopreserved for whole body antibody distribution analysis using cryo-fluorescence tomography (Invicro). A separate set of mice (n=1/group) that was injected with the same antibodies (or saline control) were sacrificed at 6 days post-injection and PBS perfused, and the following tissues were harvested for immunofluorescence analysis: tibialis anterior, gastrocnemius/plantaris/soleus complex, diaphragm, tongue, pelvic floor muscle, triceps, trapezius, liver, kidney, spleen, brown adipose. Tissues were embedded in optical coherence tomography (OCT) compound, frozen in liquid nitrogen-cooled isopentane, and subsequently cryo-sectioned at 10 um onto microscope slides. Tissue sections were then permeabilized with Triton X-100, blocked with 4% BSA, and incubated with a rabbit-derived laminin antibody (Sigma) overnight. The following day, sections were washed, stained with anti-rabbit Alexa 488 secondary antibody (Thermo Fisher), counterstained with Hoescht, washed, fixed with 4% PFA, washed, and mounted with Fluoromount-G (Thermo Fisher). Tissues were then imaged on a Zeiss Axioscan Z1 slide scanner to visualize tissue distribution of Alexa 647-conjugated antibodies.

Alexa 647-conjugated CACNG1 antibodies, REGN10728 and REGN5972, displayed clear signal in multiple skeletal muscles via cryo-fluorescence tomography imaging, while the isotype control antibody did not show muscle uptake and accumulated mostly in the bladder (FIG. 7). Saline-dosed control did not show any appreciable fluorescent signal throughout the mouse. Fluorescent signal in the muscle appeared stronger in the mouse dosed with CACNG1 antibody REGN10728 compared to REGN5972, though their overall muscle distribution pattern was similar.

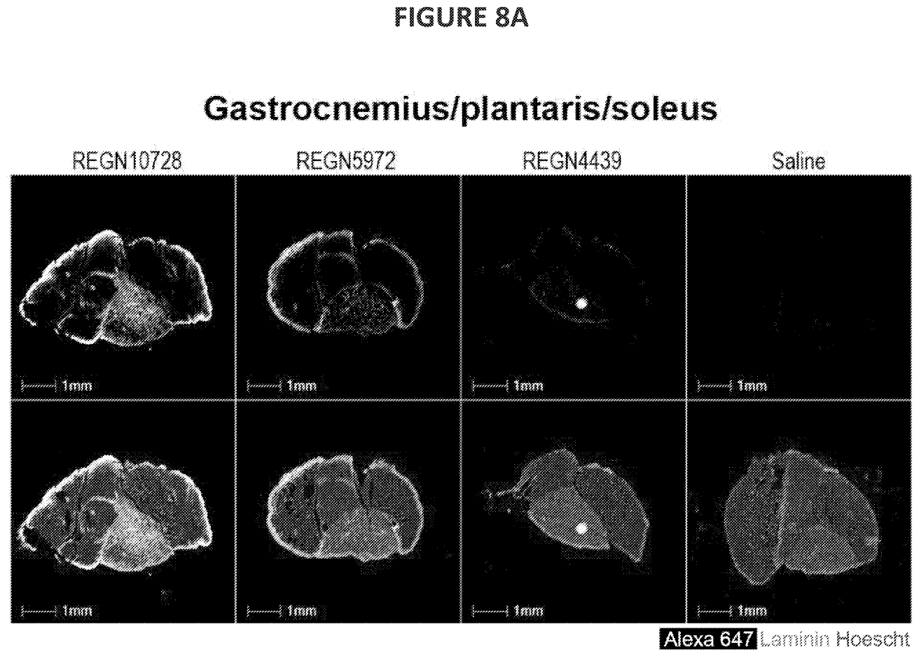

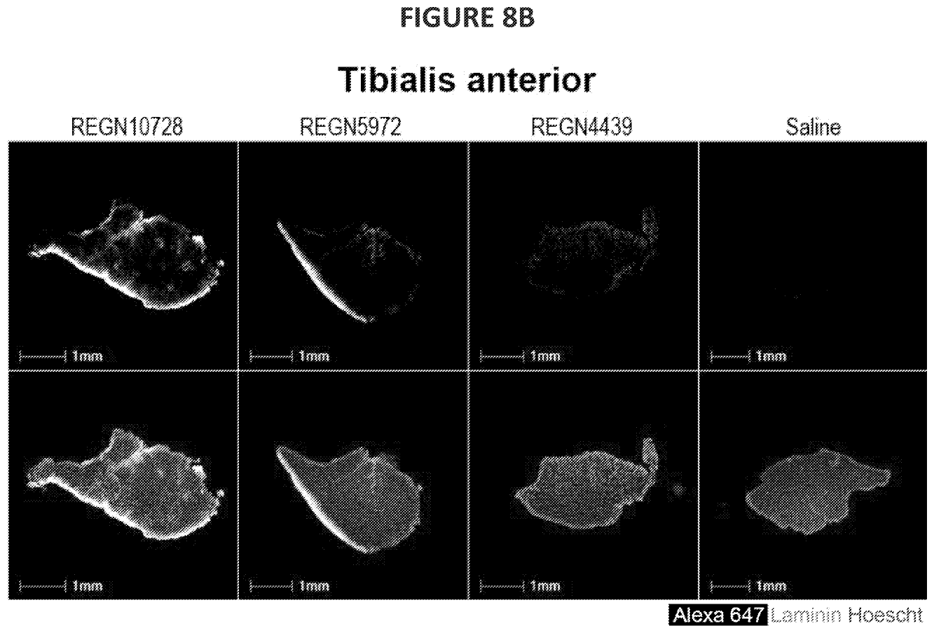

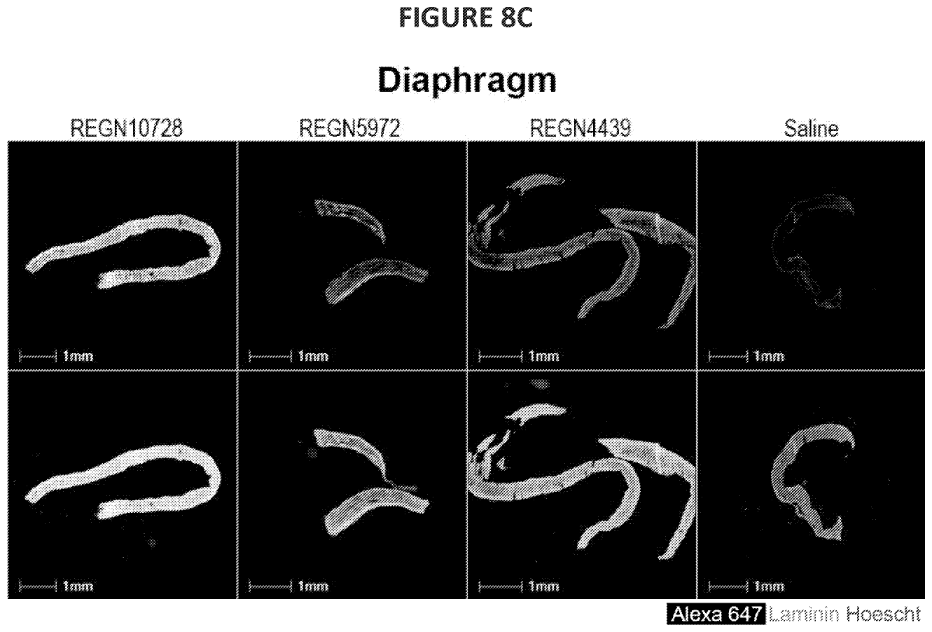

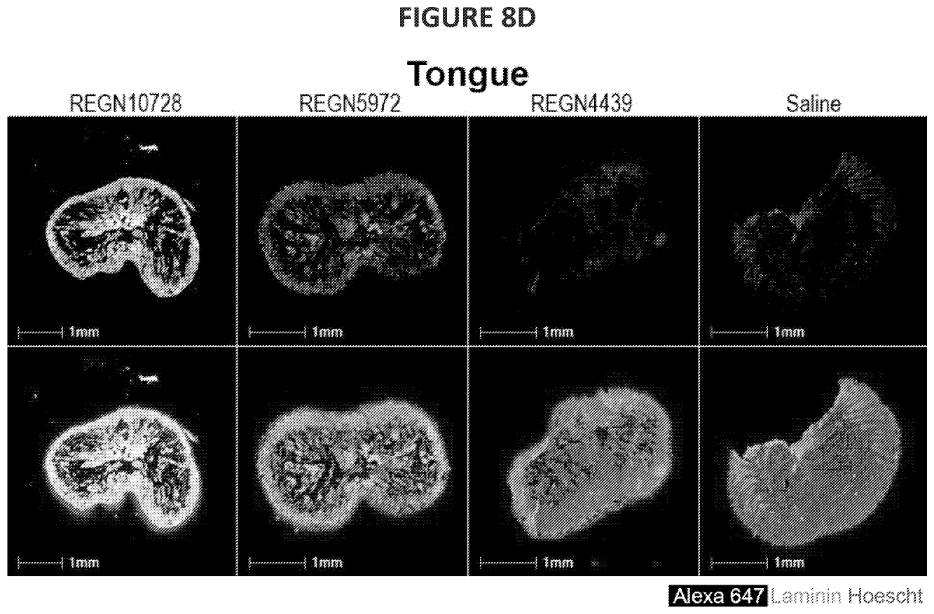

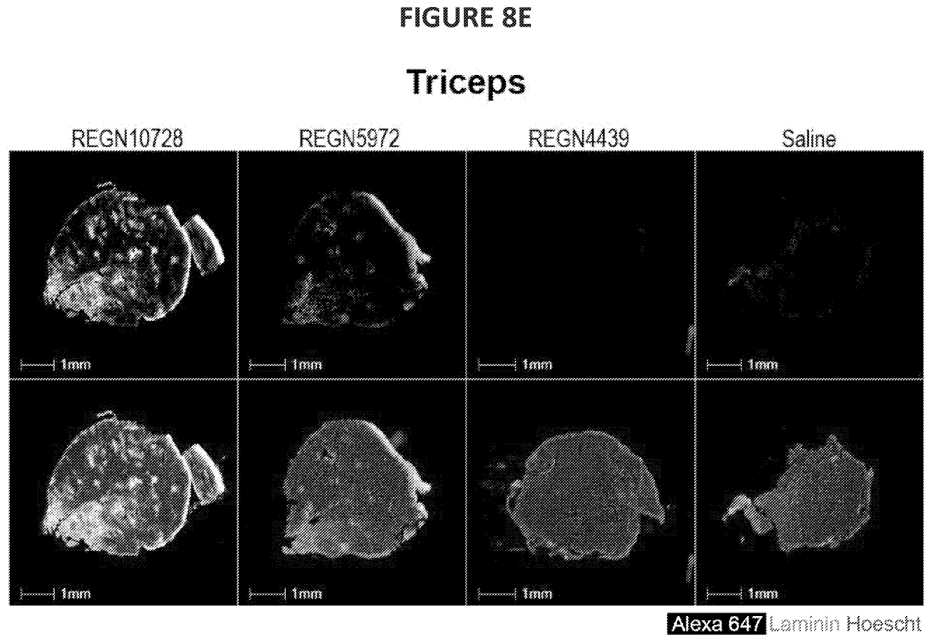

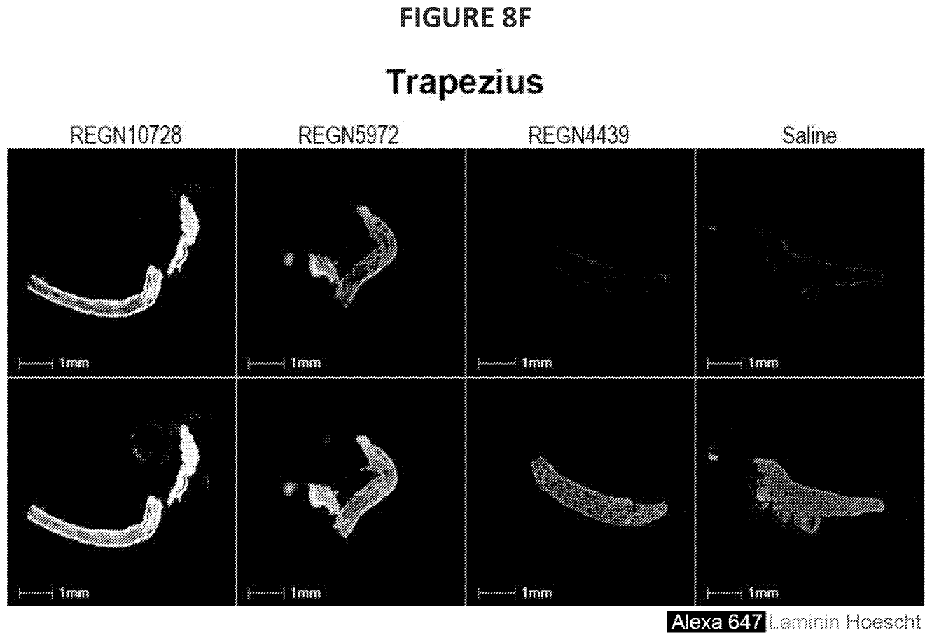

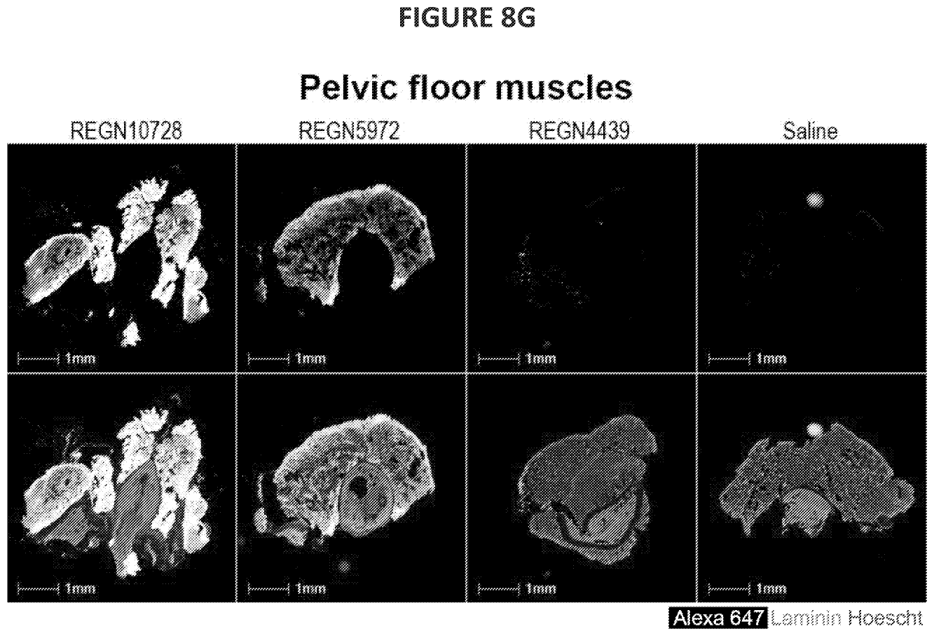

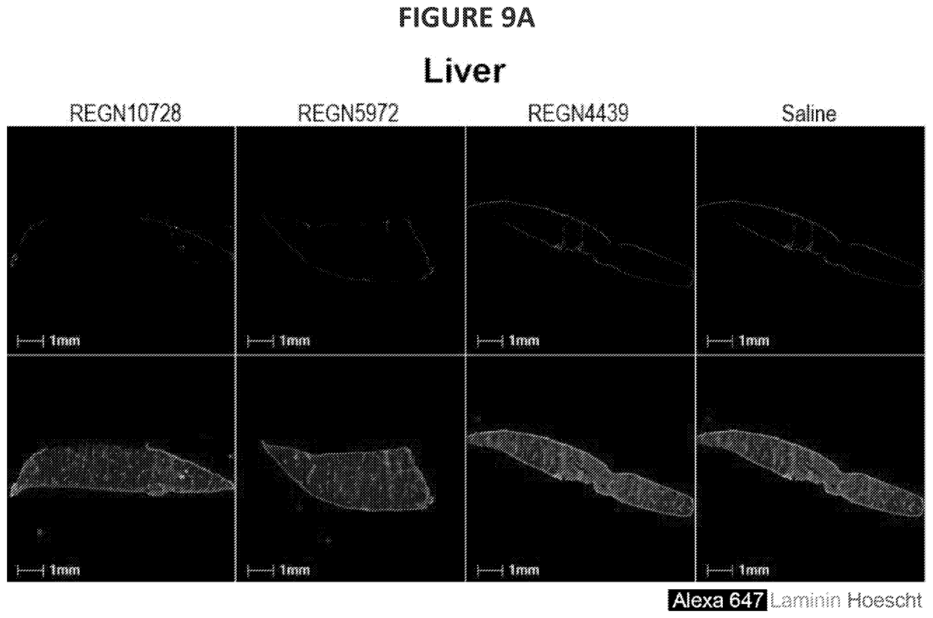

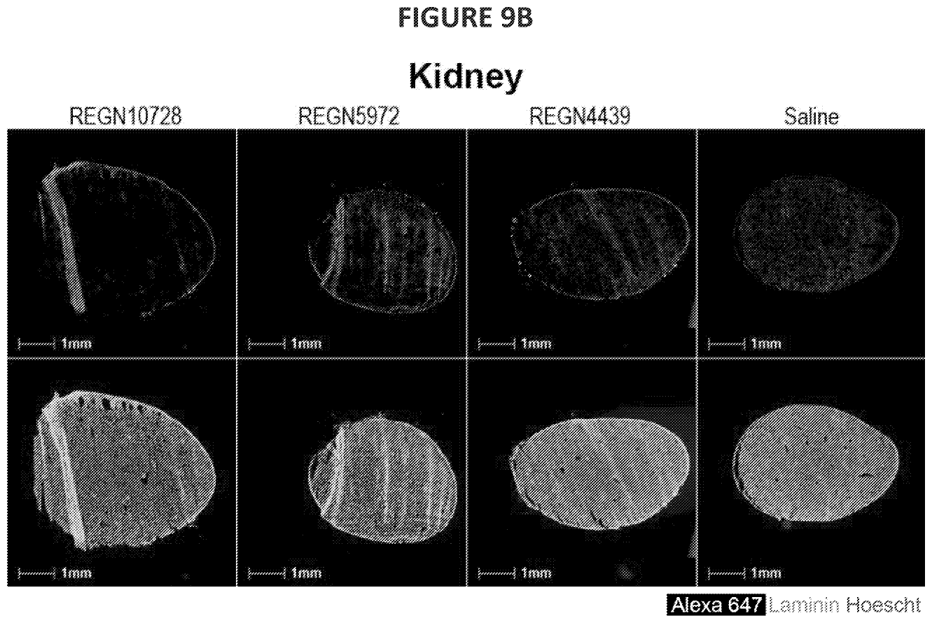

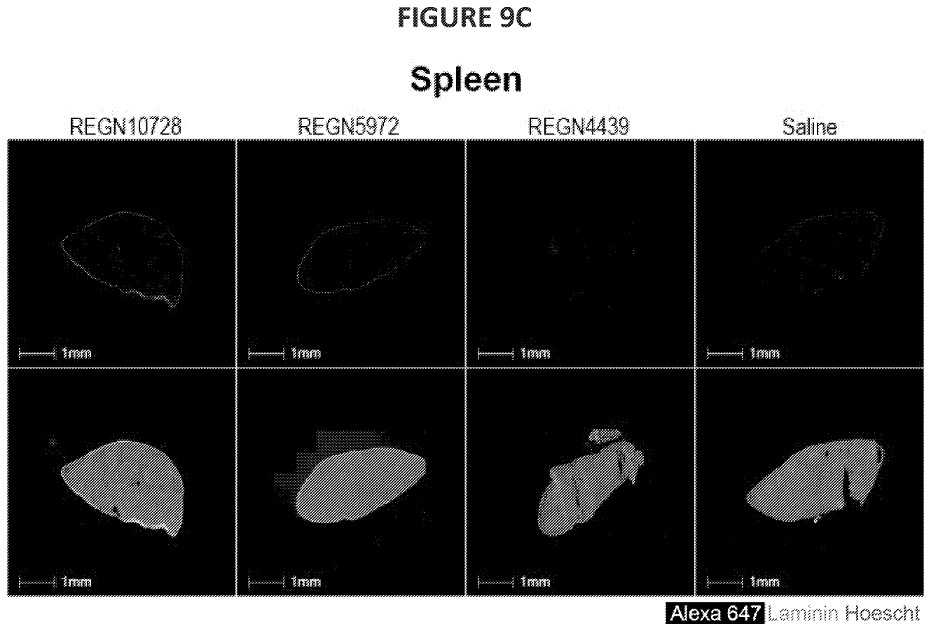

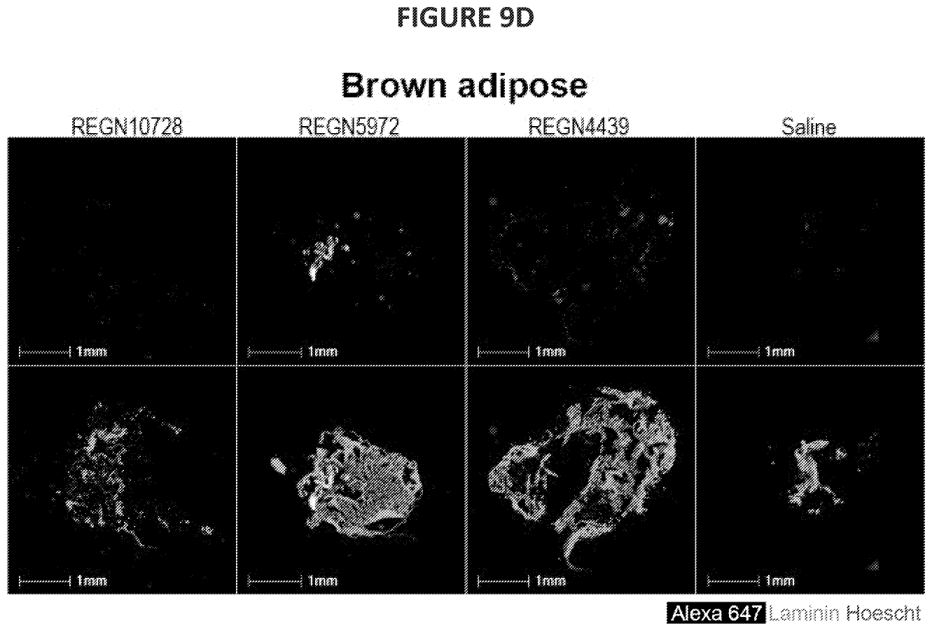

Fluorescence imaging of tissue sections revealed uptake of Alexa 647-conjugated CACNG1 antibodies REGN10728 and REGN5972 in multiple skeletal muscles, including: gastrocnemius/plantaris/soleus complex, diaphragm, tongue, pelvic floor muscle, triceps, and trapezius (FIG. 8). Similar to cryo-fluorescence tomography imaging findings, the overall signal of REGN10728 appeared stronger in the muscle sections compared to REGN5972. Neither REGN10728 and REGN9572 showed any clear staining in multiple non-muscle tissues, including: liver, kidney, spleen, and brown adipose (FIG. 9).

These in vivo biodistribution studies demonstrated that fluorophore-conjugated CACNG1 antibodies specifically target skeletal muscle, and are not taken up by other non-muscle tissues.

### Example 8: CACNG1 Antibody Distribution to Muscle is Altered by Exercise and Dose

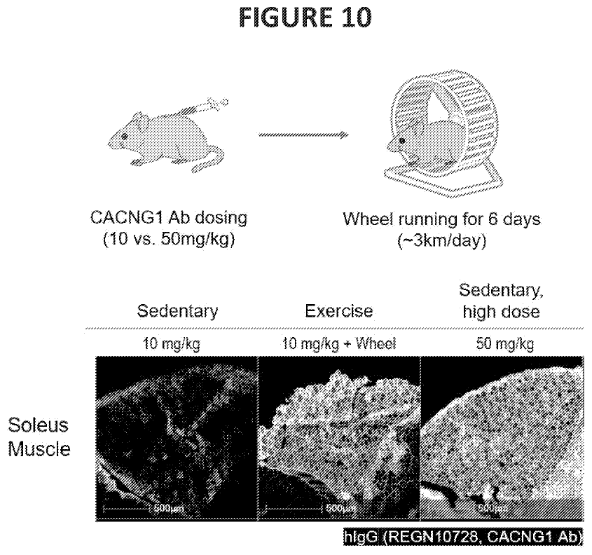

To test methods to enhance CACNG1 antibody distribution to muscle, CACNG1^{Hu/Hu} mice were dosed with 10 mg/kg or 50 mg/kg CACNG1 antibody, and a subset of mice were given access to exercise wheels (FIG. 10, top panel). Wheel running enhanced CACNG1 antibody distribution to the working soleus muscle, and 50 mg/kg dose also showed enhanced distribution throughout the soleus (FIG. 10, bottom panel).

### Example 9: Adeno-Associated Virus Retargeted to CACNG1 for Use in Treating Muscle Related Disorders

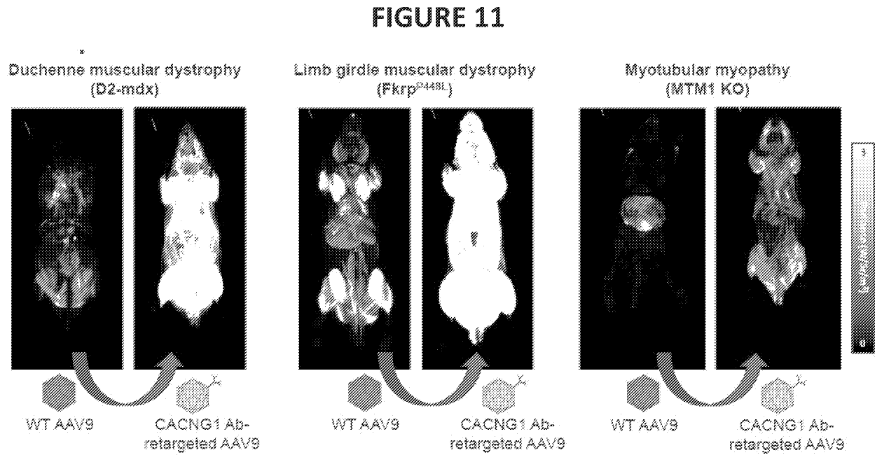

PCT/US2022/079339 provides evidence that a recombinant adeno-associated virus (AAV) particle comprising an AAV capsid protein modified to display an anti-CACNG1 antibody retargets the AAV particle to skeletal muscle in healthy mice. To determine whether such AAV may also be targeted to skeletal muscle in mice with muscle disease, 5×10¹² viral genomes/kg (vg/kg) of AAV9 particles modified with anti-CACNG1 antibodies and carrying a GFP reporter gene under the control of a CAG promoter were injected into a mouse model for Duchenne muscular dystrophy (D2-mdx), limb girdle muscular dystrophy (Fkrp^{P448L}), and myotubular myopathy (MTM1 KO), and mice were cryopreserved two weeks following injection for cryo-fluorescence tomography imaging. See, e.g., FIG. 11 showing that AAV9 viral particles comprising capsid proteins retargeted to CACNG1 are retargeted to skeletal muscle in D2-mdx mice, Fkrp^{P448L} mice, and MTM1 KO mice, as whole body GFP signal indicates increased signal in various skeletal muscles and reduced signal in other tissues (e.g., liver) compared to wildtype AAV9 treated mice.

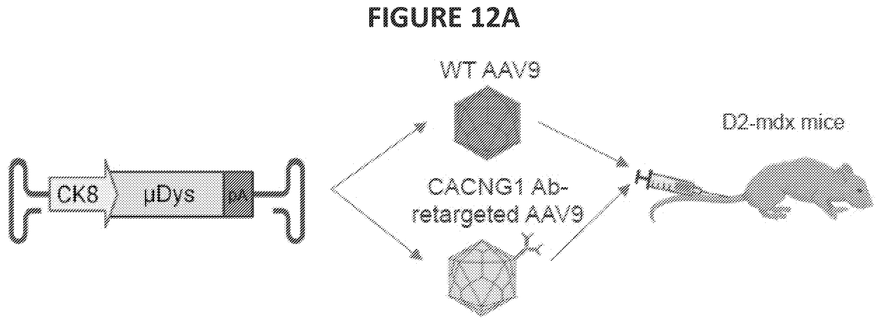

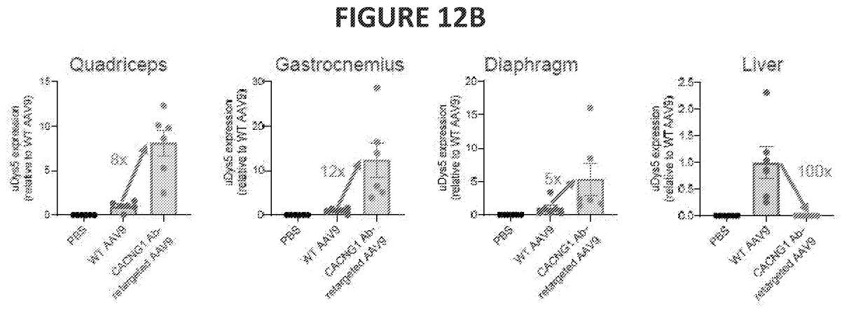

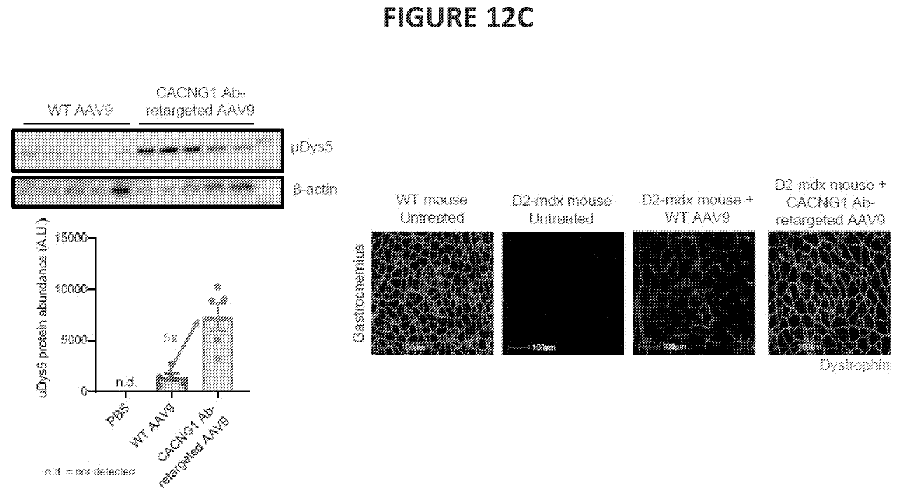

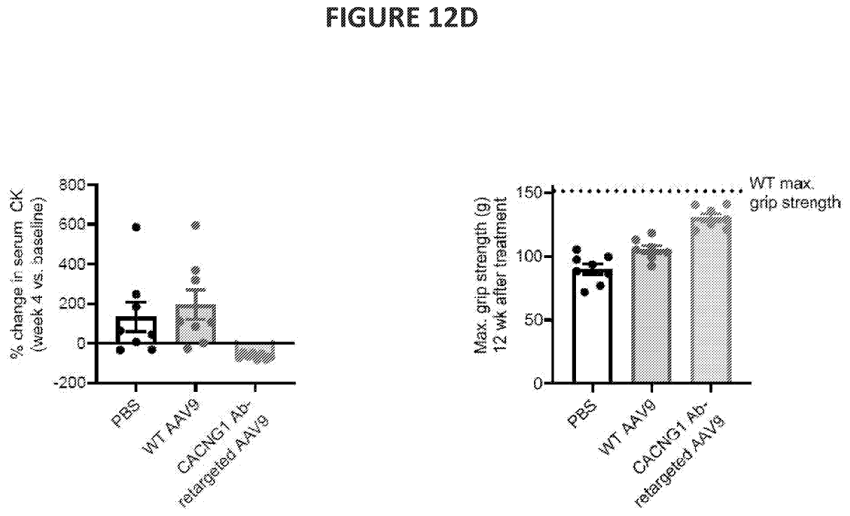

To test whether AAV9 viral particles comprising capsid proteins retargeted to CACNG1 can carry therapeutic nucleotides of interest, CACNG1 retargeted AAV particles comprising sequences encoding microdystrophin (μDys), FKRP, or MTM1 were injected into D2-mdx mice, Fkrp^{P448L} mice, or MTM1 KO mice, respectively. See, e.g., FIGS. 12A, 13A, and 14A.

In D2-mdx mice, the loss of functional dystrophin results in sarcolemmal instability, and persistent and severe damage is observed in skeletal muscles. 6-week-old D2-mdx dystrophic mice were treated intravenously with 1×10¹² vg/mouse (~5×10¹³ vg/kg body weight) of WT AAV9 or AAV9 particles comprising a N272 mutation retargeted with an anti-hCACNG1 antibody (REGN10717) encapsulating a nucleotide of interest encoding microdystrophin (μDys) under the control of a CK8 promoter, or PBS as a control. FIGS. 12B-12D show that injection with CACNG1-retargeted AAV9 particles (which may include N272A mutations in a capsid protein to further detarget from the liver) carrying therapeutic microdystrophin gene enhances microdystrophin mRNA expression in the skeletal muscle of D2-mdx mice, and reduces microdystrophin expression in the liver when compared to infection with wildtype AAV9 particles carrying the microdystrophin gene (FIG. 12B). Enhanced protein levels of microdystrophin are seen in the quadriceps and at the myofiber membrane in skeletal muscle after infection with CACNG1-retargeted AAV9 particles (which may include N272A mutations in a capsid protein to further detarget from the liver) carrying therapeutic microdystrophin gene compared to wildtype AAV9 particles (which may include N272A mutations in a capsid protein to further detarget from the liver) carrying therapeutic microdystrophin gene (FIG. 12C). Additionally, retargeting AAV9 particles to CACNG1 improved the therapeutic efficacy of AAV9 mediated microdystrophin gene therapy of D2-mdx mice, as shown by a reduction in serum creatine kinase (a marker of muscle damage) within the first 4 weeks of treatment and enhanced forelimb grip strength in treated mice 12 weeks after treatment (FIG. 12D).

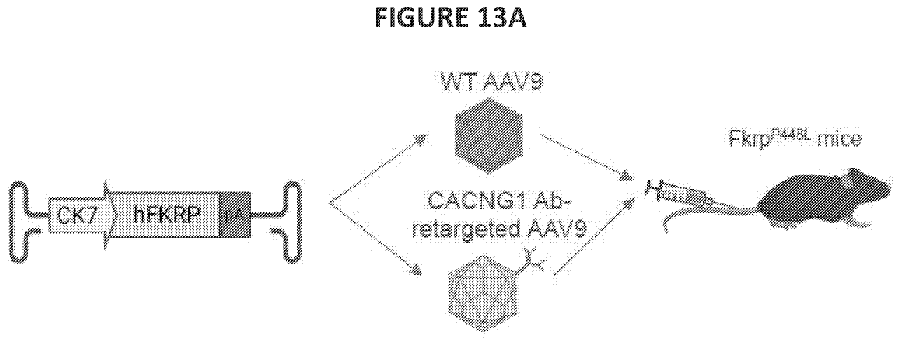

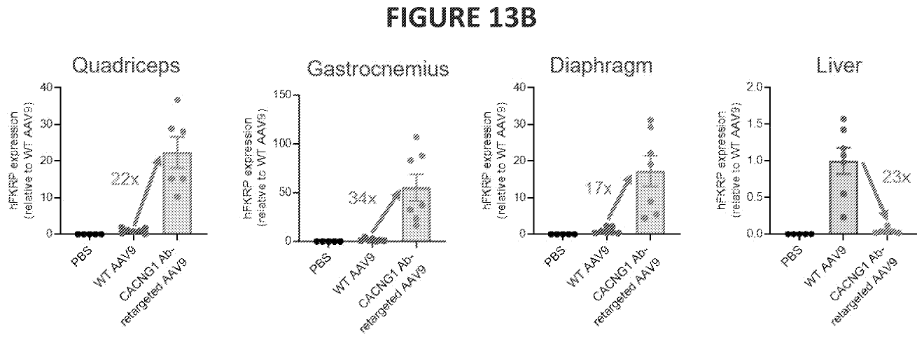

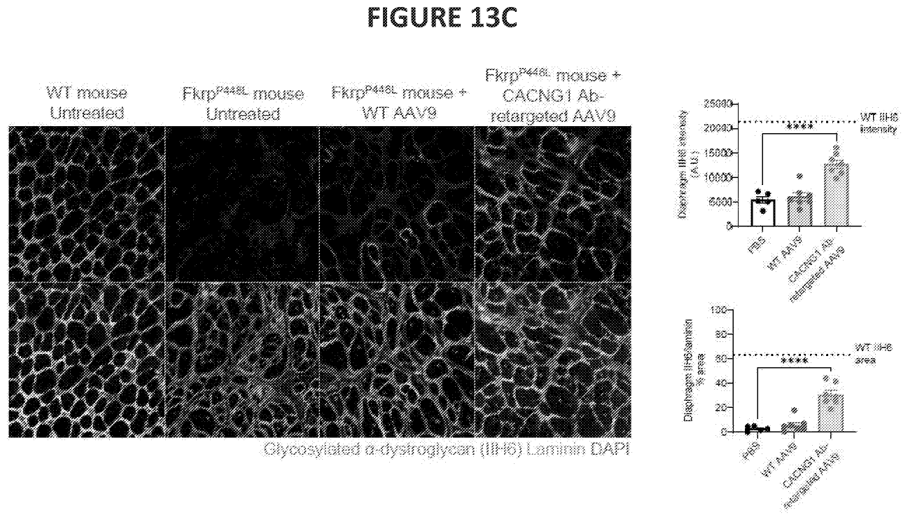

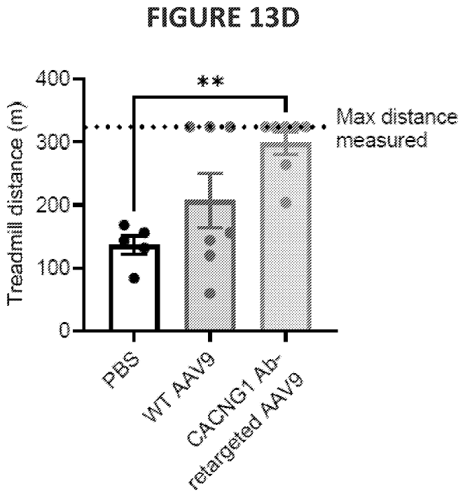

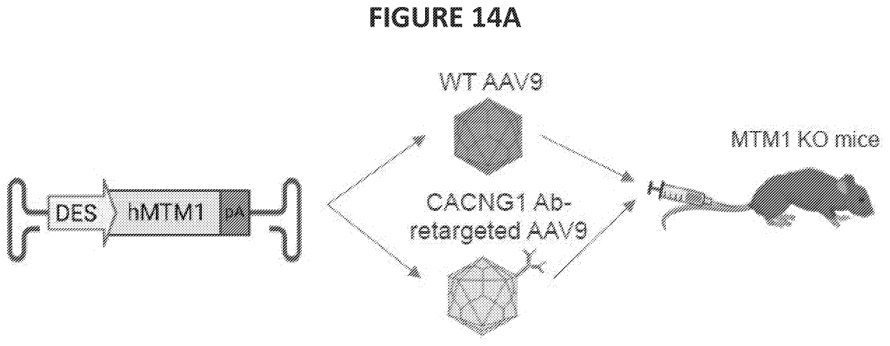

Limb girdle muscular dystrophy 2I/R9 is caused by a mutation in the FKRP gene, leading to decreased glycosylation of α-dystroglycan and muscular dystrophy. 8-10-week-old Fkrp^{P448L} mice were treated with 1×10¹¹ vg/mouse (~4×10¹² vg/kg body weight) of WT AAV9 or AAV9 particles comprising an N272A mutation retargeted with an anti-hCACNG1 antibody (REGN10717) encapsulating a nucleotide of interest encoding human FKRP (hFKRP) under the control of a CK7 promoter, or PBS as a control. FIGS. 13B-13D show that CACNG1 antibody-retargeted AAV9 enhances FKRP mRNA expression in the skeletal muscle of Fkrp^{P448L} mice7 weeks after treatment. Additionally, targeting therapeutic AAV9 particles with anti-CACNG1 antibodies reduces FKRP expression in the liver when compared to infection with wildtype AAV9 particles carrying the FKRP gene (FIG. 13B), enhances glycosylation of α-dystroglycan in the mouse diaphragm as assessed by immunofluorescence intensity and area (FIG. 13C), and enhances exercise capacity as assessed by an improved maximal downhill running distance 7 weeks following treatment (FIG. 13D).

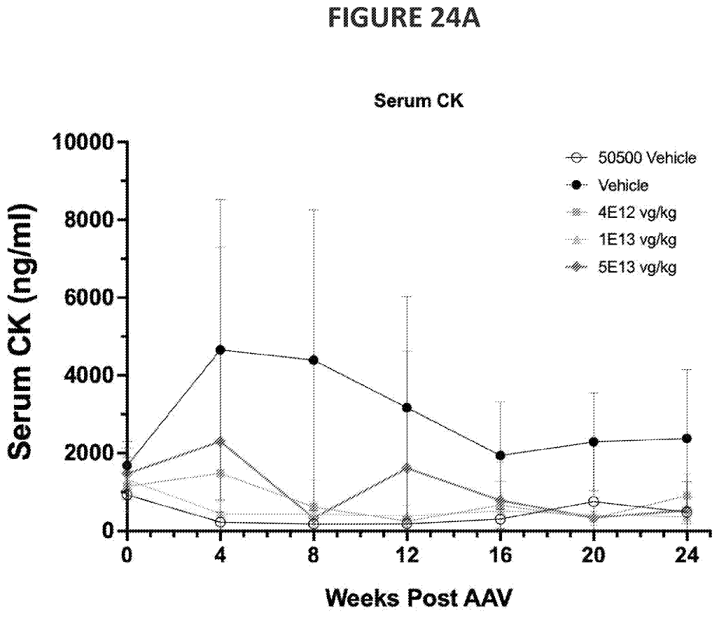

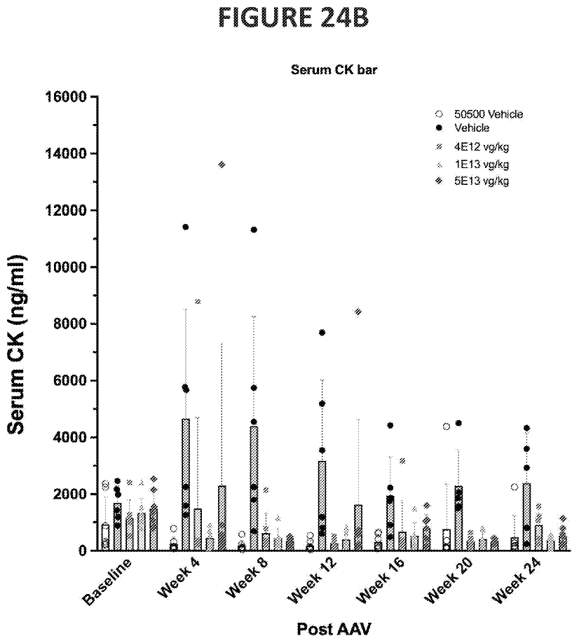

An AAV dose titration was further performed to assess the phenotypic rescue of a mouse model of limb girdle muscular dystrophy 2I/R9 (Lama2^{HU/HU}/DAG1^{HU/HU}/FKRP^{P448L/P448L}). At all doses tested, serum creatine kinase levels (a marker of skeletal muscle damage) were lower in Lama2^{HU/HU}/DAG1^{HU/HU}/FKRP^{P448L/P448L} mice treated with AAV9 particles comprising a N272A mutation retargeted with an anti-hCACNG1 antibody (REGN10717) encapsulating a nucleotide of interest encoding human FKRP (hFKRP) under the control of a CK7 promoter (REGN10717-AAV9(N272A)-hFKRP) compared to vehicle treated mice, as early as 4 weeks post-treatment, and up to 24 weeks post-treatment (FIGS. 24A-24B). These results demonstrate that treatment with doses of AAV9 particles comprising an N272A mutation retargeted with an anti-hCACNG1 antibody encapsulating a nucleotide of interest encoding hFKRP, e.g., REGN10717-AAV9(N272A)-hFKRP, as low as 4×10¹² viral genomes/kg may result in phenotypic rescue (assessed by circulating muscle damage markers) of FKRP^{P448L/P448L} mice, and restoration of serum CK levels to similar levels as wildtype mice.

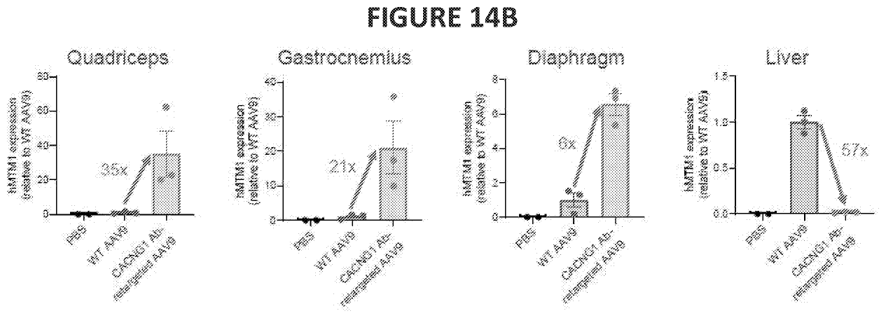

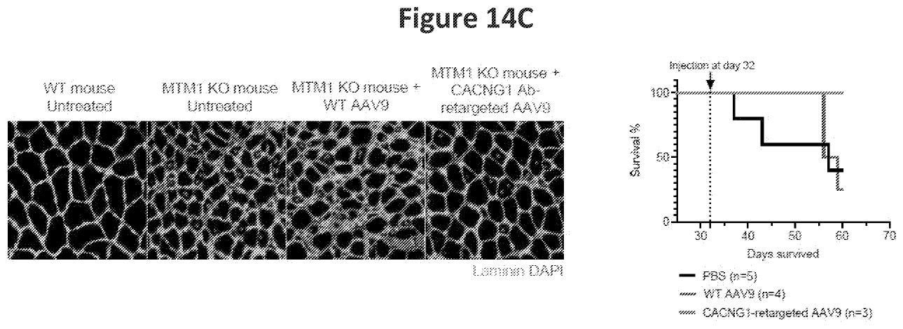

X-linked myotubular myopathy results from a mutation in the MTM1 gene (encoding myotubularin) and results in hypotrophic and centrally-nucleated myofibers, severe muscle weakness, and a shortened lifespan. 4-week-old MTM1 knockout (KO) mice were treated with 2×10¹⁰ vg/mouse (~2×10¹² vg/kg body weight) of WT AAV9 or AAV9 particles comprising an N272A mutation retargeted with an anti-hCACNG1 antibody (REGN10717) encapsulating a nucleotide of interest encoding human MTM1 (hMTM1) under the control of a desmin promoter, or PBS as a control. FIGS. 14B-14C show that CACNG1 antibody-retargeted AAV9 enhances MTM1 mRNA expression in the skeletal muscle of MTM1 KO mice compared to wildtype AAV9. Targeting therapeutic AAV9 particles with anti-CACNG1 antibodies reduces MTM1 expression in the liver when compared to infection with wildtype AAV9 particles carrying the MTM1 gene (FIG. 14B), and appears to improve muscle histopathology, e.g., of the soleus muscle (FIG. 14C, left panel), and survival of MTM1 KO mice (FIG. 14C, right panel).

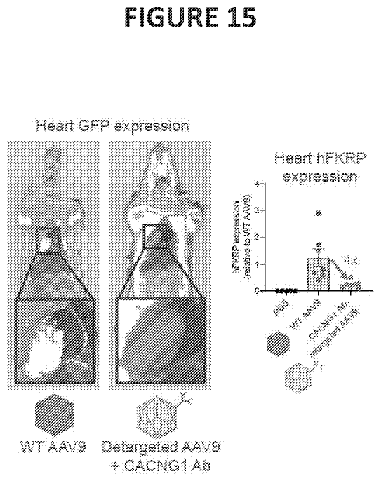

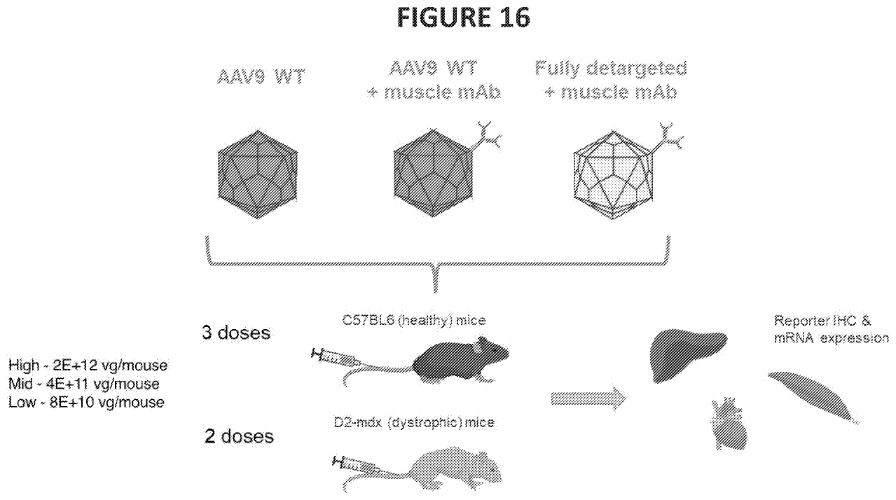

Moreover, aside from enhancing therapeutic efficacy in skeletal muscle, retargeting AAV allows for tunable heart transduction (FIG. 15 and FIG. 16). As demonstrated in FIG. 15, detargeted AAV9 (via an N272A mutation) retargeted via conjugation to an anti-hCACNG1 antibody (REGN10717) reduces heart transduction (as assessed by cryo-fluorescence tomography GFP intensity) in Fkrp^{P488L} mice as compared to wildtype AAV9 two weeks following treatment with 5×10¹² viral genomes/kg (vg/kg) of AAV9 particles encapsulating eGFP under the control of the CAG promoter. Similarly, reduced expression of hFKRP mRNA was observed in the heart of Fkrp^{P488L} mice 7 weeks after systemic treatment with 1×10¹¹ vg/mouse (~4×10¹² vg/kg body weight) of detargeted AAV9 (via an N272A mutation) retargeted via conjugation to an anti-hCACNG1 antibody (REGN10717) when compared to wildtype AAV9 encapsulating hFKRP under the control of the CK7 promoter. Additional experiments were then performed to determine if it was possible to achieve robust skeletal muscle targeting while retaining heart transduction by conjugating anti-hCACNG1 antibodies to wildtype AAV9.

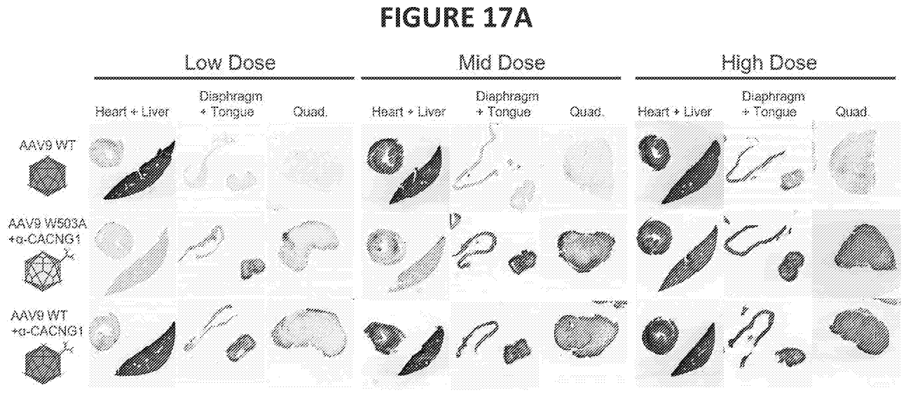

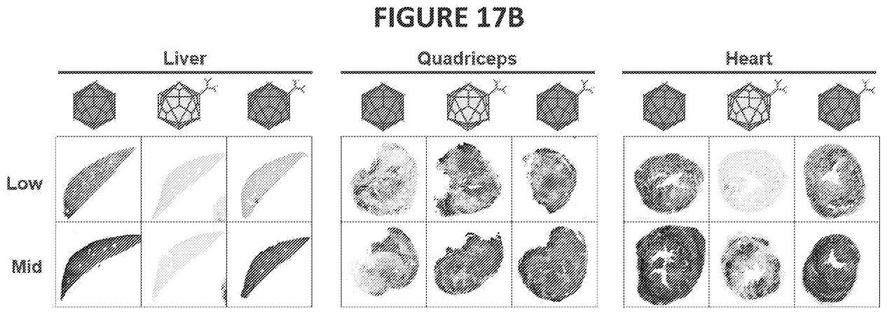

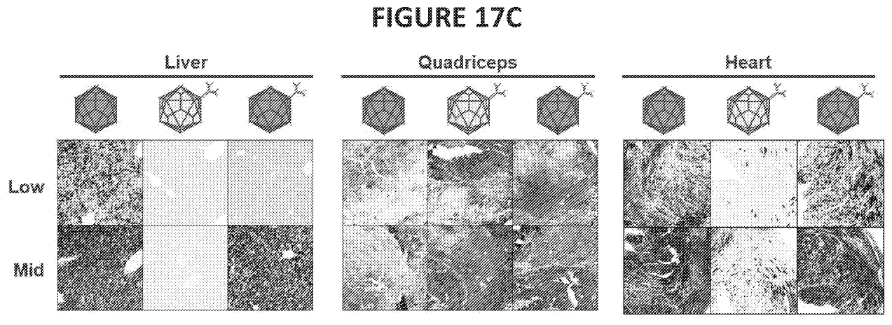

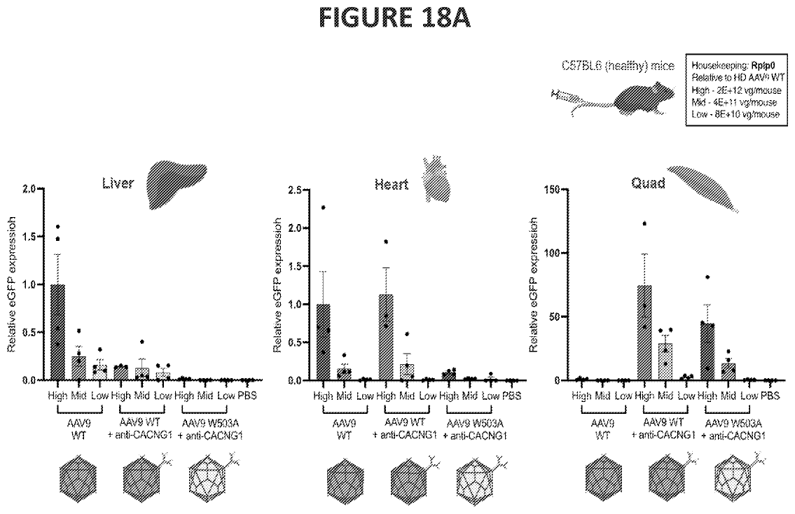

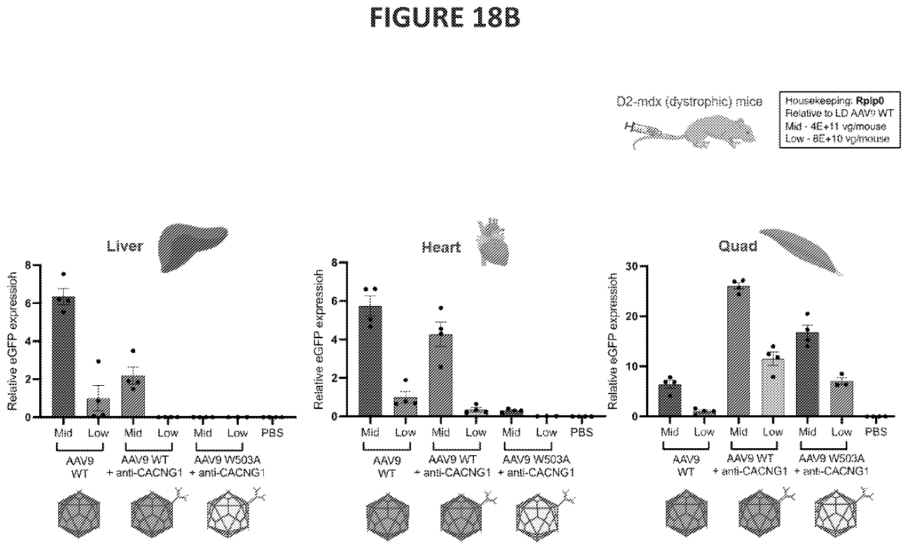

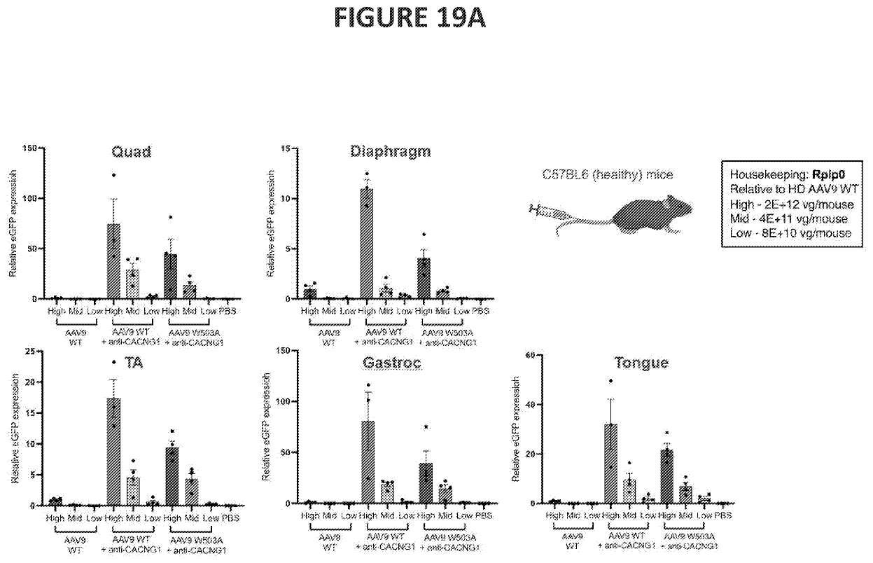

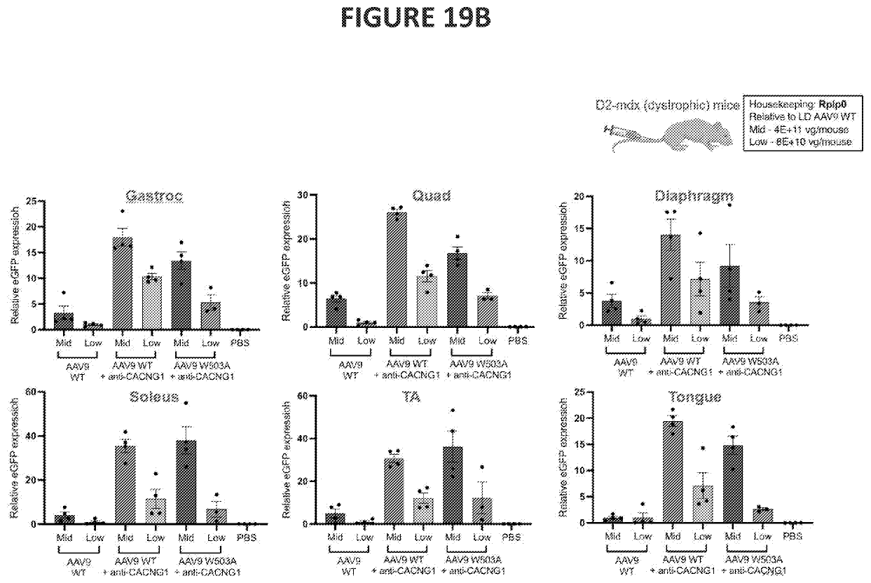

As further demonstrated in FIGS. 17 and 18, AAV9 wildtype capsids retain heart and mild liver transduction and display enhanced muscle tropism at a range of doses (e.g., 2×10¹² vg/mouse [high], 4×10¹¹ vg/mouse [mid] or 8×10¹⁰ vg/mouse [low]) when targeted via anti-CACNG1 antibodies in healthy mice (FIGS. 17A and 18A) and dystrophic mice (FIGS. 17B, 17C and 18B). Skeletal muscle transduction by both wildtype and detargeting AAV9 capsids is enhanced by conjugation to CACNG1-targeting antibodies in healthy mice (FIG. 19A) and dystrophic mice (FIG. 19B), with a slight reduction in muscle transduction observed for anti-CACNG1 antibodies conjugated to AAV9 W503A detargeted capsids.

The data provided herein show that retargeting AAV9 capsids using skeletal muscle-specific CACNG1 antibodies greatly improves muscle transduction in multiple mouse models of disease. As non-limiting examples, treatment with relatively low systemic doses of CACNG1-retargeted AAV9 comprising a microdystrophin gene reduced muscle damage and improved muscle strength in D2-mdx mice (FIGS. 12A-12D), treatment with relatively low systemic doses of CACNG1-retargeted AAV9 comprising a gene encoding human FKRP increased glycosylation of α-dystroglycan and improved exercise capacity of Fkrp^{P488L} mice (FIGS. 13A-13D), and treatment with relatively low systemic doses of CACNG1-retargeted AAV9 comprising a gene encoding human MTM1 improved muscle histopathology and survival of MTM1 knockout mice (FIGS. 14A-14C).

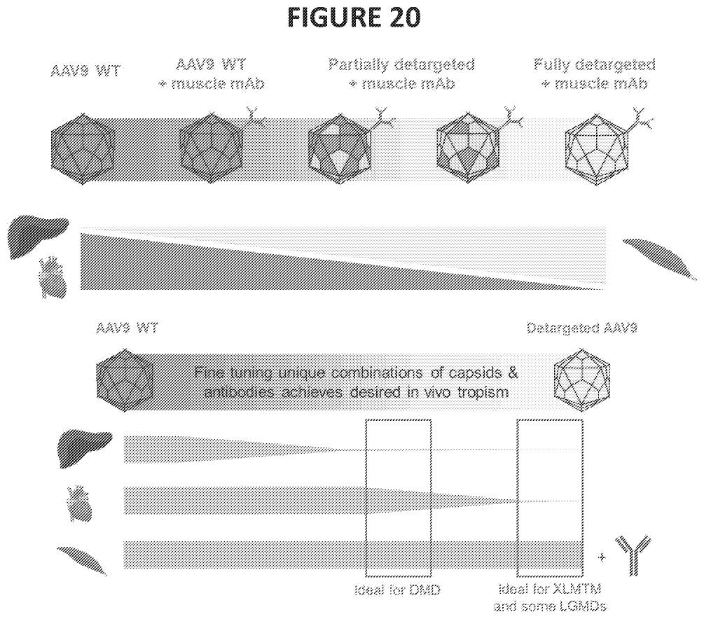

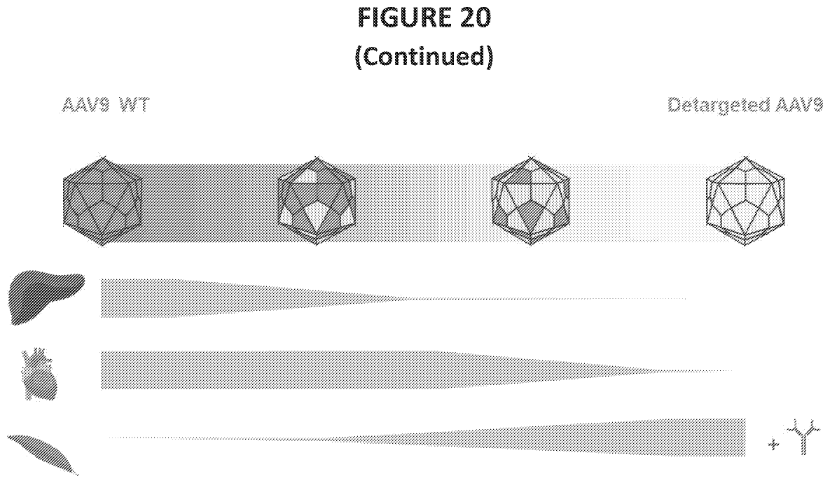

In addition to enhancing therapeutic efficacy, CACNG1 Ab-retargeted AAV9 significantly reduces liver transduction (typically by >95%) compared to WT AAV9. Since different tissues have different sensitivities to detargeting mutations and retargeting antibodies, specific disease applications may require fine tuning of the degree of detargeting and retargeting in combination to achieve the ideal in vivo tropism. The modular nature of the present AAV platform allows for this further refinement by harnessing features of the capsid protein, the retargeting antibody, or both (FIG. 20). For example, the modular nature of the present AAV retargeting platform allows for tunable heart transduction via capsid detargeting mutations for: detargeting away from the heart (for diseases like some LGMDs and XLM™), and maintaining heart tropism (for diseases like DMD, etc.) (FIG. 20). The antibody-based, modular AAV retargeting platform can be used for safer and more effective delivery of gene therapies for multiple muscle diseases.

### Preparation of AAV Viral Vectors

Virus was generated by transfecting 293T packaging cells using PEI Pro with the following plasmids: pAd Helper, an AAV2 ITR-containing genome plasmid encoding a reporter protein, and a pAAV-CAP plasmid encoding AAV Rep and Cap genes, either with or without additional plasmids encoding either the heavy and light chains of an antibody. The antibody heavy chain constructs are all fused to SpyCatcher at their C terminus as described in WO2019006046, incorporated herein in its entirety by reference. Transfection complexes were prepared in incomplete DMEM (no additional supplements) and incubated at room temperature for 10 minutes.

Each virus was generated by transfecting 15 cm plates of 293T packaging cells with the following plasmids and quantities:

### WT AAV9/N272A GFP

| | |
|---|---|
| pAd Helper | 16 ug |
| pAAV-CAG-eGFP | 8 ug |
| pAAV9-CAP or pAAV9 N272A | 8 ug |

### AAV9 Anti-Human ASGR1/Anti-Human CACNG1 GFP

| | | |
|---|---|---|
| pAd Helper | 16 | ug |
| pAAV-CAG-eGFP | 8 | ug |
| pAAV9 CAP G453 Linker 10 SpyTag W503A | 1 | ug |
| pAAV9-CAP N272A | 7 | ug |
| With | | |
| Anti-CACNG1 hIgG4US SpyCatcher Vh | 1.5 | ug |
| ULC 1-39 Vk | 3 | ug |

### WT AAV9-uDys5

| | |
|---|---|
| pAd Helper | 16 ug |
| pAAV-CK8-uDys5 | 8 ug |
| pAAV9-CAP | 8 ug |

AAV9 Anti-Human CACNG1 uDys5

| | | |
|---|---|---|
| pAd Helper | 16 | ug |
| pAAV-CK8-uDys5 | 8 | ug |
| pAAV9 CAP G453 Linker 10 SpyTag | 1 | ug |
| pAAV9-CAP N272A | 7 | ug |
| With | | |
| pAnti-CACNG1 10717 SpyCatcher Vh | 1.5 | ug |
| pAnti-CACNG1 10717 Vk | 3.0 | ug |

### AAV9 Anti-Human CACNG1 CK7-hFKRP

| | | |
|---|---|---|
| pAd Helper | 16 | ug |
| pAAV-CK7-hFKRP | 8 | ug |
| pAAV9 CAP G453 Linker 10 SpyTag | 1 | ug |
| pAAV9-CAP N272A | 7 | ug |
| With | | |
| pAnti-CACNG1 10717 SpyCatcher Vh | 1.5 | ug |
| pAnti-CACNG1 10717 Vk | 3.0 | ug |

### AAV9 Anti-Human DES-hMTM1

| | | |
|---|---|---|
| pAd Helper | 16 | ug |
| pAAV-DES-hMTM1 | 8 | ug |
| pAAV9 CAP G453 Linker 10 SpyTag | 1 | ug |
| pAAV9-CAP N272A | 7 | ug |
| With | | |
| pAnti-CACNG1 10717 SpyCatcher Vh | 1.5 | ug |
| pAnti-CACNG1 10717 Vk | 3.0 | ug |

CK8-uDys5 is described in U.S. Ser. No. 10/479,821B2, incorporated herein in its entirety by reference.

Post incubation, complexes are added to DMEM supplemented with 10% FBS, 1×NEAA, 1% Pen/Strep, and 1% L-Glutamine.

Transfected packaging cells were incubated for 3 days at 37° C., then virus was collected from cell lysates using a standard freeze-thaw protocol. In brief, packaging cells were lifted by scraping and pelleted. Supernatant was removed, and cells were resuspended in a solution of 50 mM Tris-HCl; 150 mM NaCl; and 2 mM MgCl2 [pH 8.0]. Intracellular virus particles were released by inducing cell lysis via three consecutive freeze-thaw cycles, consisting of shuttling cell suspension between dry ice/ethanol bath and 37° C. water bath with vigorous vortexing. Viscosity was reduced by treating lysate with EMD Millipore Benzonase (50 U/ml of cell lysate) for 60 min at 37° C., with occasional mixing. Debris was then pelleted by centrifugation, and the resulting supernatant was filtered through a 0.22 μm PVDF Millex-GV Filter. For crude virus to be tested in vitro, the filtered lysate is added directly to an Amicon Ultra-15 Centrifugal Filter Unit with Ultracel-100 membrane (100 KDa MWCO) filter cartridge. The filter unit was centrifuged at 5-10 minute intervals until desired volume was reached in the upper chamber, then concentrated crude virus was pipetted into a low-protein-binding tube and stored at 4° C. For virus to be tested in vivo, the clarified lysate is further purified using a four step iodixanol density gradient. Gradients are loaded into a Beckman 70Ti rotor and spun at 66,100 rpm for 1.5 h at 1° C. using and max acceleration and deceleration. After ultracentrifugation, iodixanol purified virions are extracted from the 40-60% interface. AAVs in iodixanol solution are diluted in DPBS+/+0.001% pluronic F68 so that the iodixanol is concentration is less than 1%. Purified virus is then concentrated to desired volume using a 100 kDa MWCO Amicon ultrafiltration unit.

Titer (viral genomes per milliliter or vg/mL) was determined by qPCR using a standard curve of a virus of known concentration.

Cryo-Fluorescence Tomography Imaging (see, FIGS. 11 and 15)

D2-mdx, MTM1, and Fkrp^{P448L} mice were fed alfalfa-free low fluorescence diet (AIN93G, Research Diets) for 7 days prior to being injected via the tail vein with 5E+12 vg/kg of either WT AAV9 CAG-eGFP or CACNG1-AAV9 W503A (REGN10717) CAG-eGFP. Mice were euthanized 2 weeks after AAV injection using carbon dioxide. Whole bodies of freshly euthanized mice were systematically frozen in a freezing bath made from a dewar filled with hexanes over dry ice. The frozen carcasses were stored in a −80° C. freezer until ready for CFT imaging.

For CFT imaging, the frozen carcasses were embedded in a block of Optimal Cutting Temperature (OCT) compound (Cancer Diagnostics) and allowed to freeze at −80° C. for 1-2 hours. Thereafter, the OCT block was programmed to undergo block-face imaging in the cryomacrotome—Xerra (EMIT Imaging). Serial sectioning of the entire OCT block was performed at 55 μm per section and block-face image acquisition cycles were performed through the entire sample volume. White light and fluorescence images (Excitation=470 nm; Emission=511 nm) for green fluorescent protein (GFP) were acquired at each plane. Fluorescence images were digitized at 16-bit dynamic range and acquired with consistent exposure times of 5 milliseconds (ms), 50 ms, 500 ms, 1500 ms, and 2500 ms.

The multiple fluorescence images from each plane were then combined into a single 32-bit high dynamic range output image. Flatfield, darkfield and chromatic corrections were applied to individual CFT images, and the images were segmented into separate images, each containing an individual sample. Following completion of acquisition, image data were reconstructed to provide maximum intensity projection and flythrough movies to show distribution of fluorescence throughout the sample volume for each mouse in the OCT block. The reconstructed images were visualized, rendered, and analyzed using VivoQuant software (InviCRO).

D2-mdx Mouse Experiments (see, FIG. 12)

AAV viral vectors were prepared as described above. D2-mdx mice (Jackson Labs: Strain D2.B10-Dmdmdx.J, 013141), were tail vein injected with 1E+11 vg/mouse of two different AAV9 capsids: WT AAV9 particles encapsulating a nucleotide of interest encoding microdystrophin (μDys) under the control of a CK8 promoter, or AAV9 particles comprising a N272A detargeting mutation and retargeted with an anti-hCACNG1 antibody (REGN10717) encapsulating a nucleotide of interest encoding microdystrophin (Dys) under the control of a CK8 promoter. Mice were sacrificed after either 4 weeks or 12 weeks following injection.

D2-mdx: μDys TaqMan qPCR Analysis (see, FIG. 12B)

Four weeks after dosing, the following tissues were placed into RNAlater (ThermoFisher Catalog #AM7021): quadriceps, gastrocnemius, diaphragm, and liver. Tissues were allowed to sit in RNAlater for at least 2 hours at room temperature before being transferred to −20° C. Total RNA was purified using MagMAX™-96 for Microarrays Total RNA Isolation Kit Catalog #AM1839 (Ambion by Life Technologies) according to manufacturer's specifications. DNAse I treatment of samples (Thermo Scientific Catalog #EN0525) was used as per manufacturer's specifications for samples that had enough RNA for a final concentration of 500 ng, otherwise samples did not get DNase I treatment. Samples were then reverse transcribed using SuperScript VILO MasterMix (Invitrogen Catalog #11755-250) according to manufacturer's specifications. For TaqMan gene expression analysis, TaqMan Gene Expression MasterMix (ThermoFisher Scientific Ref #4369016) was used according to manufacturer's specifications. The probes used in the assay were as followed: dystrophin (fwd: AGGGTAGCTAGCATGGAAAAACA, rev: GGGCTTGTGAGACATGAGTGAT, probe: ATTTACATTCTTATGTGCCT) and Rplp0 (ThermoFisher Mm01974474_gH). Samples were loaded into a MicroAmp 384-well plate (ThermoFisher Scientific Catalog #4309849) and ran on the QuantStudio 6 Flex system using the standard TaqMan protocol with results being analyzed using ΔΔCt method.

D2-mdxc: μDys Western Blotting (see, FIG. 12C)

Four weeks after dosing, quadriceps muscles were extracted and snap-frozen in liquid nitrogen. Muscles were homogenized in ice cold lysis buffer with protease and phosphatase inhibitors (Sigma) at 35,000 rpm, and then centrifuged at 15,000 g at 4° C. for 10 minutes, and supernatant was stored at −80° C. Protein content was determined via BCA assay, and 30 ug of protein was loaded onto a 4-20% gradient Criterion TGX gel and ran at 100V, and then transferred to PDVF membrane. Membranes were incubated with 5% milk for one hour at room temperature, and then incubated overnight at 4° C. with antibodies against dystrophin (Developmental Studies Hybridoma Bank) or b-actin (Cell Signaling Technology), and the next day washed and incubated with horseradish peroxidase-conjugated secondary antibodies (Cell Signaling Technology) and detected using an Amersham Imager 600.

D2-mdx: μDys Immunohistochemistry (see, FIG. 12C)

Four weeks after dosing, gastrocnemius muscle was submerged in Optimal Cutting Temperature (OCT) embedding medium and frozen in liquid nitrogen-cooled isopentane. Tissues were cryosectioned at 12 μm thickness and subsequently fixed with 4% PFA, washed with PBS, incubated with blocking buffer (20% Goat Serum, 0.3% Triton, in PBS) for 1 hour at room temperature and stained for laminin (Sigma-Aldrich) and dystrophin (Developmental Studies Hybridoma Bank, MANEX1011B(1C7)) overnight. The next day, tissues were washed with PBS and incubated with fluorophore-conjugated secondary antibodies, counterstained with Hoescht, washed, and mounted with Fluoromount-G (Thermo Fisher). Tissues were then imaged on a Zeiss Axioscan Z1 slide scanner DAPI (Thermo Fisher Scientific).

### D2-Mdx: Serum Creatine Kinase Analysis (See, FIG. 12D)

Blood was collected before dosing and four weeks after dosing into a 1.1 mL Z-Gel microtube (Sarstedt, 41.1378.005), and the serum was allowed to clot for a minimum of 2 hours. The blood was spun down at 4C at 12,000 RPM for 10 minutes. The supernatant was then collected and frozen at −80 C. Once all samples were collected, they were all thawed and then diluted 1:4 with DI water. Serum was analyzed using the ADVIA® Chemistry Creatine Kinase (CK_L) Reagents (REF 10729780) on the ADVIA® Chemistry XPT system.

### D2-Mdx: Forelimb Grip Strength Analysis (See, FIG. 12D)

Forelimb grip strength was assessed prior to AAV treatment and 12 weeks after treatment using a BioSEB Model GT3 grip strength test with T-bar attachment. The instrument was positioned vertically, and mice were grasped by the base of the tail and gently lowered to the T-bar until they grasped the bar, and then slowly lifted upward, in line of pull with the bar. This was repeated 3 times and the max force of the 3 pulls was recorded. The mice were allowed 2 minutes rest, and tested two more times. The average of each of the 3 tests was reported as average maximal grip strength.

D2-mdx Mouse Experiments (see, FIGS. 17-19)

AAV viral vectors were prepared as described above. Two strains of mice, C57BL/6 (Taconic: Strain C57BL/6Ntac, B6) and D2-mdx (Jackson Labs: Strain D2.B10-Dmdmdx.J, 013141), were tail vein injected and evaluated with three different AAV9 capsids at increasing doses. The AAVs tested were WT AAV9 particles encapsulating a nucleotide of interest encoding eGFP under the control of a CAG promoter, AAV9 particles comprising a W503A detargeting mutation and retargeted with an anti-hCACNG1 antibody (REGN10717) encapsulating a nucleotide of interest encoding eGFP under the control of a CAG promoter, and WT AAV9 particles retargeted with an anti-hCACNG1 antibody (REGN10717) encapsulating a nucleotide of interest encoding eGFP under the control of a CAG promoter. To evaluate liver detargeting and heart/skeletal muscle retargeting, AAVs were injected i.v. at doses of 8E+10 and 4E+11 vg/mouse in both strains with an additional dose of 2E+10 vg/mouse in the wildtype C57BL/6 cohort.

D2-mdx: Immunohistochemistry (see, FIG. 17)

Three weeks after dosing, the following tissues were dissected and subsequently fixed in fresh 10% neutral buffered formalin (NBF; VWR Cat #89370-094) for a period of 24 hours at ambient temperature: quadriceps, diaphragm, tongue, heart, and liver. Following fixation, the tissues were processed in the Leica Peloris 3 tissue processor over a duration of 7 hours. The paraffin-infused tissues were then embedded in paraffin blocks and sectioned into 4 μm sections utilizing an automatic sectioning system (AS410, Dainippon). The slides were left to air-dry overnight and subsequently baked in an oven set at 60° C. for one hour. The staining was performed on the Leica Bond Rx automatic staining platform. The primary antibody against GFP (Abcam, ab183734, 1 μg/mL) was incubated for one hour followed by Bond Polymer Refine Detection kit (DS9800). The stained sections were dehydrated, coverslipped, and scanned using a brightfield scanner (GT450, Leica).

D2-mdx: TaqMan qPCR Analysis (see. FIGS. 18 and 19)

Three weeks after dosing, the following tissues were placed into RNAlater (ThermoFisher Catalog #AM7021): liver, heart, quadriceps, diaphragm, tibialis anterior, gastrocnemius, and tongue. Tissues were allowed to sit in RNAlater for at least 2 hours at room temperature before being transferred to −20° C. Total RNA was purified using MagMAX™-96 for Microarrays Total RNA Isolation Kit Catalog #AM1839 (Ambion by Life Technologies) according to manufacturer's specifications. DNAse I treatment of samples (Thermo Scientific Catalog #EN0525) was used as per manufacturer's specifications for samples that had enough RNA for a final concentration of 500 ng, otherwise samples did not get DNase I treatment. Samples were then reverse transcribed using SuperScript VILO MasterMix (Invitrogen Catalog #11755-250) according to manufacturer's specifications. For TaqMan gene expression analysis, TaqMan Gene Expression MasterMix (ThermoFisher Scientific Ref #4369016) was used according to manufacturer's specifications. The probes used in the assay were as followed: eGFP (ThermoFisher Mr04329676_mr) and Rplp0 (ThermoFisher Mm01974474_gH). Samples were loaded into a MicroAmp 384-well plate (ThermoFisher Scientific Catalog #4309849) and ran on the QuantStudio 6 Flex system using the standard TaqMan protocol with results being analyzed using ΔΔCt method.

FKRP^{P448L} Mouse Experiments (see, FIG. 13)

AAV viral vectors were prepared as described above. Eight to ten week old male WT (MAID 50500) and Lama2^{HU/HU}/DAG1^{HU/HU}/FKRP^{P448L/P448L} mice were injected intravenously (150 ul volume) with either vehicle (PBS (Gibco Ref #20012-043)+0.001% pluronic acid), 1E+11vg of WT AAV9 particles encapsulating a nucleotide of interest encoding human FKRP (hFKRP) under the control of a CK7 promoter (WT AAV9), or 1E+11vg of AAV9 particles comprising an N272A mutation retargeted with an anti-hCACNG1 antibody (REGN10717) encapsulating a nucleotide of interest encoding human FKRP (hFKRP) under the control of a CK7 promoter (REGN10717-AAV9(N272A)-hFKRP) as follows:

$$WT 50500 + PBS + pluronic acid ( n = 5 )$$

$$Lama {2}^{HU / HU} / DAG {1}^{HU / HU} / {FKRP}^{P 448 L} + PBS + ( n = 5 ) *$$

$$Lama {2}^{HU / HU} / DAG {1}^{HU / HU} / {FKRP}^{P 448 L} + WT AAV 9 - hFKRP ( 1 E 11 vg / mouse ) ( n = 7 )$$

$$Lama {2}^{HU / HU} / DAG {1}^{HU / HU} / {FKRP}^{P 448 L} + ( 1 E 11 vg / mouse ) ( n = 7 )$$

$$* “ PBS ” Group in Figures 13 B and 13 D$$

### FKRP^{P418L}: Downhill Treadmill Running Assessment (See, FIG. 13D)

Seven weeks post AAV injections, mice were assayed on a mouse treadmill (UGO Basile Model 47300) under the following protocol: (1) Mice were acclimated for ten minutes at 0 meters/minute and −5° degrees (declining). (2) The decline was then adjusted to −15° degrees (declining). (3) The treadmill was then started at a speed of 4 meters/minute and increase by 1 meter/minute every minute until reaching top speed of 12 meters/minute. (4) The assessment ended when either 30 minutes of total running time had elapsed or if at any time mice remained on the shock grid at the end of the treadmill for more than 3 consecutive seconds, whichever came first.

### FKRP^{P448L}: IIH6 (Glycosylated α-Dystroglycan) Immunohistochemistry (See, FIG. 13C)

Mice (as described above for the downhill treadmill running assay) were euthanized 24 hours post treadmill running assessment and a subset of tissues (diaphragm, quadriceps, gastrocnemius/soleus, heart) were cryopreserved in O.C.T. compound (Tissue-Tek Catalog #4583) by freezing in liquid nitrogen-cooled isopentane. Tissues were stored at −80° C. and subsequently sectioned onto SuperFrost Plus charged glass slides (ThermoFisher Scientific Catalog #12-550-15) at 10 μm thickness. Sections were fixed with ice cold ethanol-acetic acid (1:1) for 1 minute. Slides were then washed 3 times for 5 minutes each with PBS. Tissue sections were covered with blocking buffer (20% Goat Serum, 0.3% Triton, in PBS) for 1 hour at RT. Primary antibodies were added (diluted 1:100 in blocking buffer for Abcam 234587 IIH6 and diluted 1:500 in blocking buffer for Abcam 11576 laminin) and incubated overnight. Slides were then washed with PBS and incubated for 1 hour with secondary antibody (diluted 1:250 in blocking buffer, Invitrogen A11006 and Invitrogen A21238). Slides were washed with PBS, counterstained with Hoechst 33342 (ThermoFisher, catalog #00-4958-02, 1:1000) for 5 minutes, and mounted in Fluoromount G (ThermoFisher Cat #00-4958-02). Slides were dried overnight, and subsequently imaged using a Zeiss AxioScan Z1 microscope. Images were analyzed for IIH6 intensity and area using HALO software (Indica Labs).

FKRP^{P448L}: TaqMan qPCR Analysis (see, FIGS. 13B and 15)

Mice (as described above for the downhill treadmill running assay) were euthanized 24 hours post treadmill running assessment and the following tissues were placed into RNAlater (Invitrogen Catalog #AM7021): gastrocnemius, soleus, quadricep, tibialis anterior, diaphragm, heart, liver. Tissues were allowed to sit in RNAlater for at least 2 hours at room temperature before being transferred to −80° C. Total RNA was purified using MagMAX™-96 for Microarrays Total RNA Isolation Kit Catalog #AM1839 (Ambion by Life Technologies) according to manufacturer's specifications.

Isolated RNA samples were then reverse transcribed using SuperScript VILO MasterMix (Invitrogen Catalog #11755-250) according to manufacturer's specifications. For TaqMan gene expression analysis, TaqMan Gene Expression MasterMix (ThermoFisher Scientific Ref #4369016) was used according to manufacturer's specifications. The probes used in the assay were as followed: hFKRP FAM (ThermoFisher, fwd TGCAGTACAGCGAAAGCA, rev AGGAAGTGCTCGGGAAAC, probe TCATGACCAAGGACACGTGGCTG) and Gapdh (ThermoFisher Mm99999915_g1). Samples were loaded into a MicroAmp 384-well plate (ThermoFisher Scientific Catalog #4309849) and ran on the QuantStudio 6 Flex system using the standard TaqMan protocol with results being analyzed using ΔΔCt method.

### CACNG1-AAV9-hFKRP Titration in FKRP^{P448L/P448L} Mice (See, FIG. 24)

AAV viral vectors were prepared as described above. Seven to thirteen week old male wildtype mice (50500) and Lama2^{HU/HU}/DAG1^{HU/HU}/FKRP^{P448L/P448L} mice were injected intravenously (150 ul volume) through the lateral tail vein with either vehicle (PBS (Gibco Ref #20012-043)+0.001% pluronic acid) or AAV9 particles comprising an N272A mutation retargeted with an anti-hCACNG1 antibody (REGN10717) encapsulating a nucleotide of interest encoding human FKRP (hFKRP) under the control of a CK7 promoter (REGN10717-AAV9(N272A)-hFKRP) as follows:

$$WT 50500 + PBS + pluronic acid ( n = 7 ) \qquad Group A$$

$$Lama {2}^{HU / HU} / DAG {1}^{HU / HU} / {FKRP}^{P 448 L / P 448 L} + PBS + pluronic acid ( n = 6 ) \qquad Group B$$

$$Lama {2}^{HU / HU} / DAG {1}^{HU / HU} / {FKRP}^{P 448 L / P 448 L} + REGN 10717 - AAV 9 ( N 272 A ) - hFKRP ( 4 E 12 vg / kg ( n = 7 ) ( ACV 146 ) \qquad Group C$$

$$Lama {2}^{HU / HU} / DAG {1}^{HU / HU} / {FKRP}^{P 448 L / P 448 L} + REGN 10717 - AAV 9 ( N 272 A ) - hFKRP ( 1 E 13 vg / kg ( n = 7 ) ( ACV 146 ) \qquad Group D$$

$$Lama {2}^{HU / HU} / DAG {1}^{HU / HU} / {FKRP}^{P 448 L / P 448 L} + REGN 10717 - AAV 9 ( N 272 A ) - hFKRP ( 5 E 13 vg / kg ( n = 7 ) ( ACV 146 ) \qquad Group E$$

Serum samples were collected one week prior to and at four week intervals post AAV administration. Mice were briefly anesthetized with 5% Isoflurane and ~100 ul of whole blood was collected in a serum separator tube (Sarstedt VWR REF #41.1500.005) via the submandibular vein. Blood samples were allowed to clot at room temperature for a minimum of 30 minutes. After clotting, samples were centrifuged (Eppendorf 5430R) at 10,000 RPM for 10 minutes. Serum was then collected and frozen at −80 F. Samples were later thawed and analyzed on an ADVIA Chemistry XPT (Siemens) for creatine kinase (CK) levels.

MTM1 Knockout (KO) Mouse Experiments (see, FIG. 14)

MTM1 knockout mice were tail vein injected with 2E+10 vg/mouse (~2E+12 vg/kg) of WT AAV9 encapsulating a nucleotide of interest encoding human MTM1 (hMTM1) under the control of a desmin promoter (n=4), AAV9 particles comprising an N272A mutation retargeted with an anti-hCACNG1 antibody (REGN10717) encapsulating a nucleotide of interest encoding human MTM1 (hMTM1) under the control of a desmin promoter (n=3), or PBS as a control (n=5). Surviving mice were euthanized 4 weeks post dosing; for mice that were sick or found dead, tissues and blood were collected prior to euthanasia if possible.

MTM1 KO: TaqMan qPCR Analysis (see. FIG. 14B)

The following tissues were placed into RNAlater (ThermoFisher Catalog #AM7021): gastrocnemius, soleus, quadriceps, tibialis anterior, diaphragm, tongue, heart, liver, spleen, kidney. Tissues were allowed to sit in RNAlater for at least 2 hours at room temperature before being transferred to −20° C. Total RNA was purified using MagMAX™-96 for Microarrays Total RNA Isolation Kit Catalog #AM11839 (Ambion by Life Technologies) according to manufacturer's specifications. DNAse I treatment of samples (Thermo Scientific Catalog #EN0525) was used as per manufacturer's specifications for samples that had enough RNA for a final concentration of 500 ng, otherwise samples did not get DNase I treatment. Samples were then reverse transcribed using SuperScript VILO MasterMix (Invitrogen Catalog #11755-250) according to manufacturer's specifications. For TaqMan gene expression analysis, TaqMan Gene Expression MasterMix (ThermoFisher Scientific Ref #4369016) was used according to manufacturer's specifications. The probes used in the assay were as followed: hMTM1_ABI (ThermoFisher Hs00896975_m1) and Rplp0 (ThermoFisher Mm01974474_gH). Samples were loaded into a MicroAmp 384-well plate (ThermoFisher Scientific Catalog #4309849) and ran on the QuantStudio 6 Flex system using the standard TaqMan protocol with results being analyzed using ΔΔCt method.

MTM1 KO: Immunohistochemistry (see, FIG. 14C)

The following tissues were cryopreserved in O.C.T. compound (Tissue-Tek Catalog #4583) by freezing in liquid nitrogen-cooled isopentane: gastrocnemius and soleus, diaphragm, tibialis anterior, heart, and liver. Tissues were stored at −80° C. and subsequently sectioned onto SuperFrost Plus charged glass slides (ThermoFisher Scientific Catalog #12-550-15) at 10 μm thickness. Sections were fixed with 4% paraformaldehyde (PFA) for 15 minutes, washed three times with PBS, and were then incubated in blocking solution containing 20% goat serum and 0.3% Triton X-100 in PBS for one hour. Sections were then incubated with laminin primary antibody (Sigma-Aldrich, catalog #L9393, 1:500) in blocking solution overnight, washed with PBS, and then stained with fluorophore-conjugated anti-rabbit secondary antibody (ThermoFisher, 1:250), washed, counterstained with Hoechst 33342 (ThermoFisher, catalog #00-4958-02, 1:1000) for 5 minutes, and mounted in Fluoromount G (ThermoFisher Cat #00-4958-02). Slides were dried overnight, and subsequently imaged using a Zeiss AxioScan Z1 microscope.

CK8-μDys5 Nucleotide of Interest (SEQ ID NO:270)
CCTGCAGGCAGCTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAGAGGGAGT
GGCCAACTCCATCACTAGGGGTTCCTGCGGCCGCCTAGCATGCTGCCCATGTAAGGAG
GCAAGGCCTGGGGACACCCGAGATGCCTGGTTATAATTAACCCAGACATGTGGCTGCC
CCCCCCCCCCCAACACCTGCTGCCTCTAAAAATAACCCTGCATGCCATGTTCCCGGCGA
AGGGCCAGCTGTCCCCCGCCAGCTAGACTCAGCACTTAGTTTAGGAACCAGTGAGCAA
GTCAGCCCTTGGGGCAGCCCATACAAGGCCATGGGGCTGGGCAAGCTGCACGCCTGGG
TCCGGGGTGGGCACGGTGCCCGGGCAACGAGCTGAAAGCTCATCTGCTCTCAGGGGCC
CCTCCCTGGGGACAGCCCCTCCTGGCTAGTCACACCCTGTAGGCTCCTCTATATAACCC
AGGGGCACAGGGGCTGCCCTCATTCTACCACCACCTCCACAGCACAGACAGACACTCA
GGAGCCAGCCAGCGTCGAGGTTAACCCGCGGCCGTTTTTTTTATCGCTGCCTTGATATA
CACTTTCCACCATGCTTTGGTGGGAAGAAGTAGAGGACTGTTATGAAAGAGAAGATGTT
CAAAAGAAAACATTCACAAAATGGGTAAATGCACAATTTTCTAAGTTTGGGAAGCAGCAT
ATTGAGAACCTCTTCAGTGACCTACAGGATGGGAGGCGCCTCCTAGACCTCCTCGAAGG
CCTGACAGGGCAAAAACTGCCAAAAGAAAAAGGATCCACAAGAGTTCATGCCCTGAACA
ATGTCAACAAGGCACTGCGGGTTTTGCAGAACAATAATGTTGATTTAGTGAATATTGGAA
GTACTGACATCGTAGATGGAAATCATAAACTGACTCTTGGTTTGATTTGGAATATAATCC
TCCACTGGCAGGTCAAAAATGTAATGAAAAATATCATGGCTGGATTGCAACAAACCAACA
GTGAAAAGATTCTCCTGAGCTGGGTCCGACAATCAACTCGTAATTATCCACAGGTTAATG
TAATCAACTTCACCACCAGCTGGTCTGATGGCCTGGCTTTGAATGCTCTCATCCATAGTC
ATAGGCCAGACCTATTTGACTGGAATAGTGTGGTTTGCCAGCAGTCAGCCACACAACGA
CTGGAACATGCATTCAACATCGCCAGATATCAATTAGGCATAGAGAAACTACTCGATCCT
GAAGATGTTGATACCACCTATCCAGATAAGAAGTCCATCTTAATGTACATCACATCACTC
TTCCAAGTTTTGCCTCAACAAGTGAGCATTGAAGCCATCCAGGAAGTGGAAATGTTGCCA
AGGCCACCTAAAGTGACTAAAGAAGAACATTTTCAGTTACATCATCAAATGCACTATTCT
CAACAGATCACGGTCAGTCTAGCACAGGGATATGAGAGAACTTCTTCCCCTAAGCCTCG
ATTCAAGAGCTATGCCTACACACAGGCTGCTTATGTCACCACCTCTGACCCTACACGGAG
CCCATTTCCTTCACAGCATTTGGAAGCTCCTGAAGACAAGTCATTTGGCAGTTCATTGAT
GGAGAGTGAAGTAAACCTGGACCGTTATCAAACAGCTTTAGAAGAAGTATTATCGTGGC
TTCTTTCTGCTGAGGACACATTGCAAGCACAAGGAGAGATTTCTAATGATGTGGAAGTGG
TGAAAGACCAGTTTCATACTCATGAGGGGTACATGATGGATTTGACAGCCCATCAGGGC
CGGGTTGGTAATATTCTACAATTGGGAAGTAAGCTGATTGGAACAGGAAAATTATCAGAA
GATGAAGAAACTGAAGTACAAGAGCAGATGAATCTCCTAAATTCAAGATGGGAATGCCT
CAGGGTAGCTAGCATGGAAAAACAAAGCAATTTACATTCTTATGTGCCTTCTACTTATTT
GACTGAAATCACTCATGTCTCACAAGCCCTATTAGAAGTGGAACAACTTCTCAATGCTCC
TGACCTCTGTGCTAAGGACTTTGAAGATCTCTTTAAGCAAGAGGAGTCTCTGAAGAATAT
AAAAGATAGTCTACAACAAAGCTCAGGTCGGATTGACATTATTCATAGCAAGAAGACAGC
AGCATTGCAAAGTGCAACGCCTGTGGAAAGGGTGAAGCTACAGGAAGCTCTCTCCCAGC
TTGATTTCCAATGGGAAAAAGTTAACAAAATGTACAAGGACCGACAAGGGCGATTTGAC
AGATCTGTTGAGAAATGGCGGCGTTTTCATTATGATATAAAGATATTTAATCAGTGGCTA
ACAGAAGCTGAACAGTTTCTCAGAAAGACACAAATTCCTGAGAATTGGGAACATGCTAAA
TACAAATGGTATCTTAAGGAACTCCAGGATGGCATTGGGCAGCGGCAAACTGTTGTCAG
AACATTGAATGCAACTGGGGAAGAAATAATTCAGCAATCCTCAAAAACAGATGCCAGTAT
TCTACAGGAAAAATTGGGAAGCCTGAATCTGCGGTGGCAGGAGGTCTGCAAACAGCTGT
CAGACAGAAAAAAGAGGCTAGAAGAACAATCTGACCAGTGGAAGCGTCTGCACCTTTCT
CTGCAGGAACTTCTGGTGTGGCTACAGCTGAAAGATGATGAATTAAGCCGGCAGGCACC
TATTGGAGGCGACTTTCCAGCAGTTCAGAAGCAGAACGATGTACATAGGGCCTTCAAGA
GGGAATTGAAAACTAAAGAACCTGTAATCATGAGTACTCTTGAGACTGTACGAATATTTC
TGACAGAGCAGCCTTTGGAAGGACTAGAGAAACTCTACCAGGAGCCCAGAGAGCTGCCT
CCTGAGGAGAGAGCCCAGAATGTCACTCGGCTTCTACGAAAGCAGGCTGAGGAGGTCAA
TACTGAGTGGGAAAAATTGAACCTGCACTCCGCTGACTGGCAGAGAAAAATAGATGAGA
CCCTTGAAAGACTCCAGGAACTTCAAGAGGCCACGGATGAGCTGGACCTCAAGCTGCGC
CAAGCTGAGGTGATCAAGGGATCCTGGCAGCCCGTGGGCGATCTCCTCATTGACTCTCT
CCAAGATCACCTCGAGAAAGTCAAGGCACTTCGAGGAGAAATTGCGCCTCTGAAAGAGA
ACGTGAGCCACGTCAATGACCTTGCTCGCCAGCTTACCACTTTGGGCATTCAGCTCTCAC
CGTATAACCTCAGCACTCTGGAAGACCTGAACACCAGATGGAAGCTTCTGCAGGTGGCC
GTCGAGGACCGAGTCAGGCAGCTGCATGAAGCCCACAGGGACTTTGGTCCAGCATCTCA
GCACTTTCTTTCCACGTCTGTCCAGGGTCCCTGGGAGAGAGCCATCTCGCCAAACAAAGT
GCCCTACTATATCAACCACGAGACTCAAACAACTTGCTGGGACCATCCCAAAATGACAGA
GCTCTACCAGTCTTTAGCTGACCTGAATAATGTCAGATTCTCAGCTTATAGGACTGCCAT
GAAACTCCGAAGACTGCAGAAGGCCCTTTGCTTGGATCTCTTGAGCCTGTCAGCTGCAT
GTGATGCCTTGGACCAGCACAACCTCAAGCAAAATGACCAGCCCATGGATATCCTGCAG
ATTATTAATTGTTTGACCACTATTTATGACCGCCTGGAGCAAGAGCACAACAATTTGGTC
AACGTCCCTCTCTGCGTGGATATGTGTCTGAACTGGCTGCTGAATGTTTATGATACGGGA
CGAACAGGGAGGATCCGTGTCCTGTCTTTTAAAACTGGCATCATTTCCCTGTGTAAAGCA
CATTTGGAAGACAAGTACAGATACCTTTTCAAGCAAGTGGCAAGTTCAACAGGATTTTGT
GACCAGCGCAGGCTGGGCCTCCTTCTGCATGATTCTATCCAAATTCCAAGACAGTTGGGT
GAAGTTGCATCCTTTGGGGGCAGTAACATTGAGCCAAGTGTCCGGAGCTGCTTCCAATTT
GCTAATAATAAGCCAGAGATCGAAGCGGCCCTCTTCCTAGACTGGATGAGACTGGAACC
CCAGTCCATGGTGTGGCTGCCCGTCCTGCACAGAGTGGCTGCTGCAGAAACTGCCAAGC
ATCAGGCCAAATGTAACATCTGCAAAGAGTGTCCAATCATTGGATTCAGGTACAGGAGTC
TAAAGCACTTTAATTATGACATCTGCCAAAGCTGCTTTTTTTCTGGTCGAGTTGCAAAAG
GCCATAAAATGCACTATCCCATGGTGGAATATTGCACTCCGACTACATCAGGAGAAGATG
TTCGAGACTTTGCCAAGGTACTAAAAAACAAATTTCGAACCAAAAGGTATTTTGCGAAGC
ATCCCCGAATGGGCTACCTGCCAGTGCAGACTGTCTTAGAGGGGGACAACATGGAAACT

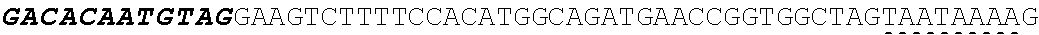

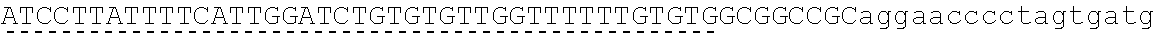

gagttggccactccctctctgcgcgctcgctcgctcactgaggccgggcgaccaaaggtcgcccgacgcccgggctttgcccgggcggcct
cagtgagcgagcgagcgcgcagctgcctgcagg
Annotations
SINGLE UNDERLINED = 5' ITR, DOUBLE UNDERLINED = K8 PROMOTER,

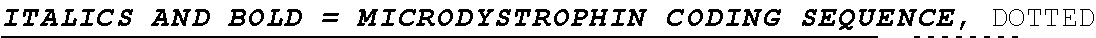

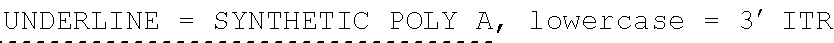

CK7-hFKRP nucleotide of interest (SEQ ID NO:271)
CCTGCAGGCAGCTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAGAGGGAGT
GGCCAACTCCATCACTAGGGGTTCCTCGGGCAAAGCCACGCGTCTAGCATGCCCCACT
ACGGGTCTAGGCTGCCCATGTAAGGAGGCAAGGCCTGGGGACACCCGAGATGCCTGG
TTATAATTAACCCAGACATGTGGCTGCCCCCGCCCCCCCAACACCTGCTGCCTCTAAAA
ATAACCCTGTCCCTGGTGGATCCCCTGCATGCGAAGATCTTCGAACAAGGCTGTGGGG
GACTGAGGGCAGGCTGTAACAGGCTTGGGGGCCAGGGCTTATACGTGCCTGGGACTCC
CAAAGTATTACTGTTCCATGTTCCCGGCGAAGGGCCAGCTGTCCCCCGCCAGCTAGAC
TCAGCACTTAGTTTAGGAACCAGTGAGCAAGTCAGCCCTTGGGGCAGCCCATACAAGG
CCATGGGGCTGGGCAAGCTGCACGCCTGGGTCCGGGGTGGGCACGGTGCCCGGGCAA
CGAGCTGAAAGCTCATCTGCTCTCAGGGGCCCCTCCCTGGGGACAGCCCCTCCTGGCT
AGTCACACCCTGTAGGCTCCTCTATATAACCCAGGGGCACAGGGGCTGCCCTCATTCT
ACCACCACCTCCACAGCACGGATCCGCCACCATGCGGCTCACCCGCTGCCAGGCTGCC
CTGGCGGCCGCCATCACCCTCAACCTTCTGGTCCTCTTCTATGTCTCGTGGCTGCAGCAC
CAGCCTAGGAATTCCCGGGCCCGGGGGCCCCGTCGTGCCTCTGCTGCCGGCCCCCGTGT
CACCGTCCTGGTGCGGGAGTTCGAGGCATTTGACAACGCGGTGCCCGAGCTGGTAGACT
CCTTCCTGCAGCAAGACCCAGCCCAGCCCGTGGTGGTGGCAGCCGACACGCTCCCCTAC
CCGCCCCTGGCCCTGCCCCGCATCCCCAACGTGCGTCTGGCGCTGCTCCAGCCCGCCCT
GGACCGGCCAGCCGCAGCCTCGCGCCCGGAGACCTACGTGGCCACCGAGTTTGTGGCC
CTAGTACCTGATGGGGCGCGGGCTGAGGCACCTGGCCTGCTGGAGCGCATGGTGGAGG
CGCTCCGCGCAGGAAGCGCACGTCTGGTGGCCGCCCCGGTTGCCACGGCCAACCCTGCC
AGGTGCCTGGCCCTGAACGTCAGCCTGCGAGAGTGGACCGCCCGCTATGGCGCAGCCCC
CGCCGCGCCCCGCTGCGACGCCCTGGACGGAGATGCTGTGGTGCTCCTGCGCGCCCGC
GACCTCTTCAACCTCTCGGCGCCCCTGGCCCGGCCGGTGGGCACCAGCCTCTTTCTGCA
GACCGCCCTTCGCGGCTGGGCGGTGCAGCTGCTGGACTTGACCTTCGCCGCGGCGCGCC
AGCCCCCGCTGGCCACGGCCCACGCGCGCTGGAAGGCTGAGCGCGAGGGACGCGCTCG
GCGGGCGGCGCTGCTCCGCGCGCTGGGCATCCGCCTAGTGAGCTGGGAAGGCGGGCGG
CTGGAGTGGTTCGGCTGCAACAAGGAGACCACGCGCTGCTTCGGAACCGTGGTGGGCG
ACACGCCCGCCTACCTCTACGAGGAGCGCTGGACGCCCCCCTGCTGCCTGCGCGCGCTG
CGCGAGACCGCCCGCTATGTGGTGGGCGTGCTGGAGGCTGCGGGCGTGCGCTACTGGC
TCGAGGGCGGCTCACTGCTGGGGGCCGCCCGCCACGGGGACATCATCCCATGGGACTA
CGACGTGGACCTGGGCATCTACTTGGAGGACGTGGGCAACTGCGAGCAGCTGCGGGGG
GCAGAGGCCGGCTCGGTGGTGGATGAGCGCGGCTTCGTATGGGAGAAGGCGGTCGAGG
GCGACTTTTTCCGCGTGCAGTACAGCGAAAGCAACCACTTGCACGTGGACCTGTGGCCC
TTCTACCCCCGCAATGGCGTCATGACCAAGGACACGTGGCTGGACCACCGGCAGGATGT
GGAGTTTCCCGAGCACTTCCTGCAGCCGCTGGTGCCCCTGCCCTTTGCCGGCTTCGTGG
CGCAGGCGCCTAACAACTACCGCCGCTTCCTGGAGCTCAAGTTCGGGCCCGGGGTCATC
GAGAACCCCCAGTACCCCAACCCGGCACTGCTGAGTCTGACGGGAAGCGGCTGATAATA

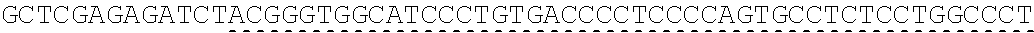

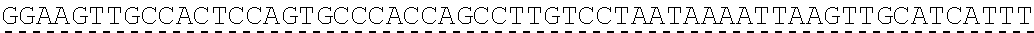

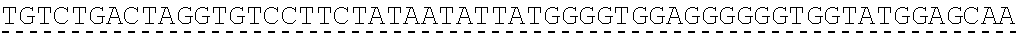

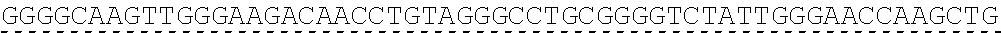

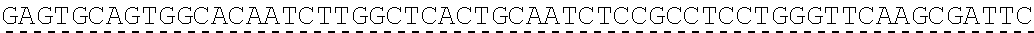

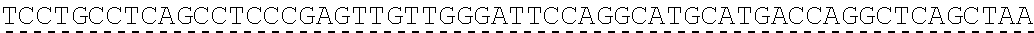

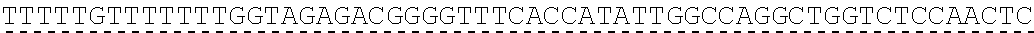

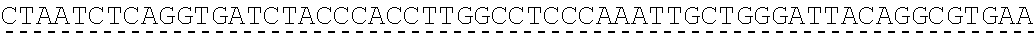

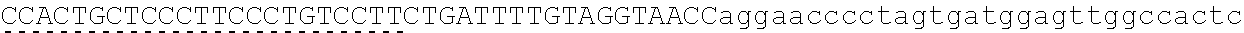

cctctctgcgcgctcgctcgctcactgaggccgggcgaccaaaggtcgcccgacgcccgggctttgcccgggcggcctcagtgagcgagc
gagcgcgcagctgcctgcagg
Annotations
SINGLE UNDERLINED = 5' ITR, DOUBLE UNDERLINED = CK7 PROMOTER,

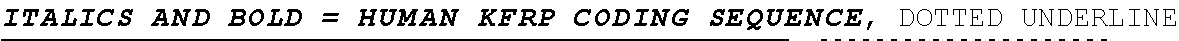

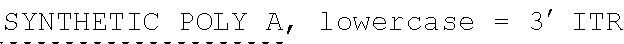

1.0DES-hMTM1 nucleotide of interest (SEQ ID NO:272)
CCTGCAGGCAGCTGCGCGCTCGCTCGCTCACTGAGGCCGCCCGGGCAAAGCCCGGGCG
TCGGGCGACCTTTGGTCGCCCGGCCTCAGTGAGCGAGCGAGCGCGCAGAGAGGGAGT
GGCCAACTCCATCACTAGGGGTTCCTCGGGCAAAGCCACGCGTTACCCCCTGCCCCCC
ACAGCTCCTCTCCTGTGCCTTGTTTCCCAGCCATGCGTTCTCCTCTATAAATACCCGCTC
TGGTATTTGGGGTTGGCAGCTGTTGCTGCCAGGGAGATGGTTGGGTTGACATGCGGCT
CCTGACAAAACACAAACCCCTGGTGTGTGTGGGCGTGGGTGGTGTGAGTAGGGGGATG
AATCAGGGAGGGGGCGGGGGACCCAGGGGGCAGGAGCCACACAAAGTCTGTGCGGG
GGTGGGAGCGCACATAGCAATTGGAAACTGAAAGCTTATCAGACCCTTTCTGGAAATC
AGCCCACTGTTTATAAACTTGAGGCCCCACCCTCGACAGTACCGGGGAGGAAGAGGGC
CTGCACTAGTCCAGAGGGAAACTGAGGCTCAGGGCTAGCTCGCCCATAGACATACATG
GCAGGCAGGCTTTGGCCAGGATCCCTCCGCCTGCCAGGCGTCTCCCTGCCCTCCCTTCC
TGCCTAGAGACCCCCACCCTCAAGCCTGGCTGGTCTTTGCCTGAGACCCAAACCTCTTC
GACTTCAAGAGAATATTTAGGAACAAGGTGGTTTAGGGCCTTTCCTGGGAACAGGCCT
TGACCCTTTAAGAAATGACCCAAAGTCTCTCCTTGACCAAAAAGGGGACCCTCAAACT
AAAGGGAAGCCTCTCTTCTGCTGTCTCCCCTGACCCCACTCCCCCCCACCCCAGGACGA
GGAGATAACCAGGGCTGAAAGAGGCCCGCCTGGGGGCTGCAGACATGCTTGCTGCCT
GCCCTGGCGAAGGATTGGCAGGCTTGCCCGTCACAGGACCCCCGCTGGCTGACTCAGG
GGCGCAGGCCTCTTGCGGGGGAGCTGGCCTCCCCGCCCCCACGGCCACGGGCCGCCCT
TTCCTGGCAGGACAGCGGGATCTTGCAGCTGTCAGGGGAGGGGAGGCGGGGGCTGAT
GTCAGGAGGGATACAAATAGTGCCGACGGCTGGGGGCCCTGTCTCCCCTCGCCGCATC
CACTCTCCGGCCGGCCGCCTGCCCGCCGCCTCCTCCGTGCGCCCGCCAGCCTCGCCCGG
GATCCGCCACCATGGCTTCTGCATCAACTTCTAAATATAATTCACACTCCTTGGAGAATG
AGTCTATTAAGAGGACGTCTCGAGATGGAGTCAATCGAGACCTCACTGAGGCTGTTCCT
CGACTTCCAGGAGAAACACTAATCACTGACAAAGAAGTTATTTACATATGTCCTTTCAAT
GGCCCCATTAAGGGAAGAGTTTACATCACAAATTATCGTCTTTATTTAAGAAGTTTGGAA
ACGGATTCTTCTCTAATACTTGATGTTCCTCTGGGTGTGATCTCGAGAATTGAAAAAATG
GGAGGCGCGACAAGTAGAGGAGAAAATTCCTATGGTCTAGATATTACTTGTAAAGACAT
GAGAAACCTGAGGTTCGCTTTGAAACAGGAAGGCCACAGCAGAAGAGATATGTTTGAGA
TCCTCACGAGATACGCGTTTCCCCTGGCTCACAGTCTGCCATTATTTGCATTTTTAAATG
AAGAAAAGTTTAACGTGGATGGATGGACAGTTTACAATCCAGTGGAAGAATACAGGAGG
CAGGGCTTGCCCAATCACCATTGGAGAATAACTTTTATTAATAAGTGCTATGAGCTCTGT
GACACTTACCCTGCTCTTTTGGTGGTTCCGTATCGTGCCTCAGATGATGACCTCCGGAGA
GTTGCAACTTTTAGGTCCCGAAATCGAATTCCAGTGCTGTCATGGATTCATCCAGAAAAT
AAGACGGTCATTGTGCGTTGCAGTCAGCCTCTTGTCGGTATGAGTGGGAAACGAAATAA
AGATGATGAGAAATATCTCGATGTTATCAGGGAGACTAATAAACAAATTTCTAAACTCAC
CATTTATGATGCAAGACCCAGCGTAAATGCAGTGGCCAACAAGGCAACAGGAGGAGGAT
ATGAAAGTGATGATGCATATCATAACGCCGAACTTTTCTTCTTAGACATTCATAATATTCA
TGTTATGCGGGAATCTTTAAAAAAAGTGAAGGACATTGTTTATCCTAATGTAGAAGAATC
TCATTGGTTGTCCAGTTTGGAGTCTACTCATTGGTTAGAACATATCAAGCTCGTTTTGAC
AGGAGCCATTCAAGTAGCAGACAAAGTTTCTTCAGGGAAGAGTTCAGTGCTTGTGCATT
GCAGTGACGGATGGGACAGGACTGCTCAGCTGACATCCTTGGCCATGCTGATGTTGGAT
AGCTTCTATAGGAGCATTGAAGGGTTCGAAATACTGGTACAAAAAGAATGGATAAGTTTT
GGACATAAATTTGCATCTCGAATAGGTCATGGTGATAAAAACCACACCGATGCTGACCGT
TCTCCTATTTTTCTCCAGTTTATTGATTGTGTGTGGCAAATGTCAAAACAGTTCCCTACAG
CTTTTGAATTCAATGAACAATTTTTGATTATAATTTTGGATCATCTGTATAGTTGCCGATT
TGGTACTTTCTTATTCAACTGTGAATCTGCTCGAGAAAGACAGAAGGTTACAGAAAGGAC
TGTTTCTTTATGGTCACTGATAAACAGTAATAAAGAAAAATTCAAAAACCCCTTCTATACT
AAAGAAATCAATCGAGTTTTATATCCAGTTGCCAGTATGCGTCACTTGGAACTCTGGGTG
AATTACTACATTAGATGGAACCCCAGGATCAAGCAACAACAGCCGAATCCAGTGGAGCA
GCGTTACATGGAGCTCTTAGCCTTACGCGACGAATACATAAAGCGGCTTGAGGAACTGC
AGCTCGCCAACTCTGCCAAGCTTTCTGATCCCCCAACTTCACCTTCCAGTCCTTCGCAAA

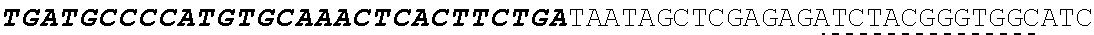

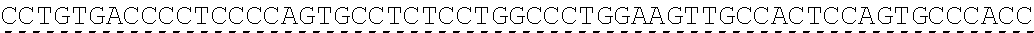

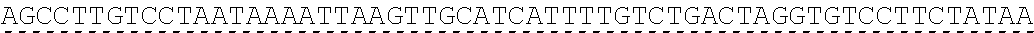

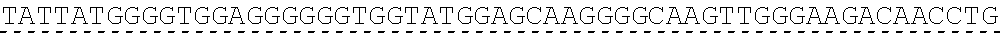

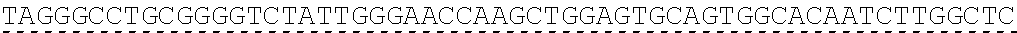

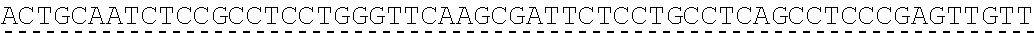

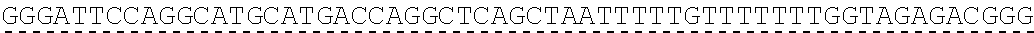

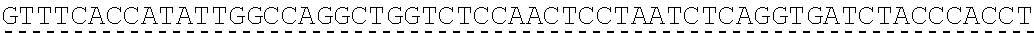

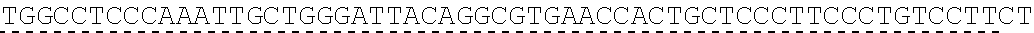

GATTTTGTAGGTAACCaggaacccctagtgatggagttggccactccctctctgcgcgctcgctcgctcactgaggccgggcga
ccaaaggtcgcccgacgcccgggctttgcccgggggcctcagtgagcgagcgagcgcgcagctgcctgcagg
Annotations
SINGLE UNDERLINED = 5' ITR, DOUBLE UNDERLINED = 1.0 DES PROMOTER,

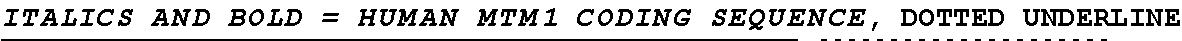

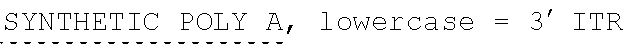

### Example 10: Systematically Delivered Adeno-Associated Virus Retargeted to CACNG1 for Antibody-Dependent Transduction of Skeletal Muscles and Reduction of Dose-Limiting Toxicities of AAV in in Non-Human Primates

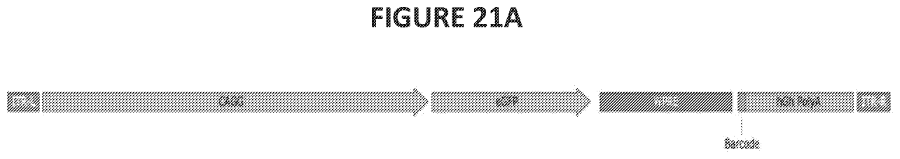

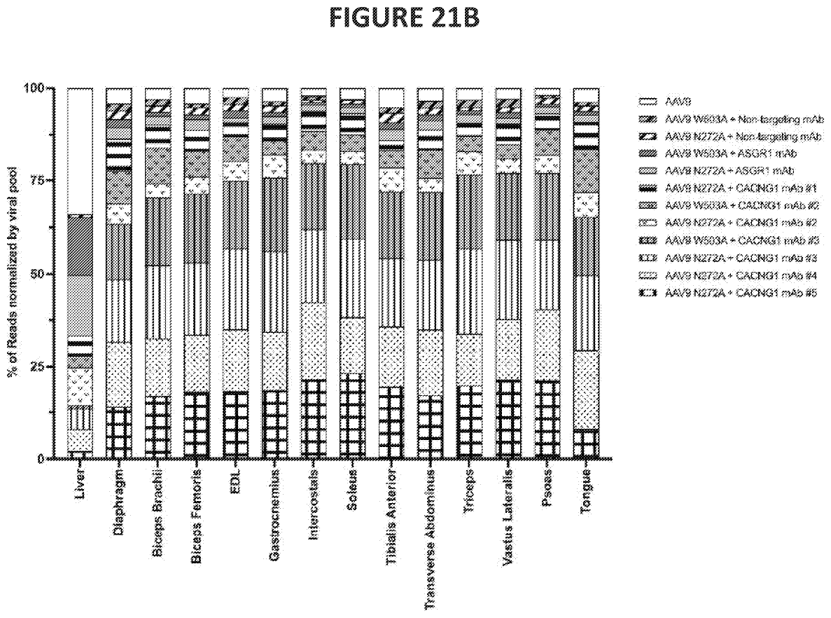

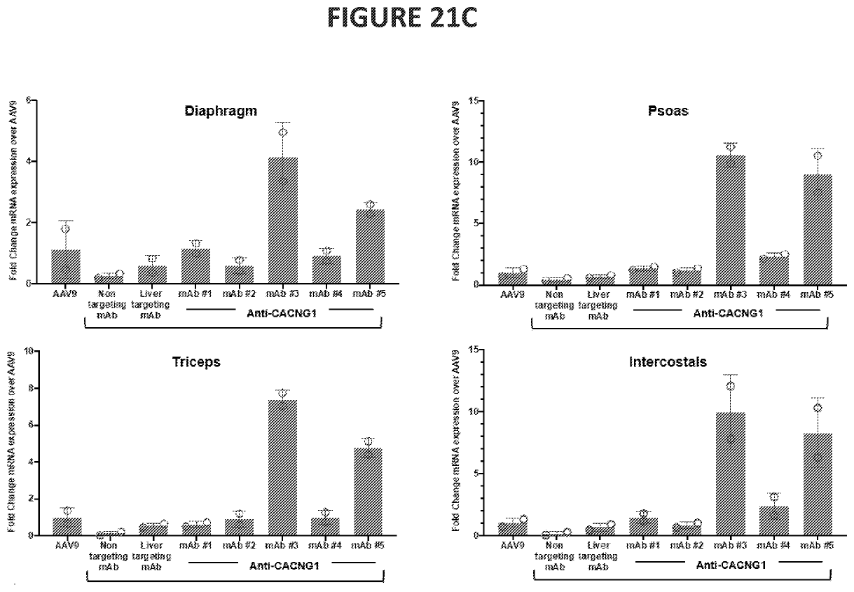

For a pooled AAV characterization experiment in cynomolgus monkey, control AAVs and AAV9 variants conjugated to the indicated antibodies were produced individually using the methods described above, but with barcoded pITR-CAG-GFP-hGHpA plasmids as the viral genome plasmids (FIG. 21A); each of the 12 viruses present in the pool was packaged with a version of pITR-CAG-GFP-hGHpA that carried a unique 32 nucleotide long barcode that was used to quantify transgene expression by that capsid variant. Two male cynomolgus macaques were given an intravenous bolus injection of 3E+13 vg/kg of the pooled virus mix. Two weeks after injection, animals were euthanized, and a set of tissues and organs were harvested for barcoding analysis. See, e.g., WO2018144813; Stoeckius et al. (2018) Genome Biol. 19:224; Stoeckius et al. (2017) Nat. Method 9:2579-10, each incorporated herein in its entirety by reference. Table 3 provides barcode number (BC #) associated with different viral particles (e.g., AAV9 cap mutation and corresponding antibody numbers where applicable).

TABLE 3

| BC# | AAV |
|---|---|
| 1 | AAV9 |
| 2 | AAV9 W503A + Non-targeting mAb |
| 3 | AAV9 W503A + ASGR1 mAb |
| 4 | AAV9 W503A + CACNG1 mAb #3 |
| 5 | AAV9 W503A + CACNG1 mAb #2 |
| 6 | AAV9 N272A + Non-targeting mAb |
| 7 | AAV9 N272A + ASGR1 mAb |
| 8 | AAV9 N272A + CACNG1 mAb #3 |
| 9 | AAV9 N272A + CACNG1 mAb #2 |
| 10 | AAV9 N272A + CACNG1 mAb #4 |
| 11 | AAV9 N272A + CACNG1 mAb #5 |
| 12 | AAV9 N272A + CACNG1 mAb #1 |

For barcoding analysis, total RNA isolated from cynomolgus monkey tissues and organs was purified using MagMAX-96 for Microarrays Total RNA Isolation Kit according to manufacturer's specifications. RNA was then treated with Turbo DNase and cDNA synthesis was performed using SuperScript IV reverse transcriptase and a hGH pA-specific primer (5′-GTCATGCATGCCTGGAATC-3′; SEQ ID NO:273). Barcoded GFP transcripts were amplified from cDNA samples with primers binding upstream (5′-TCGTCGGCAGCGTCAGATGTGTATAAGAGACAGCGAGCGCTGCTCGAGAG-3′; SEQ ID NO:274) and downstream (5′-GTCTCGTGGGCTCGGAGATGTGTATAAGAGACAGGGGTCACAGGGATGCCAC-3′; SEQ ID NO:275) of the barcodes using the Q5 High Fidelity 2× master mix. The pooled virus mix was included amongst the samples. Each sample was prepared in three technical replicates for the duration of the library preparation. Amplicons containing the Illumina adapters and unique dual indices (UDI-Illumina) were quantified using qubit and Tapestation, pooled at equimolar ratio, and sequenced on a Nextseq550 using the 300 cycles high output kit.

In the liver, AAV9 alone and AAV9 W503A or N272A conjugated to an ASGR1 mAb represent the majority of all barcodes present in the tissue, as expected (FIG. 21B). In skeletal muscle tissues, detargeted AAV9 (N272A or W503A) capsids conjugated to CACNG1 targeting antibodies represent the majority of all barcodes present in the tissue, outperforming AAV9 alone, which accounted for a small percentage of total barcodes (FIG. 21B). Similarly, FIG. 21C shows that systemically delivered detargeted AAV9 conjugated to CACNG1 demonstrates antibody-dependent transduction of skeletal muscles in non-human primates, relative to wildtype AAV9.

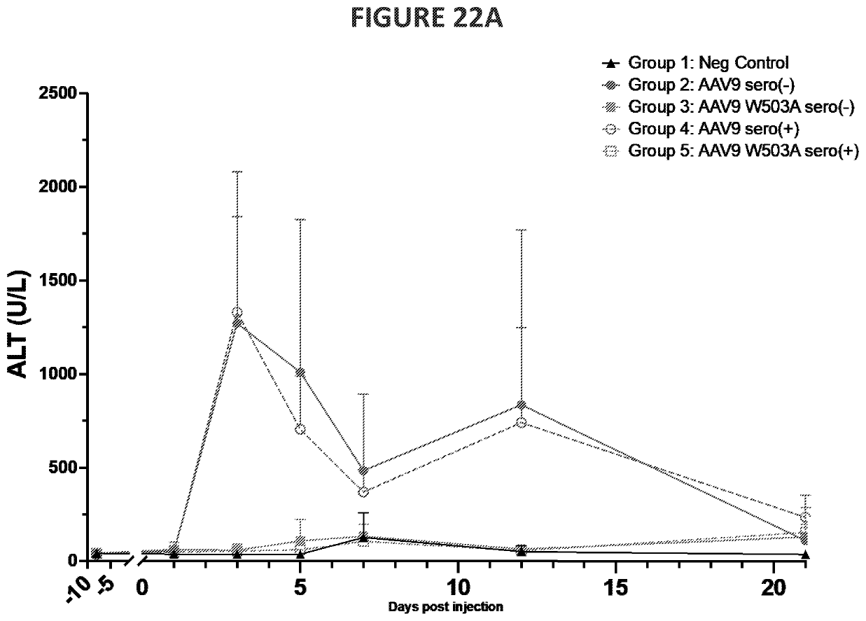

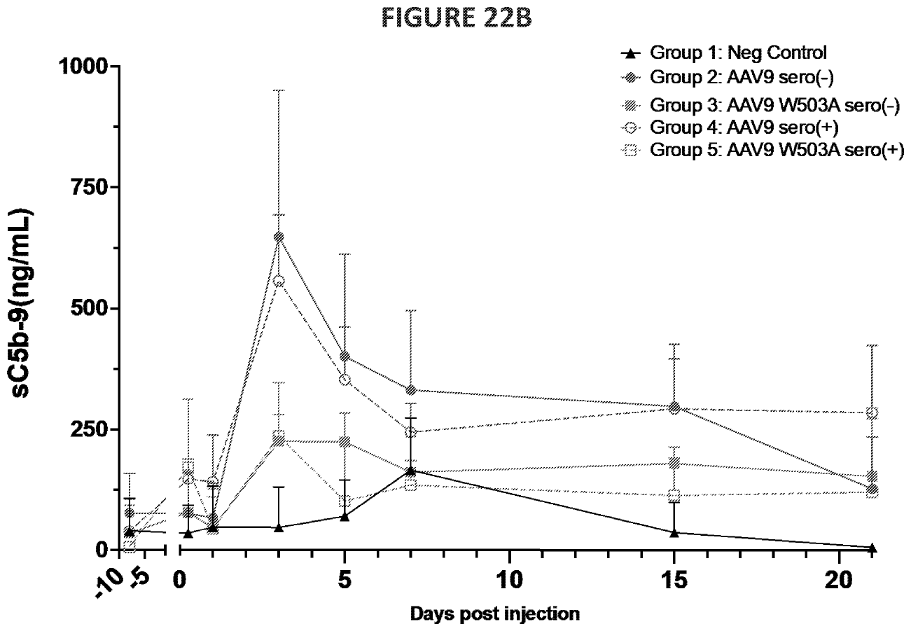

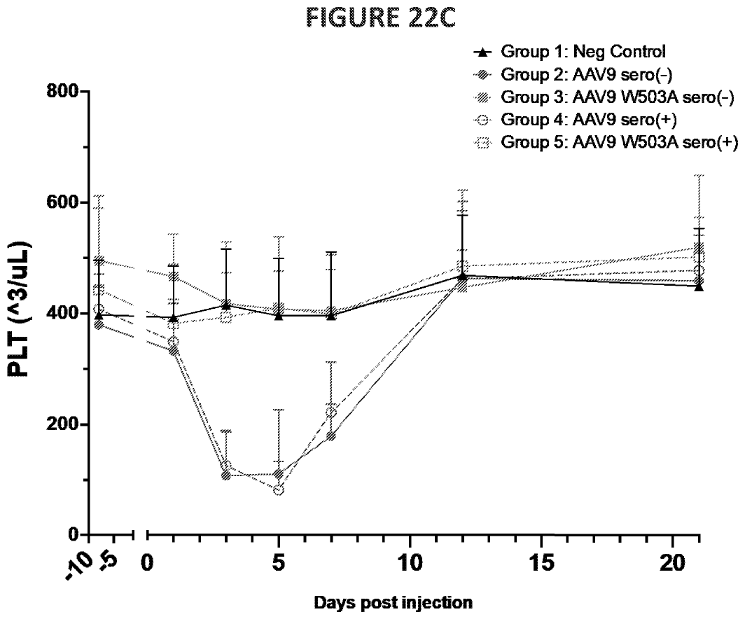

To assess serum readouts of liver health (ALT) and complement activation (sC5b-9) and assess markers of thrombotic microangiopathy (platelet counts) following administration of wildtype and detargeted AAVs, AAV9 and AAV9 W503A particles were produced according to the methods described above and packaged with pAAV CAG eGFP. Cynomolgus macaques were given an intravenous bolus injection of a high dose (2×10¹⁴ vg/kg) of the enumerated viral particle or saline as a control (N=4 per group). To additionally test the effect of pre-existing mild seropositivity of AAV9 on AAV transduction and toxicity, separate groups of neutralizing antibody positive (1:10 titer) monkeys received AAV9 and AAV9 W503A. Serum readouts of ALT and sC5b-9 and platelet counts were collected at baseline (10 days prior to dosing) as well as 24 hours, 48 hours, 72 hours, 5-days, 7 days, 15 days, and 21 days post-dosing. This high 2×10¹⁴ vg/kg dose of AAV9 is known to be more deleterious in terms of hepatotoxicity and complement-mediated toxicity in humans. Toxicity in non-human primates at doses >1×10¹⁴vg/kg have been variable, but trended towards higher acute toxicity. This study confirms that AAV9 is toxic at 2×10¹⁴ vg/kg in non-human primates. High-dose (2×10¹⁴ vg/kg) wildtype AAV9 induces rapid and severe ALT elevations indicative of hepatotoxicity, unlike detargeted AAV9 W503A (FIG. 22A). Moreover, a similar pattern was observed for serum levels of sC5b-9, a soluble marker for complement terminal membrane attack complex (FIG. 22B). Additional markers for complement pathway activation (Bb and C3a) were also more highly elevated for wildtype AAV9 but not detargeted AAV9 within the same timeframe. Accordingly, it is shown that the 2×10¹⁴ vg/kg dose induces greater levels of acute toxicity in wildtype AAV9 groups as compared to detargeted AAV9 W503A, as assessed by liver function tests and complement activity, with significant acute toxicity being observed at the 72-hour timepoint and beyond. Finally, it was shown that high-dose wildtype AAV9 induces transient thrombocytopenia, unlike detargeted AAV9 W503A (FIG. 22C). Seropositivity did not have a noticeable effect on these readouts.

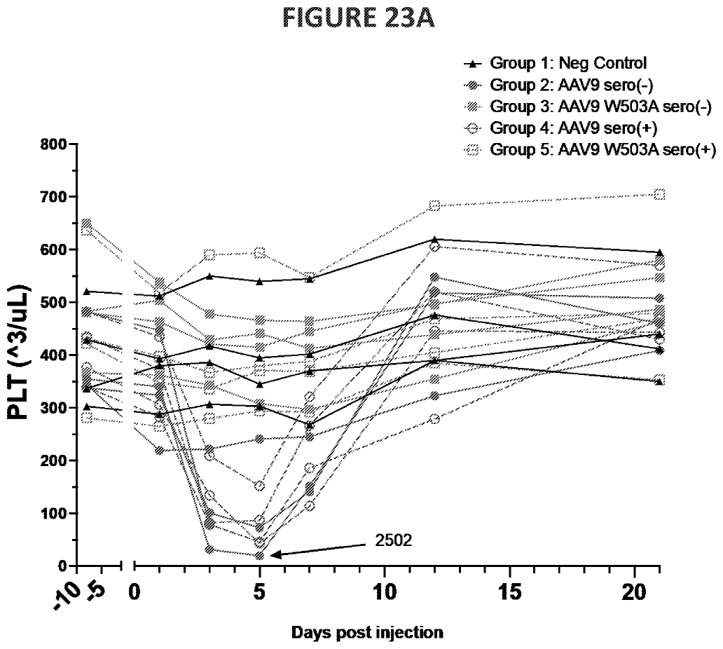

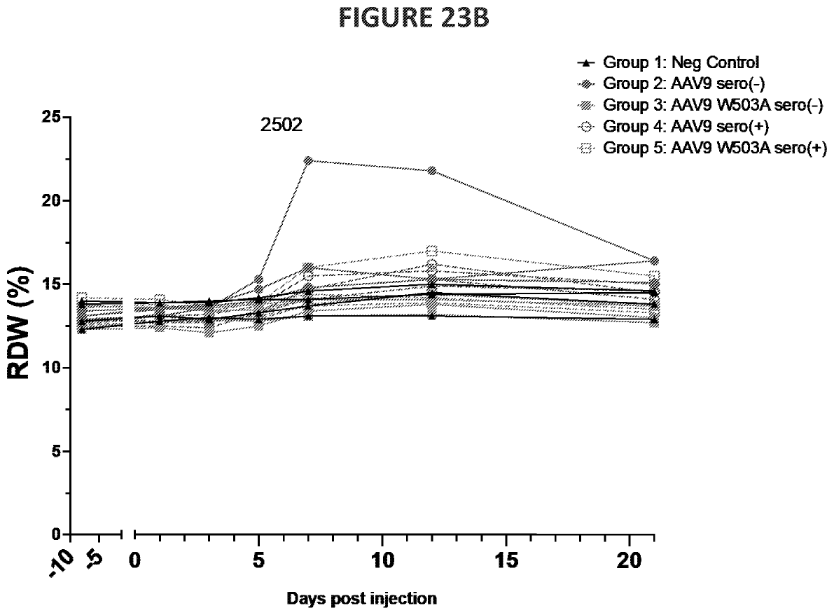

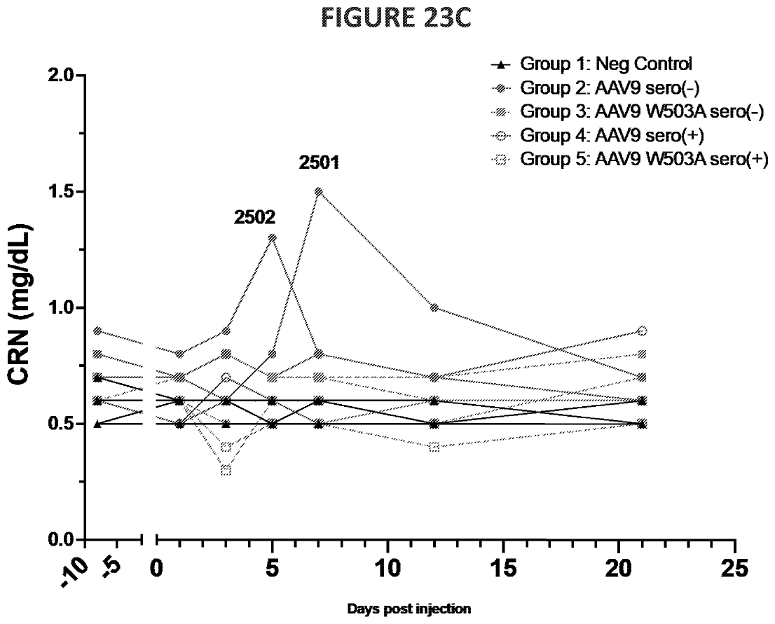

To further assess the thrombotic microangiopathy (TMA) phenotype, three markers of the triad of symptoms were assessed: thrombocytopenia (as assessed by platelet counts), hemolytic anemia (as assessed by red cell distribution width), and acute kidney injury (as assessed by serum creatinine levels). Markers of TMA were transiently observed at the 2×10¹⁴ vg/kg dose for wildtype AAV9 (thrombocytopenia, altered kidney function and schistocytes) (FIG. 23). Accordingly, cynomolgus macaques appear to reproduce some key features of AAV-induced TMA, although in a milder manner, with some wildtype AAV9-injected monkeys (e.g., 2502) displaying some symptoms of the TMA triad. Importantly, the detargeted AAV9 W503A monkeys did not have any deviations from control monkeys in these 3 markers, demonstrating improved safety by the detargeting mutation of AAV9.

What is claimed:

1. A recombinant adeno-associated virus (AAV) particle comprising:
(i) an AAV capsid protein modified with an antigen-binding protein that specifically binds to human Calcium Voltage Gated Channel Auxiliary Subunit Gamma 1 (hCACNG1), wherein the antigen-binding protein comprises an anti-hCACNG1 antibody or antigen-binding fragment thereof that comprises a set of HCDR1-HCDR2-HCDR3-LCDR1-LCDR2-LCDR3 amino acid sequences selected from the group consisting of SEQ ID NOs:4-6-8-12-14-16, SEQ ID NOs: 20-22-24-28-30-32, SEQ ID NOs:36-38-40-44-46-48, SEQ ID NOs:52-54-56-60-62-64; SEQ ID NOs: 68-70-72-76-78-80; SEQ ID NOs: 84-86-88-92-94-96; SEQ ID NOs: 100-102-104-108-110-112; SEQ ID NOs: 116-118-120-124-126-128; SEQ ID NOs:132-134-136-140-142-144; SEQ ID NOs: 148-150-152-156-158-160; SEQ ID NOs:164-166-168-172-174-176; and SEQ ID NOs: 180-182-186-188-190-192, and
(ii) a nucleotide of interest comprising a sequence comprising a coding sequence encoding microdystrophin, fukutin-related protein (FKRP), or myotubularin (MTM1),
wherein the nucleotide of interest is encapsulated by an AAV capsid comprising the AAV capsid protein modified with the antigen-binding protein that specifically binds to hCACNG1,
optionally wherein the modified AAV capsid protein comprises a first member of a protein:protein binding pair and a second member of a protein:protein binding pair, and the second member of the protein:protein binding pair comprises the anti-hCACNG1 antibody or antigen-binding fragment, and wherein the first member of the protein:protein binding pair and the second member of the protein:protein binding pair are associated to direct the tropism of the viral particle to hCACNG1.

2. The recombinant AAV particle of claim 1, wherein the anti-hCACNG1 antibody or antigen-binding fragment thereof comprises a heavy chain variable region (HCVR or VH) and/or a light chain variable region (LCVR or VL).

3. The recombinant AAV particle of claim 2, wherein the anti-hCACNG1 antibody or antigen-binding fragment thereof is selected from the group consisting of a human or humanized antibody or antigen binding fragment thereof, murine antibody or antigen binding fragment thereof, a monovalent Fab′, a divalent Fab2, a F(ab)′3 fragment, a single-chain fragment variable (scFv), a bis-scFv, a (scFv)2, a diabody, a minibody, a nanobody, a triabody, a tetrabody, a disulfide stabilized Fv protein (dsFv), a single-domain antibody (sdAb), an Ig NAR, a bispecific antibody or binding fragment thereof, a bi-specific T-cell engager (BiTE), a trispecific antibody, and chemically modified derivatives thereof.

4. The recombinant AAV particle of any one of claims 1-3, wherein the anti-hCACNG1 antibody or antigen-binding fragment thereof comprises a fragment antigen-binding region (Fab).

5. The recombinant AAV particle of any one of claims 1-3, wherein the anti-hCACNG1 antibody or antigen-binding fragment thereof comprises a single chain fragment variable (scFv).

6. The recombinant AAV particle of claim 5, wherein the scFv comprises domains arranged in the following orientation from N-terminus to C-terminus: HCVR-LCVR.

7. The recombinant AAV particle of claim 5, wherein the scFv comprises domains arranged in the following orientation from N-terminus to C-terminus: LCVR-HCVR.

8. The recombinant AAV particle of any one of claims 5-7, wherein the scFv variable regions are connected by a linker.

9. The recombinant AAV particle of claim 8, wherein the linker is a peptide linker.

10. The recombinant AAV particle of claim 9, wherein the peptide linker is -(GGGGS)n- (SEQ ID NO: 268); and wherein n is 1-10.

11. The recombinant AAV particle of any one of claims 1-10, wherein the anti-hCACNG1 antibody or antigen-binding fragment thereof binds to hCACNG1 with a K_{D} of about 1×10⁻⁷ M or a stronger affinity.

12. The recombinant AAV particle of any one of claims 1-11, wherein the anti-hCACNG1 antibody or antigen-binding fragment thereof binds to hCACNG1 with a K_{D} of about 10×10⁻⁸ to about 1×10⁻¹⁰.

13. The recombinant AAV particle of any one of claims 1-11, wherein the anti-hCACNG1 antibody or antigen-binding fragment thereof binds to hCACNG1 with a K_{D} of about 5×10⁻⁹ to about 1×10⁻¹⁰.

14. The recombinant AAV particle of any one of claims 1-13, wherein the anti-hCACNG1 antibody or antigen-binding fragment thereof comprises an HCVR/LCVR amino acid sequence pair having at least 90% sequence identity to an HCVR/LCVR amino acid sequence pair selected from the group consisting of SEQ ID NOs: 2/10, SEQ ID NOs: 18/26, SEQ ID NOs: 34/42, SEQ ID NOs: 50/58, SEQ ID NOs: 66/74, SEQ ID NOs: 82/90, SEQ ID NOs: 98/106, SEQ ID NOs: 114/122, SEQ ID NOs: 130/138, SEQ ID NOs: 146/154, SEQ ID NOs: 162/170, and SEQ ID NOs: 178/186.

15. The recombinant AAV particle of any one of claims 1-14, wherein:
(a) the first member of the protein:protein binding pair comprises SpyTag, Isopeptag, SnoopTag, SpyTag002, SpyTag003, or any biologically equivalent portions or variants thereof,
(b) the second member of the protein:protein binding pair comprises:
(i) a SpyCatcher, KTag, pilin-C, SnoopCatcher, SpyCatcher002, SpyCatcher003, or any biologically equivalent portions or variants thereof, and
(ii) the anti-hCACNG1 antibody or antigen-binding fragment thereof, and
(c) the first member of the protein:protein binding pair and the second member of the protein:protein binding pair are linked by an isopeptide bond.

16. The recombinant AAV particle of claim 15, wherein:
(a) the first member of the protein:protein binding pair comprises SpyTag, or any biologically equivalent portion or variant thereof, and
(b) the second member of the protein:protein binding pair comprises SpyCatcher, or any biologically equivalent portions or variants thereof, fused to the anti-hCACNG1 antibody or antigen-binding fragment thereof.

17. The recombinant AAV particle of any one of claims 1-16, comprising a first and/or second linker operably linking the first member of the protein:protein binding pair to the viral capsid protein.

18. The recombinant AAV particle of claim 17, wherein the first and second linker are not identical.

19. The recombinant AAV particle of claim 17, wherein the first and second linker are identical.

20. The recombinant AAV particle of any one of claims 17-19, wherein the first linker is 10 amino acids in length and/or the second linker is 10 amino acids in length.

21. The recombinant AAV particle of any one of claims 1-20, wherein the modified AAV capsid protein comprises a modified VP1 capsid protein, modified VP2 capsid protein, and/or modified VP3 capsid protein, and
wherein the modified VP1 capsid protein, modified VP2 capsid protein, and/or modified VP3 capsid protein comprises an insertion of a first member of a protein:protein binding pair and/or the anti-hCACNG1 antibody or antigen-binding fragment thereof, and
wherein a portion of the modified VP1 capsid protein, modified VP2 capsid protein, and/or modified VP3 capsid protein, that comprises the insertion of a first member of a protein:protein binding pair and/or the anti-hCACNG1 antibody or antigen-binding fragment thereof, further comprises an amino acid sequence at least 90% identical to a corresponding capsid protein of a wild-type AAV.

22. The recombinant AAV particle of claim 21, wherein the modified VP1 capsid protein, the modified VP2 capsid protein, and/or the modified VP3 capsid protein further comprises, in addition to the insertion of a first member of a protein:protein binding pair and/or the anti-hCACNG1 antibody or antigen-binding fragment thereof:
(i) a substitution, insertion, or deletion of an amino acid,
(ii) a chimeric amino acid sequence, or
(iii) any combination of (i) and (ii).

23. The recombinant AAV particle of claim 22, wherein the substitution, insertion, or deletion of an amino acid reduces the natural tropism of the viral particle and/or creates a detectable label.

24. The recombinant AAV particle of any one of claims 1-23, wherein the AAV is selected from the group consisting of AAV1, AAV2, AAV3, AAV4, AAV5, AAV6, AAV7, AAV8, AAV9, a non-primate animal AAV listed in Table 2, and any chimera thereof.

25. The recombinant AAV particle of any one of claims 1-24, wherein the AAV is AAV2.

26. The recombinant AAV particle of any one of claims 1-25, wherein the recombinant AAV particle comprises a modified AAV2 VP1 capsid protein that comprises a first member of a protein:protein binding pair inserted at an amino acid position I-453 and/or I-587, and optionally linked to the AAV sequence via a linker on one or both sides.

27. The recombinant AAV particle of claim 26, wherein the recombinant AAV particle comprises a modified AAV2 VP1 capsid protein that comprises the first member of the protein:protein binding pair inserted, optionally via a linker, at position G453,
optionally wherein the modified AAV2 VP1 capsid protein further comprises a mutation selected from R585A, R588A, R484A, R487A, K532A, and any combination thereof.

28. The recombinant AAV particle or composition of claim 26 or claim 27, wherein the recombinant AAV particle comprises a mosaic AAV capsid comprising a second set of AAV2 VP1 capsid proteins lacking the first member of the protein:protein binding pair,
optionally wherein the second set of AAV2 VP1 capsid proteins comprises a mutation selected from R585A, R588A, R484A, R487A, K532A and any combination thereof.

29. The recombinant AAV particle of any one of claims 1-24, wherein the AAV is AAV9.

30. The recombinant AAV particle of claim 29, wherein the viral capsid comprises a modified AAV9 VP1 capsid protein that comprises a first member of a specific binding pair inserted, optionally via a linker, at position I-453 or 1-589.

31. The recombinant AAV particle of claim 30, wherein the recombinant AAV particle comprises a modified AAV9 VP1 capsid protein that comprises a first member of a protein:protein binding pair inserted, optionally via a linker, at position G453,
optionally wherein the modified AAV9 VP1 capsid protein further comprises a mutation selected from N272A, W503A, and a combination thereof.

32. The recombinant AAV particle of claim 30 or claim 31, wherein the recombinant AAV particle is a mosaic viral capsid comprising a second set of AAV9 VP1 capsid proteins lacking the first member of the protein:protein binding pair,
optionally wherein the second set of AAV9 VP1 capsid proteins comprises a mutation selected from N272A, W503A, and a combination thereof.

33. The recombinant AAV particle any one of claims 1-24, wherein the AAV is a non-primate animal AAV.

34. The recombinant AAV particle of claim 33, wherein the non-primate animal AAV is an avian AAV (AAAV).

35. The recombinant AAV particle of claim 34, wherein the AAV capsid comprises a modified AAAV VP1 capsid protein that comprises the first member of the protein:protein binding pair inserted, optionally via a linker, at position I-444 or I-580.

36. The recombinant AAV particle of claim 33, wherein the non-primate animal AAV is a squamate AAV.

37. The recombinant AAV particle of claim 36, wherein the squamate AAV is a bearded dragon AAV.

38. The recombinant AAV particle of claim 37, wherein the AAV capsid comprises a modified bearded dragon AAV VP1 capsid protein that comprises the first member of the protein:protein binding pair inserted, optionally via a linker, at position I-573 or 1-436.

39. The recombinant AAV particle of claim 33, wherein the non-primate animal AAV is a non-primate mammalian AAV.

40. The recombinant AAV particle of claim 39, wherein the non-primate mammalian AAV is a sea lion AAV.

41. The recombinant AAV particle of claim 40, wherein the AAV capsid comprises a modified sea lion AAV VP1 capsid protein that comprises the first member of the protein:protein binding pair inserted, optionally via a linker, at a position selected from the group consisting of I-429, I-430, 1-431, I-432, I-433, I-434, I-436, I-437, and I-565.

42. The recombinant AAV particle of any one of claims 1-41, wherein the recombinant AAV particle comprises a mosaic AAV capsid, optionally wherein the mosaic AAV capsid comprises (i) a first plurality of reference capsid proteins, each of which is not associated with the anti-hCACNG1 antibody or antigen-binding fragment thereof, and (ii) a second plurality of capsid proteins, each of which is associated with the anti-hCACNG1 antibody or antigen-binding fragment thereof, optionally wherein the mosaic AAV particle comprises the first plurality of reference capsid proteins and the second plurality of capsid proteins at a ratio of 1:7.

43. The recombinant AAV particle of any one of claims 1-42, wherein the nucleotide of interest encodes microdystrophin.

44. The recombinant AAV particle of any one of claims 1-43, wherein the nucleotide of interest comprises a sequence set forth as SEQ ID NO:270.

45. The recombinant AAV particle of any one of claims 1-42, wherein the nucleotide of interest encodes human FKRP.

46. The recombinant AAV particle of any one of claims 1-42, and 45 wherein the nucleotide of interest comprises a sequence set forth as SEQ ID NO:271.

47. The recombinant AAV particle of any one of claims 1-42, wherein the nucleotide of interest encodes human MTM1.

48. The recombinant AAV particle of any one of claims 1-42, and 47 wherein the nucleotide of interest comprises a sequence set forth as SEQ ID NO:272.

49. A method of treating Duchenne muscular dystrophy in a patient in need thereof comprising administering to the patient the recombinant AAV particle of any one of claims 1-44, optionally at a dose greater than 3×10¹³ vg/kg (e.g., 2×10¹⁴).

50. A method of treating limb girdle muscular dystrophy in a patient in need thereof comprising administering to the patient the recombinant AAV particle of any one of claims 1-42 and 45-46, optionally at a dose greater than 3×10¹³ vg/kg (e.g., 2×10¹⁴).

51. A method of treating myotubular myopathy in a patient in need thereof comprising administering to the patient the recombinant AAV particle of any one of claims 1-42 and 47-48, optionally at a dose greater than 3×10¹³ vg/kg (e.g., 2×10¹⁴).

52. A method of treating a muscle wasting or genetic muscle disease in a subject in need thereof comprising:
administering, to the subject, a recombinant AAV particle of any one of claims 1-42, optionally at a dose greater than 3×10¹³ vg/kg,
wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

53. Use of a recombinant AAV particle of any one of claims 1-42, optionally at a dose greater than 3×10¹³ vg/kg (e.g., 2×10¹⁴), for the manufacture of a medicament for administration to a subject in the treatment of muscle wasting or a genetic muscle disease.

54. The method of claim 52 or use of claim 53, wherein the muscle wasting or genetic muscle disease is selected from the group consisting of X-linked myotubular myopathy (XLMTM), Duchenne muscular dystrophy (DMD), myotonic dystrophy (DM1), Facioscapulohumeral muscular dystrophy Type 1 (FSHD), congenital muscular dystrophy type 1A (MDC1A), Limb girdle muscular dystrophy, and dystroglycanopathy.

55. The method or use of any one of claims 50-54, wherein the administration to the subject of the recombinant AAV particle at a dose greater than 3×10¹³ vg/kg (e.g., 2×10¹⁴) does not result in:
(i) a significantly increased level of a liver enzyme (e.g., ALT) at 1, 3, 5, 7, 15 and/or 21 days post administration, compared to the corresponding level of the liver enzyme (e.g., ALT) in the subject prior to the administration,
(ii) a significantly increased level of one or more complement components (e.g., Bb, C3a, sC5b-9) at 1, 3, 5, 7, 15 and/or 21 days post administration, compared to the corresponding level of the one or more complement components (e.g., Bb, C3a, sC5b-9) in the subject prior to the administration,
(iii) a significantly decreased level of platelet counts at 1, 3, 5, 7, 15 and/or 21 days post administration, compared to the corresponding platelet count in the subject prior to the administration,
(iv) a significantly increased red cell distribution width (RDW) at 1, 3, 5, 7, 15 and/or 21 days post administration, compared to the corresponding RDW in the subject prior to the administration,
(v) a significantly increased level of serum creatinine at 1, 3, 5, 7, 15 and/or 21 days post administration, compared to the corresponding level of serum creatinine in the subject prior to the administration, or
(vi) any combination of (i)-(v).

56. The method or use of claim 55, wherein the subject is a non-human primate.

57. The method or use of claim 55, wherein the subject is a human.


SEQUENCE LISTING (275 sequences)

SEQ ID NO: 1 - DNA, 357 bp, synthetic construct
  source 1..357: mol_type = other DNA
caggtgcagctggtggagtctgggggaggcgtggtccagcctgggaggtccctgagactctcctgtacagcgtctggaatcaccttcagaaattatggcatgcactgggtccgccaggctccaggcaaggggctggagtgggtggcagttatgtggtatgatggaagtaataagtactatgcagactccgtgaagggccgtttcaccatctccggagacaattccaaggtgtatctgcaaatgaacagcctgagagccgaggacacggctgtatattactgtgcgagaaggggcactataagaacagctgccccttttgactactggggtcagggaaccctggtcaccgtctcctca

SEQ ID NO: 2 - protein, 119 aa, synthetic construct
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSS

SEQ ID NO: 3 - DNA, 24 bp, synthetic construct
  source 1..24: mol_type = other DNA
ggaatcaccttcagaaattatggc

SEQ ID NO: 4 - protein, 8 aa, synthetic construct
GITFRNYG

SEQ ID NO: 5 - DNA, 24 bp, synthetic construct
  source 1..24: mol_type = other DNA
atgtggtatgatggaagtaataag

SEQ ID NO: 6 - protein, 8 aa, synthetic construct
MWYDGSNK

SEQ ID NO: 7 - DNA, 42 bp, synthetic construct
  source 1..42: mol_type = other DNA
gcgagaaggggcactataagaacagctgccccttttgactac

SEQ ID NO: 8 - protein, 14 aa, synthetic construct
ARRGTIRTAAPFDY

SEQ ID NO: 9 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 11 - DNA, 18 bp, synthetic construct
  source 1..18: mol_type = other DNA
cagagcattagcagctat

SEQ ID NO: 12 - protein, 6 aa, synthetic construct
QSISSY

SEQ ID NO: 13-14 - skipped (2 entries)

SEQ ID NO: 15 - DNA, 30 bp, synthetic construct
  source 1..30: mol_type = other DNA
caacagagttacagtacccctccgatcacc

SEQ ID NO: 16 - protein, 10 aa, synthetic construct
QQSYSTPPIT

SEQ ID NO: 17 - DNA, 356 bp, synthetic construct
  source 1..356: mol_type = other DNA
caggtgcagctacagcagtggggcgcaggactgttgaagccttcggcgaccctgtcccgcacctgcgctgtctatggtgggtccttcagtggttactactggaactggatccgccagtccccagggaaggggctggaatggattggggaaatccttcatagtggaagaaccaactacaacccgtccctcaagagtcgagtcaccatatcagtagacacgtccaagaaccagttctccctgaagctgacctctgtgaccgccgcggacacggctgtatattactgtgcggaaggatagcagctcgtcacggctggttcgacccctggggccagggaaccctggtcaccgtctcctca

SEQ ID NO: 18 - protein, 119 aa, synthetic construct
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSS

SEQ ID NO: 19 - DNA, 24 bp, synthetic construct
  source 1..24: mol_type = other DNA
ggtgggtccttcagtggttactac

SEQ ID NO: 20 - protein, 8 aa, synthetic construct
GGSFSGYY

SEQ ID NO: 21 - DNA, 21 bp, synthetic construct
  source 1..21: mol_type = other DNA
atccttcatagtggaagaacc

SEQ ID NO: 22 - protein, 7 aa, synthetic construct
ILHSGRT

SEQ ID NO: 23 - DNA, 39 bp, synthetic construct
  source 1..39: mol_type = other DNA
gcgggaaggatagcagctcgtcacggctggttcgacccc

SEQ ID NO: 24 - protein, 13 aa, synthetic construct
AGRIAARHGWFDP

SEQ ID NO: 25 - DNA, 321 bp, synthetic construct
  source 1..321: mol_type = other DNA
gacatccagatgacccagtctccatcttccgtgtctacatctgtaggagacagagtcaccatctcttgtcgggcgagtcaggatattcgcaagtggttagcctggtatcaacagaaaccaggaaaagcccctaaactcctgatctatgctacatccagtttgcaaagtggggtcccttcaaggttcagcggcagtggatctgggacagatttcactctcaccatcagcagcctgcagcctgaggattttgcaacttacttttgtcaacaggctaacagtttcccgttcacttttggccaggggaccaagctggagatcaaa

SEQ ID NO: 26 - protein, 107 aa, synthetic construct
DIQMTQSPSSVSTSVGDRVTISCRASQDIRKWLAWYQQKPGKAPKLLIYATSSLQSGVPSRFSGSGSGTDFTLTISSLQPEDFATYFCQQANSFPFTFGQGTKLEIK

SEQ ID NO: 27 - DNA, 18 bp, synthetic construct
  source 1..18: mol_type = other DNA
caggatattcgcaagtgg

SEQ ID NO: 28 - protein, 6 aa, synthetic construct
QDIRKW

SEQ ID NO: 29-30 - skipped (2 entries)

SEQ ID NO: 31 - DNA, 27 bp, synthetic construct
  source 1..27: mol_type = other DNA
caacaggctaacagtttcccgttcact

SEQ ID NO: 32 - protein, 9 aa, synthetic construct
QQANSFPFT

SEQ ID NO: 33 - DNA, 357 bp, synthetic construct
  source 1..357: mol_type = other DNA
caggtgcagctggtggagtctgggggaggcgtggtccagcctgggaggtccctgagactctcctgtgcagcgtctggattcaccttcagtacatatggcatgcactgggtccgccaggctccaggcaaggggctggagtgggtggcagttatttggcatgatggaagtgataaatattatgtagactccgtgaagggccgattctccatcgccagagacaattccaagaacacgctttatctgcaaatgaatagtctgagagtcgaggacacgggtatatattactgtgcgagaaggggtatacgtggaaccgtttttgaccactggggcctgggaaccctggtcaccgtctcctca

SEQ ID NO: 34 - protein, 119 aa, synthetic construct
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSS

SEQ ID NO: 35 - DNA, 24 bp, synthetic construct
  source 1..24: mol_type = other DNA
ggattcaccttcagtacatatggc

SEQ ID NO: 36 - protein, 8 aa, synthetic construct
GFTFSTYG

SEQ ID NO: 37 - DNA, 24 bp, synthetic construct
  source 1..24: mol_type = other DNA
atttggcatgatggaagtgataaa

SEQ ID NO: 38 - protein, 8 aa, synthetic construct
IWHDGSDK

SEQ ID NO: 39 - DNA, 36 bp, synthetic construct
  source 1..36: mol_type = other DNA
gcgagaaggggtatacgtggaaccgtttttgaccac

SEQ ID NO: 40 - protein, 12 aa, synthetic construct
ARRGIRGTVFDH

SEQ ID NO: 41 - DNA, 321 bp, synthetic construct
  source 1..321: mol_type = other DNA
gacatccagatgacccagtctccttccaccctgtctgcatctgtaggagacagagtcaccctcacttgtcgggccagtcagagtattagtaacaagttggcctggtatcagcagaaaccagggaaagcccctaacctcctgatctataaggcgtctaatttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaa

SEQ ID NO: 42 - protein, 107 aa, synthetic construct
DIQMTQSPSTLSASVGDRVTLTCRASQSISNKLAWYQQKPGKAPNLLIYKASNLESGVPSRFSGSGSGTEFTLTISSLQPDDFATYYCQQYNSYSWTFGQGTKVEIK

SEQ ID NO: 43 - DNA, 18 bp, synthetic construct
  source 1..18: mol_type = other DNA
cagagtattagtaacaag

SEQ ID NO: 44 - protein, 6 aa, synthetic construct
QSISNK

SEQ ID NO: 45-46 - skipped (2 entries)

SEQ ID NO: 47 - DNA, 27 bp, synthetic construct
  source 1..27: mol_type = other DNA
caacagtataatagttattcgtggacg

SEQ ID NO: 48 - protein, 9 aa, synthetic construct
QQYNSYSWT

SEQ ID NO: 49 - DNA, 348 bp, synthetic construct
  source 1..348: mol_type = other DNA
caggtgcagctgcaggagtcgggcccaggactggtgaagccttcggagaccctgtccctcacctgcactgtctctggtgactccatcaataattactactggacctggctccggcagcccccagggaagggactggagtggattggttatatctattacagtgggagcgccaactacaacccctccctcaagagtcgagtcaccatatcagtagacacgtccaagaaccagttctccctgaagctaaattctgtgaccgctgcggacacggccgtgtattactgtgcgagaggggcggtcaagtacttccggcattggggccagggcaccctggtcaccgtctcctca

SEQ ID NO: 50 - protein, 116 aa, synthetic construct
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSS

SEQ ID NO: 51 - DNA, 24 bp, synthetic construct
  source 1..24: mol_type = other DNA
ggtgactccatcaataattactac

SEQ ID NO: 52 - protein, 8 aa, synthetic construct
GDSINNYY

SEQ ID NO: 53 - DNA, 21 bp, synthetic construct
  source 1..21: mol_type = other DNA
atctattacagtgggagcgcc

SEQ ID NO: 54 - protein, 7 aa, synthetic construct
IYYSGSA

SEQ ID NO: 55 - DNA, 30 bp, synthetic construct
  source 1..30: mol_type = other DNA
gcgagaggggcggtcaagtacttccggcat

SEQ ID NO: 56 - protein, 10 aa, synthetic construct
ARGAVKYFRH

SEQ ID NO: 57 - DNA, 324 bp, synthetic construct
  source 1..324: mol_type = other DNA
gaaattgtgttgacgcagtctccgggcaccctctctttgtctccaggggaaagagccaccctctcctgcagggccagtcagactattaaccacaacaacttagcctggtaccagcagagacctggccaggctcccaggctcctcatctatggtgcatccaacagggccactgccatcccagacaggttcagtggcagtgggtctgggacagacttcactctcaccatcagcagactggagcctgaagattttgaagtgtattcttgtcagcagtatggtagcttgccgctcactttcggcggagggaccaaggtggagatcaaa

SEQ ID NO: 58 - protein, 108 aa, synthetic construct
EIVLTQSPGTLSLSPGERATLSCRASQTINHNNLAWYQQRPGQAPRLLIYGASNRATAIPDRFSGSGSGTDFTLTISRLEPEDFEVYSCQQYGSLPLTFGGGTKVEIK

SEQ ID NO: 59 - DNA, 21 bp, synthetic construct
  source 1..21: mol_type = other DNA
cagactattaaccacaacaac

SEQ ID NO: 60 - protein, 7 aa, synthetic construct
QTINHNN

SEQ ID NO: 61-62 - skipped (2 entries)

SEQ ID NO: 63 - DNA, 27 bp, synthetic construct
  source 1..27: mol_type = other DNA
cagcagtatggtagcttgccgctcact

SEQ ID NO: 64 - protein, 9 aa, synthetic construct
QQYGSLPLT

SEQ ID NO: 65 - DNA, 363 bp, synthetic construct
  source 1..363: mol_type = other DNA
caggtgcagctggtggagtctgggggaggcgtggtccagcctgggacgtccctgagactctcctgtgcagcgtcaggattcaccttcagtagctatggcatgcactgggtccgccaggctccaggcaaggggctggagtgggtggcagttatatggattgatggaagtaataaatattatgcagactccgtgaagggccgattcaccatctccagagacaattccaagaacacgctgtatctgcaaatgaacagcctgagagccgaggacacggctgtgtattactgtgcgagaagggggggtatagtagtagctgccccctttgactactggggccagggaaccctggtcaccgtctcctca

SEQ ID NO: 66 - protein, 121 aa, synthetic construct
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSS

SEQ ID NO: 67 - DNA, 24 bp, synthetic construct
  source 1..24: mol_type = other DNA
ggattcaccttcagtagctatggc

SEQ ID NO: 68 - protein, 8 aa, synthetic construct
GFTFSSYG

SEQ ID NO: 69 - DNA, 24 bp, synthetic construct
  source 1..24: mol_type = other DNA
atatggattgatggaagtaataaa

SEQ ID NO: 70 - protein, 8 aa, synthetic construct
IWIDGSNK

SEQ ID NO: 71 - DNA, 42 bp, synthetic construct
  source 1..42: mol_type = other DNA
gcgagaagggggggtatagtagtagctgccccctttgactac

SEQ ID NO: 72 - protein, 14 aa, synthetic construct
ARRGGIVVAAPFDY

SEQ ID NO: 73 - DNA, 324 bp, synthetic construct; identical to SEQ ID NO: 9
  source 1..324: mol_type = other DNA

SEQ ID NO: 74 - protein, 108 aa, synthetic construct; identical to SEQ ID NO: 10

SEQ ID NO: 75 - DNA, 18 bp, synthetic construct
  source 1..18: mol_type = other DNA
cagagcattagcagctat

SEQ ID NO: 76 - protein, 6 aa, synthetic construct
QSISSY

SEQ ID NO: 77-78 - skipped (2 entries)

SEQ ID NO: 79 - DNA, 30 bp, synthetic construct; identical to SEQ ID NO: 15
  source 1..30: mol_type = other DNA

SEQ ID NO: 80 - protein, 10 aa, synthetic construct
QQSYSTPPIT

SEQ ID NO: 81 - DNA, 363 bp, synthetic construct
  source 1..363: mol_type = other DNA
caggtgcagttggtggagtctgggggaggcgtggtccagcctgggaggtccctgagactctcctgtgaagcgtctggaatcaccttcagaaactatggcatgcactgggtccgccaggctccaggcaaggggctggagtgggtggcagttatgtggtatgatggaagtaataaatactacgcagactccgtgaagggccgattcaccatctccagagacaattccaagaacacggtgtatctgcaaatgaacagcctgagagccgaagacacggctgtgtattactgtgcgagacggggtcatatagcaacagctgctccctttgactactggggccagggaaccctggtcaccgtctcctca

SEQ ID NO: 82 - protein, 121 aa, synthetic construct
QVQLVESGGGVVQPGRSLRLSCEASGITFRNYGMHWVRQAPGKGLEWVAVMWYDGSNKYYADSVKGRFTISRDNSKNTVYLQMNSLRAEDTAVYYCARRGHIATAAPFDYWGQGTLVTVSS

SEQ ID NO: 83 - DNA, 24 bp, synthetic construct
  source 1..24: mol_type = other DNA
ggaatcaccttcagaaactatggc

SEQ ID NO: 84 - protein, 8 aa, synthetic construct
GITFRNYG

SEQ ID NO: 85 - DNA, 24 bp, synthetic construct
  source 1..24: mol_type = other DNA
atgtggtatgatggaagtaataaa

SEQ ID NO: 86 - protein, 7 aa, synthetic construct
MWYDGSN

SEQ ID NO: 87 - DNA, 42 bp, synthetic construct
  source 1..42: mol_type = other DNA
gcgagacggggtcatatagcaacagctgctccctttgactac

SEQ ID NO: 88 - protein, 13 aa, synthetic construct
ARRGHIATAAPFD

SEQ ID NO: 89 - DNA, 324 bp, synthetic construct
  source 1..324: mol_type = other DNA
gacatccagatgacccagtctccatcctccctgtctgcatccgtaggagacagagtcaccatcagttgccgggcaagtcagagcattagtagttatttaaattggtatcagcagaaaccagggaaagcccctaaggtcctgatgtatgctgcatccagtttgcaaagtggggtcccatcaaggttcagtggcagtggatctgggacagatttcactctcaccatcagcagtctgcaacctgaggattttgcaacttactactgtcaacagagttacagtacccctccgatcaccttcggccaagggacacgactggagattaaa

SEQ ID NO: 90 - protein, 108 aa, synthetic construct
DIQMTQSPSSLSASVGDRVTISCRASQSISSYLNWYQQKPGKAPKVLMYAASSLQSGVPSRFSGSGSGTDFTLTISSLQPEDFATYYCQQSYSTPPITFGQGTRLEIK

SEQ ID NO: 91 - DNA, 18 bp, synthetic construct
  source 1..18: mol_type = other DNA
cagagcattagtagttat

SEQ ID NO: 92 - protein, 6 aa, synthetic construct
QSISSY

SEQ ID NO: 93-94 - skipped (2 entries)

SEQ ID NO: 95 - DNA, 30 bp, synthetic construct; identical to SEQ ID NO: 15
  source 1..30: mol_type = other DNA

SEQ ID NO: 96 - protein, 10 aa, synthetic construct
QQSYSTPPIT

SEQ ID NO: 97 - DNA, 363 bp, synthetic construct
  source 1..363: mol_type = other DNA
caggtgcagctggtggagtctgggggaggcgtggtccagcctgggaggtccctgagactctcctgtacagcgtctggattcaccttccgttcctatggcatgcactgggtccgccaggctccaggcaaggggctggagtgggtgtcagttatttggattgatggaaataatatatactatgcagactccgtgaagggccgattcaccatctccagagacaattccaagaacacgctgtatctgcaaatggacagcctgagagccgaggacacggctgtttattactgtgcgagaagactggctataacatcagctgccccctttgactactggggccagggaaccctggtcaccgtctcctca

SEQ ID NO: 98 - protein, 121 aa, synthetic construct
QVQLVESGGGVVQPGRSLRLSCTASGFTFRSYGMHWVRQAPGKGLEWVSVIWIDGNNIYYADSVKGRFTISRDNSKNTLYLQMDSLRAEDTAVYYCARRLAITSAAPFDYWGQGTLVTVSS

SEQ ID NO: 99 - DNA, 24 bp, synthetic construct
  source 1..24: mol_type = other DNA
ggattcaccttccgttcctatggc

SEQ ID NO: 100 - protein, 8 aa, synthetic construct
GFTFRSYG

SEQ ID NO: 101 - DNA, 24 bp, synthetic construct
  source 1..24: mol_type = other DNA
atttggattgatggaaataatata

SEQ ID NO: 102 - protein, 8 aa, synthetic construct
IWIDGNNI

SEQ ID NO: 103 - DNA, 42 bp, synthetic construct
  source 1..42: mol_type = other DNA
gcgagaagactggctataacatcagctgccccctttgactac

SEQ ID NO: 104 - protein, 14 aa, synthetic construct
ARRLAITSAAPFDY

SEQ ID NO: 105 - DNA, 324 bp, synthetic construct; identical to SEQ ID NO: 9
  source 1..324: mol_type = other DNA

SEQ ID NO: 106 - protein, 108 aa, synthetic construct; identical to SEQ ID NO: 10

SEQ ID NO: 107 - DNA, 18 bp, synthetic construct
  source 1..18: mol_type = other DNA
cagagcattagcagctat

SEQ ID NO: 108 - protein, 6 aa, synthetic construct
QSISSY

SEQ ID NO: 109-110 - skipped (2 entries)

SEQ ID NO: 111 - DNA, 30 bp, synthetic construct; identical to SEQ ID NO: 15
  source 1..30: mol_type = other DNA

SEQ ID NO: 112 - protein, 10 aa, synthetic construct
QQSYSTPPIT

SEQ ID NO: 113 - DNA, 378 bp, synthetic construct
  source 1..378: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttca

SEQ ID NO: 114 - protein, 126 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSS

SEQ ID NO: 115 - DNA, 24 bp, synthetic construct
  source 1..24: mol_type = other DNA
ggttacgccttcaccacctatggt

SEQ ID NO: 116 - protein, 8 aa, synthetic construct
GYAFTTYG

SEQ ID NO: 117 - DNA, 24 bp, synthetic construct
  source 1..24: mol_type = other DNA
atcagcgcttacaatggaaataca

SEQ ID NO: 118 - protein, 7 aa, synthetic construct
ISAYNGN

SEQ ID NO: 119 - DNA, 57 bp, synthetic construct
  source 1..57: mol_type = other DNA
gcgagaaagggtcactatggttcggggacttattataacccctttggttttgatttt

SEQ ID NO: 120 - protein, 19 aa, synthetic construct
CARKGHYGSGTYYNPFGFD

SEQ ID NO: 121 - DNA, 324 bp, synthetic construct
  source 1..324: mol_type = other DNA
gaaattatgttgatgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactgacatcccagacaggttcagtggcagtgggtctgggacagacttcactctcaccatcagcagactggagcctgaagattttgcagtttatttctgtcagcagtattatggctcaccttggacgttcggccaagggaccaaggtggaaatcaag

SEQ ID NO: 122 - protein, 108 aa, synthetic construct
EIMLMQSPGTLSLSPGERATLSCRASQSVSSSYLAWYQQKPGQAPRLLIYGASSRATDIPDRFSGSGSGTDFTLTISRLEPEDFAVYFCQQYYGSPWTFGQGTKVEIK

SEQ ID NO: 123 - DNA, 21 bp, synthetic construct
  source 1..21: mol_type = other DNA
cagagtgttagcagcagctac

SEQ ID NO: 124 - protein, 7 aa, synthetic construct
QSVSSSY

SEQ ID NO: 125-126 - skipped (2 entries)

SEQ ID NO: 127 - DNA, 27 bp, synthetic construct
  source 1..27: mol_type = other DNA
cagcagtattatggctcaccttggacg

SEQ ID NO: 128 - protein, 9 aa, synthetic construct
CQQYYGSPW

SEQ ID NO: 129 - DNA, 360 bp, synthetic construct
  source 1..360: mol_type = other DNA
gaggtgcagctggtggagtctgggggaggcttggtccagccgggggggtccctgaaactctcctgtacagcctctgggttgaccctcagtgactctgctatgcactgggtccgccaggcttccgggaaagggctggagtgggttggccgtataagaaataaggctaataggtacgcgacagaatatgctgcgtcggtgaaaggcaggttcaccatttcaagagatgattcaaagaacacggcgtatctacaaatgaacagcctgaaaaccgaggacacggccgtgtattattgtactagaaactggaagattttcctctttgactactggggccagggaaccctggtcaccgtctcctca

SEQ ID NO: 130 - protein, 120 aa, synthetic construct
EVQLVESGGGLVQPGGSLKLSCTASGLTLSDSAMHWVRQASGKGLEWVGRIRNKANRYATEYAASVKGRFTISRDDSKNTAYLQMNSLKTEDTAVYYCTRNWKIFLFDYWGQGTLVTVSS

SEQ ID NO: 131 - DNA, 24 bp, synthetic construct
  source 1..24: mol_type = other DNA
gggttgaccctcagtgactctgct

SEQ ID NO: 132 - protein, 8 aa, synthetic construct
GLTLSDSA

SEQ ID NO: 133 - DNA, 30 bp, synthetic construct
  source 1..30: mol_type = other DNA
ataagaaataaggctaataggtacgcgaca

SEQ ID NO: 134 - protein, 10 aa, synthetic construct
IRNKANRYAT

SEQ ID NO: 135 - DNA, 33 bp, synthetic construct
  source 1..33: mol_type = other DNA
actagaaactggaagattttcctctttgactac

SEQ ID NO: 136 - protein, 11 aa, synthetic construct
TRNWKIFLFDY

SEQ ID NO: 137 - DNA, 324 bp, synthetic construct
  source 1..324: mol_type = other DNA
gaaattgtgttgacgcagtctccaggcaccctgactttgtctccaggggaaagagccaccctctcctgcagggccagtcagagtgttggcagcaaatacttagcctggttccagcagaaacgtggccaggctcccaggctcctcatctatggtgcatccagcaggaccagtggcatccccgacaggatcagtggcagtgggtcagggacagacttcactctcaccatcagcagactggagcctgaagattttgcagtgtattactgtcagcagtatggaagttcaccctggacgttcggccaagggaccaaggtggaaatcaaa

SEQ ID NO: 138 - protein, 108 aa, synthetic construct
EIVLTQSPGTLTLSPGERATLSCRASQSVGSKYLAWFQQKRGQAPRLLIYGASSRTSGIPDRISGSGSGTDFTLTISRLEPEDFAVYYCQQYGSSPWTFGQGTKVEIK

SEQ ID NO: 139 - DNA, 21 bp, synthetic construct
  source 1..21: mol_type = other DNA
cagagtgttggcagcaaatac

SEQ ID NO: 140 - protein, 7 aa, synthetic construct
QSVGSKY

SEQ ID NO: 141-142 - skipped (2 entries)

SEQ ID NO: 143 - DNA, 27 bp, synthetic construct
  source 1..27: mol_type = other DNA
cagcagtatggaagttcaccctggacg

SEQ ID NO: 144 - protein, 9 aa, synthetic construct
QQYGSSPWT

SEQ ID NO: 145 - DNA, 375 bp, synthetic construct
  source 1..375: mol_type = other DNA
gaggtgcagctgttggagtctgggggaggcttggtacagccgggggggtccctgagactctcctgtgcagcctctggattcacctttaacaactatggcatgagctgggtccgccagggtccagggaaggggctggagtgggtctcatctattagtggtagtggtggtaccacattctacgcagactccgtgaagggccggttcaccatctccagagacaattccaagaacacgctgtatctgcaaatgaacagcctgagagccgaggacacggccgtatattactgtggcaaaggaggatattgtagtagtagcggctgccgtcactacggtatggacgtctggggccaagggaccacggtcaccgtctcctca

SEQ ID NO: 146 - protein, 125 aa, synthetic construct
EVQLLESGGGLVQPGGSLRLSCAASGFTFNNYGMSWVRQGPGKGLEWVSSISGSGGTTFYADSVKGRFTISRDNSKNTLYLQMNSLRAEDTAVYYCGKGGYCSSSGCRHYGMDVWGQGTTVTVSS

SEQ ID NO: 147 - DNA, 24 bp, synthetic construct
  source 1..24: mol_type = other DNA
ggattcacctttaacaactatggc

SEQ ID NO: 148 - protein, 8 aa, synthetic construct
GFTFNNYG

SEQ ID NO: 149 - DNA, 24 bp, synthetic construct
  source 1..24: mol_type = other DNA
attagtggtagtggtggtaccaca

SEQ ID NO: 150 - protein, 6 aa, synthetic construct
SGSGGT

SEQ ID NO: 151 - DNA, 54 bp, synthetic construct
  source 1..54: mol_type = other DNA
ggcaaaggaggatattgtagtagtagcggctgccgtcactacggtatggacgtc

SEQ ID NO: 152 - protein, 14 aa, synthetic construct
CGKGGYCSSSGCRH

SEQ ID NO: 153 - DNA, 330 bp, synthetic construct
  source 1..330: mol_type = other DNA
cagtctgtgctgactcagccaccctcagcgtctgggacccccgggcagagggtcaccatctcttgttctggaagcagctccaacatcggaaataattatatatactggtaccagcggctcccaggaacgacccccaaactcctcatctataggaataatcagcggccctcaggggtccctgaccgattctctggctccaagtctggcacctcagcctccctggccatcagtgggctccggtccgaggatgaggctgattattactgtgcagcatgggatgacaccctgagtgggtatgtcttcggaactgggaccaaggtcaccgtccta

SEQ ID NO: 154 - protein, 110 aa, synthetic construct
QSVLTQPPSASGTPGQRVTISCSGSSSNIGNNYIYWYQRLPGTTPKLLIYRNNQRPSGVPDRFSGSKSGTSASLAISGLRSEDEADYYCAAWDDTLSGYVFGTGTKVTVL

SEQ ID NO: 155 - DNA, 24 bp, synthetic construct
  source 1..24: mol_type = other DNA
agctccaacatcggaaataattat

SEQ ID NO: 156 - protein, 8 aa, synthetic construct
SSNIGNNY

SEQ ID NO: 157-158 - skipped (2 entries)

SEQ ID NO: 159 - DNA, 33 bp, synthetic construct
  source 1..33: mol_type = other DNA
gcagcatgggatgacaccctgagtgggtatgtc

SEQ ID NO: 160 - protein, 11 aa, synthetic construct
CAAWDDTLSGY

SEQ ID NO: 161 - DNA, 363 bp, synthetic construct
  source 1..363: mol_type = other DNA
gaggtgcagctggtggagtctgggggaaacttggtacagcctggggggtccctgagactctcctgtgcagcctctggattcacctttaccagccatgccatgaactgggtccgccaggctccagggaaggggctggagtgggtctcagttattactggtagaggttttgacacacactacgctgactccgtgaagggccggttcaccatctccagagacatttccaaaaacacgctgtatctgcaaatgaacagcctgagagccgaggacacggccgtttattactgtgcgaaaggtctctatgattcggggaattattatatcgattactggggccagggaaccctggtcaccgtctcctca

SEQ ID NO: 162 - protein, 121 aa, synthetic construct
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSS

SEQ ID NO: 163 - DNA, 24 bp, synthetic construct
  source 1..24: mol_type = other DNA
ggattcacctttaccagccatgcc

SEQ ID NO: 164 - protein, 8 aa, synthetic construct
GFTFTSHA

SEQ ID NO: 165 - DNA, 24 bp, synthetic construct
  source 1..24: mol_type = other DNA
attactggtagaggttttgacaca

SEQ ID NO: 166 - protein, 8 aa, synthetic construct
ITGRGFDT

SEQ ID NO: 167 - DNA, 42 bp, synthetic construct
  source 1..42: mol_type = other DNA
gcgaaaggtctctatgattcggggaattattatatcgattac

SEQ ID NO: 168 - protein, 14 aa, synthetic construct
AKGLYDSGNYYIDY

SEQ ID NO: 169 - DNA, 333 bp, synthetic construct
  source 1..333: mol_type = other DNA
cagtctgtgttgacgcagccgccctcagtgtctgcggccccaggacagaaggtcaccatctcctgctctggaagcagctccaacattgggaataattatgtttcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatctcagcctgagtttcaattgggtgttcggcggagggaccaagctgaccgtccta

SEQ ID NO: 170 - protein, 111 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLSLSFNWVFGGGTKLTVL

SEQ ID NO: 171 - DNA, 24 bp, synthetic construct
  source 1..24: mol_type = other DNA
agctccaacattgggaataattat

SEQ ID NO: 172 - protein, 8 aa, synthetic construct
SSNIGNNY

SEQ ID NO: 173-174 - skipped (2 entries)

SEQ ID NO: 175 - DNA, 36 bp, synthetic construct
  source 1..36: mol_type = other DNA
ggaacatgggatctcagcctgagtttcaattgggtg

SEQ ID NO: 176 - protein, 12 aa, synthetic construct
GTWDLSLSFNWV

SEQ ID NO: 177 - DNA, 378 bp, synthetic construct; identical to SEQ ID NO: 113
  source 1..378: mol_type = other DNA

SEQ ID NO: 178 - protein, 126 aa, synthetic construct; identical to SEQ ID NO: 114

SEQ ID NO: 179 - DNA, 24 bp, synthetic construct
  source 1..24: mol_type = other DNA
ggttacgccttcaccacctatggt

SEQ ID NO: 180 - protein, 8 aa, synthetic construct
GYAFTTYG

SEQ ID NO: 181 - DNA, 24 bp, synthetic construct
  source 1..24: mol_type = other DNA
atcagcgcttacaatggaaataca

SEQ ID NO: 182 - protein, 8 aa, synthetic construct
ISAYNGNT

SEQ ID NO: 183 - DNA, 57 bp, synthetic construct; identical to SEQ ID NO: 119
  source 1..57: mol_type = other DNA

SEQ ID NO: 184 - protein, 19 aa, synthetic construct
ARKGHYGSGTYYNPFGFDF

SEQ ID NO: 185 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactggcatcccagacaggttcagtggcagtgggtctgggacagacttcactctcaccatcagcagactggagcctgaagattttgctttgtatttctgtcagcagtattatggctcaccttggacgttcggccaagggaccaaggtggaaatcaaa

SEQ ID NO: 186 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFALYFCQQYYGSPWTFGQGTKVEIK

SEQ ID NO: 187 - DNA, 21 bp, synthetic construct
  source 1..21: mol_type = other DNA
cagagtgttagcagcagctac

SEQ ID NO: 188 - protein, 7 aa, synthetic construct
QSVSSSY

SEQ ID NO: 189-190 - skipped (2 entries)

SEQ ID NO: 191 - DNA, 27 bp, synthetic construct
  source 1..27: mol_type = other DNA
cagcagtattatggctcaccttggacg

SEQ ID NO: 192 - protein, 9 aa, synthetic construct
QQYYGSPWT

SEQ ID NO: 193 - DNA, 1338 bp, synthetic construct
  source 1..1338: mol_type = other DNA
caggtgcagctggtggagtctgggggaggcgtggtccagcctgggaggtccctgagactctcctgtacagcgtctggaatcaccttcagaaattatggcatgcactgggtccgccaggctccaggcaaggggctggagtgggtggcagttatgtggtatgatggaagtaataagtactatgcagactccgtgaagggccgtttcaccatctccggagacaattccaaggtgtatctgcaaatgaacagcctgagagccgaggacacggctgtatattactgtgcgagaaggggcactataagaacagctgccccttttgactactggggtcagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccaggcggtggcggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 194 - protein, 445 aa, synthetic construct
QVQLVESGGGVVQPGRSLRLSCTASGITFRNYGMHWVRQAPGKGLEWVAVMWYDGSNKYYADSVKGRFTISGDNSKVYLQMNSLRAEDTAVYYCARRGTIRTAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 196 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - DNA, 1341 bp, synthetic construct
  source 1..1341: mol_type = other DNA
caggtgcagctacagcagtggggcgcaggactgttgaagccttcggcgaccctgtcccgcacctgcgctgtctatggtgggtccttcagtggttactactggaactggatccgccagtccccagggaaggggctggaatggattggggaaatccttcatagtggaagaaccaactacaacccgtccctcaagagtcgagtcaccatatcagtagacacgtccaagaaccagttctccctgaagctgacctctgtgaccgccgcggacacggctgtatattactgtgcgggaaggatagcagctcgtcacggctggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 198 - protein, 446 aa, synthetic construct
QVQLQQWGAGLLKPSATLSRTCAVYGGSFSGYYWNWIRQSPGKGLEWIGEILHSGRTNYNPSLKSRVTISVDTSKNQFSLKLTSVTAADTAVYYCAGRIAARHGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 199 - DNA, 645 bp, synthetic construct
  source 1..645: mol_type = other DNA
gacatccagatgacccagtctccatcttccgtgtctacatctgtaggagacagagtcaccatctcttgtcgggcgagtcaggatattcgcaagtggttagcctggtatcaacagaaaccaggaaaagcccctaaactcctgatctatgctacatccagtttgcaaagtggggtcccttcaaggttcagcggcagtggatctgggacagatttcactctcaccatcagcagcctgcagcctgaggattttgcaacttacttttgtcaacaggctaacagtttcccgttcacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 200 - protein, 214 aa, synthetic construct
DIQMTQSPSSVSTSVGDRVTISCRASQDIRKWLAWYQQKPGKAPKLLIYATSSLQSGVPSRFSGSGSGTDFTLTISSLQPEDFATYFCQQANSFPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - DNA, 1341 bp, synthetic construct
  source 1..1341: mol_type = other DNA
caggtgcagctggtggagtctgggggaggcgtggtccagcctgggaggtccctgagactctcctgtgcagcgtctggattcaccttcagtacatatggcatgcactgggtccgccaggctccaggcaaggggctggagtgggtggcagttatttggcatgatggaagtgataaatattatgtagactccgtgaagggccgattctccatcgccagagacaattccaagaacacgctttatctgcaaatgaatagtctgagagtcgaggacacgggtatatattactgtgcgagaaggggtatacgtggaaccgtttttgaccactggggcctgggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 202 - protein, 446 aa, synthetic construct
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 203 - DNA, 645 bp, synthetic construct
  source 1..645: mol_type = other DNA
gacatccagatgacccagtctccttccaccctgtctgcatctgtaggagacagagtcaccctcacttgtcgggccagtcagagtattagtaacaagttggcctggtatcagcagaaaccagggaaagcccctaacctcctgatctataaggcgtctaatttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 204 - protein, 214 aa, synthetic construct
DIQMTQSPSTLSASVGDRVTLTCRASQSISNKLAWYQQKPGKAPNLLIYKASNLESGVPSRFSGSGSGTEFTLTISSLQPDDFATYYCQQYNSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - DNA, 1332 bp, synthetic construct
  source 1..1332: mol_type = other DNA
caggtgcagctgcaggagtcgggcccaggactggtgaagccttcggagaccctgtccctcacctgcactgtctctggtgactccatcaataattactactggacctggctccggcagcccccagggaagggactggagtggattggttatatctattacagtgggagcgccaactacaacccctccctcaagagtcgagtcaccatatcagtagacacgtccaagaaccagttctccctgaagctaaattctgtgaccgctgcggacacggccgtgtattactgtgcgagaggggcggtcaagtacttccggcattggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 206 - protein, 443 aa, synthetic construct
QVQLQESGPGLVKPSETLSLTCTVSGDSINNYYWTWLRQPPGKGLEWIGYIYYSGSANYNPSLKSRVTISVDTSKNQFSLKLNSVTAADTAVYYCARGAVKYFRHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 207 - DNA, 648 bp, synthetic construct
  source 1..648: mol_type = other DNA
gaaattgtgttgacgcagtctccgggcaccctctctttgtctccaggggaaagagccaccctctcctgcagggccagtcagactattaaccacaacaacttagcctggtaccagcagagacctggccaggctcccaggctcctcatctatggtgcatccaacagggccactgccatcccagacaggttcagtggcagtgggtctgggacagacttcactctcaccatcagcagactggagcctgaagattttgaagtgtattcttgtcagcagtatggtagcttgccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 208 - protein, 215 aa, synthetic construct
EIVLTQSPGTLSLSPGERATLSCRASQTINHNNLAWYQQRPGQAPRLLIYGASNRATAIPDRFSGSGSGTDFTLTISRLEPEDFEVYSCQQYGSLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - DNA, 1347 bp, synthetic construct
  source 1..1347: mol_type = other DNA
caggtgcagctggtggagtctgggggaggcgtggtccagcctgggacgtccctgagactctcctgtgcagcgtcaggattcaccttcagtagctatggcatgcactgggtccgccaggctccaggcaaggggctggagtgggtggcagttatatggattgatggaagtaataaatattatgcagactccgtgaagggccgattcaccatctccagagacaattccaagaacacgctgtatctgcaaatgaacagcctgagagccgaggacacggctgtgtattactgtgcgagaagggggggtatagtagtagctgccccctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 210 - protein, 448 aa, synthetic construct
QVQLVESGGGVVQPGTSLRLSCAASGFTFSSYGMHWVRQAPGKGLEWVAVIWIDGSNKYYADSVKGRFTISRDNSKNTLYLQMNSLRAEDTAVYYCARRGGIVVAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 211 - DNA, 648 bp, synthetic construct; identical to SEQ ID NO: 195
  source 1..648: mol_type = other DNA

SEQ ID NO: 212 - protein, 215 aa, synthetic construct; identical to SEQ ID NO: 196

SEQ ID NO: 213 - DNA, 1356 bp, synthetic construct
  source 1..1356: mol_type = other DNA
caggtgcagttggtggagtctgggggaggcgtggtccagcctgggaggtccctgagactctcctgtgaagcgtctggaatcaccttcagaaactatggcatgcactgggtccgccaggctccaggcaaggggctggagtgggtggcagttatgtggtatgatggaagtaataaatactacgcagactccgtgaagggccgattcaccatctccagagacaattccaagaacacggtgtatctgcaaatgaacagcctgagagccgaagacacggctgtgtattactgtgcgagacggggtcatatagcaacagctgctccctttgactactggggccagggaaccctggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 214 - protein, 451 aa, synthetic construct
QVQLVESGGGVVQPGRSLRLSCEASGITFRNYGMHWVRQAPGKGLEWVAVMWYDGSNKYYADSVKGRFTISRDNSKNTVYLQMNSLRAEDTAVYYCARRGHIATAAPFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 215 - DNA, 648 bp, synthetic construct
  source 1..648: mol_type = other DNA
gacatccagatgacccagtctccatcctccctgtctgcatccgtaggagacagagtcaccatcagttgccgggcaagtcagagcattagtagttatttaaattggtatcagcagaaaccagggaaagcccctaaggtcctgatgtatgctgcatccagtttgcaaagtggggtcccatcaaggttcagtggcagtggatctgggacagatttcactctcaccatcagcagtctgcaacctgaggattttgcaacttactactgtcaacagagttacagtacccctccgatcaccttcggccaagggacacgactggagattaaacgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 216 - protein, 215 aa, synthetic construct
DIQMTQSPSSLSASVGDRVTISCRASQSISSYLNWYQQKPGKAPKVLMYAASSLQSGVPSRFSGSGSGTDFTLTISSLQPEDFATYYCQQSYSTPPITFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 217 - DNA, 1347 bp, synthetic construct
  source 1..1347: mol_type = other DNA
caggtgcagctggtggagtctgggggaggcgtggtccagcctgggaggtccctgagactctcctgtacagcgtctggattcaccttccgttcctatggcatgcactgggtccgccaggctccaggcaaggggctggagtgggtgtcagttatttggattgatggaaataatatatactatgcagactccgtgaagggccgattcaccatctccagagacaattccaagaacacgctgtatctgcaaatggacagcctgagagccgaggacacggctgtttattactgtgcgagaagactggctataacatcagctgccccctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 218 - protein, 448 aa, synthetic construct
QVQLVESGGGVVQPGRSLRLSCTASGFTFRSYGMHWVRQAPGKGLEWVSVIWIDGNNIYYADSVKGRFTISRDNSKNTLYLQMDSLRAEDTAVYYCARRLAITSAAPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 219 - DNA, 648 bp, synthetic construct; identical to SEQ ID NO: 195
  source 1..648: mol_type = other DNA

SEQ ID NO: 220 - protein, 215 aa, synthetic construct; identical to SEQ ID NO: 196

SEQ ID NO: 221 - DNA, 1353 bp, synthetic construct
  source 1..1353: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagtccaactgggaggcaggaaatactttcacctgctctgtgttacatgagggcctgcacaaccaccatactgagaagtccctctcccactctcctggtaaatga

SEQ ID NO: 222 - protein, 450 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 223 - DNA, 648 bp, synthetic construct
  source 1..648: mol_type = other DNA
gaaattatgttgatgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactgacatcccagacaggttcagtggcagtgggtctgggacagacttcactctcaccatcagcagactggagcctgaagattttgcagtttatttctgtcagcagtattatggctcaccttggacgttcggccaagggaccaaggtggaaatcaagcgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 224 - protein, 215 aa, synthetic construct
EIMLMQSPGTLSLSPGERATLSCRASQSVSSSYLAWYQQKPGQAPRLLIYGASSRATDIPDRFSGSGSGTDFTLTISRLEPEDFAVYFCQQYYGSPWTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 225 - DNA, 1344 bp, synthetic construct
  source 1..1344: mol_type = other DNA
gaggtgcagctggtggagtctgggggaggcttggtccagccgggggggtccctgaaactctcctgtacagcctctgggttgaccctcagtgactctgctatgcactgggtccgccaggcttccgggaaagggctggagtgggttggccgtataagaaataaggctaataggtacgcgacagaatatgctgcgtcggtgaaaggcaggttcaccatttcaagagatgattcaaagaacacggcgtatctacaaatgaacagcctgaaaaccgaggacacggccgtgtattattgtactagaaactggaagattttcctctttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 226 - protein, 447 aa, synthetic construct
EVQLVESGGGLVQPGGSLKLSCTASGLTLSDSAMHWVRQASGKGLEWVGRIRNKANRYATEYAASVKGRFTISRDDSKNTAYLQMNSLKTEDTAVYYCTRNWKIFLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 227 - DNA, 648 bp, synthetic construct
  source 1..648: mol_type = other DNA
gaaattgtgttgacgcagtctccaggcaccctgactttgtctccaggggaaagagccaccctctcctgcagggccagtcagagtgttggcagcaaatacttagcctggttccagcagaaacgtggccaggctcccaggctcctcatctatggtgcatccagcaggaccagtggcatccccgacaggatcagtggcagtgggtcagggacagacttcactctcaccatcagcagactggagcctgaagattttgcagtgtattactgtcagcagtatggaagttcaccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 228 - protein, 215 aa, synthetic construct
EIVLTQSPGTLTLSPGERATLSCRASQSVGSKYLAWFQQKRGQAPRLLIYGASSRTSGIPDRISGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - DNA, 1368 bp, synthetic construct
  source 1..1368: mol_type = other DNA
gaggtgcagctgttggagtctgggggaggcttggtacagccgggggggtccctgagactctcctgtgcagcctctggattcacctttaacaactatggcatgagctgggtccgccagggtccagggaaggggctggagtgggtctcatctattagtggtagtggtggtaccacattctacgcagactccgtgaagggccggttcaccatctccagagacaattccaagaacacgctgtatctgcaaatgaacagcctgagagccgaggacacggccgtatattactgtggcaaaggaggatattgtagtagtagcggctgccgtcactacggtatggacgtctggggccaagggaccacggtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 230 - protein, 455 aa, synthetic construct
EVQLLESGGGLVQPGGSLRLSCAASGFTFNNYGMSWVRQGPGKGLEWVSSISGSGGTTFYADSVKGRFTISRDNSKNTLYLQMNSLRAEDTAVYYCGKGGYCSSSGCRHYGMDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 231 - DNA, 654 bp, synthetic construct
  source 1..654: mol_type = other DNA
cagtctgtgctgactcagccaccctcagcgtctgggacccccgggcagagggtcaccatctcttgttctggaagcagctccaacatcggaaataattatatatactggtaccagcggctcccaggaacgacccccaaactcctcatctataggaataatcagcggccctcaggggtccctgaccgattctctggctccaagtctggcacctcagcctccctggccatcagtgggctccggtccgaggatgaggctgattattactgtgcagcatgggatgacaccctgagtgggtatgtcttcggaactgggaccaaggtcaccgtcctacgagctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgttga

SEQ ID NO: 232 - protein, 217 aa, synthetic construct
QSVLTQPPSASGTPGQRVTISCSGSSSNIGNNYIYWYQRLPGTTPKLLIYRNNQRPSGVPDRFSGSKSGTSASLAISGLRSEDEADYYCAAWDDTLSGYVFGTGTKVTVLRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 233 - DNA, 1347 bp, synthetic construct
  source 1..1347: mol_type = other DNA
gaggtgcagctggtggagtctgggggaaacttggtacagcctggggggtccctgagactctcctgtgcagcctctggattcacctttaccagccatgccatgaactgggtccgccaggctccagggaaggggctggagtgggtctcagttattactggtagaggttttgacacacactacgctgactccgtgaagggccggttcaccatctccagagacatttccaaaaacacgctgtatctgcaaatgaacagcctgagagccgaggacacggccgtttattactgtgcgaaaggtctctatgattcggggaattattatatcgattactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 234 - protein, 448 aa, synthetic construct
EVQLVESGGNLVQPGGSLRLSCAASGFTFTSHAMNWVRQAPGKGLEWVSVITGRGFDTHYADSVKGRFTISRDISKNTLYLQMNSLRAEDTAVYYCAKGLYDSGNYYIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 235 - DNA, 654 bp, synthetic construct
  source 1..654: mol_type = other DNA
cagtctgtgttgacgcagccgccctcagtgtctgcggccccaggacagaaggtcaccatctcctgctctggaagcagctccaacattgggaataattatgtttcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatctcagcctgagtttcaattgggtgttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 236 - protein, 217 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDLSLSFNWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 237 - DNA, 1362 bp, synthetic construct
  source 1..1362: mol_type = other DNA
caggttcagctggtgcagtctggagctgaggtgaagaagcctggggcctcagtgaaggtctcctgcaaggcttctggttacgccttcaccacctatggtatcacctgggtgcgacaggcccctggacaaggacttgagtggatgggatggatcagcgcttacaatggaaatacaaactatgcagagaaggtccagggcagattcaccatgaccacagacacatccacgaatacagcctacatggagctgaggagcctgagatccgacgacacggccgtgtatttctgtgcgagaaagggtcactatggttcggggacttattataacccctttggttttgatttttggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 238 - protein, 453 aa, synthetic construct
QVQLVQSGAEVKKPGASVKVSCKASGYAFTTYGITWVRQAPGQGLEWMGWISAYNGNTNYAEKVQGRFTMTTDTSTNTAYMELRSLRSDDTAVYFCARKGHYGSGTYYNPFGFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 239 - DNA, 648 bp, synthetic construct
  source 1..648: mol_type = other DNA
gaaattgtgttgacgcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactggcatcccagacaggttcagtggcagtgggtctgggacagacttcactctcaccatcagcagactggagcctgaagattttgctttgtatttctgtcagcagtattatggctcaccttggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 240 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFALYFCQQYYGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - DNA, 1302 bp, Homo sapiens
  source 1..1302: mol_type = genomic DNA
actcccagctcgacaaccactgccaccccccaagctcggcttgtcacctgccctaggagacgcagccgccggaccctgcccagggcacccacgcctcggcgaccaccatgtcccagaccaaaatgctgaaggtccgcgtgaccctcttctgcatcctggcaggcatcgtgctggccatgacagccgtggtaaccgaccactgggctgtgctgagcccccacatggagcaccacaacactacctgcgaggcggcccacttcggcctctggcggatttgtaccaagcgcatccccatggacgacagcaagacctgcgggcccatcaccctgcccggggagaagaactgttcctacttcaggcattttaaccccggcgagagctcggagatcttcgaattcaccactcagaaggagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcactaaccctcctgcggccctagcgaccctcaggcttcttccccaggaagcggggtcttggcctggaaccttccagagaggaggcgggagcaattttagccccaccctgctcccatctgcccccctgcaacagtcgcaggctgcttcctctctctgagttcctctgggctgccgcaggctcccctgggaatagagcaagacgtgagtcctaacctggccacagttgggggaggcagagccagcaggtggacaggtgtttgcaggggcccaacttcccctggagctcagaggtgtccccactgtaccagcctctgataagctgcctccagttgtcctttatgaacattgcagggacaacctgtgtttgccagctgggtgttccgtgtaaatagccagcctgtctctttctcggtgataaaacacaccctctctggtgagcccagcgtcccctccttggcttccaggagccctgggaagcatttttaactgggtagaatctgactgtggcttgaaataaaaagctctcagaaaa

SEQ ID NO: 242 - protein, 222 aa, Homo sapiens
MSQTKMLKVRVTLFCILAGIVLAMTAVVTDHWAVLSPHMEHHNTTCEAAHFGLWRICTKRIPMDDSKTCGPITLPGEKNCSYFRHFNPGESSEIFEFTTQKEYSISAAAIAIFSLGFIILGSLCVLLSLGKKRDYLLRPASMFYAFAGLCILVSVEVMRQSVKRMIDSEDTVWIEYYYSWSFACACAAFILLFLGGLALLLFSLPRMPRNPWESCMDAEPEH

SEQ ID NO: 243 - protein, 330 aa, synthetic construct [sequence omitted]

SEQ ID NO: 244 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 245 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 246 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNRFTQKSLSLSPGK

SEQ ID NO: 247 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 248 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 249 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 251 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 252 - protein, 326 aa, synthetic construct; identical to SEQ ID NO: 251

SEQ ID NO: 253 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 254 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 255 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 256 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 258 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 259 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNRFTQKSLSLSPGK

SEQ ID NO: 260 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 261 - protein, 231 aa, synthetic construct
EPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 231 aa, synthetic construct
EPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 263 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 264 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 265 - protein, 228 aa, synthetic construct
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 266 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 267 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 268 - protein, 5 aa, synthetic construct
GGGGS

SEQ ID NO: 269 - protein, 446 aa, synthetic construct
QVQLVESGGGVVQPGRSLRLSCAASGFTFSTYGMHWVRQAPGKGLEWVAVIWHDGSDKYYVDSVKGRFSIARDNSKNTLYLQMNSLRVEDTGIYYCARRGIRGTVFDHWGLGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 270 - DNA, 4700 bp, synthetic construct
  source 1..4700: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgaggttaacccgcggccgttttttttatcgctgccttgatatacactttccaccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacattcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaatctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatgtaggaagtcttttccacatggcagatgaaccggtggctagtaataaaagatccttattttcattggatctgtgtgttggttttttgtgtggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 271 - DNA, 2869 bp, synthetic construct
  source 1..2869: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgggcaaagccacgcgtctagcatgccccactacgggtctaggctgcccatgtaaggaggcaaggcctggggacacccgagatgcctggttataattaacccagacatgtggctgcccccgcccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacggatccgccaccatgcggctcacccgctgccaggctgccctggcggccgccatcaccctcaaccttctggtcctcttctatgtctcgtggctgcagcaccagcctaggaattcccgggcccgggggccccgtcgtgcctctgctgccggcccccgtgtcaccgtcctggtgcgggagttcgaggcatttgacaacgcggtgcccgagctggtagactccttcctgcagcaagacccagcccagcccgtggtggtggcagccgacacgctcccctacccgcccctggccctgccccgcatccccaacgtgcgtctggcgctgctccagcccgccctggaccggccagccgcagcctcgcgcccggagacctacgtggccaccgagtttgtggccctagtacctgatggggcgcgggctgaggcacctggcctgctggagcgcatggtggaggcgctccgcgcaggaagcgcacgtctggtggccgccccggttgccacggccaaccctgccaggtgcctggccctgaacgtcagcctgcgagagtggaccgcccgctatggcgcagcccccgccgcgccccgctgcgacgccctggacggagatgctgtggtgctcctgcgcgcccgcgacctcttcaacctctcggcgcccctggcccggccggtgggcaccagcctctttctgcagaccgcccttcgcggctgggcggtgcagctgctggacttgaccttcgccgcggcgcgccagcccccgctggccacggcccacgcgcgctggaaggctgagcgcgagggacgcgctcggcgggcggcgctgctccgcgcgctgggcatccgcctagtgagctgggaaggcgggcggctggagtggttcggctgcaacaaggagaccacgcgctgcttcggaaccgtggtgggcgacacgcccgcctacctctacgaggagcgctggacgcccccctgctgcctgcgcgcgctgcgcgagaccgcccgctatgtggtgggcgtgctggaggctgcgggcgtgcgctactggctcgagggcggctcactgctgggggccgcccgccacggggacatcatcccatgggactacgacgtggacctgggcatctacttggaggacgtgggcaactgcgagcagctgcggggggcagaggccggctcggtggtggatgagcgcggcttcgtatgggagaaggcggtcgagggcgactttttccgcgtgcagtacagcgaaagcaaccacttgcacgtggacctgtggcccttctacccccgcaatggcgtcatgaccaaggacacgtggctggaccaccggcaggatgtggagtttcccgagcacttcctgcagccgctggtgcccctgccctttgccggcttcgtggcgcaggcgcctaacaactaccgccgcttcctggagctcaagttcgggcccggggtcatcgagaacccccagtaccccaacccggcactgctgagtctgacgggaagcggctgataatagctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 272 - DNA, 3698 bp, synthetic construct
  source 1..3698: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctcgggcaaagccacgcgttaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgggatccgccaccatggcttctgcatcaacttctaaatataattcacactccttggagaatgagtctattaagaggacgtctcgagatggagtcaatcgagacctcactgaggctgttcctcgacttccaggagaaacactaatcactgacaaagaagttatttacatatgtcctttcaatggccccattaagggaagagtttacatcacaaattatcgtctttatttaagaagtttggaaacggattcttctctaatacttgatgttcctctgggtgtgatctcgagaattgaaaaaatgggaggcgcgacaagtagaggagaaaattcctatggtctagatattacttgtaaagacatgagaaacctgaggttcgctttgaaacaggaaggccacagcagaagagatatgtttgagatcctcacgagatacgcgtttcccctggctcacagtctgccattatttgcatttttaaatgaagaaaagtttaacgtggatggatggacagtttacaatccagtggaagaatacaggaggcagggcttgcccaatcaccattggagaataacttttattaataagtgctatgagctctgtgacacttaccctgctcttttggtggttccgtatcgtgcctcagatgatgacctccggagagttgcaacttttaggtcccgaaatcgaattccagtgctgtcatggattcatccagaaaataagacggtcattgtgcgttgcagtcagcctcttgtcggtatgagtgggaaacgaaataaagatgatgagaaatatctcgatgttatcagggagactaataaacaaatttctaaactcaccatttatgatgcaagacccagcgtaaatgcagtggccaacaaggcaacaggaggaggatatgaaagtgatgatgcatatcataacgccgaacttttcttcttagacattcataatattcatgttatgcgggaatctttaaaaaaagtgaaggacattgtttatcctaatgtagaagaatctcattggttgtccagtttggagtctactcattggttagaacatatcaagctcgttttgacaggagccattcaagtagcagacaaagtttcttcagggaagagttcagtgcttgtgcattgcagtgacggatgggacaggactgctcagctgacatccttggccatgctgatgttggatagcttctataggagcattgaagggttcgaaatactggtacaaaaagaatggataagttttggacataaatttgcatctcgaataggtcatggtgataaaaaccacaccgatgctgaccgttctcctatttttctccagtttattgattgtgtgtggcaaatgtcaaaacagttccctacagcttttgaattcaatgaacaatttttgattataattttggatcatctgtatagttgccgatttggtactttcttattcaactgtgaatctgctcgagaaagacagaaggttacagaaaggactgtttctttatggtcactgataaacagtaataaagaaaaattcaaaaaccccttctatactaaagaaatcaatcgagttttatatccagttgccagtatgcgtcacttggaactctgggtgaattactacattagatggaaccccaggatcaagcaacaacagccgaatccagtggagcagcgttacatggagctcttagccttacgcgacgaatacataaagcggcttgaggaactgcagctcgccaactctgccaagctttctgatcccccaacttcaccttccagtccttcgcaaatgatgccccatgtgcaaactcacttctgataatagctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 273 - DNA, 19 bp, synthetic construct
  source 1..19: mol_type = other DNA
gtcatgcatgcctggaatc

SEQ ID NO: 274 - DNA, 50 bp, synthetic construct
  source 1..50: mol_type = other DNA
tcgtcggcagcgtcagatgtgtataagagacagcgagcgctgctcgagag

SEQ ID NO: 275 - DNA, 52 bp, synthetic construct
  source 1..52: mol_type = other DNA
gtctcgtgggctcggagatgtgtataagagacaggggtcacagggatgccac